(19) United States
(12) Patent Application Publication
(10) Pub. No.: US 2024/0343713 A1
(43) Pub. Date: Oct. 17, 2024

(54) TELOMERASE INHIBITOR COMPOUNDS

(71) Applicant: Geron Corporation, Foster City, CA (US)

(72) Inventors: Manoj Kumar Khera, New Delhi (IN); Ajay Soni, New Delhi (IN)

(21) Appl. No.: 18/436,936

(22) Filed: Feb. 8, 2024

Related U.S. Application Data
(60) Provisional application No. 63/444,467, filed on Feb. 9, 2023.

(51) Int. Cl. C07D 405/10; A61K 31/4155; A61P 35/00; C07D 403/10

(52) U.S. Cl. CPC C07D 405/10; A61K 31/4155; A61P 35/00; C07D 403/10

(57) ABSTRACT

Provided are telomerase inhibitor compounds. Some compounds include a lactone or lactam group that is covalently bonded to a phenyl ring, which is itself bonded to a pyrazole group. In other cases, a sulfonamide-containing moiety is covalently bonded to a phenyl ring, which is itself bonded to a pyrazole group. In some embodiments, the telomerase inhibitor compound has a vinyl sulfonamide group bonded to an amide moiety and an aromatic group. In other cases, the inhibitor compound has an isothiazolidine 1,1-dioxide core that is bonded to a phenyl group. Aspects of the invention also include pharmaceutical compositions comprising the subject telomerase inhibitor compounds, as well as methods of treating telomerase-related diseases or conditions.

### CROSS-REFERENCE TO RELATED APPLICATIONS

This application claims the benefit of U.S. Provisional Application No. 63/444,467, filed Feb. 9, 2023, the disclosure of which is incorporated herein by reference.

### INTRODUCTION

Telomerase is a ribonucleoprotein that is known to add telomere repeat sequences to the 3′ end of telomeres on chromosomes. One function of telomeres is to protect the ends of chromosomal DNA from degradation by various biochemical processes. It has been observed that telomerase is normally absent or present in low concentrations in normal, somatic cells. In contrast, telomerase is more abundant in gametes, embryotic stem cells, male sperm cells, epidermal cells, certain immune cells, and most cancer cells. Without telomerase, repeated cell divisions can erode the length of telomeres until the Hayflick limit is reached, at which cells become senescent and cell division stops (Hayflick et al, doi: 10.1016/0014-4827 (61)90192-6). However, cancer cells can repeatedly divide and reproduce because their use of telomerase continually replenishes the lost telomeres (Hanahan et al, doi:10.1016/j.cell.2011.02.013). Telomerase has therefore been identified as an excellent target for cancer therapeutic agents. It has been proposed that inhibiting telomerase can be used to inhibit cancer (Williams, doi:10.1038/nm0113-6).

Genes encoding both the protein and RNA components of human telomerase have been cloned and sequenced. The telomerase holoenzyme is composed of the telomerase protein component (the human form of which is known as human telomerase reverse transcriptase or hTERT) and the RNA component (the human form of which is known as human telomerase RNA or hTR). Much effort has been spent in the search for telomerase inhibitors. Telomerase inhibitors identified to date include small molecule compounds and oligonucleotides. Given the close connection between telomerase and cell proliferative disorders, such as cancer, what is needed are compounds useful for inhibiting telomerase in proliferative cells and uses of the same to treat proliferative diseases such as cancer.

Hematologic malignancies are forms of cancer that begin in the cells of blood-forming tissue, such as the bone marrow, or in the cells of the immune system. Examples of hematologic malignancies are acute and chronic leukemias, lymphomas, multiple myeloma, and myeloproliferative neoplasms.

Myeloproliferative neoplasms, or MPNs, are hematologic neoplasms that arise from neoplastic hematopoietic myeloid progenitor cells in the bone marrow, such as the precursor cells of red cells, platelets, and granulocytes. Proliferation of neoplastic progenitor cells leads to an overproduction of any combination of white cells, red cells and/or platelets, depending on the disease. These overproduced cells may also be abnormal, leading to additional clinical complications. There are various types of chronic myeloproliferative disorders. Included in the MPN disease spectrum are Essential Thrombocythemia (ET), Polycythemia vera (PV), Chronic Myelogenous Leukemia (CML), myelofibrosis (MF), chronic neutrophilic leukemia, chronic eosinophilic leukemia and acute myelogenous leukemia (AML).

Myelodysplastic syndromes (MDS) represent a rare heterogeneous group of hemopoietic clonal disorders that are characterized by ineffective hemopoiesis resulting in anemia and other cytopenias and are serious and life threatening with a high risk of leukemic transformation. Myelodysplastic syndromes (MDS) include diseases such as, refractory anemia, refractory anemia with excess blasts, refractory cytopenia with multilineage dysplasia, refractory cytopenia with unilineage dysplasia, and chronic myelomonocytic leukemia (CMML).

### SUMMARY

Provided are telomerase inhibitor compounds. Some compounds include a lactone or lactam group that is covalently bonded to a phenyl ring, which is itself bonded to a pyrazole group. In other cases, a sulfonamide-containing moiety is covalently bonded to a phenyl ring, which is itself bonded to a pyrazole group. In some embodiments, the telomerase inhibitor compound has a vinyl sulfonamide group bonded to an amide moiety and an aromatic group. In other cases, the inhibitor compound has an isothiazolidine 1,1-dioxide core that is bonded to a phenyl group. Aspects of the invention also include pharmaceutical compositions comprising the subject telomerase inhibitor compounds, as well as methods of treating telomerase-related diseases or conditions.

### BRIEF DESCRIPTION OF THE DRAWINGS

FIG. 1 shows in vitro data for telomerase inhibitor compounds.

FIG. 2 shows additional in vitro data for additional telomerase inhibitor compounds.

FIG. 3 shows a third set of in vitro data for telomerase inhibitor compounds.

FIG. 4 shows a fourth set of in vitro data for telomerase inhibitor compounds.

FIG. 5 shows a fifth set of in vitro data for telomerase inhibitor compounds.

FIG. 6 shows the imaging and observation plan.

FIG. 7 shows effects of telomerase inhibition over three weeks on the bioluminescence signal (BLI) of LUC-MOLM13 tumor-bearing mice

FIG. 8 shows effects of telomerase inhibition over three weeks on the bioluminescence signal (BLI) of overall group.

FIG. 9 shows effects of telomerase inhibition over three weeks on the bioluminescence signal (BLI) of oral dose group 5 (Compound 3).

FIG. 10 shows effects of telomerase inhibition over three weeks on the bioluminescence signal (BLI) of Group 6 (Compound 2).

FIG. 11A shows effect of telomerase inhibitors administered on the bioluminescence signal of LUC-MOLM13 tumor-bearing mice on Day 0 (a) Average BLI across groups.

FIG. 11B shows effect of telomerase inhibitors administered on the bioluminescence signal of LUC-MOLM13 tumor-bearing mice on Day 0 (b) Distribution of orthotropically growing LUC-MOLM13 tumors across groups. In FIG. 11B, fluorescence in the top chest area is visible.

FIG. 12A shows effect of telomerase inhibitors administered on the bioluminescence signal of LUC-MOLM13 tumor-bearing mice on Day 5 (a) Average BLI across groups.

FIG. 12B shows effect of telomerase inhibitors administered on the bioluminescence signal of LUC-MOLM13 tumor-bearing mice on Day 5 (b) Distribution of orthotopically growing LUC-MOLM13 tumors across groups. In FIG. 12B, little or zero fluorescence is shown.

FIG. 13A show effect of telomerase inhibitors administered on the bioluminescence signal of LUC-MOLM13 tumor-bearing mice on Day 10 (a) Average BLI across groups.

FIG. 13B show effect of telomerase inhibitors administered on the bioluminescence signal of LUC-MOLM13 tumor-bearing mice on Day 10 (b) Distribution of orthotopically growing LUC-MOLM13 tumors across groups.

FIG. 14A shows effect of telomerase inhibitors administered on the bioluminescence signal of LUC-MOLM13 tumor-bearing mice on Day 15 (a) Average BLI across groups.

FIG. 14B show effect of telomerase inhibitors administered on the bioluminescence signal of LUC-MOLM13 tumor-bearing mice on Day 15 (b) Distribution of orthotopically growing LUC-MOLM13 tumors across groups.

FIG. 15A show effect of telomerase inhibitors administered on the bioluminescence signal of LUC-MOLM13 tumor-bearing mice on Day 18 (a) Average BLI across groups.

FIG. 15B show Effect of telomerase inhibitors administered on the bioluminescence signal of LUC-MOLM13 tumor-bearing mice on Day 18 (b) Distribution of orthotopically growing LUC-MOLM13 tumors across groups.

FIG. 16A show effect of telomerase inhibitors administered on the bioluminescence signal of LUC-MOLM13 tumor-bearing mice on Day 21 (a) Average BLI across groups.

FIG. 16B show Effect of telomerase inhibitors administered on the bioluminescence signal of LUC-MOLM13 tumor-bearing mice on Day 21 (b) Distribution of orthotopically growing LUC-MOLM13 tumors across groups.

FIG. 17A shows effect of telomerase inhibitors administered on the bioluminescence signal of LUC-MOLM13 tumor-bearing mice on Day 28 (a) Average BLI across groups.

FIG. 17B show effect of telomerase inhibitors administered on the bioluminescence signal of LUC-MOLM13 tumor-bearing mice on Day 28 (b) Distribution of orthotopically growing LUC-MOLM13 tumors across groups.

FIG. 18 shows Effect of telomerase inhibitors administered on the life span of LUC-MOLM13 tumor-bearing mice.

FIG. 19 shows Effect of telomerase inhibitors administered on average body weights of LUC-MOLM13 tumor-bearing mice.

FIG. 20A shows bioluminescent signal from tissues obtained from the effect of telomerase inhibitors on Day 34 of necropsy: group-control.

FIG. 20B shows bioluminescence data for Group 7 (BIRB-1532)

FIG. 20C shows bioluminescence data for Group 5 (Compound 3).

FIG. 20D shows bioluminescence data for Group 4 (Compound 1).

FIG. 20E shows bioluminescence data for Group 6 (Compound 2).

FIG. 20F shows a sample with tissue labeling: 1. Brain 2. Axillary lymph node 3. Eyes 4. Branchial lymph node 5. Lungs 6. Heart 7. Stomach 8. Liver 9. Kidney 10. Spleen 11. Cervical lymph node 12. Mesenteric lymph node 13. Inguinal lymph node 14. GI tract 15. Male reproductive organ.

### DETAILED DESCRIPTION

Provided are telomerase inhibitor compounds. Some compounds include a lactone or lactam group that is covalently bonded to a phenyl ring, which is itself bonded to a pyrazole group. In other cases, a sulfonamide-containing moiety is covalently bonded to a phenyl ring, which is itself bonded to a pyrazole group. In some embodiments, the telomerase inhibitor compound has a vinyl sulfonamide group bonded to an amide moiety and an aromatic group. In other cases, the inhibitor compound has an isothiazolidine 1,1-dioxide core that is bonded to a phenyl group. Aspects of the invention also include pharmaceutical compositions comprising the subject telomerase inhibitor compounds, as well as methods of treating telomerase-related diseases or conditions.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“Alkyl” refers to a monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases, the alkyl group has 1 to 24 carbon atoms, e.g. 1 to 12, 1 to 6, or 1 to 3.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl.

“Alkynyl” refers to a monoradical, branched or linear, non-cyclic hydrocarbonyl group that comprises a carbon-carbon triple bond. Exemplary alkynyl groups include ethynyl and n-propynyl.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g., O, S, N) as a ring atom and that is not aromatic (i.e., distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e., none of the ring atoms are heteroatoms (e.g., O, S, N). In some cases, the aryl group has a second aromatic ring, e.g., that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the aromatic ring is a heteroatom (e.g., O, S, N). Exemplary heteroaryl groups include those obtained from removing a hydrogen atom from pyridine, pyrimidine, furan, thiophene, or benzothiophene.

The term “substituted” refers the removal of one or more hydrogens from an atom (e.g., from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g., giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no longer be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

The term “substituted versions thereof” refers to both substituted and unsubstituted categories being named. For instance, the recitation of “alkyl, aryl, heteroaryl, halo, nitro and substituted versions thereof” refers to the groups alkyl, substituted alkyl, aryl, substituted aryl, heteroaryl, substituted heteroaryl, halo, and nitro.

Exemplary substituents include deuterium (D), alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, thiol, thioether, and substituted versions thereof.

In some cases, the substitutions can themselves be further substituted with one or more groups. For example, the group —C₆H₄CH₂CH₃ can be considered as substituted aryl, i.e., an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein. In some cases, the substituents are not substituted with any other groups.

Diradical groups are also described herein, i.e., in contrast to the monoradical groups such as alkyl and aryl described above. The term “alkylene” refers to the diradical version of an alkyl group, i.e., an alkylene group is a diradical, branched or linear, cyclic, or non-cyclic, saturated hydrocarbon group. Exemplary alkylene groups include diylmethane (—CH₂—, which is also known as a methylene group), 1,2-diylethane (—CH₂CH₂—), and 1,1-diylethane (i.e., a CHCH₃ fragment where the first atom has two single bonds to other two different groups). The term “arylene” refers to the diradical version of an aryl group, e.g., 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein.

“Acyl” refers to a group of formula —C(O)R wherein R is alkyl, alkenyl, alkynyl, or substituted versions thereof. For example, the acetyl group has formula —C(O)CH₃. “Carbonyl” refers to a diradical group of formula —C(O)—.

“Alkoxy” refers to a group of formula —O(alkyl). Similar groups can be derived from alkenyl, alkynyl, aryl, heteroaryl, and other groups.

“Amino” refers to the group —NR^{X}R^{Y} wherein R^{X} and R^{Y} are each independently H or a non-hydrogen substituent. Exemplary non-hydrogen substituents include alkyl groups (e.g., methyl, ethyl, and isopropyl).

“Carbonyl” refers to a diradical group of formula —C(O)—.

“Carboxy” is used interchangeably with carboxyl and carboxylate to refer to the —CO₂H group and salts thereof.

“Ether” refers to a diradical group of formula —O—. For instance, if the ether group is connected to an alkyl group, then the overall group is an alkoxy group (e.g., —OCH₃ or methoxy). If the ether is connected to a carbonyl group, then the overall group is an ester group of formula —OC(O)—.

“Halo” and “halogen” refer to the chloro, bromo, fluoro, and iodo groups.

“Nitro” refers to the group of formula —NO₂.

Unless otherwise specified, reference to an atom is meant to include all isotopes of that atom. For example, reference to H includes ¹H, ²H (i.e., D or deuterium) and ³H (i.e., tritium), and reference to C is includes both ¹²C and all other isotopes of carbon (e.g., ¹³C). Unless specified otherwise, groups include all possible stereoisomers.

The terms “subject” and “patient” are used interchangeably herein.

“Imetelstat” is a compound with the Chemical Abstract Services (CAS) number of 868169-64-6.

“Imetelstat sodium” is the sodium salt of imetelstat.

### Formula (I) Compounds

Provided are compounds of formula (I):

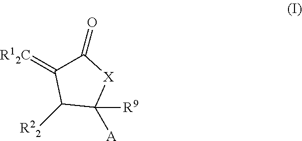

wherein:

X is O or NR^{X};

R^{X} is selected from the group consisting of H, D, alkyl, alkenyl, alkynyl, cycloalkyl, heterocyclyl, aryl, heteroaryl, acyl, carbonyl, carboxy, and substituted versions thereof;

each R¹ and R² is independently selected from the group consisting of H, D, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, thiol, thioether, and substituted versions thereof;

R⁹ is H, D, or alkyl; and

A is a 5- or 6-membered aromatic ring that is optionally substituted or a heterocyclic ring that is optionally substituted.

In some cases, A is a 5- or 6-membered heteroaryl ring that is optionally substituted. In some cases, A is a heterocyclic ring, e.g. with 5- or 6-members. In some cases, A is an aromatic ring that is further substituted at two adjacent points, e.g., optionally forming a second ring that is fused to the first ring. For example, A can be an indole ring, i.e. a benzene ring fused to a 5-membered pyrrole ring. In this example, A can be considered a phenyl ring that is substituted at two adjacent points with a —CH═CH—NH— group, thereby forming a pyrrole ring that is fused to the parent benzene ring.

As described above, each R¹ and R² is independently selected from the group consisting of H, D, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, thiol, thioether, and substituted versions thereof. For instance, R¹ can be alkyl, such as a C₁₋₆ alkyl such as methyl, ethyl, n-propyl, and i-propyl. In some cases, R¹ is methyl. In some cases, R² is H, D, alkyl, or substituted alkyl. As discussed above, R³ is H, D, or alkyl. For instance, R³ can be H. In some cases, R³ is methyl.

As discussed above, X is O or NR^{X}. In some embodiments, X is 0, and in such cases the inhibitor compounds can be referred to as having a lactone group. In other cases, X is NR⁶, and thus the compound can be referred to as having a lactam group. As described above, to R⁶ is independently selected from the group consisting of H, D, alkyl, alkenyl, alkynyl, cycloalkyl, heterocyclyl, aryl, heteroaryl, acyl, carbonyl, carboxy, and substituted versions thereof. In some embodiments, R⁶ is H, alkyl, or substituted alkyl, such as a C₁₋₆ alkyl, such as methyl.

As stated above, R⁹ is H, D, or alkyl. In some cases, R⁹ is H, D, or methyl.

In some cases, the compound has formula (Ia-A):

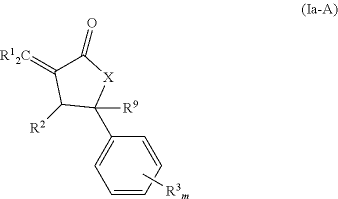

wherein:

m is 0 or an integer from 1 to 5; and

each R³ is independently alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof, provided that two adjacent R³ groups along with the atoms to which they are attached can form a cyclic ring.

In some cases, the compound has formula (Ia):

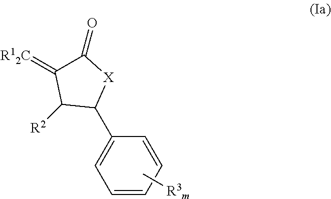

wherein:

m is 0 or an integer from 1 to 5; and

each R³ is independently alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof, provided that two adjacent R³ groups along with the atoms to which they are attached can form a cyclic ring.

In formulas (Ia-A) and (Ia), variable m is 0 or an integer from 1 to 5, e.g., m is 0 or m is 1. Each R³ is independently alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof, provided that two adjacent R³ groups along with the atoms to which they are attached can form a cyclic ring.

Furthermore, in some cases the compound has at least one R³ group located at the ortho position, e.g., as shown in formula (Ib-A):

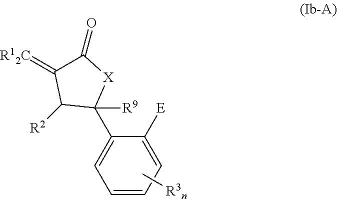

wherein:

n is 0 or an integer from 1 to 4; and

E is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some cases, the compound has formula (Ib):

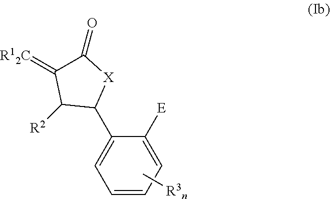

wherein:

n is 0 or an integer from 1 to 4; and

E is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some embodiments, E is selected from the group consisting of alkyl, aryl, heteroaryl, alkyloxy, aryloxy, heteroaryloxy, heterocyclyl, and substituted versions thereof. For instance, E can be a 5-membered heteroaryl or substituted heteroaryl group, e.g., E can be pyrazole or substituted pyrazole. For example, the compound can have formula (Ib-1-A):

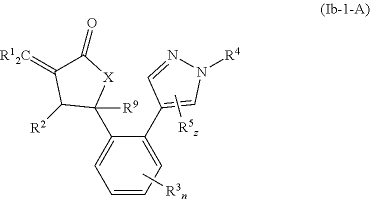

R⁴ is selected from the group consisting of H, alkyl, alkenyl, alkynyl, cycloalkyl, heterocyclyl, aryl, heteroaryl, acyl, carbonyl, carboxy, and substituted versions thereof;

z is 0, 1, or 2; and

each R⁵ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some embodiments of formula (Ib-1-A):

each R¹ is H;

R² is H or OH;

n is 1 or 2;

each R³ is located para or meta to the ring containing X;

each R³ is independently selected from F, Cl, and COOH;

R⁴ is H, methyl, ethyl, n-propyl, i-propyl, or a substituted alkyl group with 2 or 3 carbon atoms;

z is 0.

In some cases, the compound can have formula (Ib-1):

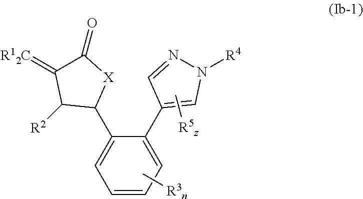

wherein:

R⁴ is selected from the group consisting of H, alkyl, alkenyl, alkynyl, cycloalkyl, heterocyclyl, aryl, heteroaryl, acyl, carbonyl, carboxy, and substituted versions thereof;

z is 0, 1, or 2; and

each R⁵ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some embodiments of formula (Ib-1-A) or (Ib-1), the E group is selected from the group consisting of triazole, substituted triazole, 6-membered aryl, 6-membered substituted aryl, 6-membered heteroaryl, and 6-membered substituted heteroaryl.

In some embodiments of formula (I), the compound has formula (Ic-A):

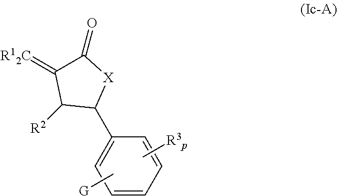

wherein:

p is 0 or an integer ranging from 1 to 4; and

G is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some embodiments, of formula (I), the compound has formula (Ic):

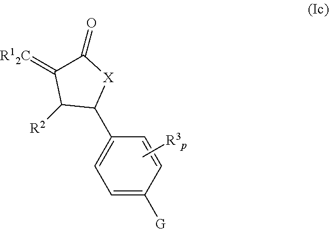

wherein:

p is 0 or an integer ranging from 1 to 4; and

G is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

For example, in some instances of formula (Ic), the G group is selected from the group consisting of alkyl, aryl, heteroaryl, alkyloxy, aryloxy, heteroaryloxy, heterocyclyl, and substituted versions thereof.

In some cases, the compound has formula (Id-A):

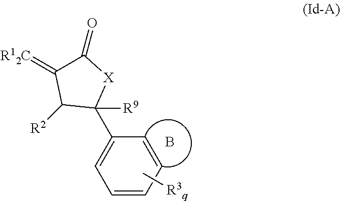

wherein:

q is 0 or an integer from 1 to 3; and

ring B is a 5- or 6-membered aryl or heteroaryl ring.

In some cases, the compound has formula (Id):

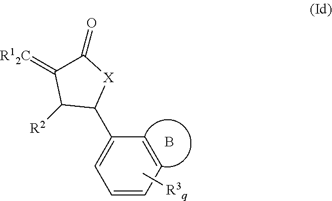

wherein:

q is 0 or an integer from 1 to 3; and

ring B is a 5- or 6-membered aryl or heteroaryl ring.

As described above, B is a 5- or 6-membered aryl or heteroaryl ring. For example, in some cases ring B is heteroaryl, e.g., pyridine, pyrrole, imidazole, or pyrrolidine.

In some embodiments, the telomerase inhibitor compound of formula (I) has a structure selected from the group consisting of:

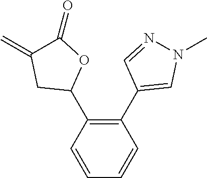

### 5-(2-(1-methyl-1H-pyrazol-4-yl)phenyl)-3-methylenedihydrofuran-2 (3H)-one

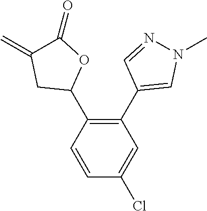

### 5-(4-chloro-2-(1-methyl-1H-pyrazol-4-yl)phenyl)-3-methylenedihydrofuran-2 (3H)-one

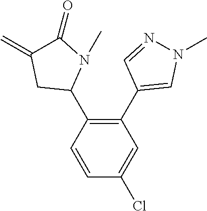

### 5-(4-chloro-2-(1-methyl-1H-pyrazol-4-yl)phenyl)-1-methyl-3-methylenepyrrolidin-2-one

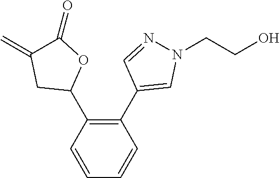 [SMILES: C=C1CC(c2ccccc2-c2cnn(CCO)c2)OC1=O]

### 5-(2-(1-(2-hydroxyethyl)-1H-pyrazol-4-yl)phenyl)-3-methylenedihydrofuran-2 (3H)-one

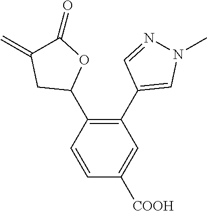

### 3-(1-methyl-1H-pyrazol-4-yl)-4-(4-methylene-5-oxotetrahydrofuran-2-yl)benzoic acid

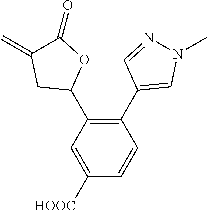

### 4-(1-methyl-H-pyrazol-4-yl)-3-(4-methylene-5-oxotetrahydrofuran-2-yl)benzoic acid

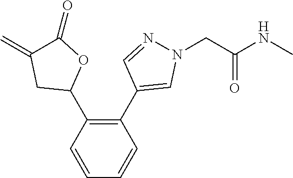

### N-methyl-2-(4-(2-(4-methylene-5-oxotetrahydrofuran-2-yl)phenyl)-1H-pyrazol-1-yl)acetamide

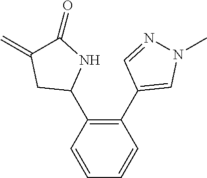

### 5-(2-(1-methyl-11H-pyrazol-4-yl)phenyl)-3-methylenepyrrolidin-2-one

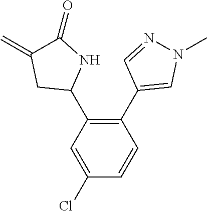

### 5-(5-chloro-2-(1-methyl-1H-pyrazol-4-yl)phenyl)-3-methylenepyrrolidin-2-one

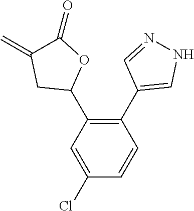 [SMILES: C=C1CC(c2cc(Cl)ccc2-c2cn[nH]c2)OC1=O]

### 5-(5-chloro-2-(1H-pyrazol-4-yl)phenyl)-3-methylenedihydrofuran-2 (3H)-one

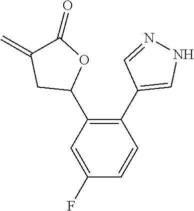 [SMILES: C=C1CC(c2cc(F)ccc2-c2cn[nH]c2)OC1=O]

### 5-(5-fluoro-2-(1H-pyrazol-4-yl)phenyl)-3-methylenedihydrofuran-2 (3H)-one

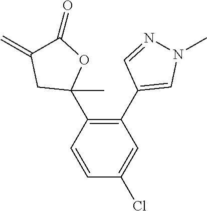

### 5-(4-chloro-2-(1-methyl-1H-pyrazol-4-yl)phenyl)-5-methyl-3-methylenedihydrofuran-2 (3H)-one

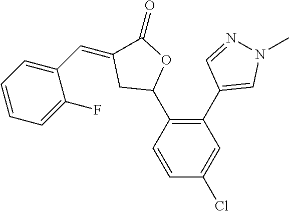 [SMILES: Cn1cc(-c2cc(Cl)ccc2C2C/C(=C\c3ccccc3F)C(=O)O2)cn1]

### (E)-5-(4-chloro-2-(1-methyl-1H-pyrazol-4-yl)phenyl)-3-(2-fluorobenzylidene)dihydrofuran-2 (3H)-one

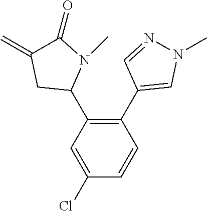

### 5-(5-chloro-2-(1-methyl-1H-pyrazol-4-yl)phenyl)-1-methyl-3-methylenepyrrolidin-2-one

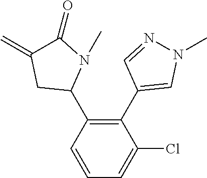

### 5-(3-chloro-2-(1-methyl-1H-pyrazol-4-yl)phenyl)-1-methyl-3-methylenepyrrolidin-2-one

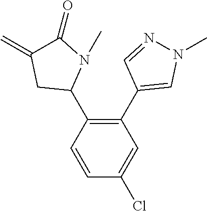

### 5-(4-chloro-2-(1-methyl-1H-pyrazol-4-yl)phenyl)-1-methyl-3-methylenepyrrolidin-2-one

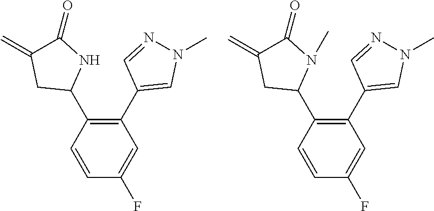

### 5-(4-fluoro-2-(1-methyl-1H-pyrazol-4-yl)phenyl)-3-methylenepyrrolidin-2-one

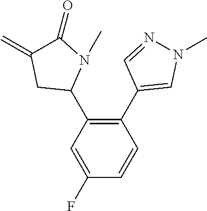

### 5-(5-fluoro-2-(1-methyl-1H-pyrazol-4-yl)phenyl)-1-methyl-3-methylenepyrrolidin-2-one

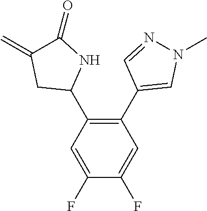

### 5-(4,5-difluoro-2-(1-methyl-1H-pyrazol-4-yl)phenyl)-3-methylenepyrrolidin-2-one

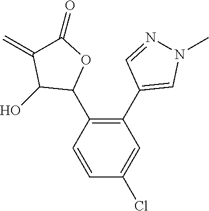

### 5-(4-chloro-2-(1-methyl-1H-pyrazol-4-yl)phenyl)-4-hydroxy-3-methylenedihydrofuran-2 (3H)-one

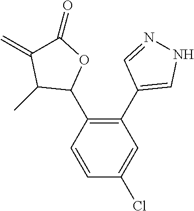 [SMILES: C=C1C(=O)OC(c2ccc(Cl)cc2-c2cn[nH]c2)C1C]

### 5-(4-chloro-2-(1H-pyrazol-4-yl)phenyl)-4-methyl-3-methylenedihydrofuran-2 (3H)-one

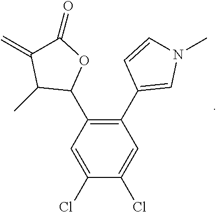

### 5-(4,5-dichloro-2-(1-methyl-1H-pyrrol-3-yl)phenyl)-4-methyl-3-methylenedihydrofuran-2 (3H)-one

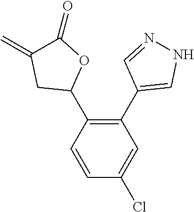 [SMILES: C=C1CC(c2ccc(Cl)cc2-c2cn[nH]c2)OC1=O]

### 5-(4-chloro-2-(1H-pyrazol-4-yl)phenyl)-3-methylenedihydrofuran-2 (3H)-one and

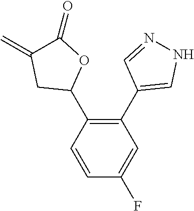 [SMILES: C=C1CC(c2ccc(F)cc2-c2cn[nH]c2)OC1=O]

### 5-(4-fluoro-2-(1H-pyrazol-4-yl)phenyl)-3-methylenedihydrofuran-2 (3H)-one

### Formula (II) Compounds

In some embodiments the compound is of formula (II):

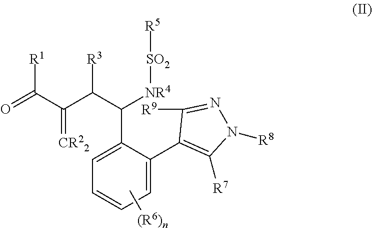

wherein:

n is 0, 1, 2, or 3;

each R¹, R², R⁶, R⁷, and R⁹ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and

each R³, R⁴, R⁵, and R⁸ is independently selected from the group consisting of H, alkyl, alkenyl, alkynyl, cycloalkyl, heterocyclyl, aryl, heteroaryl, acyl, and substituted versions thereof.

R¹ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. For instance, in some cases R¹ is H, alkyl, or substituted alkyl. In some cases, R¹ is alkyl, such as methyl or ethyl. In some cases, R¹ is H.

Each R² is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, each R² is H or alkyl, e.g., wherein each R² is H.

R³ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, R³ is H.

R⁴ is selected from the group consisting of H, alkyl, alkenyl, alkynyl, cycloalkyl, heterocyclyl, aryl, heteroaryl, acyl, and substituted versions thereof. In some embodiments, R⁴ is H or alkyl (e.g., methyl, ethyl). In some cases, R⁴ is H or methyl.

R⁵ is selected from the group consisting of H, alkyl, alkenyl, alkynyl, cycloalkyl, heterocyclyl, aryl, heteroaryl, acyl, and substituted versions thereof. In some cases, R⁵ is H or alkyl (e.g., methyl, ethyl). In some embodiments, R⁵ is H or methyl.

n is 0, 1, 2, or 3 and each R⁶ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, n ranges from 1 to 3, e.g., and at least one R⁶ is halo. For instance, in some cases n is 2 and each R⁶ is independently halo, such as one R⁶ is Cl and one R⁶ is F.

R⁷ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, R⁷ is H.

R⁸ is selected from the group consisting of H, alkyl, alkenyl, alkynyl, cycloalkyl, heterocyclyl, aryl, heteroaryl, acyl, and substituted versions thereof. In some cases, R⁸ is H or alkyl (e.g., methyl, ethyl).

R⁹ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, R⁹ is H or alkyl (e.g., methyl, ethyl).

In some embodiments, the telomerase inhibitor compound of formula (II) has a structure selected from the group consisting of:

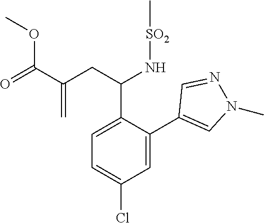 [SMILES: C=C(CC(NS(C)(=O)=O)c1ccc(Cl)cc1-c1cnn(C)c1)C(=O)OC]

### methyl-4-(4-chloro-2-(1-methyl-1H-pyrazol-4-yl)phenyl)-2-methylene-4-(methylsulfonamido)butanoate and

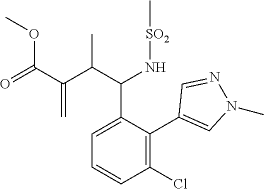 [SMILES: C=C(C(=O)OC)C(C)C(NS(C)(=O)=O)c1cccc(Cl)c1-c1cnn(C)c1]

### methyl-4-(3-chloro-2-(1-methyl-1H-pyrazol-4-yl)phenyl)-3-methyl-2-methylene-4-(methylsulfonamido)butanoate

### Formula (III) Compounds

Also provided compounds of formula (III):

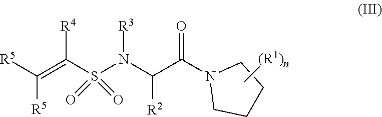

wherein:

n is 0 or an integer ranging from 1 to 4;

each R¹, R⁴, and R⁵ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof;

R² is selected from the group consisting of aryl, substituted aryl, heteroaryl, and substituted heteroaryl; and

R³ is selected from the group consisting of H, alkyl, alkenyl, alkynyl, cycloalkyl, heterocyclyl, aryl, heteroaryl, acyl, and substituted versions thereof.

In some embodiments, n is 0 or an integer ranging from 1 to 4. In some cases, n is 0. In some cases, n is 1 or 2.

Each R¹ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

R² is selected from the group consisting of aryl, substituted aryl, heteroaryl, and substituted heteroaryl. In some cases, R² is an aryl group or a substituted aryl group.

R³ is selected from the group consisting of H, alkyl, alkenyl, alkynyl, cycloalkyl, heterocyclyl, aryl, heteroaryl, acyl, and substituted versions thereof. In some embodiments, R³ is H.

R⁴ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, R⁴ is H.

Each R⁵ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, R⁵ is H.

In some embodiments, the telomerase inhibitor compound of formula (III) has a structure selected from the group consisting of:

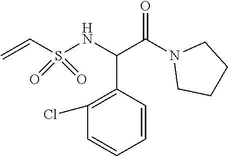 [SMILES: C=CS(=O)(=O)NC(C(=O)N1CCCC1)c1ccccc1Cl]

### N-(1-(2-chlorophenyl)-2-oxo-2-(pyrrolidin-1-yl)ethyl)ethenesulfonamide

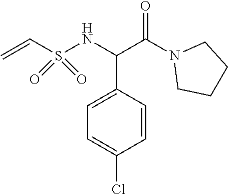 [SMILES: C=CS(=O)(=O)NC(C(=O)N1CCCC1)c1ccc(Cl)cc1]

### N-(1-(4-chlorophenyl)-2-oxo-2-(pyrrolidin-1-yl)ethyl)ethenesulfonamide

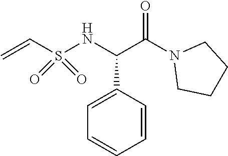 [SMILES: C=CS(=O)(=O)N[C@H](C(=O)N1CCCC1)c1ccccc1]

### (S)—N-(2-oxo-1-phenyl-2-(pyrrolidin-1-yl)ethyl)ethenesulfonamide and

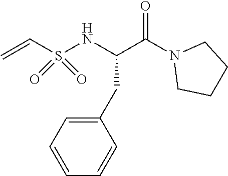 [SMILES: C=CS(=O)(=O)N[C@@H](Cc1ccccc1)C(=O)N1CCCC1]

### (S)—N-(1-oxo-3-phenyl-1-(pyrrolidin-1-yl)propan-2-yl)ethenesulfonamide

In some cases, the compound has a structure of formula (III) and R² is an aryl group or a substituted aryl group. In some cases, the compound has formula (IIIa):

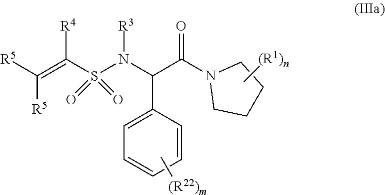

wherein:

m is 0 or an integer ranging from 1 to 5; and

each R²² is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

m is 0 or an integer ranging from 1 to 5. In some cases, m is 0. In some cases, m is 1 or 2.

Each R²² is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, each R²² is

In some cases, the compound has formula (IIIb):

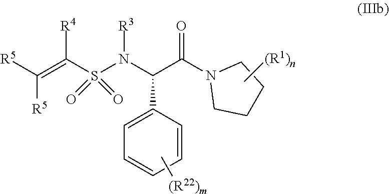

wherein:

m is 0 or an integer ranging from 1 to 5; and

each R²² is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

m is 0 or an integer ranging from 1 to 5 and each R²² is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, m ranges from 1 to 5, e.g., and at least one R²² is halo. For instance, in some cases m is 2 and one R²² is Cl and one R²² is F.

### Formula (IV) Compounds

Also provided are compounds of formula (IV):

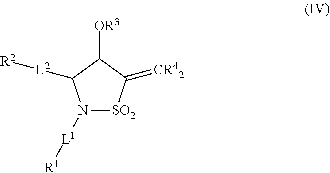

R¹ and R² are each independently selected from H, alkyl, alkenyl, alkynyl, cycloalkyl, heterocyclyl, aryl, heteroaryl, acyl, and substituted versions thereof, provided that at least one of R¹ and R² is aryl, heteroaryl, or a substituted version thereof;

L¹ and L² are each independently absent, an alkylene group, or a substituted alkylene group;

R³ is selected from the group consisting of H, alkyl, alkenyl, alkynyl, cycloalkyl, heterocyclyl, aryl, heteroaryl, acyl, and substituted versions thereof; and

each R⁴ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

R¹ is selected from H, alkyl, alkenyl, alkynyl, cycloalkyl, heterocyclyl, aryl, heteroaryl, acyl, and substituted versions thereof. In some cases, R¹ is aryl, substituted aryl, heteroaryl, or substituted heteroaryl. In some cases, R¹ is H.

R² is selected from H, alkyl, alkenyl, alkynyl, cycloalkyl, heterocyclyl, aryl, heteroaryl, acyl, and substituted versions thereof. In some cases, R² is aryl, substituted aryl, heteroaryl, or substituted heteroaryl. In some cases, R² is H.

L¹ and L² are each independently absent, an alkylene group, or a substituted alkylene group. The L¹ and L² groups can also be referred to as “linker” or “linking” groups. If the L group is absent then the corresponding R group is covalently bonded directly to the 5-membered ring. The L groups can be an alkylene group, which can be referred to as a diradical version of a (monoradical) alkyl group. Exemplary alkylene groups that can be used as L groups herein include —CH₂—, —CH₂CH₂—, —CH(CH₃)CH₂—, and —CH₂CH₂CH₂—.

R³ is selected from the group consisting of H, alkyl, alkenyl, alkynyl, cycloalkyl, heterocyclyl, aryl, heteroaryl, acyl, and substituted versions thereof. In some cases, R³ is H. In some embodiments, R³ is alkyl, e.g., methyl.

Each R⁴ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, each R⁴ is H.

In some embodiments, the telomerase inhibitor compound of formula (IV) has a structure selected from the group consisting of:

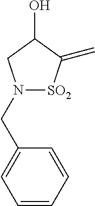 [SMILES: C=C1C(O)CN(Cc2ccccc2)S1(=O)=O]

### 2-benzyl-4-hydroxy-5-methyleneisothiazolidine 1,1-dioxide

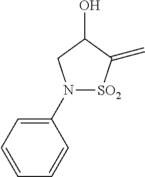

### 4-hydroxy-5-methylene-2-phenylisothiazolidine 1,1-dioxide and

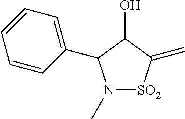 [SMILES: C=C1C(O)C(c2ccccc2)N(C)S1(=O)=O]

### 4-hydroxy-2-methyl-5-methylene-3-phenylisothiazolidine 1,1-dioxide

As described above, at least one of R¹ and R² is aryl, substituted aryl, heteroaryl, or substituted heteroaryl. In some cases, R¹ is aryl, substituted aryl, heteroaryl, or substituted heteroaryl. For instance, in some embodiments the compound has formula (IVa):

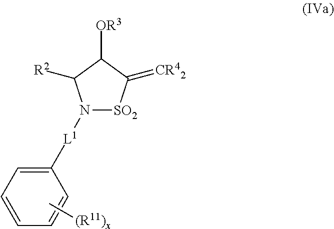

wherein:

x is 0 or an integer ranging from 1 to 5; and

each R¹¹ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

x is 0 or an integer ranging from 1 to 5 and each R¹¹ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, x is 0. In some embodiments, x is an integer ranging from 1 to 5, e.g., and at least one R¹¹ is halo. For instance, x can be 2 and one R¹¹ can be F and one R¹¹ can be Cl. In some cases, R² is H or alkyl, e.g., methyl.

L¹ is independently absent, an alkylene group, or a substituted alkylene group and each R⁴ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, L¹ is absent. In some cases, L¹ has the formula —CH₂—. In some cases, each R⁴ is H.

R² is selected from H, alkyl, alkenyl, alkynyl, cycloalkyl, heterocyclyl, aryl, heteroaryl, acyl, and substituted versions thereof. In some cases, R² is aryl, substituted aryl, heteroaryl, or substituted heteroaryl. For instance, in some embodiments the compound has formula (IVb):

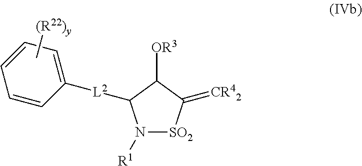

wherein:

y is 0 or an integer ranging from 1 to 5; and

each R²² is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

y is 0 or an integer ranging from 1 to 5 and each R²² is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, y is 0. In some cases, y is an integer ranging from 1 to 5, e.g., and at least one R²² is halo. For instance, in some cases y is 2 and one R²² is Cl and one R²² is F.

### Formula (V) Compounds

Also provided are compounds of formula (V):

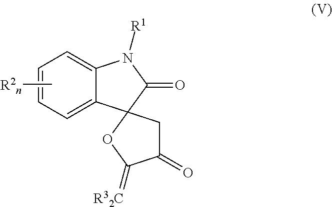

wherein:

R¹ is selected from the group consisting of H, alkyl, alkenyl, alkynyl, cycloalkyl, heterocyclyl, aryl, heteroaryl, acyl, carbonyl, carboxy, and substituted versions thereof;

each R² and R³ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and

n is 0 or an integer from 1 to 4.

For instance, in some cases both R³ groups are H and hence the compound has formula (Va):

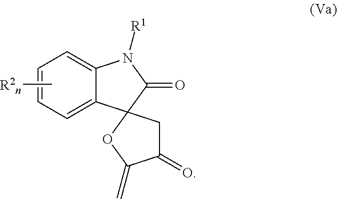

As described above, R¹ is selected from the group consisting of H, alkyl, alkenyl, alkynyl, cycloalkyl, heterocyclyl, aryl, heteroaryl, acyl, carbonyl, carboxy, and substituted versions thereof. In some cases, R¹ is alkyl, e.g., methyl. In some embodiments, n is 0 and therefore the compound does not have an R² group.

In some embodiments, the telomerase inhibitor compound of formula (V) has a structure selected from the group consisting of:

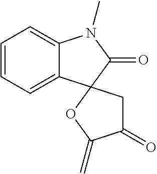 [SMILES: C=C1OC2(CC1=O)C(=O)N(C)c1ccccc12]

### 1′-methyl-5-methylene-3H-spiro[furan-2,3′-indoline]-2′,4 (5H)-dione

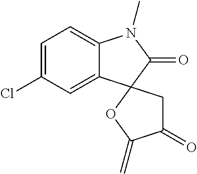 [SMILES: C=C1OC2(CC1=O)C(=O)N(C)c1ccc(Cl)cc12]

### 5′-chloro-1′-methyl-5-methylene-3H-spiro[furan-2,3′-indoline]-2′,4 (5H)-dione

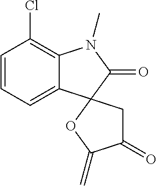 [SMILES: C=C1OC2(CC1=O)C(=O)N(C)c1c(Cl)cccc12]

### 7′-chloro-1′-methyl-5-methylene-3H-spiro[furan-2,3′-indoline]-2′,4 (5H)-dione and

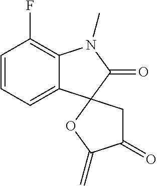 [SMILES: C=C1OC2(CC1=O)C(=O)N(C)c1c(F)cccc12]

### 7′-fluoro-1′-methyl-5-methylene-3H-spiro[furan-2,3′-indoline]-2′,4 (5H)-dione

### Formula (VI) Compounds

Provided are compounds of formula (VI):

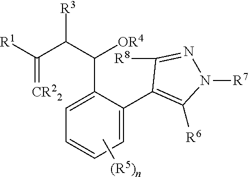

wherein:

n is 0, 1, 2, or 3;

each R¹, R², R³, R⁵, R⁶, and R⁸ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and

R⁴ and R⁷ are independently selected from the group consisting of H, alkyl, alkenyl, alkynyl, cycloalkyl, heterocyclyl, aryl, heteroaryl, acyl, carbonyl, carboxy, and substituted versions thereof.

R¹ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. For instance, in some cases R¹ is H, alkyl, or substituted alkyl. In some cases, R¹ is a carboxylic acid group (i.e. —COOH) or a salt thereof. In some cases, R¹ is cyano.

Each R² is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, each R² is H or alkyl, e.g., wherein each R² is H.

R³ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, R³ is H.

R⁴ is selected from the group consisting of H, alkyl, alkenyl, alkynyl, cycloalkyl, heterocyclyl, aryl, heteroaryl, acyl, and substituted versions thereof. In some embodiments, R⁴ is H or alkyl (e.g., methyl, ethyl). In some cases, R⁴ is H or methyl.

n is 0, 1, 2, or 3 and each R⁵ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, n ranges from 1 to 3, e.g., and at least one R⁵ is halo. For instance, in some cases n is 2 and each R⁵ is independently halo, such as one R⁵ is Cl and one R⁵ is F.

R⁶ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some embodiments, R⁶ is H.

R⁷ is selected from the group consisting of H, alkyl, alkenyl, alkynyl, cycloalkyl, heterocyclyl, aryl, heteroaryl, acyl, and substituted versions thereof. In some cases, R⁷ is H or alkyl (e.g., methyl, ethyl).

R⁸ is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof. In some cases, R⁸ is H or alkyl (e.g., methyl, ethyl).

In some cases, the compound has a structure selected from the group consisting of:

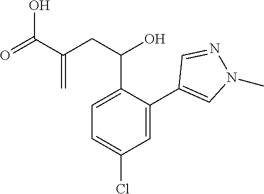 [SMILES: C=C(CC(O)c1ccc(Cl)cc1-c1cnn(C)c1)C(=O)O]

### 4-(4-chloro-2-(1-methyl-1H-pyrazol-4-yl)phenyl)-4-hydroxy-2-methylenebutanoic acid

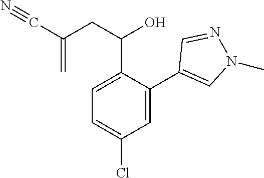 [SMILES: C=C(C#N)CC(O)c1ccc(Cl)cc1-c1cnn(C)c1]

### 4-(4-chloro-2-(1-methyl-1H-pyrazol-4-yl)phenyl)-4-hydroxy-2-methylenebutanenitrile

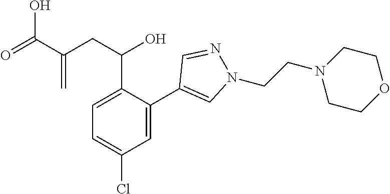 [SMILES: C=C(CC(O)c1ccc(Cl)cc1-c1cnn(CCN2CCOCC2)c1)C(=O)O]

### 4-(4-chloro-2-(1-(2-morpholinoethyl)-1H-pyrazol-4-yl)phenyl)-4-hydroxy-2-methylenebutanoic acid and

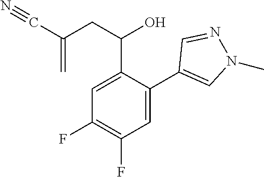 [SMILES: C=C(C#N)CC(O)c1cc(F)c(F)cc1-c1cnn(C)c1]

### 4-(4,5-difluoro-2-(1-methyl-1H-pyrazol-4-yl)phenyl)-4-hydroxy-2-methylenebutanenitrile

### Compositions and Formulations

Also provided are compositions comprising a compound as disclosed herein. For example, the compound can have formula (I), formula (Ia), formula (Ib), formula (Ic), formula (Id), formula (II), formula (III), formula (IIIa), formula (IIIb), formula (IV), formula (IVa), formula (IVb), formula (V), formula (Va), or formula (VI), as described above. In some cases, the composition includes a racemic mixture of stereoisomers. In some embodiments, the composition is enriched in a particular stereoisomer, e.g., the composition is enriched in a first enantiomer relative to a second enantiomer. The term “enantiomeric excess” is used herein to quantify the relative amount of the first enantiomer compared to the second enantiomer, wherein enantiomeric excess is the absolute difference between the mole fraction of each enantiomer. For instance, if 70% of a compound is a first enantiomer and 30% of the compound is the second enantiomer, then the enantiomeric excess is 40% (i.e., 70% minus 40%). In some cases, the composition has an enantiomeric excess of the first enantiomer of 1% or more, such as 10% or more, 20% or more, 30% or more, or 40% or more. In some embodiments, the composition is an aqueous solution of the compound.

In certain embodiments, the disclosed compounds are useful for the treatment of a disease or disorder. Accordingly, pharmaceutical compositions comprising at least one disclosed compound are also described herein. For example, the present disclosure provides pharmaceutical compositions that include a therapeutically effective amount of a compound of the present disclosure (or a pharmaceutically acceptable salt or solvate or hydrate or stereoisomer thereof) and a pharmaceutically acceptable excipient.

A pharmaceutical composition that includes a subject compound may be administered to a patient alone, or in combination with other supplementary active agents. For example, one or more compounds according to the present disclosure can be administered to a patient with or without supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, spray dried dispersion, lyophilisate, tablet, microtablets, pill, pellet, capsule, powder, syrup, elixir or any other dosage form suitable for administration.

A compound or prodrug of the present disclosure may be administered to a subject using any convenient means capable of resulting in the desired reduction in disease condition or symptom. Thus, a compound or prodrug can be incorporated into a variety of formulations for therapeutic administration. More particularly, a compound or prodrug can be formulated into pharmaceutical compositions by combination with appropriate pharmaceutically acceptable excipients, carriers or diluents, and may be formulated into preparations in solid, semi-solid, liquid or gaseous forms, such as tablets, capsules, powders, granules, ointments, creams, gels, foams, solutions, suppositories, injections, inhalants, aerosols, and the like.

Formulations for pharmaceutical compositions are described in, for example, Remington's Pharmaceutical Sciences, by E. W. Martin, Mack Publishing Co., Easton, Pa., 19th Edition, 1995, which describes examples of formulations (and components thereof) suitable for pharmaceutical delivery of the disclosed compounds. Pharmaceutical compositions that include at least one of the compounds or prodrugs can be formulated for use in human or veterinary medicine. Particular formulations of a disclosed pharmaceutical composition may depend, for example, on the mode of administration and/or on the location of the subject to be treated. In some embodiments, formulations include a pharmaceutically acceptable excipient in addition to at least one active ingredient, such as a compound of the present disclosure. In other embodiments, other medicinal or pharmaceutical agents, for example, with similar, related or complementary effects on the disease or condition being treated can also be included as active ingredients in a pharmaceutical composition.

Pharmaceutically acceptable carriers useful for the disclosed methods and compositions may depend on the particular mode of administration being employed. In addition to biologically neutral carriers, pharmaceutical compositions to be administered can optionally contain non-toxic auxiliary substances (e.g., excipients), such as wetting or emulsifying agents, preservatives, and pH buffering agents, and the like.

The disclosed pharmaceutical compositions may be formulated as a pharmaceutically acceptable salt of a disclosed compound.

The term “unit dosage form,” as used herein, refers to physically discrete units suitable as unitary dosages for human and animal subjects, each unit containing a predetermined quantity of a compound or prodrug calculated in an amount sufficient to produce the desired effect in association with a pharmaceutically acceptable diluent, excipient, carrier or vehicle. The specifications for a compound or prodrug depend on the particular compound or prodrug employed and the effect to be achieved, and the pharmacodynamics associated with each compound in the subject.

The dosage form of a disclosed pharmaceutical composition may be determined by the mode of administration chosen. For example, in addition to injectable fluids, topical or oral dosage forms may be employed. Topical preparations may include eye drops, ointments, sprays and the like. Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions that include a subject compound or prodrug may be formulated in unit dosage form suitable for individual administration of precise dosages. The amount of active ingredient administered may depend on the subject being treated, the severity of the affliction, and the manner of administration, and is known to those skilled in the art. In certain instances, the formulation to be administered contains a quantity of the compound or prodrug disclosed herein in an amount effective to achieve the desired effect in the subject being treated.

Each therapeutic compound can independently be in any dosage form, such as those described herein, and can also be administered in various ways, as described herein. For example, the compounds or prodrugs may be formulated together, in a single dosage unit (that is, combined together in one form such as capsule, tablet, powder, or liquid, etc.) as a combination product. Alternatively, when not formulated together in a single dosage unit, an individual compound or prodrug may be administered at the same time as another therapeutic compound or sequentially, in any order thereof.

A disclosed compound can be administered alone, as the sole active pharmaceutical agent, or in combination with one or more additional compounds or prodrugs of the present disclosure or in conjunction with other agents. When administered as a combination, the therapeutic agents can be formulated as separate compositions that are administered simultaneously or at different times, or the therapeutic agents can be administered together as a single composition combining two or more therapeutic agents. Thus, the pharmaceutical compositions disclosed herein containing a compound of the present disclosure optionally include other therapeutic agents. Accordingly, certain embodiments are directed to such pharmaceutical compositions, where the composition further includes a therapeutically effective amount of an agent selected as is known to those of skill in the art.

In some embodiments, the pharmaceutical composition comprises disclosed compounds (e.g., of formula (I), (II), (III), (IV)), (V), or (VI), or any other formulae described herein) along with a second telomerase inhibitor, e.g. imetelstat or imetelstat sodium. Imetelstat has been described as a telomerase inhibitor that has been studied for its ability to inhibit various cancers, such as pancreatic cancer (Burchett et al, PLoS One, 2014, 9(1), e85155, doi:10.1371/journal.pone.0085155).

### Methods of Treatment

Also provided are methods of treating a patient for a telomerase-related disease or condition. Such telomerase-related conditions are characterized by telomerase activity or increased telomerase activity or expression or over-expression of telomerase in a cell that does not express or expresses at very low levels telomerase or has little or very low telomerase activity. The method can include administering a compound as described above to the patient. In some cases, the telomerase-related condition is cancer. In some cases, the condition is Hoyeraal-Hreidarsson syndrome, dyskeratosis congenita, pulmonary fibrosis, aplastic anemia, or liver fibrosis.

In some cases, the cancer is a hematological malignancy. In some cases, the cancer is selected from the group consisting of acute and chronic leukemias, lymphomas, multiple myeloma and myelodysplastic syndromes, myeloproliferative neoplasms (MPNs), essential thrombocythemia (ET), polycythemia vera (PV), Chronic Myelogenous Leukemia (CML), myelofibrosis (MF), acute myelogenous leukemia (AML), myelodysplastic syndromes (MDS).

### Essential Thrombocythemia

Circulating blood platelets are anucleate, although they retain small amounts of megakaryocyte-derived mRNAs and a fully functional protein biosynthetic capacity (Gnatenko et al., Blood 101, 2285-2293 (2003)). Essential Thrombocythemia (ET) is a myeloproliferative disorder subtype, characterized by increased neoplastic proliferation of megakaryocytes, elevated numbers of circulating platelets, and considerable thrombohemorrhagic events, not infrequently neurological (Nimer, Blood 93, 415-416 (1999)). ET is seen with equal frequency in males and females, although an additional female incidence peak at age 30 may explain the apparent higher disease prevalence in females after this age. The molecular basis of ET remains to be established, although historically it has been considered a “clonal” disorder (El-Kassar et al., Blood 89, 128 (1997); “Evidence that ET is a clonal disorder with origin in a multipotent stem cell” PJ Fialkow, Blood 1981 58: 916-919). Other than the exaggerated platelet volume evident in subsets of ET platelets, the cells remain morphologically indistinguishable from their normal counterparts. No functional or diagnostic test is currently available for ET, and it remains to be diagnosed by exclusion of other potential hematological disorders. Incidence estimates of 2-3 cases per 100,000 per year are consistent with other types of leukemia, but prevalence rates are at least ten times higher due to the low mortality rates associated with ET.

Current therapies for ET focus primarily on prevention of thrombotic/hemorrhagic occurrence and involve non-specific reduction of blood platelet levels. Additionally, many individuals with ET develop resistance to front-line treatments such as hydroxyurea or discontinue use of these drugs altogether due to adverse side effects.

### Polycythemia Vera

Patients with Polycythemia Vera (PV) have marked increases of red blood cell production. Treatment is directed at reducing the excessive numbers of red blood cells. PV can develop a phase late in their course that resembles primary myelofibrosis with cytopenias and marrow hypoplasia and fibrosis. The Janus Kinase 2 gene (JAK2) gene mutation on chromosome 9 which causes increased proliferation and survival of hematopoietic precursors in vitro has been identified in most patients with PV. Patients with PV have an increased risk of cardiovascular and thrombotic events and transformation to acute myelogenous leukemia or primary myelofibrosis. The treatment for PV includes intermittent chronic phlebotomy to maintain the hematocrit below 45% in men and 40% in women. Other possible treatments include hydroxyurea, interferon-alpha, and low-dose aspirin.

### Myelofibrosis

Myelofibrosis or MF, or primary myelofibrosis is a myeloproliferative neoplasm in the same spectrum of diseases as ET. Patients with MF often carry the JAK2 V617F mutation in their bone marrow. Occasionally ET evolves into MF. JAK2 inhibition is currently considered a standard of care for MF in countries where ruxolitinib (Jakafi®), a janus kinase inhibitor, is approved. There is no evidence that JAK2 inhibitors, such as Jakafi®, selectively inhibit proliferation of the leukemic clone responsible for the disease and thus, they may not be “disease modifying”.

### Acute Myelogenous Leukemia

Acute Myelogenous Leukemia (AML) is a cancer of the myeloid line of blood cells. AML is the most common acute leukemia affecting adults. Patients with AML have a rapid growth of abnormal white blood cells that accumulate in the bone marrow and interfere with the production of normal blood cells. Replacement of normal bone marrow with leukemic cells causes a drop in red blood cells, platelets, and normal white blood cells. The symptoms of AML include fatigue, shortness of breath, easy bruising and bleeding, and increased risk of infection. As an acute leukemia, AML progresses rapidly and is typically fatal within weeks or months if left untreated. The standard of care for AML is treatment with chemotherapy aimed at inducing a remission; patients may go on to receive a hematopoietic stem cell transplant.

### Myelodysplastic Syndrome

Myelodysplastic syndromes (MDS) represent a rare heterogenous group of hemopoietic clonal disorders that are characterized by ineffective hemopoiesis resulting in anemia and other cytopenias and are serious and life threatening with a high risk of leukemic transformation. Myelodysplastic syndromes (MDS) include diseases such as, refractory anemia, refractory anemia with excess blasts, refractory cytopenia with multilineage dysplasia, refractory cytopenia with unilineage dysplasia, and chronic myelomonocytic leukemia. The immature blood stem cells (blasts) do not become healthy red blood cells, white blood cells or platelets. The blasts die in the bone marrow or soon after they travel to the blood. This leaves less room for healthy white cells, red cells and/or platelets to form in the bone marrow.

The myelodysplastic syndromes (MDS) are a collection of hematological medical conditions that involve ineffective production of the myeloid class of blood cells. Patients with MDS often develop severe anemia and require frequent blood transfusions. Bleeding and risk of infections also occur due to low or dysfunctional platelets and neutrophils, respectively. In some cases the disease worsens and the patient develops cytopenias (low blood counts) caused by progressive bone marrow failure. In some cases the disease transforms into acute myelogenous leukemia (AML). If the overall percentage of bone marrow myeloblasts rises over a particular cutoff (20% for WHO and 30% for FAB), then transformation to acute myelogenous leukemia (AML) is said to have occurred.

The compound can be administered in a therapeutically effective amount. As such, the administration can inhibit the telomerase in cancer cells of the patient, thereby inhibiting the cancer. For instance, the cancer can be inhibited by inhibiting the division of its cells. In some cases, the disclosed telomerase inhibitor compounds can be administered and the method further comprises administering a second telomerase inhibitor, e.g. imetelstat or imetelstat sodium.

By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

The subject compounds find use for treating a disease or disorder in a subject. The route of administration may be selected according to a variety of factors including, but not limited to, the condition to be treated, the formulation and/or device used, the subject to be treated, and the like. Routes of administration useful in the disclosed methods include, but are not limited to, oral and parenteral routes, such as intravenous (iv), intraperitoneal (ip), rectal, topical, ophthalmic, nasal, otic, intrathecal, and transdermal. Formulations for these dosage forms are described herein.

An effective amount of a subject compound or prodrug may depend, at least, on the particular method of use, the subject being treated, the severity of the affliction, and the manner of administration of the therapeutic composition. A “therapeutically effective amount” of a composition is a quantity of a specified compound or prodrug sufficient to achieve a desired effect in a subject (e.g., patient) being treated. For example, this may be the amount of a subject compound necessary to prevent, inhibit, reduce or relieve a disease or disorder in a subject. Ideally, a therapeutically effective amount of a compound or prodrug is an amount sufficient to prevent, inhibit, reduce or relieve a disease or disorder in a subject without causing a substantial cytotoxic effect on normal? host cells in the subject.

Therapeutically effective doses of a subject compound or prodrug or pharmaceutical composition can be determined by one of skill in the art. For example, in some instances, a therapeutically effective dose of a compound or prodrug or pharmaceutical composition is administered with a goal of achieving local (e.g., tissue) or plasma concentrations that are at least as high as the IC₅₀ of an applicable compound disclosed herein.

The specific dose level and frequency of dosage for any particular subject may be varied and may depend upon a variety of factors, including the activity of the subject compound or prodrug, the metabolic stability and length of action of that compound or prodrug, the age, body weight, general health, sex and diet of the subject, mode and time of administration, rate of excretion, drug combination, and severity of the condition of the host undergoing therapy.

In some embodiments, multiple doses of a compound or prodrug are administered. The frequency of administration of a compound can vary depending on any of a variety of factors, e.g., severity of the symptoms, condition of the subject, etc. For example, in some embodiments, a compound is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### EMBODIMENTS

Embodiments of the invention include, but are not limited to, the embodiments described in the following clauses:

1. A compound of formula (I):

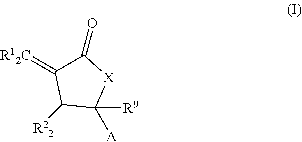

wherein:

X is O or NR^{X};

R^{X} is selected from the group consisting of H, D, alkyl, alkenyl, alkynyl, cycloalkyl, heterocyclyl, aryl, heteroaryl, acyl, carbonyl, carboxy, and substituted versions thereof;

each R¹ and R² is independently selected from the group consisting of H, D, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, thiol, thioether, and substituted versions thereof;

R⁹ is H, D, or alkyl; and

A is a 5- or 6-membered aromatic ring that is optionally substituted or a heterocyclic ring that is optionally substituted.

2. The compound of clause 1, wherein X is O.
3. The compound of clause 1, wherein X is NR^{X}.
4. The compound of any one of clauses 1-3, wherein the compound has formula (Ia-A):

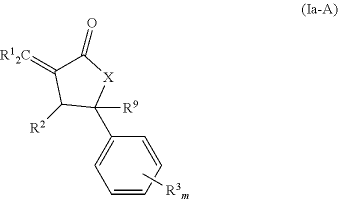

wherein:

m is 0 or an integer from 1 to 5; and

each R³ is independently alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof, provided that two adjacent R³ groups along with the atoms to which they are attached can form a cyclic ring.

5. The compound of any one of clauses 1-3, wherein the compound has formula (Ib-A)

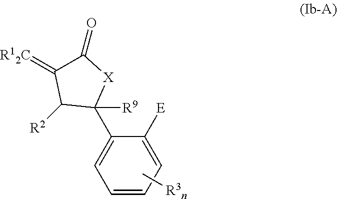

wherein:

n is 0 or an integer from 1 to 4; and

E is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

6. The compound of clause 5, wherein E is selected from the group consisting of alkyl, aryl, heteroaryl, alkyloxy, aryloxy, heteroaryloxy, heterocyclyl, and substituted versions thereof.
7. The compound of clause 6, wherein E is a 5-membered heteroaryl or substituted heteroaryl group.
8. The compound of clause 7, wherein E is pyrazole or substituted pyrazole.
9. The compound of clause 8, wherein the compound has formula (Ib-1-A):

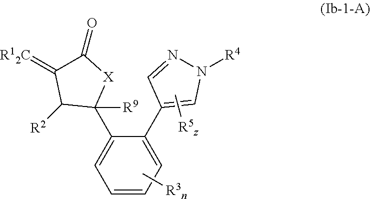

wherein:

R⁴ is selected from the group consisting of H, alkyl, alkenyl, alkynyl, cycloalkyl, heterocyclyl, aryl, heteroaryl, acyl, carbonyl, carboxy, and substituted versions thereof;

z is 0, 1, or 2; and

each R⁵ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

10. The compound of clause 7, wherein E is selected from the group consisting of triazole, substituted triazole, 6-membered aryl, 6-membered substituted aryl, 6-membered heteroaryl, and 6-membered substituted heteroaryl.
11. The compound of any one of clauses 1-3, wherein the compound has formula (Ic-A):

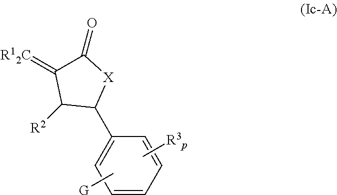

wherein:

p is 0 or an integer ranging from 1 to 4; and

G is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

12. The compound of clause 11, wherein G is selected from the group consisting of alkyl, aryl, heteroaryl, alkyloxy, aryloxy, heteroaryloxy, heterocyclyl, and substituted versions thereof.
13. The compound of any one of clauses 1-3, wherein the compound has formula (Id-A):

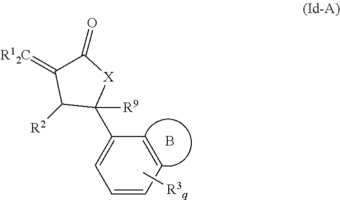

wherein:

q is 0 or an integer from 1 to 3; and

ring B is a 5- or 6-membered aryl or heteroaryl ring.

14. The compound of clause 13, wherein ring B is heteroaryl.
15. The compound of clause 14, wherein ring B is pyridine, pyrrole, imidazole, or pyrrolidine.
16. The compound of clause 1, wherein the compound has a structure selected from the group consisting of:

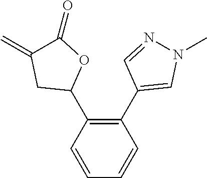

### 5-(2-(1-methyl-1H-pyrazol-4-yl)phenyl)-3-methylenedihydrofuran-2 (3H)-one

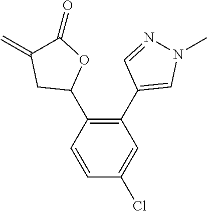

### 5-(4-chloro-2-(1-methyl-1H-pyrazol-4-yl)phenyl)-3-methylenedihydrofuran-2 (3H)-one

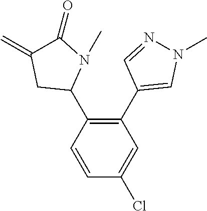

### 5-(4-chloro-2-(1-methyl-1H-pyrazol-4-yl)phenyl)-1-methyl-3-methylenepyrrolidin-2-one

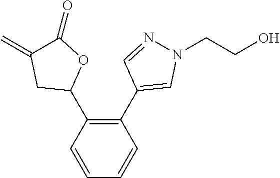 [SMILES: C=C1CC(c2ccccc2-c2cnn(CCO)c2)OC1=O]

### 5-(2-(1-(2-hydroxyethyl)-1H-pyrazol-4-yl)phenyl)-3-methylenedihydrofuran-2 (3H)-one

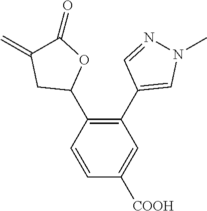

### 3-(1-methyl-1H-pyrazol-4-yl)-4-(4-methylene-5-oxotetrahydrofuran-2-yl)benzoic acid

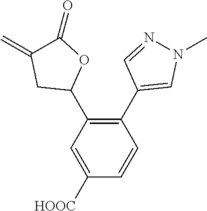

### 4-(1-methyl-1H-pyrazol-4-yl)-3-(4-methylene-5-oxotetrahydrofuran-2-yl)benzoic acid

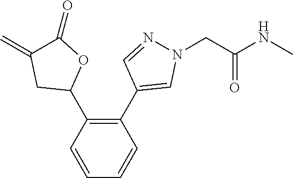

### N-methyl-2-(4-(2-(4-methylene-5-oxotetrahydrofuran-2-yl)phenyl)-1H-pyrazol-1-yl)acetamide

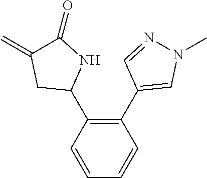

### 5-(2-(1-methyl-1H-pyrazol-4-yl)phenyl)-3-methylenepyrrolidin-2-one

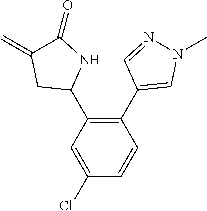 [0314] 5-(5-chloro-2-(1-methyl-1H-pyrazol-4-yl)phenyl)-3-methylenepyrrolidin-2-one

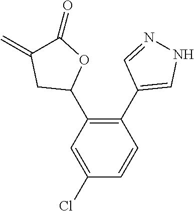 [SMILES: C=C1CC(c2cc(Cl)ccc2-c2cn[nH]c2)OC1=O]

### 5-(5-chloro-2-(1H-pyrazol-4-yl)phenyl)-3-methylenedihydrofuran-2 (3H)-one

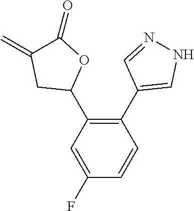 [SMILES: C=C1CC(c2cc(F)ccc2-c2cn[nH]c2)OC1=O]

### 5-(5-fluoro-2-(1H-pyrazol-4-yl)phenyl)-3-methylenedihydrofuran-2 (3H)-one

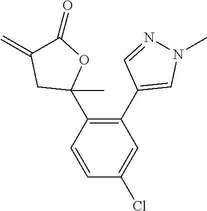

### 5-(4-chloro-2-(1-methyl-1H-pyrazol-4-yl)phenyl)-5-methyl-3-methylenedihydrofuran-2 (3H)-one

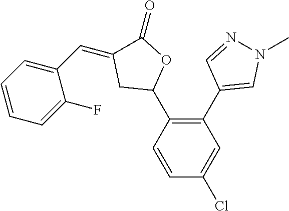 [SMILES: Cn1cc(-c2cc(Cl)ccc2C2C/C(=C\c3ccccc3F)C(=O)O2)cn1]

### (E)-5-(4-chloro-2-(1-methyl-1H-pyrazol-4-yl)phenyl)-3-(2-fluorobenzylidene)dihydrofuran-2 (3H)-one

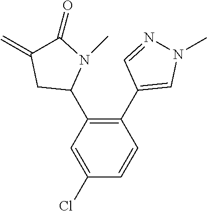

### 5-(5-chloro-2-(1-methyl-1H-pyrazol-4-yl)phenyl)-1-methyl-3-methylenepyrrolidin-2-one

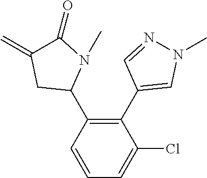

### 5-(3-chloro-2-(1-methyl-1H-pyrazol-4-yl)phenyl)-1-methyl-3-methylenepyrrolidin-2-one

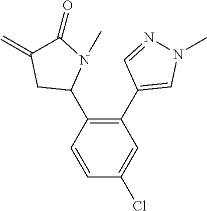

### 5-(4-chloro-2-(1-methyl-1H-pyrazol-4-yl)phenyl)-1-methyl-3-methylenepyrrolidin-2-one

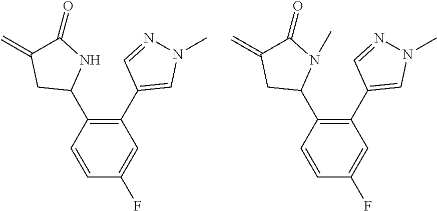

### 5-(4-fluoro-2-(1-methyl-1H-pyrazol-4-yl)phenyl)-3-methylenepyrrolidin-2-one

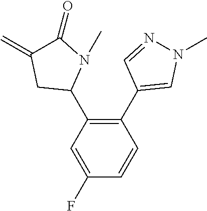

### 5-(5-fluoro-2-(1-methyl-1H-pyrazol-4-yl)phenyl)-1-methyl-3-methylenepyrrolidin-2-one

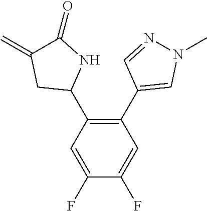

### 5-(4,5-difluoro-2-(1-methyl-1H-pyrazol-4-yl)phenyl)-3-methylenepyrrolidin-2-one

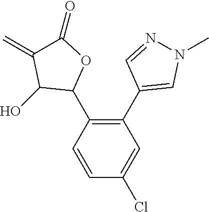

### 5-(4-chloro-2-(1-methyl-1H-pyrazol-4-yl)phenyl)-4-hydroxy-3-methylenedihydrofuran-2 (3H)-one

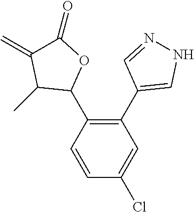 [SMILES: C=C1C(=O)OC(c2ccc(Cl)cc2-c2cn[nH]c2)C1C]

### 5-(4-chloro-2-(1H-pyrazol-4-yl)phenyl)-4-methyl-3-methylenedihydrofuran-2 (3H)-one

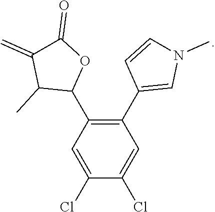

### 5-(4,5-dichloro-2-(1-methyl-1H-pyrrol-3-yl)phenyl)-4-methyl-3-methylenedihydrofuran-2 (3H)-one

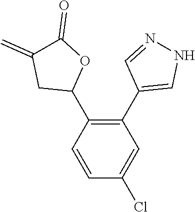 [SMILES: C=C1CC(c2ccc(Cl)cc2-c2cn[nH]c2)OC1=O]

### 5-(4-chloro-2-(1H-pyrazol-4-yl)phenyl)-3-methylenedihydrofuran-2 (3H)-one and

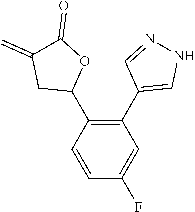 [SMILES: C=C1CC(c2ccc(F)cc2-c2cn[nH]c2)OC1=O]

17. A compound of formula (II):

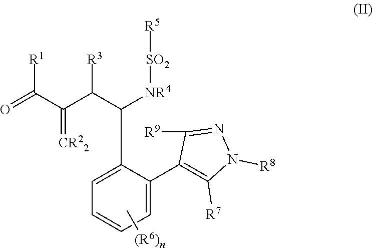

wherein:

n is 0, 1, 2, or 3;

each R¹, R², R⁶, R⁷, and R⁹ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and

each R³, R⁴, R⁵, and R⁸ is independently selected from the group consisting of H, alkyl, alkenyl, alkynyl, cycloalkyl, heterocyclyl, aryl, heteroaryl, acyl, carbonyl, carboxy, and substituted versions thereof.

18. The compound of clause 17, wherein R¹ is H.
19. The compound of any one of clauses 17-18, wherein each R² is H.
20. The compound of any one of clauses 17-19, wherein R³ is H.
21. The compound of any one of clauses 17-20, wherein R⁴ is H or alkyl.
22. The compound of any one of clauses 17-21, wherein R⁵ is H or alkyl.
23. The compound of any one of clauses 17-22, wherein n ranges from 1 to 3 and at least one R⁶ is halo.
24. The compound of any one of clauses 17-23, wherein R⁷ is H.
25. The compound of any one of clauses 17-24, wherein R⁸ is H or alkyl.
26. The compound of any one of clauses 17-25, wherein R⁹ is H.
27. The compound of clause 17, wherein the compound has a structure selected from the group consisting of:

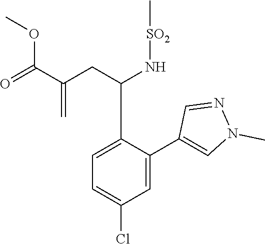 [SMILES: C=C(CC(NS(C)(=O)=O)c1ccc(Cl)cc1-c1cnn(C)c1)C(=O)OC]

### methyl-4-(4-chloro-2-(1-methyl-1H-pyrazol-4-yl)phenyl)-2-methylene-4-(methylsulfonamido)butanoate

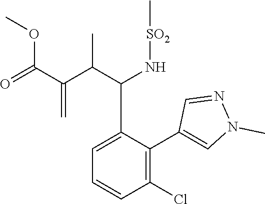 [SMILES: C=C(C(=O)OC)C(C)C(NS(C)(=O)=O)c1cccc(Cl)c1-c1cnn(C)c1]

### methyl-4-(3-chloro-2-(1-methyl-1H-pyrazol-4-yl)phenyl)-3-methyl-2-methylene-4-(methylsulfonamido)butanoate

28. A compound of formula (III):

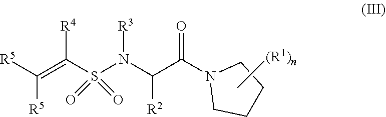

wherein:

n is 0 or an integer ranging from 1 to 4;

each R¹, R⁴, and R⁵ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof;

R² is selected from the group consisting of aryl, substituted aryl, heteroaryl, and substituted heteroaryl; and

R³ is selected from the group consisting of H, alkyl, alkenyl, alkynyl, cycloalkyl, heterocyclyl, aryl, heteroaryl, acyl, carbonyl, carboxy, and substituted versions thereof.

29. The compound of clause 28, wherein the compound has formula (IIIa):

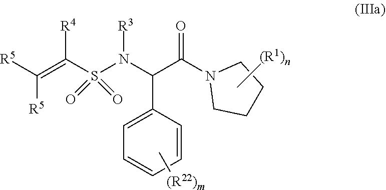

wherein:

m is 0 or an integer ranging from 1 to 5; and

each R²² is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

30. The compound of clause 28, wherein the compound has formula (IIIb):

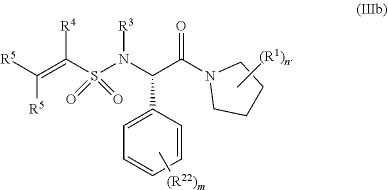

31. The compound of any one of clauses 29-30, wherein m ranges from 1 to 5 and at least one R²² is halo.
32. The compound of any one of clauses 29-31, wherein n is 0.
33. The compound of any one of clauses 29-32, wherein R³ is H.
34. The compound of any one of clauses 29-33, wherein R⁴ is H.
35. The compound of any one of clauses 29-34, wherein each R⁵ is H.
36. The compound of clause 28, wherein the compound has a structure selected from the group consisting of:

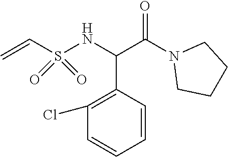 [SMILES: C=CS(=O)(=O)NC(C(=O)N1CCCC1)c1ccccc1Cl]

### N-(1-(2-chlorophenyl)-2-oxo-2-(pyrrolidin-1-yl)ethyl)ethenesulfonamide

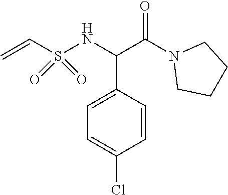 [SMILES: C=CS(=O)(=O)NC(C(=O)N1CCCC1)c1ccc(Cl)cc1]

### N-(1-(4-chlorophenyl)-2-oxo-2-(pyrrolidin-1-yl)ethyl)ethenesulfonamide

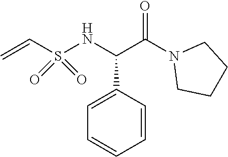 [SMILES: C=CS(=O)(=O)N[C@H](C(=O)N1CCCC1)c1ccccc1]

### (S)—N-(2-oxo-1-phenyl-2-(pyrrolidin-1-yl)ethyl)ethenesulfonamide and

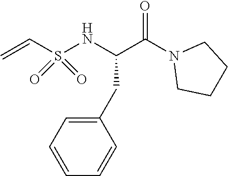 [SMILES: C=CS(=O)(=O)N[C@@H](Cc1ccccc1)C(=O)N1CCCC1]

### (S)—N-(1-oxo-3-phenyl-1-(pyrrolidin-1-yl)propan-2-yl)ethenesulfonamide

37. A compound of formula (IV):

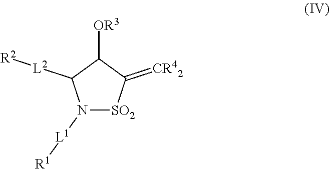

wherein:

R¹ and R² are each independently selected from H, alkyl, alkenyl, alkynyl, cycloalkyl, heterocyclyl, aryl, heteroaryl, acyl, and substituted versions thereof, provided that at least one of R¹ and R² is aryl, heteroaryl, or a substituted version thereof;

L¹ and L² are each independently absent or an alkylene group;

R³ is selected from the group consisting of H, alkyl, alkenyl, alkynyl, cycloalkyl, heterocyclyl, aryl, heteroaryl, acyl, and substituted versions thereof; and

R⁴ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

38. The compound of clause 37, wherein R¹ is aryl, substituted aryl, heteroaryl, or substituted heteroaryl.
39. The compound of clause 38, wherein the compound has formula (IVa):

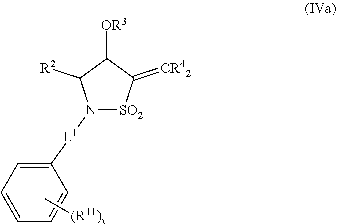

wherein:

x is 0 or an integer ranging from 1 to 5; and

each R¹¹ is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

40. The compound of clause 39, wherein x is an integer ranging from 1 to 5 and at least one R¹¹ is halo.
41. The compound of any one of clauses 39-40, wherein R² is H or alkyl.
42. The compound of any one of clauses 39-41, wherein L¹ is absent.
43. The compound of any one of clauses 39-41, wherein L¹ has the formula —CH₂—.
44. The compound of any one of clauses 39-43, wherein each R⁴ is H.
45. The compound of any one of clauses 39-40 or 42-44, wherein R² is aryl, substituted aryl, heteroaryl, or substituted heteroaryl.
46. The compound of clause 37, wherein the compound has formula (IVb):

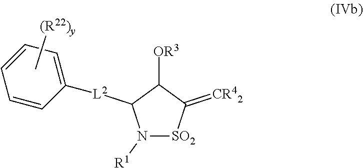

wherein:

y is 0 or an integer ranging from 1 to 5; and

each R²² is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

47. The compound of clause 46, wherein y is an integer ranging from 1 to 5 and at least one R²² is halo.
48. The compound of any one of clauses 46-47, wherein R¹ is H or alkyl.
49. The compound of any one of clauses 46-48, wherein L² is absent.
50. The compound of any one of clauses 46-48, wherein L² has the formula —CH₂—.
51. The compound of any one of clauses 46-50, wherein R³ is H or alkyl.
52. The compound of any one of clauses 46-51, wherein each R⁴ is H.
53. The compound clause 37, wherein the compound has a structure selected from the group consisting of:

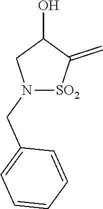 [SMILES: C=C1C(O)CN(Cc2ccccc2)S1(=O)=O]

### 2-benzyl-4-hydroxy-5-methyleneisothiazolidine 1,1-dioxide

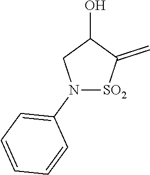

### 4-hydroxy-5-methylene-2-phenylisothiazolidine 1,1-dioxide and

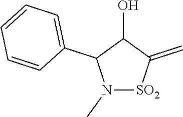 [SMILES: C=C1C(O)C(c2ccccc2)N(C)S1(=O)=O]

### 4-hydroxy-2-methyl-5-methylene-3-phenylisothiazolidine 1,1-dioxide

54. A compound of formula (V):

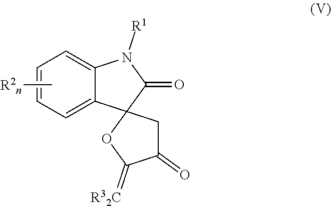

wherein:

R¹ is selected from the group consisting of H, alkyl, alkenyl, alkynyl, cycloalkyl, heterocyclyl, aryl, heteroaryl, acyl, carbonyl, carboxy, and substituted versions thereof;

each R² and R³ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and

n is 0 or an integer from 1 to 4.

55. The compound of clause 54, wherein the compound has formula (Va)

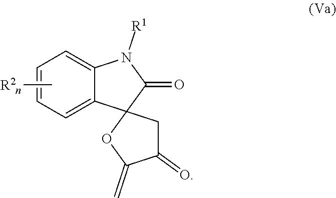

56. The compound of any one of clauses 54-55, wherein R¹ is alkyl.
57. The compound of clause 56, wherein R¹ is methyl.
58. The compound of any one of clauses 54-57, wherein n is 0.
59. The compound of clause 54, wherein the compound has a structure selected from the group consisting of:

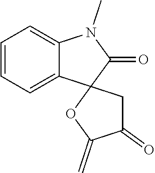 [SMILES: C=C1OC2(CC1=O)C(=O)N(C)c1ccccc12]

### 1′-methyl-5-methylene-3H-spiro[furan-2,3′-indoline]-2′,4 (5H)-dione

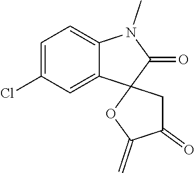 [SMILES: C=C1OC2(CC1=O)C(=O)N(C)c1ccc(Cl)cc12]

### 5′-chloro-1′-methyl-5-methylene-3H-spiro[furan-2,3′-indoline]-2′,4 (5H)-dione

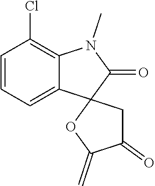 [SMILES: C=C1OC2(CC1=O)C(=O)N(C)c1c(Cl)cccc12]

### 7′-chloro-1′-methyl-5-methylene-3H-spiro[furan-2,3′-indoline]-2′,4 (5H)-dione and

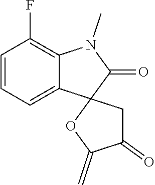 [SMILES: C=C1OC2(CC1=O)C(=O)N(C)c1c(F)cccc12]

### 7′-fluoro-1′-methyl-5-methylene-3H-spiro[furan-2,3′-indoline]-2′,4 (5H)-dione

60. A compound of formula (VI):

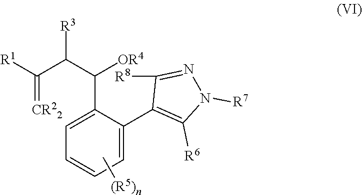

wherein:

n is 0, 1, 2, or 3;

each R¹, R², R³, R⁵, R⁶, and R⁸ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and

R⁴ and R⁷ are independently selected from the group consisting of H, alkyl, alkenyl, alkynyl, cycloalkyl, heterocyclyl, aryl, heteroaryl, acyl, carbonyl, carboxy, and substituted versions thereof.

61. The compound of clause 60, wherein R¹ is cyano or carboxy.
62. The compound of any one of clauses 60-61, wherein each R² is H and R³ is H.
63. The compound of any one of clauses 60-62, wherein R⁴ is H.
64. The compound of any one of clauses 60-63, wherein n is 1, 2, or 3 and at least one R^{s} is halo.
65. The compound of any one of clauses 60-64, wherein R⁶ is H and R⁸ is H.
66. The compound of any one of clauses 60-65, wherein R⁷ is alkyl or substituted alkyl.
67. The compound of clause 60, wherein the compound has a structure selected from the group consisting of:

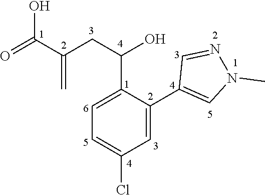 [SMILES: C=C(CC(O)c1ccc(Cl)cc1-c1cnn(C)c1)C(=O)O]

### 4-(4-chloro-2-(1-methyl-1H-pyrazol-4-yl)phenyl)-4-hydroxy-2-methylenebutanoic acid

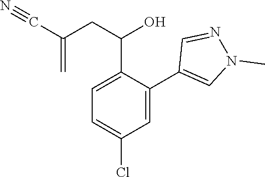 [SMILES: C=C(C#N)CC(O)c1ccc(Cl)cc1-c1cnn(C)c1]

### 4-(4-chloro-2-(1-methyl-1H-pyrazol-4-yl)phenyl)-4-hydroxy-2-methylenebutanenitrile

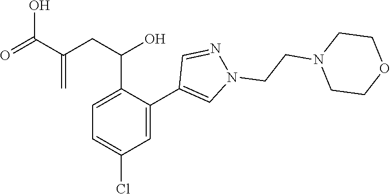 [SMILES: C=C(CC(O)c1ccc(Cl)cc1-c1cnn(CCN2CCOCC2)c1)C(=O)O]

### 4-(4-chloro-2-(1-(2-morpholinoethyl)-1H-pyrazol-4-yl)phenyl)-4-hydroxy-2-methylenebutanoic acid and

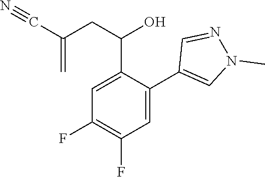 [SMILES: C=C(C#N)CC(O)c1cc(F)c(F)cc1-c1cnn(C)c1]

### 4-(4,5-difluoro-2-(1-methyl-1H-pyrazol-4-yl)phenyl)-4-hydroxy-2-methylenebutanenitrile

68. A pharmaceutical composition comprising a compound of any one of clauses 1-67.
69. The pharmaceutical composition of clause 68, further comprising a second compound.
70. The pharmaceutical composition of clause 69, wherein the second compound is imetelstat or imetelstat sodium.
71. A method of treating a patient for a telomerase-related condition, the method comprising: administering a pharmaceutical composition of any one of clauses 68-70.
72. The method of clause 71, wherein the compound is a telomerase inhibitor.
73. The method of clause 72, wherein the telomerase-related condition is a cancer.
74. The method of clause 73, wherein the cancer is a hematological malignancy.
75. The method of clause 73, wherein the cancer is selected from the group consisting of acute and chronic leukemias, lymphomas, multiple myeloma and myelodysplastic syndromes, myeloproliferative neoplasms (MPNs), essential thrombocythemia (ET), polycythemia vera (PV), Chronic Myelogenous Leukemia (CML), myelofibrosis (MF), acute myelogenous leukemia (AML), and myelodysplastic syndromes (MDS).
76. A method of treating a patient for a telomerase-related condition, the method comprising: administering a compound of any one of clauses 1-67.
77. The method of clause 76, wherein the compound is a telomerase inhibitor.
78. The method of clause 77, wherein the telomerase-related condition is a cancer.
79. The method of clause 78, wherein the cancer is a hematological malignancy.
80. The method of clause 79, wherein the cancer is selected from the group consisting of acute and chronic leukemias, lymphomas, multiple myeloma and myelodysplastic syndromes, myeloproliferative neoplasms (MPNs), essential thrombocythemia (ET), polycythemia vera (PV), Chronic Myelogenous Leukemia (CML), myelofibrosis (MF), acute myelogenous leukemia (AML), and myelodysplastic syndromes (MDS).

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); and the like.

### Example 1

### Synthesis of 3-methylene-5-(2-(1-(2-morpholinoethyl)-1H-pyrazol-4-yl)phenyl)dihydrofuran-2 (3H)-one

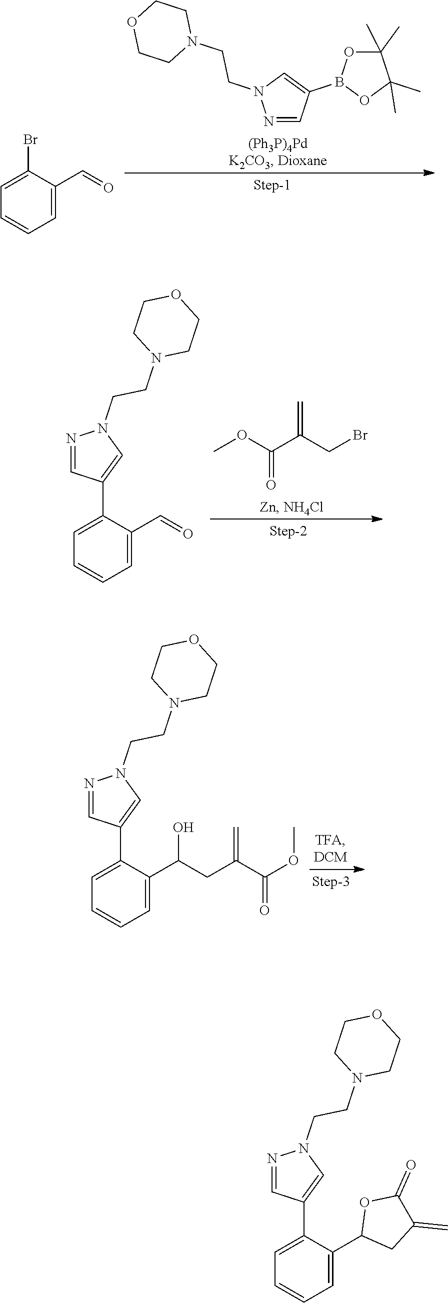

### Step-1: 2-(1-(2-morpholinoethyl)-1H-pyrazol-4-yl)benzaldehyde

To a stirred solution of 2-bromobenzaldehyde (0.2 g 1.08 mmol) 4-(2-(4-(4,4,5,5-tetramethyl-1,3,2-dioxaborolan-2-yl)-1H-pyrazol-1-yl)ethyl)morpholine (0.365 g 1.18 mmol) in 1,4-dioxane-H₂O (7 mL+3 mL) was added K₂CO₃ (0.447.5 g, 3.24 mmol). The reaction mixture was degassed with N₂ for 10 min and Pd(PPh₃)₄ (0.125 g, 0.010 mmol) was added. The _{reaction} mixture was stirred at 110° C. for overnight. The progress of the reaction was monitored by TLC. After completion of the starting material, it was diluted with H₂O (50 mL) and extracted with EtOAc (2×20 mL). The combined organic layer was dried over Na₂SO₄, filtered and evaporated under reduced pressure to afford 2-(1-(2-morpholinoethyl)-1H-pyrazol-4-yl)benzaldehyde. Yield 0.14 g (45.45%)

¹H-NMR (300 MHz, CDCl₃) δ 10.21 (s, 1H), 7.97 (dd, J=1.5, 8.7 Hz, 1H), 7.61 (d, J=5.1 Hz, 3H), 7.46 (d, J=2.7 Hz, 3H), 4.31 (t, J=6.3 Hz, 2H), 3.70 (t, J=4.5 Hz, 4H), 2.86 (t, J=6.6 Hz, 2H), 2.51 (t, J=4.8 Hz, 3H),

LCMS: Rt 1.08 min (91.27% purity), m/z 286.22 [M+H]⁺.

### Step-2: methyl 4-hydroxy-2-methylene-4-(2-(1-(2-morpholinoethyl)-1H-pyrazol-4-yl)phenyl)butanoate

To a stirred solution of 2-(1-(2-morpholinoethyl)-1H-pyrazol-4-yl)benzaldehyde (0.2 g 0.70 mmol) in THF (7 mL) was added methyl 2-(bromomethyl) acrylate (0.138 g, 0.77 mmol), sat.NH₄Cl (3 mL) and zinc (0.055 g, 0.84 mmol). Then the reaction mixture was stirred at room temperature for 16 h. The progress of the reaction was monitored by TLC. After completion of the starting material, the reaction mixture was diluted with ethyl acetate (50 mL) and washed with water (2×50 mL). The combined organic layer was dried over Na₂SO₄, filtered and evaporated under reduced pressure to get the crude compound, which was purified by silica gel column chromatography (eluent: 30% ethyl acetate in hexane) to afford methyl 4-hydroxy-2-methylene-4-(2-(1-(2-morpholinoethyl)-1H-pyrazol-4-yl)phenyl)-butanoate. Yield 0.18 g (66.6%).

¹H-NMR (300 MHz, CDCl₃) (7.65 (d, J=8.1 Hz, 1H), 7.38 (d, J=3.6 Hz, 2H), 7.35-7.28 (m, 3H), 6.25 (d, J=1.2 Hz, 1H), 5.65 (s, 1H), 5.08 (dd, J=3.0, 8.7 Hz, 1H), 4.37 (t, J=5.7 Hz, 2H), 3.79 (s, 7H), 2.96 (t, J=6.0 Hz, 2H), 2.78-2.62 (m, 7H).

LCMS: Rt 1.79 min (96.97% purity), m/z 386.31 [M+H]⁺.

### Step-3: 3-methylene-5-(2-(1-(2-morpholinoethyl)-1H-pyrazol-4-yl)phenyl)dihydrofuran-2 (3H)-one

To a stirred solution of methyl 4-hydroxy-2-methylene-4-(2-(1-(2-morpholinoethyl)-1H-pyrazol-4-yl)phenyl)butanoate (0.12 g 0.311 mmol) in DCM (10 mL), TFA (0.025 g) was added at room temperature and it was stirred for 16 h. The progress of the reaction was monitored by TLC. After completion of the starting material, the reaction mixture was concentrated under reduced pressure to get the crude compound. The crude obtained was purified by silica gel column chromatography (eluent: 15% EtOAc in hexane) to afford 3-methylene-5-(2-(1-(2-morpholinoethyl)-1H-pyrazol-4-yl)phenyl)dihydrofuran-2 (3H)-one. Yield 0.111 g (26.08%).

¹H-NMR (300 MHz, CDCl₃) δ 7.72 (s, 1H), 7.67 (s, 1H), 7.42-7.37 (m, 3H), 7.36-7.27 (m, 1H), 6.29 (t, J=2.7 Hz, 1H), 5.70-7.67 (m, 1H), 5.65-5.62 (m 1H), 4.72 (d, J=5.7 Hz, 2H), 3.95 (t, J=4.5 Hz, 4H), 3.61 (s, 2H), 3.37-3.29 (m, 2H), 3.10 (bs, 5H), 3.00-2.91 (m, 2H).

LCMS: Rt 1.88 min (97.72% purity), m/z 354.25 [M+H]⁺.

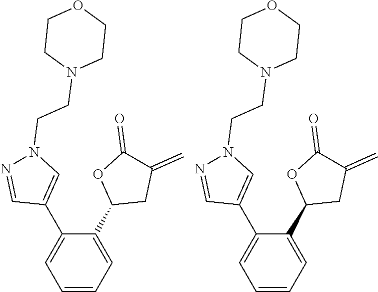

Note: Both enantiomers were_{synthesized} by chiral HPLC purification (stereochemistry of both enantiomers was tentatively assigned).

Peak-1 (left structure above):

¹H-NMR (300 MHz, CDCl3) δ 8.01 (s, 1H), 7.73 (s, 1H), 7.39 (d, J=5.7 Hz, 4H), 6.14 (t, J=2.4 Hz, 1H), 5.81-5.77 (m, 2H), 4.58 (t, J=6.0 Hz, 2H), 3.80 (bs, 4H), 3.46-3.37 (m, 2H), 3.16 (bs, 4H), 2.94-2.87 (m, 2H),

LCMS: Rt 1.84 min (97.20% purity), m/z 354.30 [M+H]⁺.

Peak-2 (right structure above):

¹H-NMR (300 MHz, CDCl3) δ 7.67 (s, 1H), 7.63 (s, 1H), 7.44-7.28 (m, 4H), 6.30 (t, J=2.7 Hz, 1H), 5.70-5.63 (m, 2H), 4.69 (t, J=6.0 Hz, 2H), 3.93 (t, J=4.5 Hz, 4H), 3.61 (t, J=5.7 Hz, 2H), 3.37-3.29 (m, 1H), 3.08 (bs, 4H), 2.98-2.90 (m, 1H).

LCMS: Rt 1.84 min (98.55% purity), m/z 354.30 [M+H]⁺.

Following analogous compounds were synthesized using this protocol:

### Methyl 4-hydroxy-2-methylene-4-(2-(1-(2-(piperidin-1-yl) ethyl)-1H-pyrazol-4-yl) phenyl) butanoate

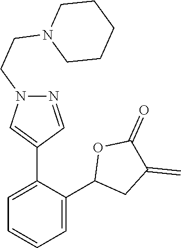 [SMILES: C=C1CC(c2ccccc2-c2cnn(CCN3CCCCC3)c2)OC1=O]

¹H-NMR (300 MHz, CDCl₃) δ: 7.70 (s, 1H), 7.61 (s, 1H), 7.44-7.30 (m, 4H), 6.30 (t, J=1.8 Hz, 1H), 5.70-5.63 (m, 2H), 4.72 (q, J=6.3 Hz, 2H), 3.62-3.57 (m, 2H), 3.42-3.33 (m, 3H). 2.96-2.89 (m, 1H), 1.75 (bs, 4H), 1.41 (bs, 2H).

LCMS: Rt 1.92 min (99.04% purity), m/z 352.23[M+H]⁺.

### 3-Methylene-5-(2-(1-(prop-2-yn-1-yl)-1H-pyrazol-4-yl) phenyl) dihydrofuran-2 (3H)-one

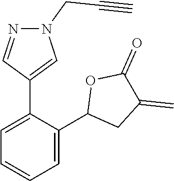 [SMILES: C#CCn1cc(-c2ccccc2C2CC(=C)C(=O)O2)cn1]

¹H-NMR (300 MHz, CDCl₃) δ: 7.70 (s, 1H), 7.57 (s, 1H), 7.44-7.31 (m, 4H), 6.30 (t, J=2.7 Hz, 1H), 5.74-5.66 (m, 2H), 5.01 (d, J=2.7 Hz, 2H), 3.27-2.18 (m, 1H), 2.97-2.89 (m, 1H), 2.55 (t, J=2.4 Hz, 1H).

LCMS: Rt 2.42 min (99.63% purity), m/z 279.16 [M+H]⁺.

### 5-(2-(2-methyl-1H-imidazol-1-yl) phenyl)-3-methylenedihydrofuran-2 (3H)-one

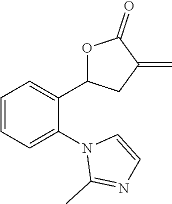 [SMILES: C=C1CC(c2ccccc2-n2ccnc2C)OC1=O]

¹H-NMR (300 MHz, CDCl₃) δ:7.62-7.50 (m, 3H), 7.28 (s, 1H), 7.21 (s, 1H), 7.05-6.92 (m, 1H), 6.31 (s, 1H), 5.69 (d, J=2.1 Hz, 1H), 5.29 (t, J=7.2 Hz, 1H), 5.11 (t, J=7.5 Hz, 1H). 3.17-2.84 (m, 1H), 2.82-2.77 (m, 1H), 2.77-2.69 (m, 3H).

LCMS: Rt 069 min (99.29% purity), m/z 255.11 [M+H]⁺.

### 5-(2-(2-methoxypyrimidin-4-yl)phenyl)-3-methylenedihydrofuran-2 (3H)-one

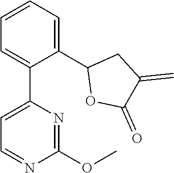 [SMILES: C=C1CC(c2ccccc2-c2ccnc(OC)n2)OC1=O]

¹H-NMR (300 MHz, CDCl₃) 8.51 (s, 2H), 7.54-7.42 (m, 3H), 7.42-7.23 (m, 1H), 6.31 (t, J=2.7 Hz, 2H), 5.68 (t, J=2.4 Hz, 1H), 5.47 (q, J=6.6 Hz, 1H), 4.08 (s, 3H), 3.23-3.13 (m, 1H), 2.95-2.87 (m, 1H);

LCMS (m/z): 282 [M+H]⁺.

### 5-(4′-methoxy-[1,1′-biphenyl]-2-yl)-3-methylenedihydrofuran-2 (3H)-one

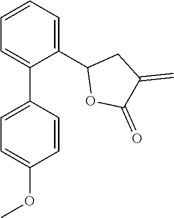 [SMILES: C=C1CC(c2ccccc2-c2ccc(OC)cc2)OC1=O]

¹H-NMR (300 MHz, CDCl₃) 7.47-7.35 (m, 3H), 7.24-7.20 (m, 2H), 6.99-6.95 (m, 2H), 6.26 (t, J=2.7 Hz, 3H), 5.62-5.57 (m, 2H), 3.86 (s, 3H), 3.13-3.031 (m, 1H), 2.88-2.78 (m, 1H);

Mass: 354.3 [M+H]⁺.

### 3-methylene-5-(2-(1-propyl-1H-pyrazol-4-yl)phenyl)dihydrofuran-2 (3H)-one

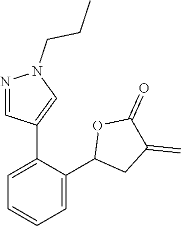 [SMILES: C=C1CC(c2ccccc2-c2cnn(CCC)c2)OC1=O]

¹H-NMR (300 MHz, CDCl₃) δ: 7.53-7.48 (m, 2H), 7.44-7.41 (m, 1H), 7.38-7.29 (m, 3H), 6.29 (t, J=3.0 Hz, 1H), 5.75-5.70 (m, 1H), 5.66 (t, J=2.4 Hz, 1H), 4.13 (t, J=6.9 Hz, 2H), 3.26-3.18 (m, 1H), 2.97-2.89 (m, 1H), 1.94 (q, J=7.5 Hz, 2H), 0.96 (t, J=7.5 Hz, 3H),

LCMS: Rt 2.49 min (98.12% purity), m/z 283.19 [M+H]⁺.

### 5-(2-(1-cyclopropyl-1H-pyrazol-4-yl)phenyl)-3-methylenedihydrofuran-2 (3H)-one

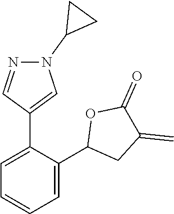 [SMILES: C=C1CC(c2ccccc2-c2cnn(C3CC3)c2)OC1=O]

¹H-NMR (300 MHz, CDCl₃) δ: 7.54-7.49 (m, 2H), 7.42-7.39 (m, 1H), 7.36-7.27 (m, 3H), 6.27 (t, J=2.7 Hz, 1H), 5.71-5.64 (m, 2H), 3.67-3.60 (m, 1H), 3.26-3.17 (m, 1H), 2.96-2.87 (m, 1H) 1.20-1.15 (m, 2H), 1.03-1.09 (m, 2H)

LCMS (m/z): 281.24 [M+H]

### 3-methylene-5-(2-(thiazol-2-yl)phenyl)dihydrofuran-2 (3H)-one

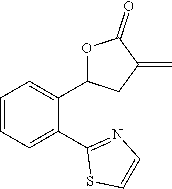 [SMILES: C=C1CC(c2ccccc2-c2nccs2)OC1=O]

¹H-NMR (300 MHz, CDCl₃) δ: 7.92 (d, J=1.8 Hz, 1H), 7.68 (dd, J=1.2&7.5 Hz, 1H), 7.57 (dd, J=1.5&4.8 Hz, 1H), 7.53-7.46 (m, 1H), 7.46-7.39 (m, 2H), 6.31-6.24 (m, 2H), 5.65 (t, J=2.4 Hz, 1H), 3.64-3.54 (m, 1H), 2.85-2.76 (m, 1H);

LCMS (m/z): 258.14 [M+H]

### 5-(2-(1-isopropyl-1H-pyrazol-4-yl)phenyl)-3-methylenedihydrofuran-2 (3H)-one

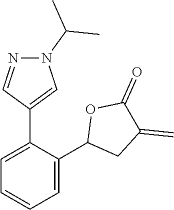 [SMILES: C=C1CC(c2ccccc2-c2cnn(C(C)C)c2)OC1=O]

¹H-NMR (300 MHz, CDCl₃) δ: 7.57-7.53 (m, 2H), 7.43-7.41 (m, 1H), 7.37-7.30 (m, 3H), 6.30 (t, J=2.7 Hz, 1H), 5.73-7.66 (m, 2H), 4.62-4.53 (m, 1H), 3.28-3.17 (m, 1H), 2.99-2.89 (m, 1H), 1.56 (s, 6H):

LCMS: Rt 2.79 min (98.25% purity), m/283.24 [M+H]⁺.

### 3-Methylene-5-(3′-(trifluoromethyl)-[1,1′-biphenyl]-2-yl) dihydrofuran-2 (3H)-one

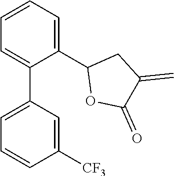 [SMILES: C=C1CC(c2ccccc2-c2cccc(C(F)(F)F)c2)OC1=O]

300 M Hz, CDCl₃) 7.58 (d, J=3 Hz, 1H), 7.56-7.51 (m, 3H), 7.49-7.38 (m, 3H), 7.28 (d, J=1.5 Hz, 1H), 6.28 (t, J=3 Hz, 1H), 5.64 (t, J=2.4 Hz, 1H), 5.47 (q, J=6.6 Hz, 3H), 3.14-3.04 (m, 1H), 2.92-2.82 (m, 1H);

LCMS (m/z): 318 [M+H]⁺.

### 5-(2-(6-Methoxypyridin-3-yl) phenyl)-3-methylenedihydrofuran-2 (3H)-one

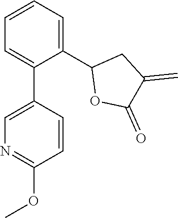

¹H-NMR (300 MHz, CDCl₃) δ: 8.15 (d, J=2.1 Hz, 1H), 7.63 (dd, J=2.4, 8.4 Hz, 1H), 7.50-7.38 (m, 3H), 7.23 (d, J=1.5 Hz, 2H), 6.87 (dd, J=0.6, 8.7 Hz, 1H), 6.28 (t, J=2.7 Hz, 1H), 5.65 (t, J=2.4 Hz, 1H), 5.51 (q, J=6.6 Hz, 1H), 4.01 (s, 3H), 3.20-312 (m, 1H), 2.92-2.84 (m, 1H);

MS (m/z): 281 [M+H]⁺.

### 5-(2-methoxyphenyl)-3-methylenedihydrofuran-2 (3H)-one

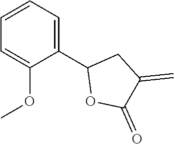

1H-NMR (300 M Hz, CDCl₃) δ: 7.34-7.28 (m, 2H), 6.99-6.91 (m, 1H), 6.88 (d, J=0.9 Hz, 1H), 6.28 (t, J=3 Hz, 1H), 5.72 (q, J=2.4 Hz, 1H), 5.62 (t, J=2.4 Hz, 1H), 3.83 (s, 3H), 3.47-3.37 (m, 1H), 2.88-2.79 (m, 1H);

MS (m/z): 204 [M+H]⁺.

### 3-methylene-5-(3-phenoxyphenyl) dihydrofuran-2 (3H)-one

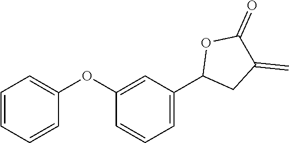 [SMILES: C=C1CC(c2cccc(Oc3ccccc3)c2)OC1=O]

¹H-NMR (300 MHz, CDCl₃) δ: 7.37-7.31 (m, 3H), 7.15-7.04 (m, 1H), 7.02-6.94 (m, 5H), 6.30 (t, J=3.0 Hz, 1H), 5.68 (t, J=2.4 Hz, 1H), 5.48 (t, J=7.2 Hz, 1H), 3.42-3.34 (m, 1H), 3.33-2.86 (m, 1H),

LCMS: Rt 6.31 min (95.71% purity); m/z 267.1 [M+H]⁺.

### 3-methylene-5-(4-phenoxyphenyl)dihydrofuran-2 (3H)-one

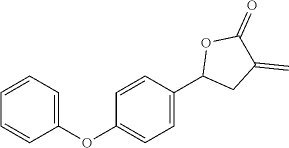 [SMILES: C=C1CC(c2ccc(Oc3ccccc3)cc2)OC1=O]

¹H-NMR (300 MHz, CDCl₃) δ: 7.37-7.27 (m, 4H), 7.15-7.02 (m, 1H), 7.02-6.99 (m, 4H), 6.31 (t, J=2.7 Hz, 1H), 5.50 (t, J=6.6 Hz, 1H), 3.43-3.34 (m, 1H), 2.97-289 (m, 1H),

LCMS: Rt 2.72 min (94.91% purity), m/z 267.14 [M+H]⁺.

### 3-methylene-5-(quinolin-8-yl)dihydrofuran-2 (3H)-one

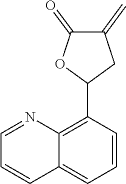 [SMILES: C=C1CC(c2cccc3cccnc23)OC1=O]

¹H-NMR (300 MHz, CDCl₃) δ: 8.89 (q, J=1.8 Hz, 1H), 8.18 (dd, J=1.8&8.4 Hz, 1H), 7.78 (q, J=8.4 Hz, 2H), 7.58-7.52 (m, 1H), 7.47-7.43 (m, 1H), 6.60-6.58 (m, 1H), 6.31 (t, J=2.7 Hz, 1H), 5.63 (t, J=2.4 Hz, 1H), 3.83-3.73 (m, 1H), 2.94-2.85 (m, 1H):

MS (m/z): 226.2 [M+H]⁺.

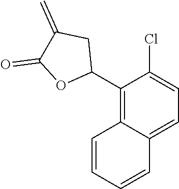 [SMILES: C=C1CC(c2c(Cl)ccc3ccccc23)OC1=O]

### 5-(2-chloronaphthalen-1-yl)-3-methylenedihydrofuran-2 (3H)-one

¹H-NMR (300 MHz, CDCl₃) δ: 7.91-7.83 (m, 2H), 7.78 (d, J=8.7 Hz, 1H), 7.56-7.43 (m, 3H), 6.56 (t, J=8.4 Hz, 1H), 6.47 (q, J=2.4 Hz, 1H), 5.80 (t, J=2.4 Hz, 1H), 3.47-3.25 (m, 2H),

LCMS: Rt 2.68 min (97.47% purity), m/z 300.12 [M+H]⁺.

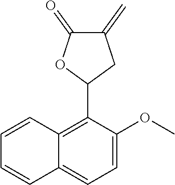 [SMILES: C=C1CC(c2c(OC)ccc3ccccc23)OC1=O]

### 5-(2-methoxynaphthalen-1-yl)-3-methylenedihydrofuran-2 (3H)-one

¹H-NMR (300 MHz, CDCl₃) δ: 7.86-7.81 (m, 3H), 7.53 (t, J=1.2 Hz, 1H), 7.48 (d, J=1.5 Hz, 1H), 7.40-7.27 (m, 1H), 6.50 (t, J=6.3 Hz, 1H), 6.36 (t, J=3.0 Hz, 1H), 5.67 (t, J=2.4 Hz, 1H), 3.91 (s, 3H), 3.42-3.21 (m, 2H):

LCMS: Rt 2.57 min (96.48% purity), m/z 255.10 [M+H]⁺.

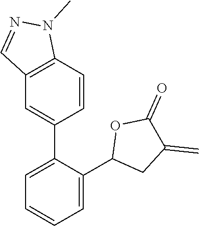 [SMILES: C=C1CC(c2ccccc2-c2ccc3c(cnn3C)c2)OC1=O]

### 5-(2-(1-methyl-1H-indazol-5-yl)phenyl)-3-methylenedihydrofuran-2 (3H)-one

¹H-NMR (300 MHz, CDCl₃) δ: 8.09 (s, 1H), 7.67 (t, J=0.6 Hz, 1H), 7.51-7.45 (m, 2H), 7.43-7.38 (m, 2H), 7.32-7.25 (m, 2H), 5.60-5.55 (m, 2H), 4.15 (s, 3H), 3.07-2.98 (m, 1H), 2.91-2.82 (m, 1H).

LCMS (m/z): 305.17 [M+H]⁺.

### 3-methylene-5-(2-(1-propyl-1H-pyrazol-4-yl)phenyl)dihydrofuran-2 (3H)-one)

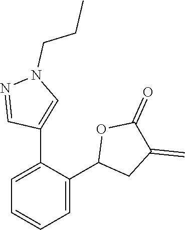 [SMILES: C=C1CC(c2ccccc2-c2cnn(CCC)c2)OC1=O]

¹H-NMR (300 MHz, CDCl₃) δ: 7.53-7.48 (m, 2H), 7.44-7.41 (m, 1H), 7.38-7.29 (m, 3H), 6.29 (t, J=3.0 Hz, 1H), 5.75-5.70 (m, 1H), 5.66 (t, J=2.4 Hz, 1H), 4.13 (t, J=6.9 Hz, 2H), 3.26-3.18 (m, 1H), 2.97-2.89 (m, 1H), 1.94 (q, J=7.5 Hz, 2H), 0.96 (t, J=7.5 Hz, 3H),

LCMS: Rt 2.49 min (98.12% purity), m/z 283.19 [M+H]+

### 5-(2-(2-methyl-2H-indazol-5-yl)phenyl)-3-methylenedihydrofuran-2 (3H)-one

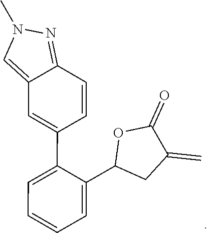

¹H-NMR (300 MHz, CDCl₃) δ: 7.93 (s, 1H), 7.73 (d, J=8.7 Hz, 2H), 7.68-7.61 (m, 1H), 7.54-7.46 (m, 2H), 7.46-7.37 (m, 2H), 7.37-7.33 (m, 2H), 7.31-7.28 (m, 1H), 7.19 (dd, J=3&9 Hz, 1H):

LCMS (m/z): 305.16 [M+H]⁺.

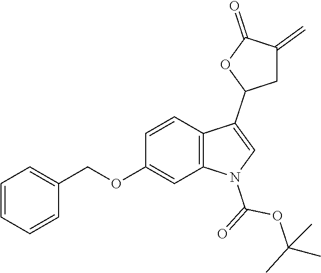 [SMILES: C=C1CC(c2cn(C(=O)OC(C)(C)C)c3cc(OCc4ccccc4)ccc23)OC1=O]

### tert-butyl-6-(benzyloxy)-3-(4-methylene-5-oxotetrahydrofuran-2-yl)-1H-indole-1-carboxylate

¹H-NMR (300 MHz, CDCl₃) δ: 7.86 (bs, 1H), 7.46 (d, J=8.9 Hz, 3H), 7.42-7.32 (m, 5H), 6.96 (dd, J=2.4&8.7 Hz, 1H), 6.35 (t, J=3.0 Hz, 1H), 5.77-5.72 (m, 2H), 5.13 (s, 1H), 3.81-3.45 (m, 1H), 3.18-3.09 (m, 1H), 1.65 (s, 9H),

LCMS: Rt 3.12 min (99.71% purity), m/z 413.26 [M+H]⁺.

### 5-(2-(1,3-dimethyl-1H-pyrazol-4-yl)phenyl)-3-methylenedihydrofuran-2 (3H)-one

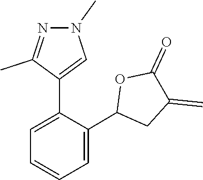

¹H-NMR (300 MHz, CDCl₃) δ:7.45-7.30 (m, 3H), 7.30 (s, 1H), 7.21-7.18 (m, 1H), 6.27-6.21 (m, 1H), 5.62 (t, J=2.4 Hz, 1H), 5.54 (q, J=6.6 Hz, 1H), 3.95 (s, 3H), 3.72 (s, 2H), 3.16-3.07 (m, 1H), 2.86-2.78 (m, 1H), 2.63-2.54 (m, 1H),

LCMS: Rt 2.63 min (96.31% purity), m/z 269.15 [M+H]⁺.

### 3-methylene-5-(2-(1-phenethyl-1H-pyrazol-4-yl)phenyl)dihydrofuran-2 (3H)-one

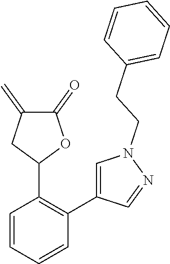 [SMILES: C=C1CC(c2ccccc2-c2cnn(CCc3ccccc3)c2)OC1=O]

¹H-NMR (400 MHz, CDCl₃): 7.56 (s, 1H), 7.55-7.37 (m, 1H), 7.36-7.27 (m, 1H), 7.24-7.17 (m, 4H), 7.11 (s, 1H), 7.09-7.07 (m, 2H), 6.32-6.30 (t, 1H), 5.66-5.65 (t, 1H), 5.44-5.41 (m, 1H), 4.43-4.39 (t, 2H), 3.22-3.19 (t, 2H), 3.05-2.99 (m, 1H), 2.86-2.80 (m, 1H).

### 5-(4-methyl-2-(1-methyl-1H-pyrazol-4-yl)phenyl)-3-methylenedihydrofuran-2 (3H)-one

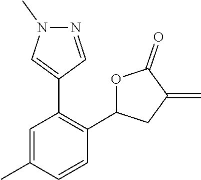

¹H-NMR (400 MHz, CDCl₃): 7.525 (s, 1H), 7.46 (s, 1H), 7.32-7.30 (d, J=8 Hz, 1H), 7.18-7.16 (d, J=8.4 Hz, 1H), 7.12 (s, 1H), 6.29-6.28 (t, 1H), 5.70-5.65 (m, 2H), 3.97 (s, 3H), 3.24-3.16 (m, 1H), 2.97-2.90 (m, 1H), 2.36 (s, 3H).

### 5-(5-methyl-2-(1-methyl-1H-pyrazol-4-yl)phenyl)-3-methylenedihydrofuran-2 (3H)-one

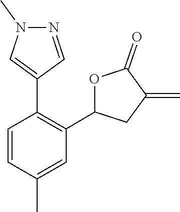

¹H-NMR (400 MHz, CDCl₃): 7.50 (s, 1H), 7.43 (s, 1H), 7.22 (s, 1H), 7.19-7.14 (m, 2H), 6.30-6.29 (t, 1H), 5.71-5.66 (m, 2H), 3.97 (s, 3H), 3.25-3.17 (m, 1H), 2.95-2.88 (m, 1H), 2.39 (s, 3H).

### 5-(3-methyl-2-(1-methyl-1H-pyrazol-4-yl)phenyl)-3-methylenedihydrofuran-2 (3H)-one

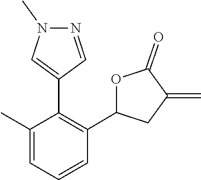

1H-NMR (400 MHz, CDCl3): 7.38 (s, 1H), 7.31-7.23 (m, 4H), 6.25-6.23 (t, 1H), 5.60-5.59 (t, 1H), 3.90 (s, 3H), 3.10-3.04 (m, 1H), 2.82-2.76 (m, 1H), 2.13 (s, 3H).

### 5-(2-(1-methyl-1H-pyrazol-4-yl)-4-phenoxyphenyl)-3-methylenedihydrofuran-2 (3H)-one

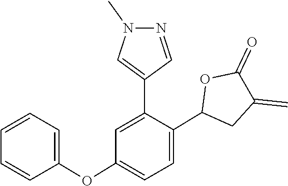 [SMILES: C=C1CC(c2ccc(Oc3ccccc3)cc2-c2cnn(C)c2)OC1=O]

¹H-NMR (300 MHz, CDCl₃) δ: 7.48 (d, J=10.5 Hz, 2H), 7.38-7.32 (m, 3H), 7.15-7.10 (m, 1H), 7.03 (d, J=7.8 Hz, 2H), 6.98-6.92 (m, 2H), 6.29 (t, J=2.4 Hz, 1H), 5.67 (t, J=9.0 Hz, 2H), 3.94 (s, 3H), 3.26-3.18 (m, 1H), 3.01-2.94 (m, 1H).

LCMS: Rt 2.65 min (99.57% purity), m/z 347.25 [M+H]⁺.

### 5-(3-(1-methyl-1H-pyrazol-4-yl)-[1,1′-biphenyl]-4-yl)-3-methylenedihydrofuran-2 (3H)-one

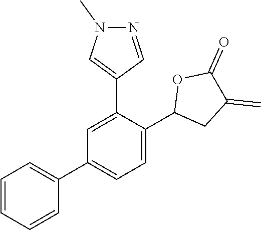 [SMILES: C=C1CC(c2ccc(-c3ccccc3)cc2-c2cnn(C)c2)OC1=O]

¹H-NMR (300 MHz, CDCl3) δ 7.61-7.57 (m, 4H), 7.52 (t, J=1.8 Hz, 2H), 7.49-7.42 (m, 2H), 7.39-7.34 (m, 1H), 6.31 (t, J=3.0 Hz, 1H), 5.76 (q, J=6.0 Hz, 1H), 5.69 (t, J=2.4 Hz, 1H), 3.99 (s, 3H), 3.31-3.20 (m, 1H), 3.03-2.93 (m, 1H).

LCMS: Rt 2.67 min (98.85% purity), m/z 331.21 [M+H]⁺.

### 5-(4,5-dichloro-2-(1-methyl-1H-pyrazol-4-yl)phenyl)-3-methylenedihydrofuran-2 (3H)-one

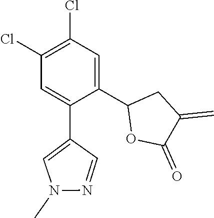 [SMILES: C=C1CC(c2cc(Cl)c(Cl)cc2-c2cnn(C)c2)OC1=O]

1H-NMR (400 MHz, CDCl3): 7.53-7.50 (s, 2H), 7.48-7.46 (s, 1H), 7.42-7.38 (s, 1H), 6.35-6.32 (t, 1H), 5.7 (t, 1H), 5.66-5.59 (m, 1H), 4.0 (s, 3H), 3.39-3.29 (m, 1H), 2.8-2.6 (m, 1H).

### 5-(1,1′-dimethyl-1H, 1′H-[4,4′-bipyrazol]-5-yl)-3-methylenedihydrofuran-2 (3H)-one

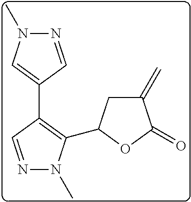 [SMILES: C=C1CC(c2c(-c3cnn(C)c3)cnn2C)OC1=O]

¹H-NMR (300 MHz, CDCl₃) δ: 7.40 (dd, J=0.6 & 5.1 Hz, 2H), 7.31 (s, 1H), 6.33 (t, J=2.7 Hz, 1H), 5.71-5.65 (m, 2H), 3.91 (s, 6H), 3.27-3.18 (m, 1H), 3.13-3.02 (m, 1H).

LCMS: Rt 1.55 min (95.51% purity), m/z 259.15[M+H]⁺.

### Synthesis of Lactam Analogs

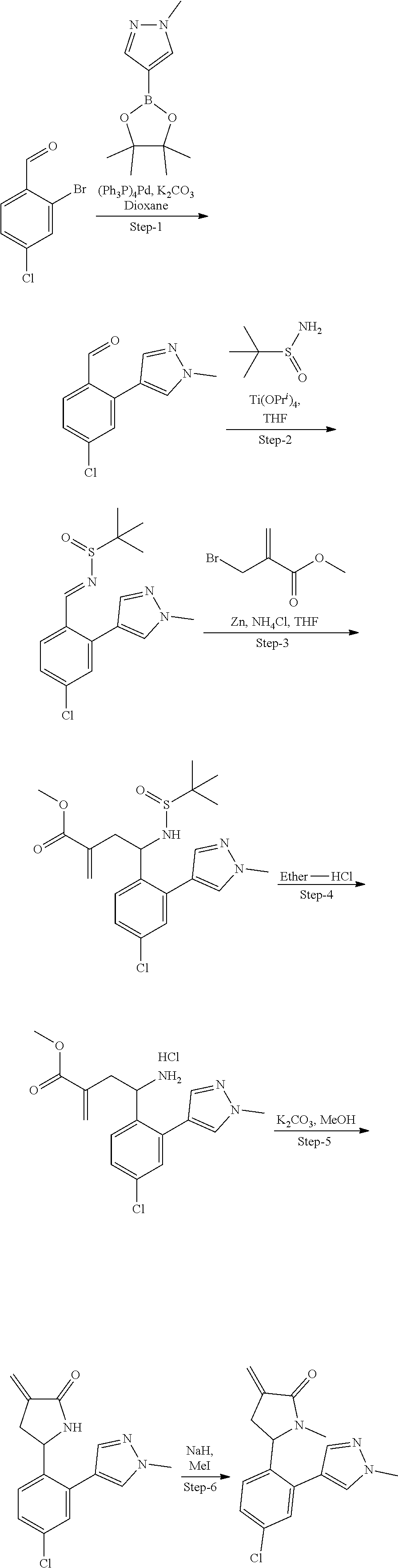

### Step-1: Synthesis of 4-chloro-2-(1-methyl-1H-pyrazol-4-yl)benzaldehyde

To a stirred solution of 2-bromo-4-chlorobenzaldehyde (1 g, 4.5 mmol) and 1-methyl-4-(4,4,5,5-tetramethyl-1,3,2-dioxaborolan-2-yl)-1H-pyrazole (1.18 g, 5.46 mmol) in 1,4-Dioxanne (15 mL), potassium carbonate (1.5 g, 11.83 mmol) in water (10 mL) was added. The resulting mixture was degassed for 5 min, and tetrakis(triphenylphosphine)-palladium (100 mg, 10% mol) was added. The reaction mixture was again degassed for additional 5 min. The reaction mixture was heated at 90° C. for 2 h. Reaction progress was monitored by TLC. Upon disappearance of starting material, the reaction mixture was diluted with ethyl acetate (40 mL) and washed with water (2×30 mL). The organic layer was dried over Na₂SO₄, filtered and then solvent was evaporated under reduced pressure to yield the crude material. It was purified by column chromatography using silica gel (35-50% ethyl acetate and petroleum ether as eluent) to afford 4-chloro-2-(1-methyl-1H-pyrazol-4-yl)benzaldehyde as pale yellow solid (0.9 g, 89%). 1H-NMR (400 MHz, CDCl3): 10.159 (s, 1H), 7.89-7.87 (d, J=8.8 Hz, 1H), 7.6 (s, 1H), 7.49 (s, 1H), 7.26-7.22 (m, 2H), 3.99 (s, 3H), 2.43 (s, 3H).

### Step-2: Synthesis of N-(4-chloro-2-(1-methyl-1H-pyrazol-4-yl)benzylidene)-2-methylpropane-2-sulfinamide

To a stirred solution of 4-chloro-2-(1-methyl-1H-pyrazol-4-yl) benzaldehyde (0.6 g, 2.7 mmol) in tetrahydrofuran (10 mL), were added 2-methylpropane-2-sulfinamide (0.39 g, 3.26 mmol), and Titanium isopropoxide (1.95 g, 6.8 mmol) at room temperature. The resulting reaction mixture was stirred at room temperature for 16 h. Reaction progress was monitored by TLC. Upon disappearance of starting material, the reaction mixture was quenched with water (50 mL) and the solid was filtered. The filtrate was extracted with ethyl acetate (2×25 mL). The combined organic layer was dried over Na₂SO₄, filtered and then solvent was evaporated under reduced pressure to afford the crude material. It was purified by column chromatography using silica mesh (35-40% ethyl acetate in petroleum ether as eluent) to afford N-(4-chloro-2-(1-methyl-1H-pyrazol-4-yl)benzylidene)-2-methylpropane-2-sulfinamide as a pale yellow gum (550 mg, 62.5%).

### Step-3: Synthesis of methyl 4-((tert-butylsulfinyl)amino)-4-(4-chloro-2-(1-methyl-1H-pyrazol-4-yl)phenyl)-2-methylenebutanoate

To a stirred solution of N-(4-chloro-2-(1-methyl-1H-pyrazol-4-yl)benzylidene)-2-methylpropane-2-sulfinamide (0.4 g, 1.23 mmol) and methyl 2-(bromomethyl)acrylate (280 mg, 1.66 mmoL) in THF (4 mL), Zinc (290 mg, 4.48 mmol) and saturated NH₄Cl in water (2 mL) was added. The resulting mixture was stirred at RT for 16 h. Reaction progress was monitored by TLC. 50% of starting material consumed by TLC, the reaction mixture was filtered, and filtrate was extracted with ethyl acetate (20 mL) and washed with water (2×10 mL). The organic layer was dried over Na₂SO₄, filtered and then solvent was evaporated under reduced pressure to afford crude material as dark yellow gum. It was purified by column chromatography using silica gel column chromatography (5-10% MeOH in DCM as eluent) to afford (E)-N-(4-chloro-2-(1-methyl-1H-pyrazol-4-yl)benzylidene)-2-methylpropane-2-sulfinamide as a pale-yellow gum (150 mg, 30%).

### Step-4: Synthesis of methyl 4-amino-4-(4-chloro-2-(1-methyl-1H-pyrazol-4-yl)phenyl)-2-methylenebutanoate

To a stirred solution of methyl 4-((tert-butylsulfinyl)amino)-4-(4-chloro-2-(1-methyl-1H-pyrazol-4-yl)phenyl)-2-methylenebutanoate (0.15 g, 0.354 mmol) in diethyl ether (2 mL) and THF (2 mL), HCl (2M in diethyl ether, 1.5 mL) was added at room temperature and the resulting mixture was stirred at RT for 15 min. The progress of the reaction was monitored by TLC. Upon disappearance of starting material, the reaction mixture was concentrated under reduced pressure and low temperature to afford product as a methyl 4-amino-4-(4-chloro-2-(1-methyl-1H-pyrazol-4-yl)phenyl)-2-methylenebutanoate as an off-white gum (100 mg, 88%).

### Step-5: Synthesis of 5-(4-chloro-2-(1-methyl-1H-pyrazol-4-yl)phenyl)-3-methylenepyrrolidin-2-one

To a stirred solution of methyl 4-amino-4-(4-chloro-2-(1-methyl-1H-pyrazol-4-yl)phenyl)-2-methylenebutanoate (0.1 g, 0.31 mmol)) in MeOH (4 mL), K₂CO₃ (90 mg, 0.62 mmol) was added at room temperature and the resulting mixture was stirred at RT for 16 h. Reaction progress was monitored by TLC. Upon disappearance of starting material, the reaction mixture was concentrated under reduced pressure and residue was diluted with DCM (20 mL) and washed with water (2×10 mL). The organic layer was dried over Na₂SO₄, filtered and evaporated under reduced pressure to afford crude 5-(4-chloro-2-(1-methyl-1H-pyrazol-4-yl)phenyl)-3-methylenepyrrolidin-2-one as pale-yellow gum. The crude compound thus obtained was purified by column chromatography using silica mesh column chromatography (2-5% MeOH in DCM as eluent) to afford N-(4-chloro-2-(1-methyl-1H-pyrazol-4-yl)benzylidene)-2-methylpropane-2-sulfinamide as an off white gum (60 mg, 67%).

¹H-NMR (400 MHz, CDCl₃): 7.48 (s, 1H), 7.40 (s, 1H), 7.36-7.26 (m, 2H), 6.13-6.07 (m, 2H), 5.40 (m, 2H), 3.97 (s, 1H), 4.97-4.94 (m, 1H), 3.23-3.18 (m, 1H), 2.7-2.6 (m, 1H).

MS: 287.9

### Step-6: Synthesis of 5-(4-chloro-2-(1-methyl-1H-pyrazol-4-yl)phenyl)-1-methyl-3-methylenepyrrolidin-2-one

To a stirred solution of N-(4-chloro-2-(1-methyl-1H-pyrazol-4-yl)benzylidene)-2-methylpropane-2-sulfinamide (50 mg, 0.31 mmol) in THF (4 mL), NaH (90 mg, 0.62 mmol) was added followed by iodomethane (22 mg, 0.62 mmoL) at room temperature.

The resulting mixture was stirred at RT for 16 h. The progress of the reaction was monitored by TLC. Upon disappearance of starting material, the reaction mixture was quenched with water and extracted with ethyl acetate (2×10 mL). The organic layer was dried over Na2SO4, filtered and then solvent was evaporated under reduced pressure to afford crude as pale-yellow gum. The crude material was purified by column chromatography using silica mesh (2-5% MeOH in DCM as eluent) to afford 5-(4-chloro-2-(1-methyl-1H-pyrazol-4-yl)phenyl)-1-methyl-3-methylenepyrrolidin-2-one as an off-white gum (60 mg, 67%). 1H-NMR (400 MHz, CDCl3): 7.46 (s, 1H), 7.39 (s, 1H), 7.31-7.30 (m, 2H), 7.07-7.04 (m, 1H), 6.08 (brs, 1H), 5.36 (m, 1H), 4.61-4.78 (m, 1H), 3.97 (s, 3H), 3.24 (m, 1H), 2.97-2.90 (m, 1H), 2.36 (s, 3H).

MS: 302.2

### Synthetic Scheme for Amide Analogs

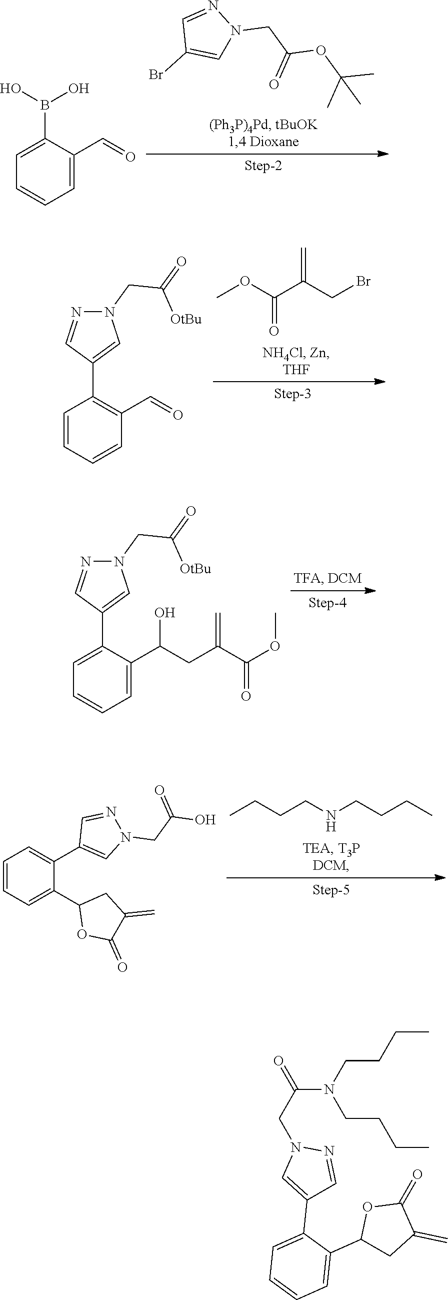

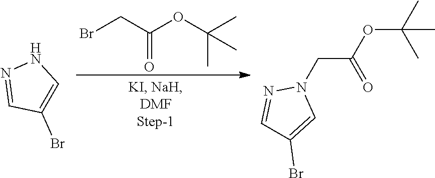

### Step-1: tert-butyl 2-(4-bromo-1H-pyrazol-1-yl)acetate

To a stirred solution of 4-bromo-1H-pyrazole (3.0 g, 20.4 mmol) in DMF (25 mL) at 0° C., was added sodium hydride (0.58 g 24.49 mmol) and it was stirred for 30 mins. Subsequently, tert-butyl 2-bromoacetate (4.3 g 22.45 mmol) and KI (3.38 g 2.04 mmol) were added to the reaction mixture. It was stirred for 20 hours at room temperature. The progress of the reaction was Reaction progress was monitored by TLC. Upon disappearance of starting material, the reaction mixture was diluted with ethyl acetate (2×100 mL) and washed with water (2×50 mL). The organic layer was dried over Na₂SO₄, filtered and evaporated under reduced pressure to afford the crude material. It was purified by column chromatography by using 30% ethyl acetate and hexane as eluent to afford tert-butyl 2-(4-bromo-1H-pyrazol-1-yl)acetate. Yield 4.5 g (84.42%): ¹H-NMR (300 MHz, CDCl₃) δ: 7.49 (s, 2H), 4.77 (s, 2H), 1.47 (s, 9H); LCMS: Rt 2.55 min (99.67% purity), m/z 206.98 [M−18]⁻

### Step-2: tert-butyl 2-(4-(2-formylphenyl)-1H-pyrazol-1-yl)acetate

To a stirred solution of (2-formylphenyl)boronic acid (0.76 g, 5.07 mmol), tert-butyl 2-(4-bromo-1H-pyrazol-1-yl)acetate (1 g, 5.07 mmol) in 1,4 dioxane (20 mL), was added potassium tertiary butoxide (1.49 g, 15.21 mmol) under nitrogen purged for 30 min. The reaction mixture was again degassed wing nitrogen gas and Pd(Ph3P)4 (0.29 g, 0.25 mmol) was added at room temperature. The reaction mixture was stirred at 110° C. for 16 h. The progress of the reaction was monitored by TLC. Upon disappearance of the starting material, the reaction mixture was diluted with ethyl acetate (2×100 mL) and washed with water (2×50 mL). The organic layer was dried over Na₂SO₄, filtered and evaporated under reduced pressure to afford the crude material. It was purified by column chromatography (30% ethyl acetate and hexane as eluent) to afford tert-butyl 2-(4-(2-formylphenyl)-1H-pyrazol-1-yl)acetate. Yield 0.574 g (39.58%): ¹H-NMR (300 MHz, CDCl₃) δ: 10.23 (d, J=0.6 Hz, 1H), 7.98 (dd, J=0.9&7.8 Hz, 1H), 7.68 (s, 1H), 7.61-7.58 (m, 2H), 7.47-7.43 (m, 2H), 4.89 (s, 2H), 1.49 (s, 9H); LCMS: Rt 2.59 min (89.67% purity), m/z 287.15 [M+1]⁺

### Step-3: methyl 4-(2-(1-(2-(tert-butoxy)-2-oxoethyl)-1H-pyrazol-4-yl)phenyl)-4-hydroxy-2-methylenebutanoate

To a stirred solution of tert-butyl 2-(4-(2-formylphenyl)-1H-pyrazol-1-yl)acetate. (0.57 g, 2.00 mmol)), in dry THF (10 mL), was added methyl 2-(bromomethyl) acrylate (0.395 g, 2.2 mmol), saturated ammonium chloride (4 mL) and zinc (0.154 g, 2.4 mmol). The reaction mixture was stirred at room temperature for 16 h. The progress of the reaction was monitored by TLC. Upon disappearance of starting material, the reaction mixture was diluted with ethyl acetate (2×100 mL) and washed with water (2×50 mL). The organic layer was dried over Na₂SO₄, filtered and evaporated under reduced pressure to afford the crude material. It was purified by column chromatography (30% ethyl acetate in hexane as eluent) to afford methyl 4-(2-(1-(2-(tert-butoxy)-2-oxoethyl)-1H-pyrazol-4-yl)phenyl)-4-hydroxy-2-methylenebutanoate. Yield 0.820 g (98%).

¹H-NMR (300 MHz, CDCl₃) δ: 7.74-7.63 (m, 3H), 7.37-7.28 (m, 3H), 6.23 (d, J=1.2 Hz, 1H), 5.63 (d, J=0.9 Hz, 2H), 4.90 (d, J=7.8 Hz, 2H), 3.73 (s, 3H), 2.79-2.63 (m, 2H) 2.59 (s, 1H), 1.48 (s, 9H).

### Step-4: 2-(4-(2-(4-methylene-5-oxotetrahydrofuran-2-yl)phenyl)-1H-pyrazol-1-yl)acetic acid

To a stirred solution of methyl 4-(2-(1-(2-(tert-butoxy)-2-oxoethyl)-1H-pyrazol-4-yl)phenyl)-4-hydroxy-2-methylenebutanoate (0.820 g, 2.12 mmol) in DCM (10 mL), was added trifluroacetic acid (0.1 mL). The reaction mixture was stirred for overnight at room temperature. The progress of the reaction was monitored by TLC. Upon disappearance of starting material, the reaction mixture was concentrated under reduced pressure, diluted with dichloromethane (2×100 mL) and washed with water (2×50 mL). The organic layer was dried over Na₂SO₄, filtered and evaporated under reduced pressure to obtain the crude material. It was purified by column chromatography (15% ethyl acetate in hexane as eluent) to afford 2-(4-(2-(4-methylene-5-oxotetrahydrofuran-2-yl)phenyl)-1H-pyrazol-1-yl)acetic acid Yield 0.750 g (90%): ¹H-NMR (300 MHz, CDCl₃) δ: 7.54 (s, 2H), 7.35-7.30 (m, 2H), 7.19 (bs, 1H), 6.20 (s, 1H), 5.60 (s, 2H), 5.29-4.94 (m, 4H), 3.15 (bs, 1H) 2.86 (d, J=12.9 Hz, 1H). LCMS: Rt 4.24 min (99.09% purity), m/z 299.10 [M+1]⁺

### Step-5: N,N-dibutyl-2-(4-(2-(4-methylene-5-oxotetrahydrofuran-2-yl)phenyl)-1H-pyrazol-1-yl)acetamide

To a stirred solution of 2-(4-(2-(4-methylene-5-oxotetrahydrofuran-2-yl)phenyl)-1H-pyrazol-1-yl)acetic acid (0.4 g, 1.47 mmol) in dichloromethane (10 mL), were added triethylamine (0.54 g, 5.3 mmol), dibutyl amine (0.542 g, 5.3 mmol) and propylphosphonic anhydride solution 50% in ethyl acetate (0.64 g 2.01 mmol) at 0° C. The reaction mixture was stirred at 50° C. for 2 h. The progress of the reaction was monitored by TLC. Upon disappearance of starting material, it was diluted with dichloromethane (2×100 mL) and washed with water (2×50 mL). The organic layer was dried over Na₂SO₄, filtered and evaporated under reduced pressure to obtain the crude material. It was purified by prep HPLC to afford N,N-dibutyl-2-(4-(2-(4-methylene-5-oxotetrahydrofuran-2-yl)phenyl)-1H-pyrazol-1-yl)acetamide. Yield 0.420 g (84.63%)¹H-NMR (300 MHz, CDCl₃) δ: 7.55 (s, 1H), 7.50 (s, 1H), 7.37-7.33 (m, 1H), 7.29-7.26 (m, 2H), 6.20 (t, J=2.7 Hz, 1H), 5.70 (q, J=6.0 Hz, 1H), 5.57 (t, J=2.4 Hz, 1H), 5.69 (t, J=2.1 Hz, 1H) 5.57 (t, J=2.4 Hz, 1H), 4.95 (s, 2H), 3.65-3.14 (m, 4H), 2.87-2.77 (m, 1H), 1.57-1.43 (m, 4H), 1.43-1.32 (m, 4H), 1.29-1.21 (m, 6H).

LCMS: Rt 2.78 min (99.67% purity), m/z 410.26 [M+1]⁺

Following analogous compounds were synthesized using this scheme:

### 3-methylene-5-(2-(1-(2-oxo-2-(piperidin-1-yl) ethyl)-1H-pyrazol-4-yl) phenyl) dihydrofuran-2 (3H)-one

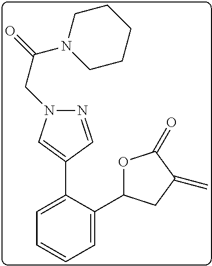 [SMILES: C=C1CC(c2ccccc2-c2cnn(CC(=O)N3CCCCC3)c2)OC1=O]

¹H-NMR (300 MHz, DMSO-d₆) δ: 7.86 (s, 1H), 7.61 (s, 1H), 7.38 (s, 4H), 6.12 (s, 1H), 5.77 (s, 2H), 5.16 (s, 2H), 3.75-3.65 (m, 1H), 3.44 (s, 4H), 2.88 (d, J=18.0 Hz, 1H), 1.57 (s, 4H), 1.45 (s, 2H).

LCMS: RT 2.36 min (99.42% purity), m/z 366.18 [M+H]⁺.

### 3-methylene-5-(2-(1-(2-oxo-2-(4-phenylpiperazin-1-yl)ethyl)-1H-pyrazol-4-yl)phenyl)dihydrofuran-2 (3H)-one

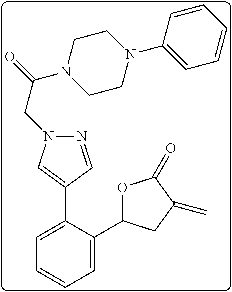 [SMILES: C=C1CC(c2ccccc2-c2cnn(CC(=O)N3CCN(c4ccccc4)CC3)c2)OC1=O]

¹H-NMR (300 MHz, DMSO-d₆) δ: 7.89 (s, 1H), 7.63 (s, 1H), 7.39 (s, 4H), 7.24 (q, J=7.5 Hz, 2H), 6.98 (d, J=7.8 Hz, 2H), 6.82 (t, J=7.2 Hz, 1H), 6.12 (t, J=2.7 Hz, 1H), 5.82-5.75 (m, 2H), 5.26 (s, 2H), 3.65 (q, J=4.2 Hz, 4H), 3.47-3.38 (m, 1H), 3.24-3.14 (m, 4H), 2.92-2.83 (m, 1H).

LCMS: Rt 2.55 min (99.58% purity), m/z 443.38 [M+H]⁺.

### 3-methylene-5-(2-(1-(2-oxo-2-(pyrrolidin-1-yl) ethyl)-1H-pyrazol-4-yl) phenyl) dihydrofuran-2 (3H)-one

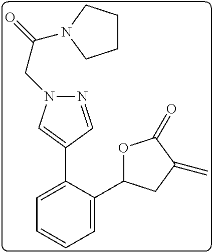 [SMILES: C=C1CC(c2ccccc2-c2cnn(CC(=O)N3CCCC3)c2)OC1=O]

¹H-NMR (300 MHz, CDCl3) δ 7.87 (s, 1H), 7.62 (s, 1H), 7.38 (s, 4H), 6.12 (t, J=2.7 Hz, 1H), 5.82-5.75 (m, 2H), 5.07 (s, 2H), 3.50 (t, J=6.9 Hz, 2H), 3.46-3.32 (m, 3H), 2.91-2.84 (m, 1H), 1.96-1.88 (m, 2H), 1.83-1.75 (m, 2H).

LCMS: Rt 2.19 min (98.27% purity), m/z 352.19 [M+H]⁺.

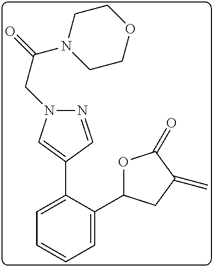 [SMILES: C=C1CC(c2ccccc2-c2cnn(CC(=O)N3CCOCC3)c2)OC1=O]

### 3-methylene-5-(2-(1-(2-morpholino-2-oxoethyl)-1H-pyrazol-4-yl)phenyl)dihydrofuran-2 (3H)-one

1H-NMR (300 MHz, CDCl3) δ 7.817 (s, 1H), 7.62 (s, 1H), 7.38 (s, 4H), 6.12 (t, J=2.7 Hz, 1H), 5.81-2.77 (m, 1H), 5.21 (d, J=5.4 Hz, 2H), 3.63-3.41 (m, 9H), 2.92-2.83 (m, 1H).

LCMS: Rt 2.11 min (97.06% purity), m/z 366.26 [M+H]⁺.

### Synthesis of N-methyl-2-(4-(2-(4-methylene-5-oxotetrahydrofuran-2-yl)phenyl)-1H-pyrazol-1-yl)acetamide

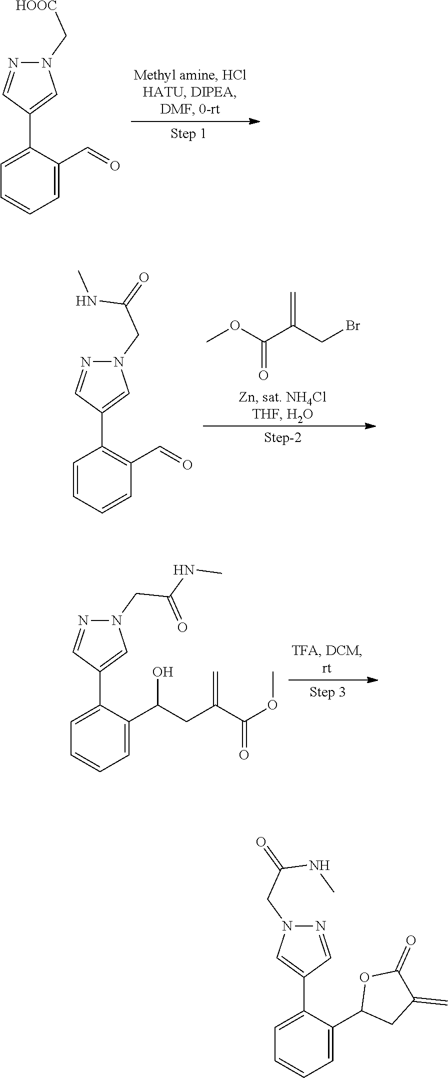

### Step-1: Synthesis of 2-(4-(2-formylphenyl)-1H-pyrazol-1-yl)-N-methylacetamide

To a stirred solution of 2-(4-(2-formylphenyl)-1H-pyrazol-1-yl)acetic acid (0.3 g 1.30 mmol) and Methyl amine (0.170 g 1.95 mmol) in DMF (5 mL) at 0° C. was added DIPEA (0.504 g 3.91 mmol), followed by HATU (0.743 g 1.95 mmol). The reaction mixture was stirred at room temperature for 16 h. The progress of the reaction was monitored by TLC. After completion of the starting material, the reaction mixture was diluted with ethyl acetate (50 mL) and washed with water (2×50 mL). The organic layer was dried over Na₂SO₄, filtered and evaporated under reduced pressure to get the crude compound. The crude obtained was purified by SiO₂ column chromatography (eluent: 15% ethyl acetate in hexane) to afford 2-(4-(2-formylphenyl)-1H-pyrazol-1-yl)-N-methylacetamide as brown colored gummy solid (0.3 g, 99.59%).

¹H-NMR (300 MHz, CDCl₃) δ:10.16 (s, 1H), 8.07 (s, 1H), 8.02-8.01 (m, 1H), 7.87 (d, J=7.8 Hz, 1H), 7.76 (s, 1H), 7.73-7.67 (m, 1H), 7.56-7.42 (m, 2H), 5.75 (s, 2H), 2.64 (d, J=4.5 Hz, 3H).

### Step-2: Synthesis of methyl 4-hydroxy-4-(2-(1-(2-(methylamino)-2-oxoethyl)-1H-pyrazol-4-yl) phenyl)-2-methylenebutanoate

To a stirred solution of 2-(4-(2-formylphenyl)-1H-pyrazol-1-yl)-N-methylacetamide (0.3 g 1.23 mmol) in THF (2 mL) was added saturated ammonium chloride (5 mL) and zinc (0.096 g, 1.48 mmol). The reaction mixture stirred at room temperature for 16 h. The progress of the reaction was monitored by TLC. After completion of the starting material, the reaction mixture was diluted with ethyl acetate (50 mL) and washed with water (2×50 mL). The organic layer was dried over Na₂SO₄, filtered and evaporated under reduced pressure to get the crude compound. The crude obtained was purified by SiO₂ column chromatography (eluent: 50% ethyl acetate in hexane) to afford methyl 4-hydroxy-4-(2-(1-(2-(methylamino)-2-oxoethyl)-1H-pyrazol-4-yl) phenyl)-2-methylenebutanoate as brown colored gum (0.180 g, 42.66%).

¹H-NMR (300 MHz, CDCl₃) δ: 8.17 (bs, 1H), 8.08-7.92 (m, 2H), 7.86 (s, 1H), 7.58 (s, 1H), 7.38 (s, 1H), 7.33-7.23 (m, 2H), 6.05 (s, 1H), 5.77 (d, J=7.5 Hz, 1H), 5.58 (s, 1H), 5.15 (d, J=4.5 Hz, 1H), 4.80 (t, J=6.9 Hz, 2H), 3.62 (s, 3H), 3.15-3.12 (m, 2H), 2.64 (d, J=4.5 Hz, 2H).

LCMS: Rt 1.98 min (84.35% purity), m/z 344.22 [M+H]⁺.

### Step-3: Synthesis of N-methyl-2-(4-(2-(4-methylene-5-oxotetrahydrofuran-2-yl)phenyl)-1H-pyrazol-1-yl)acetamide

To a stirred solution of methyl 4-hydroxy-4-(2-(1-(2-(methylamino)-2-oxoethyl)-1H-pyrazol-4-yl) phenyl)-2-methylenebutanoate (0.180 g 0.52 mmol) in DCM (10 mL) at 0° C. was added trifluoracetic acid (0.5 mL). Then the reaction mixture stirred at room temperature for 12 h. The progress of the reaction was monitored by TLC. After completion of the starting material, the reaction mixture was concentrated under reduced pressure to get the crude compound. The crude obtained was purified by prep. HPLC to afford N-methyl-2-(4-(2-(4-methylene-5-oxotetrahydrofuran-2-yl) phenyl)-1H-pyrazol-1-yl)acetamide as off white gummy solid (0.050 g, 30.9%).

¹H-NMR (300 MHz, DMSO-d₆) δ:7.99 (s, 1H), 7.92 (s, 1H), 7.63 (s, 1H), 7.38 (s, 4H), 6.13 (s, 1H), 5.83-5.77 (m, 2H), 4.82 (s, 2H), 3.46 (d, J=6.3 Hz, 1H), 2.91-2.85 (m, 1H), 2.63 (d, J=3.9 Hz, 3H).

LCMS: Rt 2.04 min (99.85% purity), m/z 312.17 [M+H]⁺.

### Synthesis of Open Chain Amide Analogs

### 4-hydroxy-4-(2-(1-methyl-1H-pyrazol-4-yl) phenyl)-2-methylenebutanoic acid

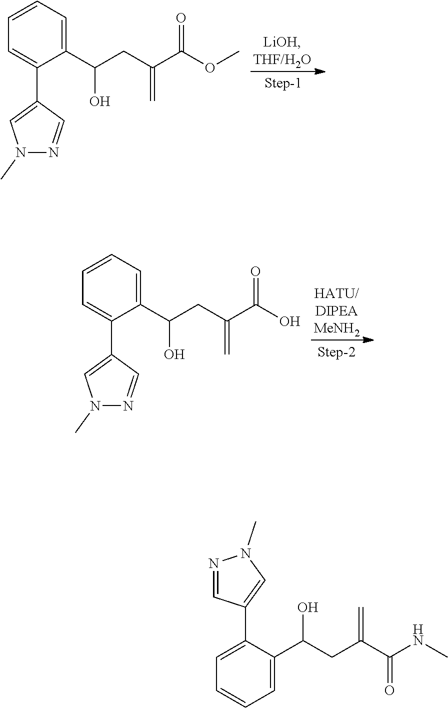

### Step-1: 4-hydroxy-4-(2-(1-methyl-1H-pyrazol-4-yl) phenyl)-2-methylenebutanoic acid

To a stirred solution of methyl 4-hydroxy-4-(2-(1-methyl-1H-pyrazol-4-yl) phenyl)-2-methylenebutanoate (0.080 g, 2.7 mmol) in mixture of solvent THF (3 mL), methanol (1 mL) at 0° C., was added aqueous lithium hydroxide (0.014 g, 3.3 mmol in 1 mL water). The reaction mixture was allowed to stir at room temperature for 2 h. The progress of the reaction was monitored by TLC. Upon disappearance of starting material, the reaction mixture was concentrated under reduced pressure. Reaction mass acidified with potassium hydrogen sulfate and extracted with 5% methanol and dichloromethane (2×50 mL). The organic layer evaporated under reduced pressure to afford the crude material. It was purified by column chromatography (10-15% methanol in dichloromethane as eluent) to afford 4-hydroxy-4-(2-(1-methyl-1H-pyrazol-4-yl) phenyl)-2-methylenebutanoic acid. Yield 0.250 g (64.9%, gummy)

¹H-NMR (300 MHz, CDCl₃) δ:7.65 (d, J=8.1 Hz, 1H), 7.48 (d, J=11.4 Hz, 2H), 7.38-7.32 (m, 1H), 7.29-7.23 (m, 1H), 7.20 (dd, J=1.5&7.8 Hz, 1H), 6.25 (s, 1H), 5.87 (bs, 2H), 5.16 (t, J=6.6 Hz, 1H), 3.85 (s, 3H), 3.46 (s, 1H);

LCMS (m/z): 273 [M+H]⁺.

### Step-2: 4-hydroxy-N-methyl-4-(2-(1-methyl-1H-pyrazol-4-yl)phenyl)-2-methylenebutanamide

A solution of 4-hydroxy-4-(2-(1-methyl-1H-pyrazol-4-yl)phenyl)-2-methylenebutanoic acid (0.1 g, 0.36 mmol) in DMF (6 mL), methyl amine (0.248 g, 0.36 mmol) and N, N-Diisopropylethylamine (0.47 g, 0.36 mmol) was added stirred for 30 min. The reaction mixture was cooled to 0° C. and HATU (0.209 g, 0.055 mmol) was added and it was stirred at RT for 1 h. Subsequently, it was diluted with cold water and extracted with dichloromethane (2×50 mL). The organic layer was evaporated under reduced pressure to afford the crude material. It was purified by column chromatography (10-15% methanol in dichloromethane as eluent) to afford 4-hydroxy-N-methyl-4-(2-(1-methyl-1H-pyrazol-4-yl)phenyl)-2-methylenebutanamide. Yield 0.030 g (54%).

¹H-NMR (300 MHz, CDCl₃) δ:7.67-7.65 (m, 1H), 7.57-7.55 (m, 2H), 7.36-7.28 (m, 1H), 7.28-7.26 (m, 1H), 7.25 (d, J=1.2 Hz, 1H), 6.07 (s, 1H), 5.55 (s, 1H), 5.13 (s, 1H), 4.65 (s, 1H), 3.96 (s, 3H), 3.73 (s, 1H), 2.83 (s, 3H), 2.67-2.52 (m, 2H);

LCMS (m/z): 286 [M+H]⁺.

### Synthesis of Additional Compounds

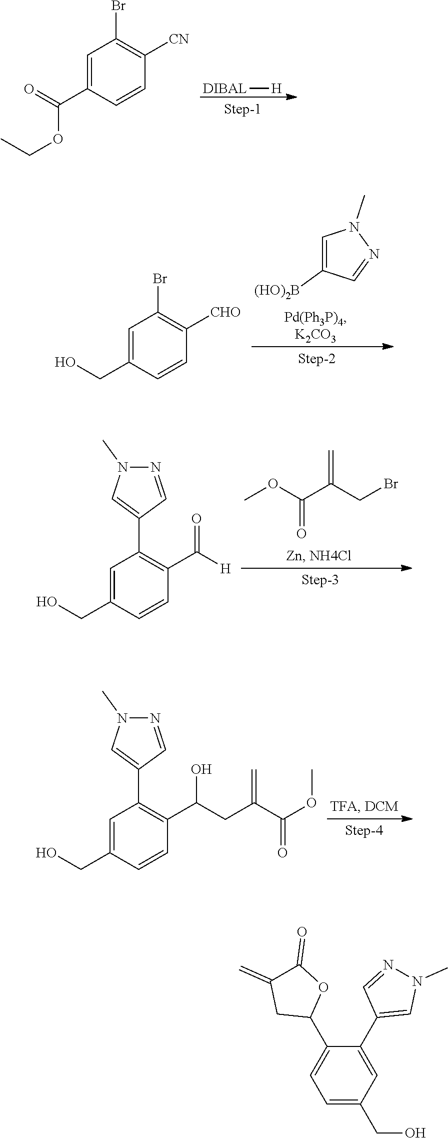

### Step-1: Synthesis of: 2-bromo-4-(hydroxymethyl) benzaldehyde

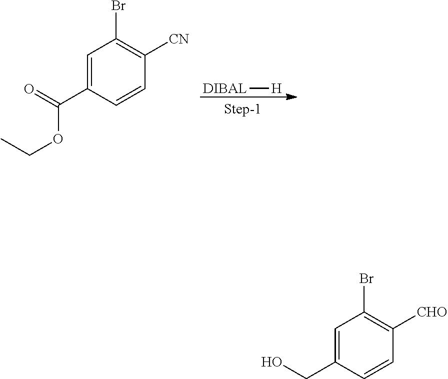

To a stirred solution of ethyl 3-bromo-4-cyanobenzoate (200 mg, 0.78 mmol) in THF (10 mL) was added DIBAL-H (0.59 mmol) at 0° C. The reaction mixture was stirred at RT for 2 h. The reaction progress was monitored by TLC. Upon disappearance of starting material, the reaction mixture was diluted with ethyl acetate (20 mL) and washed with water (2×10 mL). The organic layer was dried over Na₂SO₄, filtered and then solvent was evaporated under reduced pressure. The residue thus obtained purified by column chromatography using silica gel (mesh: 60-120) and 15-20% ethyl acetate and petroleum ether, as eluent to afford 2-bromo-4-(hydroxymethyl) benzaldehyde as an off-white gum (80 mg, 47.27%).

### Step-2: Synthesis of 4-(hydroxymethyl)-2-(1-methyl-1H-pyrazol-4-yl)benzaldehyde

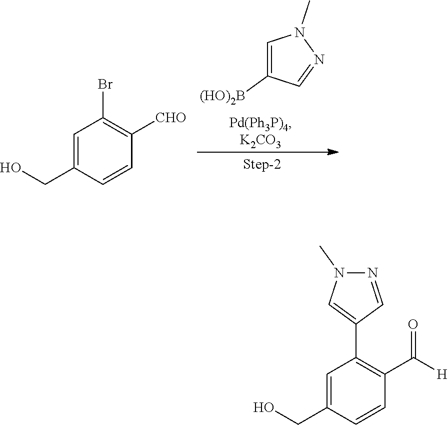

Procedure: A suspension of 2-bromo-4-(hydroxymethyl) benzaldehyde (80 mg, 0.37 mmol), 1-methyl-1H-pyrazol-4-yl)boronic acid (60.89 mg, 0.48 mmol) and potassium carbonate (134.27 mg, 0.96 mmol) in 1,4-Dioxanne (10 mL) and water (5 mL) was degassed for 5 min, and tetrakis(triphenylphosphine)-palladium (8 mg, 10% mol) was added The reaction mixture was degassed for additional 5 min and heated at 90° C. for 2 h. The reaction progress was monitored by TLC. Upon disappearance of starting material, the reaction mixture was diluted with ethyl acetate (20 mL) and washed with water (2×10 mL). The organic layer was dried over Na₂SO₄, filtered and then solvent was evaporated under reduced pressure to obtain a crude mixture. It was purified by column chromatography using silica gel (mesh: 60-120) and the required compound was eluted at 15-20% ethyl acetate and petroleum ether to afford 4-(hydroxymethyl)-2-(1-methyl-1H-pyrazol-4-yl)benzaldehyde as an off-white gum (60 mg, 59.67%).

### Step-3: Synthesis of: methyl 4-hydroxy-4-(4-(hydroxymethyl)-2-(1-methyl-1H-pyrazol-4-yl)phenyl)-2-methylenebutanoate

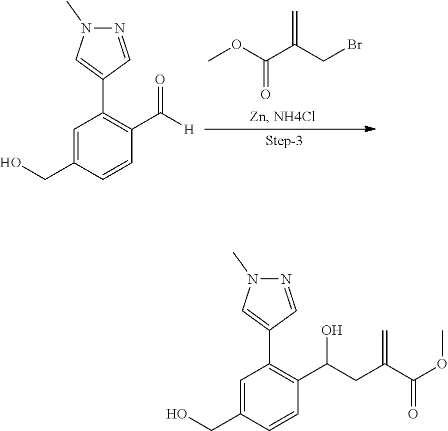

To a stirred solution of 4-(hydroxymethyl)-2-(1-methyl-1H-pyrazol-4-yl)benzaldehydee (60 mg, 0.27 mmol) in tetrahydrofuran (10 mL), were added methyl 2-(bromomethyl) acrylate (59.60 mg, 0.33 mmol), saturated ammonium chloride (2 mL) and zinc (65.29 mg, 0.99 mmol). The resulting reaction mixture was stirred at room temperature for 12 h. The progress of the reaction was monitored by TLC. Upon disappearance of starting material, the reaction mixture was diluted with ethyl acetate (20 mL) and washed with water (2×10 mL). The organic layer was dried over Na₂SO₄, filtered and then solvent was evaporated under reduced pressure to afford methyl 4-hydroxy-4-(4-(hydroxymethyl)-2-(1-methyl-1H-pyrazol-4-yl)phenyl)-2-methylenebutanoate (60 mg, 69%).

### Step-4: 5-(4-(hydroxymethyl)-2-(1-methyl-1H-pyrazol-4-yl)phenyl)-3-methylenedihydrofuran-2 (3H)-one

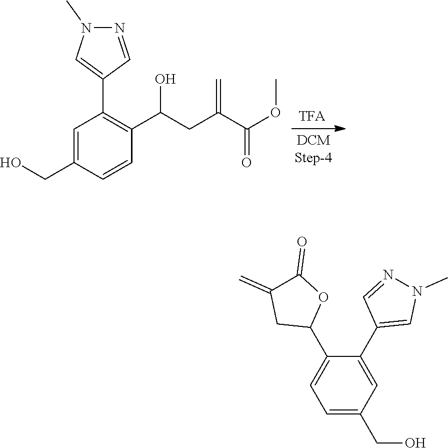

To a stirred solution of methyl 4-hydroxy-4-(4-(hydroxymethyl)-2-(1-methyl-1H-pyrazol-4-yl)phenyl)-2-methylenebutanoate (60 mg, 0.08 mmol) in dry dichloromethane (10 mL) was added trifluoroacetic acid (0.3 mL). Then reaction mixture was stirred overnight at room temperature. The progress of the reaction was monitored by TLC. Upon disappearance of starting material, the reaction mixture was evaporated under reduced pressure to afford crude compound, which was purified by column chromatography using silica gel (mesh 100-200). The desired compound was eluted at 30% ethyl acetate and hexane to afford 5-(4-(hydroxymethyl)-2-(1-methyl-1H-pyrazol-4-yl)phenyl)-3-methylenedihydrofuran-2 (3H)-one as an off-white gum (30 mg, 55%).

¹H-NMR (400 MHz, CDCl₃): 7.52 (s, 1H), 7.47 (s, 1H), 7.44-7.36 (d, 1H), 7.34 (d, 1H), 7.32 (s, 1H), 6.30 (t, 1H), 5.73-5.70 (t, 1H), 5.67 (s, 1H), 4.72 (s, 2H), 3.97 (s, 3H), 3.25-3.19 (m, 1H), 2.95-2.89 (m, 1H).

LCMS (M+H=285.1, 82.53%).

### 5-(1,1′-dimethyl-1H, 1′H-[3,4′-bipyrazol]-4-yl)-3-methylenedihydrofuran-2 (3H)-one

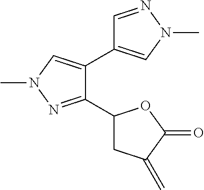 [SMILES: C=C1CC(c2nn(C)cc2-c2cnn(C)c2)OC1=O]

¹H-NMR (400 MHz, CDCl₃): 7.74 (s, 1H), 7.68 (s, 1H), 7.39 (s, 1H), 6.30-6.29 (t, 1H), 5.70-5.68 (m, 1H), 5.56-5.52 (t, 1H), 3.98 (s, 3H), 3.91 (s, 3H), 3.37-3.27 (m, 1H), 3.02-2.95 (m, 1H).

### Synthesis of (S)-3-acryloyl-4-benzyloxazolidin-2-one

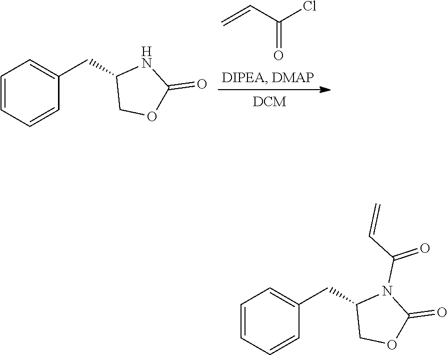

To a stirred solution of (S)-4-benzyloxazolidin-2-one (250 mg, 1.41 mmoL) in DCM (15 mL), DIPEA (364 mg, 2.82 mmol) and DMAP (2 mg, 0.014 mmoL). The resulting mixture was cooled to 0° C. and acrloyl chloride (191 mg, 2.16 mmoL) was added. The resulting reaction mixture was stirred at RT for 16 h. The progress of the reaction was monitored by TLC. Upon disappearance of starting material, the reaction mixture was quenched with ice cold water and extracted with diethyl ether (2×10 mL). The organic layer was dried over Na₂SO₄, filtered and then solvent was evaporated under reduced pressure. The crude mixture thus obtained was purified by column chromatography by using silica gel (mesh: 100-200) and the required compound was eluted at 20-30% ethyl acetate in petroleum ether to afford (S)-3-acryloyl-4-benzyloxazolidin-2-one as a pale yellow gum (36 mg, 12%).

¹H-NMR (400 MHz, CDCl3): 7.55-7.48 (m, 1H), 7.35-7.32 (m, 1H), 7.29-7.27 (d, 1H), 7.25-7.21 (m, 2H), 6.63-6.58 (m, 1H), 5.95-5.92 (m, 1H), 4.76-4.72 (m, 1H), 4.25-4.17 (m, 2H), 3.37-3.33 (m, 1H), 2.84-2.78 (m, 1H).

LCMS (M+H=232.0, 98.82%).

### Synthesis of methyl acryloylphenylalaninate

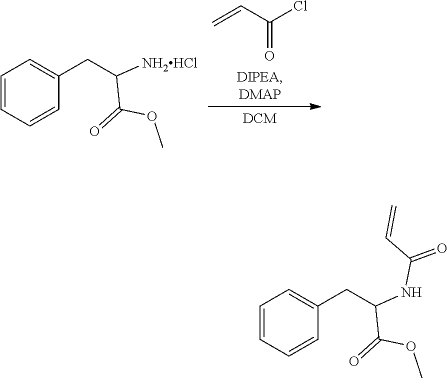

To a stirred solution of methyl phenylalaninate hydrochloride (250 mg, 1.16 mmoL) in DCM (15 mL) DIPEA (300 mg, 2.32 mmol) was added followed by DMAP (3 mg, 0.016 mmoL). The resulting mixture was cooled to 0° C. and acrloyl chloride (160 mg, 1.74 mmoL) was added. The resulting reaction mixture was stirred at RT for 16 h. The progress of the reaction was monitored by TLC. Upon disappearance of starting material, the reaction mixture was quenched with ice cold water and extracted with diethyl ether (2×10 mL). The organic layer was dried over Na₂SO₄, filtered and the solvent was evaporated under reduced pressure to afford the crude mixture. It was purified by column chromatography by using silica gel (mesh: 100-200) and the required compound was eluted at 20-30% ethyl acetate in petroleum ether to afford methyl acryloylphenylalaninate as a pale yellow gum (40 mg, 15%).

¹H-NMR (400 MHz, CDCl₃): 7.30-0.26 (m, 2H), 7.25-7.24 (d, 1H), 7.09-7.07 (d, 1H), 6.31-6.26 (d, 1H), 6.12-6.05 (m, 1H), 6.00-5.98 (brs, 1H), 5.68-5.66 (d, 1H), 4.99-4.95 (m, 1H), 3.74-3.71 (s, 3H), 3.19-3.16 (m, 2H).

LCMS (M+H=234.1, 92.74%).

### Synthesis of methyl 2-acrylamido-2-phenylacetate

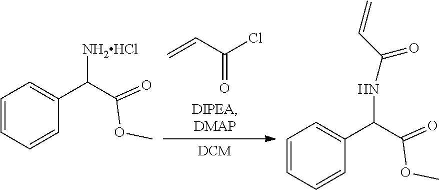

To a stirred solution of methyl 2-amino-2-phenylacetate hydrochloride (250 mg, 1.14 mmol) in DCM (20 mL), DIPEA (367.74 mg, 2.850 mmoL) was added followed by DMAP (13.93 mg, 0.11 mmoL). The reaction mixture was cooled to 0° C. and acryloyl chloride (154.19 mg, 1.71 mmoL) was added drop wise. The resulting reaction mixture was stirred at RT for 16 h. The progress of the reaction was monitored by TLC. Upon disappearance of starting material, the reaction mixture was quenched with water (20 mL) and extracted with diethyl ether (2×20 mL). The organic layer was dried over Na₂SO₄, filtered and then solvent was evaporated under reduced pressure to afford the crude mixture. It was purified by column chromatography using silica gel (mesh: 100-200) and the required compound was eluted at 10-12% ethyl acetate and petroleum ether to afford methyl 2-acrylamido-2-phenylacetateas pale as a yellow solid (57.1 mg, 52.52%). ¹H-NMR (400 MHz, CDCl₃): 7.39-7.29 (m, 5H), 6.56-6.54 (brs, 1H), 6.34-6.29 (dd, 1H), 6.18-6.12 (m, 1H), 5.70-5.65 (m, 2H), 3.74 (s, 3H).

LCMS (M+H=220.0, 91.53%).

### Synthesis of 4-(1-methyl-1H-pyrazol-4-yl)-3-(4-methylene-5-oxotetrahydrofuran-2-yl)benzoic acid

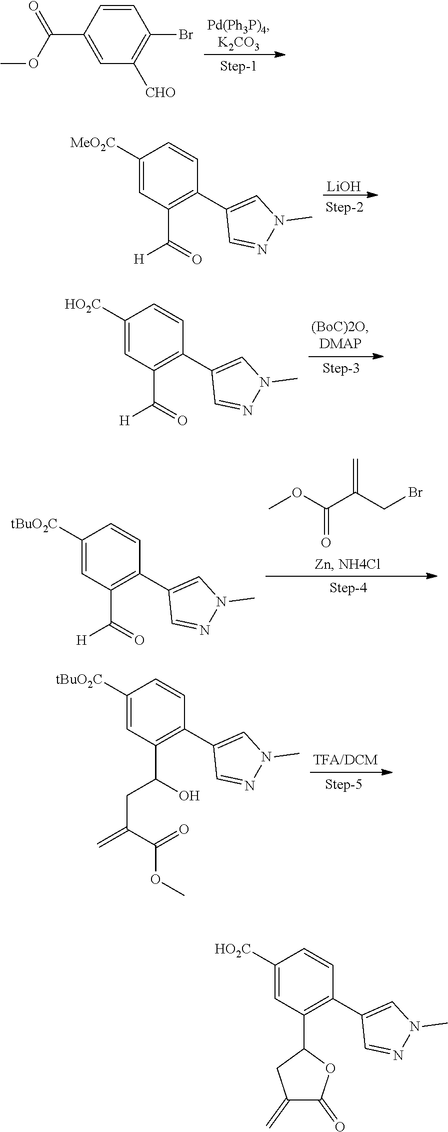

### Step-1: Synthesis of methyl 3-formyl-4-(1-methyl-1H-pyrazol-4-yl)benzoate

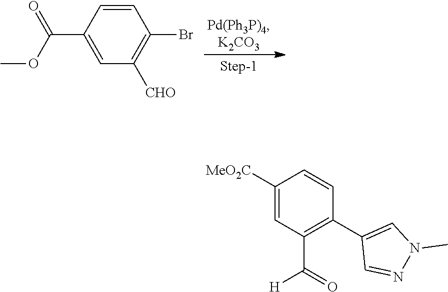 [SMILES: COC(=O)c1ccc(Br)c(C=O)c1 ; COC(=O)c1ccc(-c2cnn(C)c2)c(C=O)c1]

To a stirred solution of methyl 4-bromo-3-formylbenzoate (0.2 g, 0.83 mmol) and 1-methyl-1H-pyrazol-4-yl)boronic acid (210 mg, 0.99 mmol) in 1,4-Dioxanne (7 mL) and potassium carbonate (300 mg, 2.15 mmol) in water (4 mL) was added. The resulting mixture was degassed for 5 min, and tetrakis(triphenylphosphine)-palladium (20 mg, 10% mol) was added. The reaction mixture was degassed for additional 5 min and it was heated at 90° C. for 2 h. The progress of the reaction was monitored by TLC. Upon disappearance of starting material, the reaction mixture was diluted with ethyl acetate (20 mL) and washed with water (2×10 mL). The organic layer was dried over Na₂SO₄, filtered and then solvent was evaporated under reduced pressure to obtain the crude mixture. It was purified by column chromatography using silica gel (mesh: 60-120) and the required compound was eluted at 15-20% ethyl acetate and petroleum ether to afford methyl 3-formyl-4-(1-methyl-1H-pyrazol-4-yl)benzoate as an off-white gum (180 mg, 90%).

### Step-2: Synthesis of 3-formyl-4-(1-methyl-1H-pyrazol-4-yl)benzoic acid

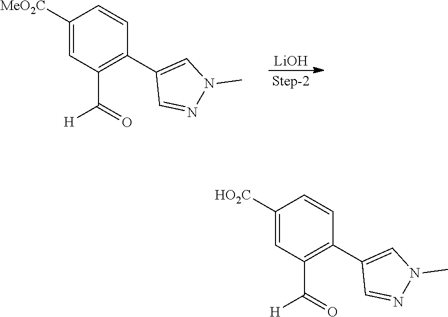

To a stirred solution of methyl 3-formyl-4-(1-methyl-1H-pyrazol-4-yl)benzoate (0.18 g, 1.75 mmol) in tetrahydrofuran (10 mL), Methanol (2 mL), was added solution of LiOH·H₂O (160 mg, 1.10 mmoL) in water at room temperature and the resulting reaction mixture was stirred at RT for 2 h. The progress of the reaction was monitored by TLC. Upon disappearance of starting material, the reaction mixture was concentrated and diluted with water (25 mL) and washed with ethyl acetate (2×25 mL). The aqueous layer was acidified with concentrated HCL and extracted with ethyl acetate (2×25 mL). The organic layer was dried over Na₂SO₄, filtered and the solvent was evaporated under reduced pressure, to afford 3-formyl-4-(1-methyl-1H-pyrazol-4-yl)benzoic acid as an off white solid (100 mg, 58.9%)

### Step-3: Synthesis of tert-butyl 3-formyl-4-(1-methyl-1H-pyrazol-4-yl)benzoate

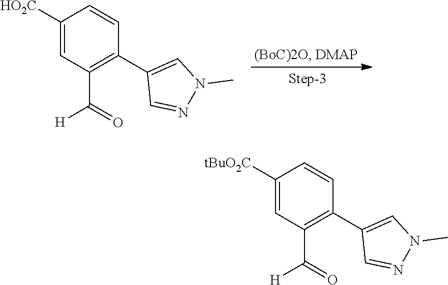 [SMILES: Cn1cc(-c2ccc(C(=O)O)cc2C=O)cn1 ; Cn1cc(-c2ccc(C(=O)OC(C)(C)C)cc2C=O)cn1]

To a stirred solution of 3-formyl-4-(1-methyl-1H-pyrazol-4-yl)benzoicacid (100 mg, 0.43 mmol) in tetrahydrofuran (10 mL), BOC anhydride (142.2 mg, 0.65 mmol) was added followed by DMAP (5.30 mg, 0.04 mmol) at RT and the resulting reaction mixture stirred at room temperature for 12 h. The progress of the reaction was monitored by TLC. Upon disappearance of starting material, the reaction mixture was diluted with ethyl acetate (50 mL) and washed with water (2×25 mL). The organic layer was dried over Na₂SO₄, filtered and then solvent was evaporated under reduced pressure, the obtain the crude mixture. It was purified by column chromatography using silica gel (mesh 60-120) and the required compound was eluted at 35-40% ethyl acetate and petroleum ether to afford tert-butyl 3-formyl-4-(1-methyl-1H-pyrazol-4-yl)benzoateas an pale yellow gum (100 mg, 80.4%)

### Step-4: 3-(1-hydroxy-3-(methoxycarbonyl)but-3-en-1-yl)-4-(1-methyl-1H-pyrazol-4-yl)benzoic acid

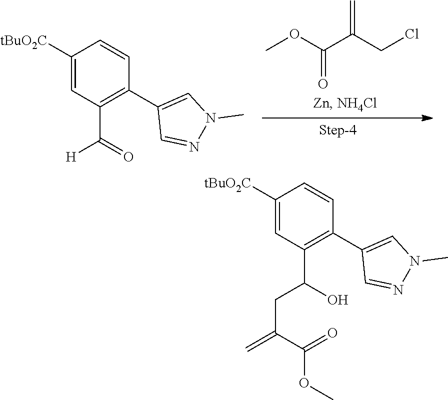

To a stirred solution of tert-butyl 3-formyl-4-(1-methyl-1H-pyrazol-4-yl)benzoate (100 mg, 0.34 mmol) in tetrahydrofuran (10 mL), were added methyl 2-(bromomethyl) acrylate (75.02 mg, 0.41 mmol), saturated ammonium chloride (2 mL) and zinc (82.1 mg, 1.257 mmol). The resulting reaction mixture stirred at room temperature for 12 h. The progress of the reaction was monitored by TLC. Upon disappearance of starting material, the reaction mixture was diluted with ethyl acetate (50 mL) and washed with water (2×25 mL). The organic layer was dried over Na₂SO₄, filtered and then solvent was evaporated under reduced pressure to afford 3-(1-hydroxy-3-(methoxycarbonyl)but-3-en-1-yl)-4-(1-methyl-1H-pyrazol-4-yl)benzoic acid (100 mg, 86.67%)

### Step 5: 4-(1-methyl-1H-pyrazol-4-yl)-3-(4-methylene-5-oxotetrahydrofuran-2-yl)benzoic acid

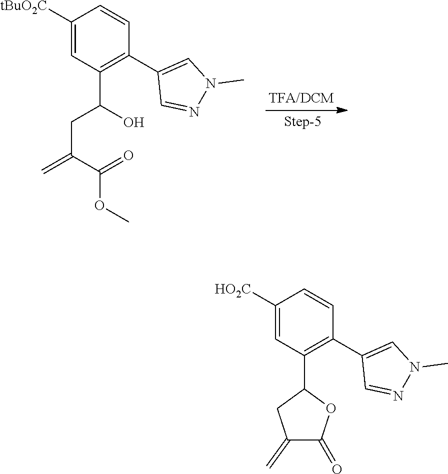 [SMILES: C=C(CC(O)c1cc(C(=O)OC(C)(C)C)ccc1-c1cnn(C)c1)C(=O)OC ; C=C1CC(c2cc(C(=O)O)ccc2-c2cnn(C)c2)OC1=O]

To a stirred solution of 3-(1-hydroxy-3-(methoxycarbonyl)but-3-en-1-yl)-4-(1-methyl-1H-pyrazol-4-yl)benzoic acid (100 mg, 0.335 mmol) in dry dichloromethane (10 mL) was added tri fluoroacetic acid (0.3 mL). The reaction mixture was stirred overnight at room temperature. Upon disappearance of starting material on TLC, the reaction mixture was evaporated under reduced pressure to afford the crude compound. It was purified by column chromatography using silica gel (mesh 100-200) and the required compound was eluted at 30% ethyl acetate and hexane to afford 4-(1-methyl-1H-pyrazol-4-yl)-3-(4-methylene-5-oxotetrahydrofuran-2-yl)benzoic acid as off white gum (23 g, 70%).

¹H-NMR (400 MHz, DMSO): 13.2 (bS, 1H), 8.04 (s, 1H), 8.01-7.90 (t, 2H), 7.70-7.68 (d, 1H), 7.54-7.52 (m, 1H), 6.15 (s, 1H) 5.93-5.91 (t, 1H), 5.19 (s, 1H), 3.91 (s, 3H), 3.52-3.46 (m, 1H), 2.86-2.80 (m, 1H).

LCMS (M+H=299.1, 90.15%).

### Synthesis of 3-ethylidene-1-phenethylindolin-2-one and 1-methylspiro[indoline-3,2′-pyran]-2,4′(3′H)-dione

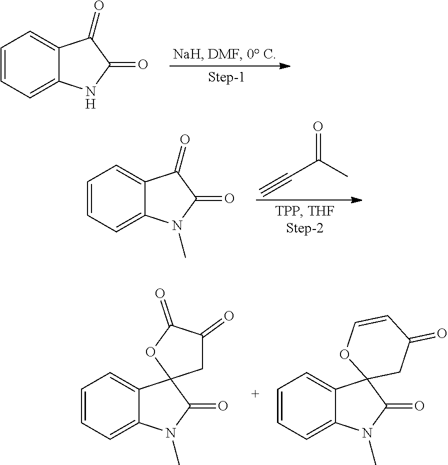

### STEP-1: Synthesis of 1-methylindoline-2,3-dione

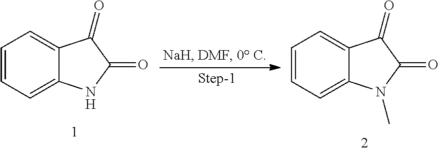

To a stirred solution of Isatin (1.0 g, 6.80 mmol) in DMF (2 mL), NaH (0.163 g, 6.80 mmol) was added under argon atmosphere at 0° C. The reaction mixture was stirred at 0° C. for 30 min. Methyl iodide (1.06 g) was added to the reaction mixture at 0° C. After completion of the reaction formation of non-polar spot with respect to starting material observed on TLC. The reaction mixture was added to water (5 mL) and it was extracted with EtOAc (5 mL×3). The organic layer was separated, dried over anhydrous Na₂SO₄ and concentrated under reduced pressure to afford 1-methylindoline-2,3-dione as a crude product. The crude product was purified by silica gel column chromatography (100-200 mesh), by using 15-20% Ethyl acetate and Pet ether as an eluent to afford the desired product (0.80 g), as an orange solid.

### STEP-2: Synthesis of 3-ethylidene-1-phenethylindolin-2-one

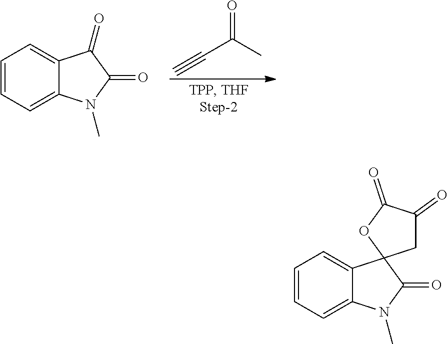

To a stirred solution of 1-methylindoline-2,3-dione (0.1 g, 0.621 mmol) in THF (2.0 mL), but-3-yn-2-one (0.063 g, 0.931 mmol), Triphenyl phosphine (33 mg, 0.124 mmol) were added Under argon atmosphere. The mixture was stirred at room temperature until the reaction was completed. After completion of the reaction (formation of non-polar spot with respective to starting material 1-methylindoline-2,3-dione on TLC), the solvent was removed under reduced pressure. The residue thus obtained was purified by silica gel column chromatography (100-200 mesh), by using 7-12% Ethyl acetate and Pet ether as an eluent to afford the desired products (0.02 g each) as off-white solids.

The product formation was confirmed by ¹HNMR and LCMS.

### Synthesis of 3-ethylidene-1-phenethylindolin-2-one

¹H NMR (400 MHz, CDCl₃): δ=7.44-7.04 (m, 1H), 7.37-7.35 (m, 1H), 7.16-7.13 (m, 1H), 6.90-6.88 (d, 1H, J=7.6 Hz), 5.15 (d, 1H, J=2.0 Hz), 4.73 (d, 1H, J=2.4 Hz), 3.22 (s, 3H), 3.04 (d, 1H, J=18.4 Hz), 2.89 (d, 1H, J=18.4 Hz).

LC-MS: 99.81%. m/z 230.9 [M+1]+

### 1-methylspiro[indoline-3,2′-pyran]-2,4′(3′H)-dione

¹H NMR (400 MHz, CDCl₃): δ=7.44 (d, 1H, J=0.8 Hz), 7.48-7.26 (m, 2H), 7.09-7.05 (m, 1H), 7.09-7.05 (m, 1H), 6.89 (d, 1H, J=8.0 Hz), 3.23 (s, 3H), 3.18 (d, 1H), 2.68-2.63 (m, 1H),

m/z 230.9 [M+1]⁺ LC-MS, purity by LC-MS −99.74%.

### 1′-benzyl-5-methylene-3H-spiro[furan-2,3′-indoline]-2′,4 (5H)-dione

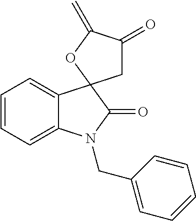 [SMILES: C=C1OC2(CC1=O)C(=O)N(Cc1ccccc1)c1ccccc12]

¹H NMR (400 MHz, CDCl 3): δ=7.37-7.27 (m, 7H), 7.12-7.08 (m, 1H), 6.78 (d, 1H, J=8.0 Hz), 5.18 (d, 1H, J=2.8 Hz), 4.90 (q, 2H, J=15.2 Hz), 4.77 (d, 1H, J=2.4 Hz), 3.10 (d, 1H, J=18.4 Hz), 2.94 (d, 1H, J=18.8 Hz),

m/z 305.9 [M⁺+H]

### 1-benzylspiro[indoline-3,2′-pyran]-2,4′(3′H)-dione

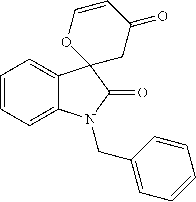 [SMILES: O=C1C=COC2(C1)C(=O)N(Cc1ccccc1)c1ccccc12]

¹H NMR (400 MHz, CDCl₃): δ=7.50 (d, 1H, J=6.8 Hz), 7.48 (d, 1H, J=17.2 Hz), 7.36-7.27 (m, 6H), 6.76 (d, 1H, J=8.0 Hz), 5.65 (d, 1H, J=6.4 Hz), 4.95 (q, 2H, J=15.6 Hz), 3.27 (d, 1H, J=14.0 Hz), 2.75 (d, 1H, J=16.8 Hz), 2.89 (d, 1H, J=18.4 Hz).

m/z 305.9 [M+1]; purity by LC: 99.84%.

### Synthesis of N-(2-(1-methyl-1H-pyrazol-4-yl)benzyl)ethenesulfonamide

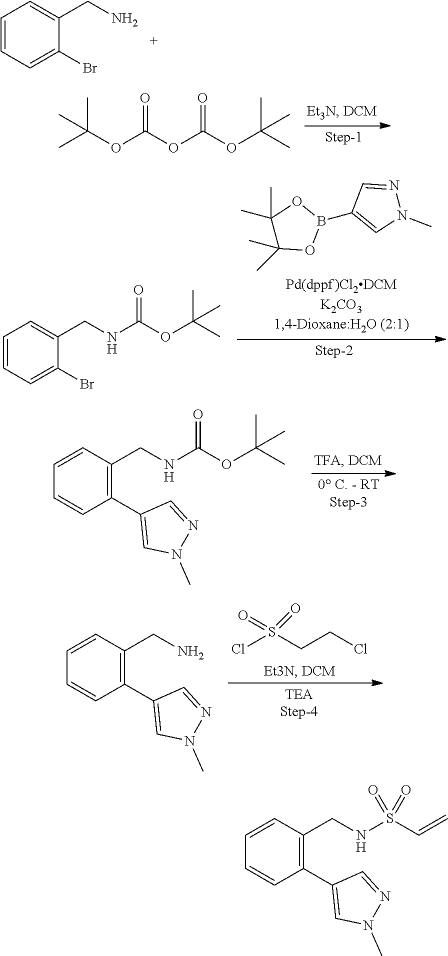

### STEP-1: Synthesis of tert-butyl 2-bromobenzylcarbamate

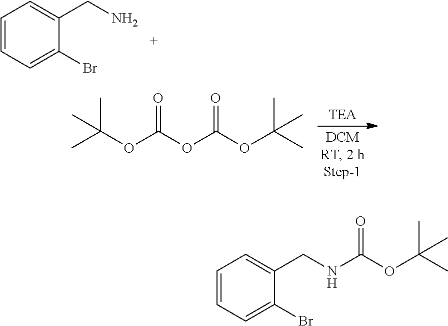

To a solution of (2-bromophenyl)methanamine (1.0 g, 0.005 mol) and Boc anhydride (1.29 g, 0.0059) in DCM (10 ml), Et₃N (0.54 g, 0.0059 mol) were added. The reaction mixture was stirred at RT for 2 h. After completion of the reaction (formation of non-polar spot with respective to starting material on TLC), the reaction mixture was diluted with water and extracted with EtOAc. The organic layer was separated, dried over anhydrous Na₂SO₄ and concentrated under reduced pressure to afford tert-butyl 2-bromobenzylcarbamate as a crude product (semi solid). It was purified by silica gel column chromatography (100-200 mesh), by using 28-30% Ethyl acetate and Pet ether as an eluent to afford the desired product (1.74 g), as a colourless gum. The product formation was confirmed by TLC.

### STEP-2: Synthesis of tert-butyl 2-(1-methyl-1H-pyrazol-4-yl)benzylcarbamate

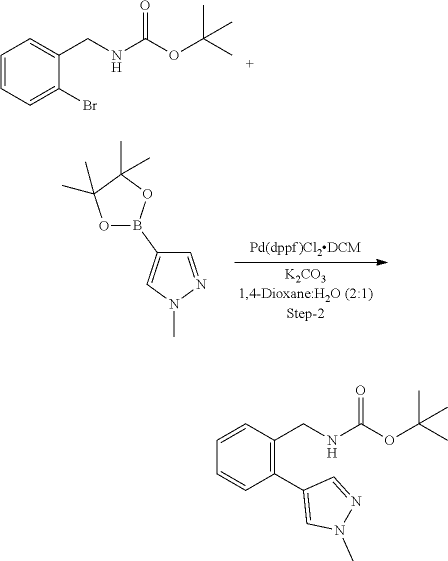 [SMILES: CC(C)(C)OC(=O)NCc1ccccc1Br ; Cn1cc(B2OC(C)(C)C(C)(C)O2)cn1 ; Cn1cc(-c2ccccc2CNC(=O)OC(C)(C)C)cn1]

To a mixture of tert-butyl 2-(1-methyl-1H-pyrazol-4-yl)benzylcarbamate (1.74 g, 0.006 mol) and Pd(dppf)Cl₂·DCM (0.25 g, 0.0003 mol) in 1,4-Dioxane and water (13:6 mL) under Nitrogen atmosphere, K₂CO₃ (2.1 g, 0.015 mol) and 1-methyl-4-(4,4,5,5-tetramethyl-1,3,2-dioxaborolan-2-yl)-1H-pyrazole (1.26 g, 0.006 mol) were added. The reaction mixture was degassed with nitrogen for 5 min. The reaction mixture was stirred under 80° C. for 2 h. After completion of the reaction (formation of polar spot with respect to starting material tert-butyl 2-(1-methyl-1H-pyrazol-4-yl)benzylcarbamate on TLC), the reaction mixture was diluted with water and extracted with EtOAc. The organic layer was separated, dried over anhydrous Na₂SO₄ and concentrated under reduced pressure to afford tert-butyl 2-(1-methyl-1H-pyrazol-4-yl)benzylcarbamate as a crude product. It was isolated by silica gel column chromatography (100-200 mesh), by using 15-20% Ethyl acetate and Pet ether as an eluent to afford the desired product (0.9 g), as an off white gum. The product formation was confirmed by TLC.

### STEP-3: Synthesis of (2-(1-methyl-1H-pyrazol-4-yl)phenyl)methanamine

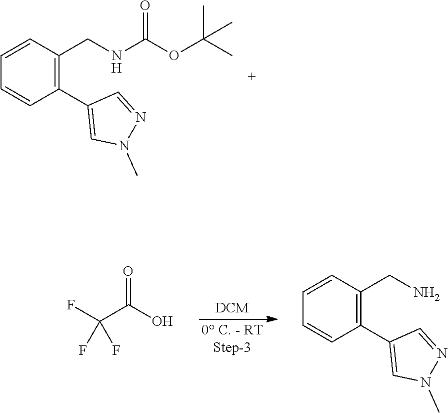 [SMILES: Cn1cc(-c2ccccc2CNC(=O)OC(C)(C)C)cn1 ; O=C(O)C(F)(F)F ; Cn1cc(-c2ccccc2CN)cn1]

To a stirred solution of tert-butyl 2-(1-methyl-1H-pyrazol-4-yl)benzylcarbamate (0.05 g, 0.0001 mol) in DCM (10 mL), TFA (2 mL) was added at 0° C. The resulting mixture was allowed to stir ar RT. After completion of reaction (formation of polar spot with respective to starting material tert-butyl 2-(1-methyl-1H-pyrazol-4-yl)benzylcarbamate on TLC), the reaction mixture was quenched with water (15 mL) and extracted with EtOAc. The organic layer was separated, dried over anhydrous Na₂SO₄ and concentrated under reduced pressure to afford (2-(1-methyl-1H-pyrazol-4-yl)phenyl)methanamine as a TFA Salt (semi solid). The pure product was isolated by silica gel column chromatography (100-200 mesh), by using 18-20% Ethyl acetate in Pet ether as eluent to afford the desired product (0.02 g), as an off white semi solid. The product formation was confirmed by TLC.

### STEP-4: Synthesis of N-(2-(1-methyl-1H-pyrazol-4-yl)benzyl)ethenesulfonamide

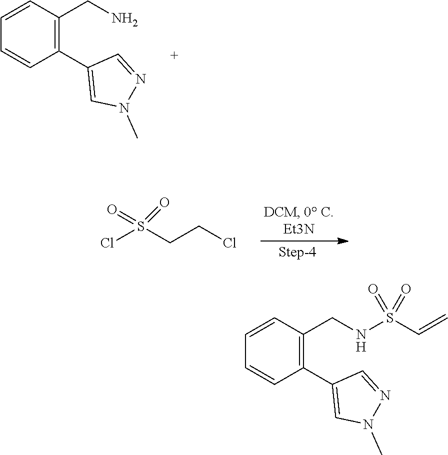 [SMILES: Cn1cc(-c2ccccc2CN)cn1 ; O=S(=O)(Cl)CCCl ; C=CS(=O)(=O)NCc1ccccc1-c1cnn(C)c1]

To a stirred solution of (2-(1-methyl-1H-pyrazol-4-yl)phenyl)methanamine (0.3 g, 0.001 mol) in DCM (3 mL), TEA (0.48 g, 0.004 mol) was added. The reaction mixture was cooled to 0° C. and 2-chloroethanesulfonyl chloride (0.36 g, 0.002 mol) was added. The resulting mixture was allowed to stir for 1 h at 0° C. After completion of the reaction (formation of non-polar spot with respective to starting material (2-(1-methyl-1H-pyrazol-4-yl)phenyl)methanamine on TLC), the reaction mixture was quenched with water and extracted with DCM. The organic layer was separated, dried over anhydrous Na₂SO₄ and concentrated under reduced pressure to afford the desired compound, as crude. It was purified by silica gel column chromatography (100-200 mesh), by using 60-70% Ethyl acetate in Pet ether as eluent to afford the desired product (0.05 g), as an off white semi solid.

¹H NMR (400 MHz, CDCl₃): δ=7.57 (d, 2H, J=4.4 Hz), 7.41 (d, 2H, J=6.8 Hz), 7.34-7.36 (m, 3H), 6.43 (q, 1H, J=9.6 Hz), 6.25 (d, 1H, J=16.8 Hz), 5.93 (d, 1H, J=9.6 Hz), 4.35 (bs, 1H), 4.27 (d, 1H, J=6.0 Hz), 3.98 (s, 3H),

LC-MS, purity by LC-MS−99.25%. m/z 278.3 [M+1]⁺

### Synthesis of N-(1-(2-(1-methyl-1H-pyrazol-4-yl)phenyl)ethyl)ethenesulfonamide

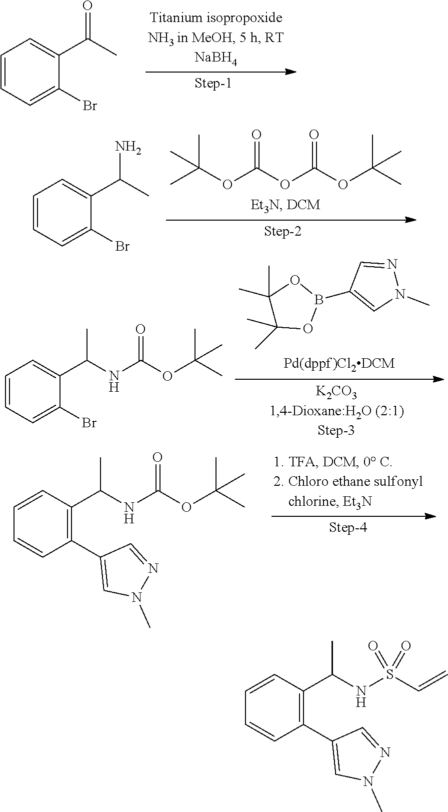

### STEP-1: Synthesis of 1-(2-bromophenyl)ethanamine

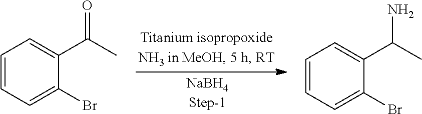

To a solution of 1-(2-bromophenyl)ethanone 1 (3.0 g, 0.015 mol) in methanolic NH₃ (0.26 g, 0.150 mol), titanium isopropoxide (5.99 g, 0.021 mol) was added. The reaction mixture was stirred under RT for 5 h. After completion of the reaction, NaBH₄ was slowly added at RT for 1 h. After completion of the reaction (formation of polar spot with respective to starting material on TLC) the reaction mixture was diluted with water and extracted with EtOAc. The organic layer was dried over anhydrous Na₂SO₄ and concentrated under reduced pressure to afford crude product. It was purified by silica gel column chromatography (100-200 mesh), by using 15-20% Ethyl acetate and Pet ether as an eluent to afford 1-(2-bromophenyl)ethanamine (2.54 g) as an off-white semi solid.

### STEP-2: Synthesis of tert-butyl 1-(2-bromophenyl)ethylcarbamate

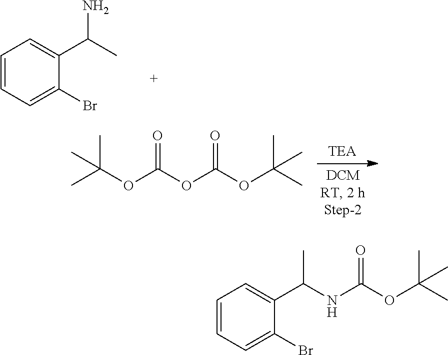

To a mixture of (2-bromophenyl)methanamine (0.5 g, 0.004 mol) and Di-tert-butyl dicarbonate (1.04 g, 0.0047 mol) in DCM (5 mL), TEA (0.44 g, 0.0043 mol) were added. The reaction mixture was stirred under RT for 2 h. After completion of the reaction (formation of non-polar spot with respect to starting material on TLC), the reaction mixture was diluted with water and extracted with DCM. The organic layer was dried over anhydrous Na₂SO₄ and concentrated under reduced pressure to afford tert-butyl 1-(2-bromophenyl)ethylcarbamate as a crude product. It was purified by silica gel column chromatography (100-200 mesh), by using 16-20% Ethyl acetate and Pet ether as an eluent to afford desired product (0.87 g) as an off-white semi solid.

### STEP-3: Synthesis of tert-butyl 1-(2-(1-methyl-1H-pyrazol-4-yl)phenyl)ethylcarbamate

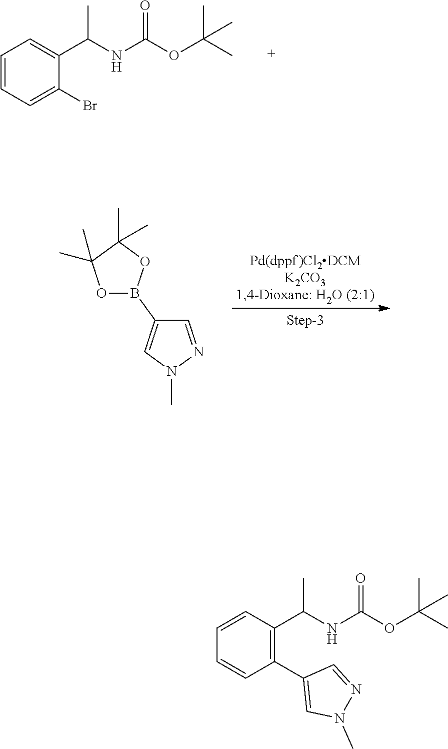 [SMILES: CC(NC(=O)OC(C)(C)C)c1ccccc1Br ; Cn1cc(B2OC(C)(C)C(C)(C)O2)cn1 ; CC(NC(=O)OC(C)(C)C)c1ccccc1-c1cnn(C)c1]

To a mixture of tert-butyl 1-(2-bromophenyl)ethylcarbamate (0.87 g, 0.0040 mol) and Pd(dppf)Cl₂·DCM (0.16 g, 0.0002 mol) in 1,4-Dioxane and water (8 ml) under Nitrogen atmosphere, K₂CO₃ (1.39 g, 0.010 mol) and 1-methyl-4-(4,4,5,5-tetramethyl-1,3,2-dioxaborolan-2-yl)-1H-pyrazole (0.84 g, 0.004 mol) were added. The reaction mixture was degassed with nitrogen for 5 min. The reaction mixture was stirred at 80° C. for 3 h. After completion of the reaction (formation of polar spot with respect to starting material on TLC), the reaction mixture was diluted with water and extracted with EtOAc. The organic layer was separated, dried over anhydrous Na₂SO₄ and concentrated under reduced pressure to afford tert-butyl 1-(2-(1-methyl-1H-pyrazol-4-yl)phenyl)ethylcarbamate as a crude product. The pure product was isolated by silica gel column chromatography (100-200 mesh), by using 18-25% Ethyl acetate and Pet ether as an eluent to afford desired product (0.81 g) as an off-white semi solid. The product formation was confirmed by TLC.

### STEP-4: Synthesis of N-(1-(2-(1-methyl-1H-pyrazol-4-yl)phenyl)ethyl)ethenesulfonamide

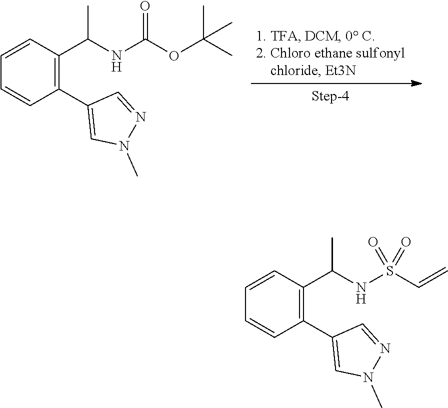 [SMILES: CC(NC(=O)OC(C)(C)C)c1ccccc1-c1cnn(C)c1 ; C=CS(=O)(=O)NC(C)c1ccccc1-c1cnn(C)c1]

To a stirred solution of tert-butyl 1-(2-(1-methyl-1H-pyrazol-4-yl)phenyl)ethylcarbamate (0.09 g, 0.30 mmol) in DCM (2 mL), Trifluoro acetic acid (1.0 mL) was added at 0° C. The resulting reaction mixture was allowed to stir for 2 h at ambient temperature. The reaction mass was concentrated under reduced pressure. The residue was dissolved in dichloromethane (5 mL), cooled to 0° C. and TEA (0.29 g, 1.82 mmol) was added slowly at 0° C. under nitrogen atmosphere. 2-chloroethanesulfonyl chloride (0.03 g, 0.30 mmol) was added at 0° C. The resulting mixture was allowed to stir for 1 h at 0° C. After completion of the reaction, water was added to the reaction mixture and it was extracted with dichloromethane. The organic layer was separated, dried over anhydrous Na₂SO₄ and concentrated under reduced pressure to afford the desired product as crude material. It was isolated by silica gel column chromatography (100-200 mesh), by using 60-70% Ethyl acetate in Pet ether as an eluent to afford desired product (0.05 g) as an off-white semi solid.

The product formation was confirmed by ¹HNMR and LCMS.

¹H NMR (400 MHz, CDCl₃): δ=7.52 (d, 2H, J=2.8 Hz), 7.40 (d, 1H, J=7.6 Hz), 7.34 (t, 1H, J=6.8 Hz), 7.29-7.22 (m, 2H), 6.16 (q, 1H, J=10 Hz), 6.02 (d, 1H, J=16.8 Hz), 5.66 (d, 1H, J=10.0 Hz), 4.92 (q, 1H, J=6.8 Hz), 4.68 (d, 1H, J=6.4 Hz), 3.98 (s, 3H), 1.45 (d, 3H, J=6.8 Hz).

LC-MS−97.58%. m/z 292.2 [M+1]+

### 5-(3-chloro-2-(1-methyl-1H-pyrazol-4-yl)phenyl)-3-methylenepyrrolidin-2-one

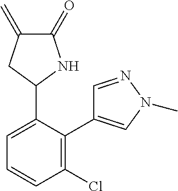

¹H-NMR (400 MHz, CDCl₃): 7.42-7.40 (m, 1H), 7.36 (s, 1H), 7.32-7.26 (m, 2H), 6.07-6.04 (m, 2H), 5.40 (s, 1H), 4.8 (m, 1H), 3.97 (s, 3H), 3.23-3.18 (m, 1H), 2.7-2.6 (m, 1H),

m/z: 287.9 (M+H)

### 5-(3-chloro-2-(1-methyl-1H-pyrazol-4-yl)phenyl)-1-methyl-3-methylenepyrrolidin-2-one

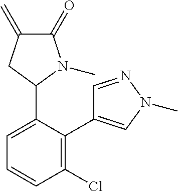

¹H-NMR (400 MHz, CDCl₃): 7.43-7.42 (m, 2H), 7.41-7.40 (m, 1H), 7.31 (s, 1H), 7.04-7.02 (m, 1H), 6.07-6.04 (m, 1H), 5.40 (s, 1H), 4.6 (m, 1H), 4.01 (s, 3H), 3.23-3.18 (m, 1H), 2.75 (s, 3H), 2.6-2.5 (m, 1H).

m/z: 301.9 (M+H).

### Synthesis of Methyl (vinylsulfonyl)-L-tryptophanate

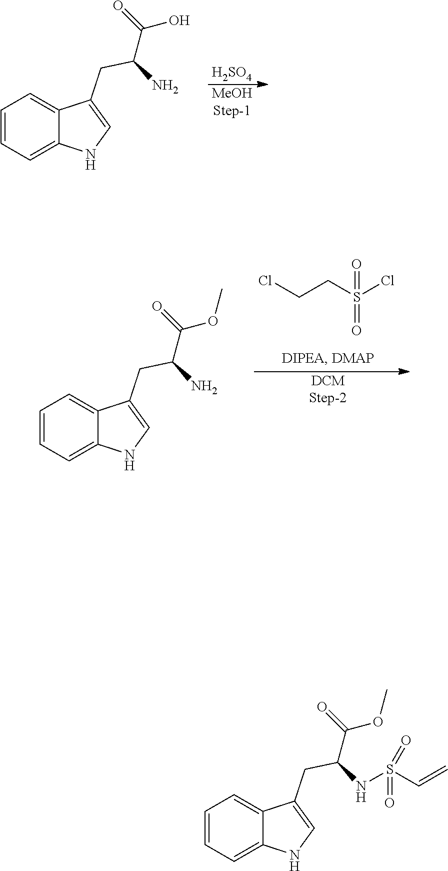

### Step-1: Synthesis of methyl L-tryptophanate

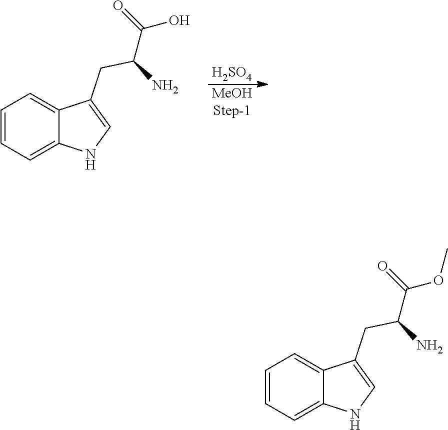

To a stirred solution of L-tryptophan (500 mg, 2.44 mmol) in MeOH (10 mL) H₂SO₄ (300 mg, 2.93 mmoL), at 0° C. Then the resulting reaction mixture was stirred at RT for 16 h. The Reaction progress was monitored by TLC. Upon disappearance of starting material, the reaction mixture was evaporated under reduced pressure and temperature. The obtained crude was purified by column chromatography using silica gel (mesh: 100-200) required compound was eluted at 50-60% ethyl acetate and petroleum ether to afford methyl L-tryptophanate as a thick yellow gummy (420 mg, 78%).

### Step-2: Synthesis of methyl (vinylsulfonyl)-L-tryptophanate

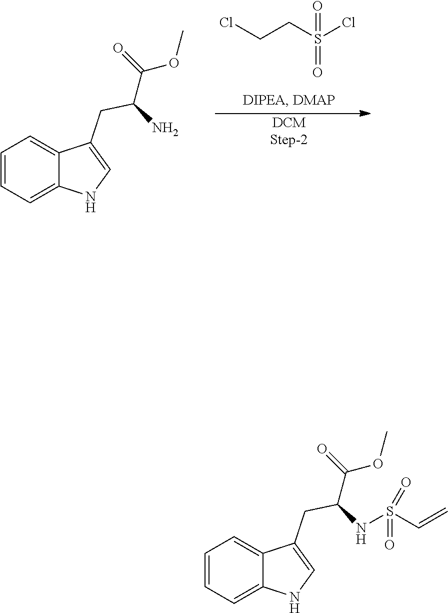

To a stirred solution of methyl L-tryptophanate (200 mg, 0.92 mmol) in DCM (10 mL) DIPEA (180 mg, 1.38 mmoL) was added followed by DMAP (11 mg, 0.09 mmoL), after addition reaction mixture cooled to 0° C. and then added 2-chloroethane-1-sulfonyl chloride (195 mg, 1.19 mmoL) drop wise. Then the resulting reaction mixture was stirred at RT for 16 h. The reaction progress was monitored by TLC. Upon disappearance of starting material, the reaction mixture was quenched with water (20 mL) and extracted with ethyl acetate (2×20 mL). The organic layer was dried over Na₂SO₄, filtered and then solvent was evaporated under reduced pressure and temperature, the obtained crude was purified by column chromatography using silica gel (mesh: 100-200) required compound was eluted at 30-40% ethyl acetate and petroleum ether to afford methyl (vinylsulfonyl)-L-tryptophanate as pale yellow solid (100 mg, 20%). 1H-NMR-CDCl3: 8.15 (bs, 1H), 7.56-7.54 (d, 1H), 7.37-7.35 (d, 1H), 7.21-7.09 (m, 3H), 6.33-6.26 (m, 1H), 6.15-6.11 (d, 1H), 5.75-5.73 (m, 1H), 4.33-4.28 (m, 1H), 3.68 (s, 3H), 3.32-3.31 (m, 2H). LCMS (M+1=309.1, 87.12%).

### Synthesis of Methyl 4-(3-chloro-2-(1-methyl-1H-pyrazol-4-yl)phenyl)-2-methylene-4-(methylsulfonamido)butanoate

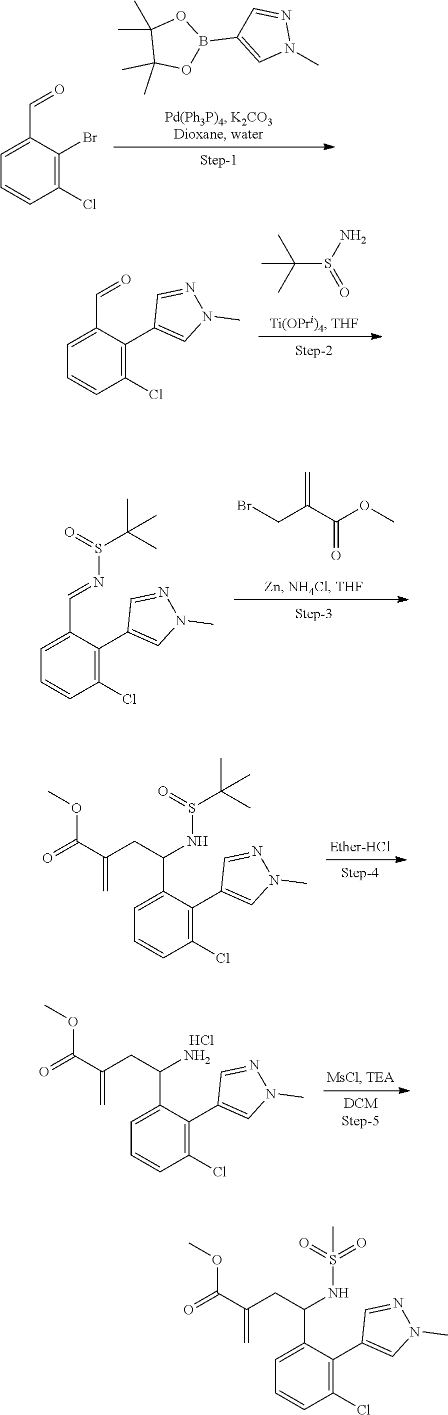

### Step-1: Synthesis of 3-chloro-2-(1-methyl-1H-pyrazol-4-yl)benzaldehyde

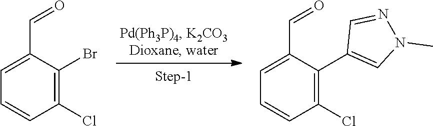

To a stirred solution of 2-bromo-3-chlorobenzaldehyde (0.3 g, 1.37 mmoL) and 1-methyl-4-(4,4,5,5-tetramethyl-1,3,2-dioxaborolan-2-yl)-1H-pyrazole (0.34 g, 1.64 mmoL) in 1,4-Dioxanne (10 mL) and then potassium carbonate (0.47 g, 3.42 mmol) in water (5 mL) was added, the resulting mixture was degassed for 5 min, after that Tetrakis(triphenylphosphine)-palladium(30 mg, 10% moL) was added, and then degassed for further 5 min. The reaction mixture was heated at 90° C. for 2 h. Reaction progress was monitored by TLC. Upon disappearance of starting material, the reaction mixture was diluted with ethyl acetate (40 mL) and washed with water (2×30 mL). The organic layer was dried over Na₂SO₄, filtered and then solvent was evaporated under reduced pressure, the obtained crude was purified by column chromatography using silica gel (mesh: 60-120) required compound was eluted at 35-50% ethyl acetate and petroleum ether to afford 3-chloro-2-(1-methyl-1H-pyrazol-4-yl)benzaldehyde as pale-yellow solid (0.3 g, 96%).

### Step-2: Synthesis of (E)-N-(3-chloro-2-(1-methyl-1H-pyrazol-4-yl)benzylidene)-2-methylpropane-2-sulfinamide

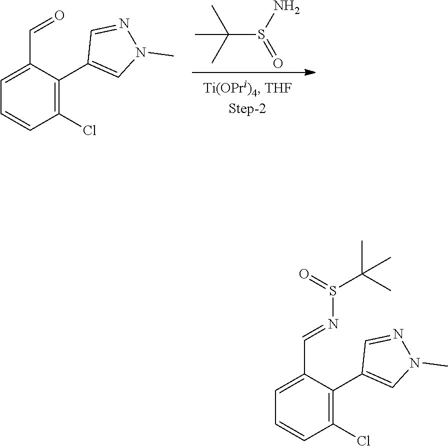

To a stirred solution of 3-chloro-2-(1-methyl-1H-pyrazol-4-yl) benzaldehyde (0.3 g, 1.35 mmoL) in tetrahydrofuran (10 mL), were added 2-methylpropane-2-sulfinamide (0.22 g, 1.76 mmoL), and Titanium isopropoxide (1.0 mL, 3.37 mmoL) at room temperature. The resulting reaction mixture was stirred at room temperature for 16 h. Reaction progress was monitored by TLC. Upon disappearance of starting material, the reaction mixture was quenched with water (50 mL) and filtered the solid. The obtained filterate was extracted with ethyl acetate (2×25 mL). The organic layer was dried over Na₂SO₄, filtered and then solvent was evaporated under reduced pressure, the obtained crude was purified by column chromatography using silica mesh (mesh 60-120) required compound was eluted at 35-40% ethyl acetate and petroleum ether afford (E)-N-(3-chloro-2-(1-methyl-1H-pyrazol-4-yl)benzylidene)-2-methylpropane-2-sulfinamide as a pale yellow gummy (0.24 g, 54%)

### Step-3: Synthesis of methyl 4-((tert-butylsulfinyl)amino)-4-(3-chloro-2-(1-methyl-1H-pyrazol-4-yl)phenyl)-2-methylenebutanoate

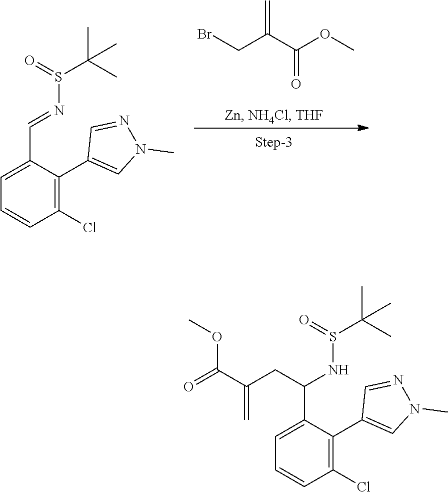

To a stirred solution of (E)-N-(3-chloro-2-(1-methyl-1H-pyrazol-4-yl)benzylidene)-2-methylpropane-2-sulfinamide (0.24 g, 0.74 mmol) and methyl 2-(bromomethyl)acrylate (0.18 mL, 0.96 mmoL) in THF (4 mL), Zinc (174 mg, 2.671 mmoL) and saturated NH₄Cl in water (1 mL) was added, the resulting mixture was stirred at RT for 16 h. Reaction progress was monitored by TLC. 50% of starting material consumed by TLC, the reaction mixture was filtered and filtrate was extracted with ethyl acetate (150 mL) and washed with water (2×10 mL). The organic layer was dried over Na₂SO₄, filtered and then solvent was evaporated under reduced pressure to afford crude as a dark yellow gummy. The obtained crude was purified by column chromatography using silica mesh (mesh 60-120) required compound was eluted at 5-6% MeOH in DCM to afford methyl 4-((tert-butylsulfinyl)amino)-4-(3-chloro-2-(1-methyl-1H-pyrazol-4-yl)phenyl)-2-methylenebutanoateas a pale-yellow solid (0.24 g, 75%).

### Step-4: Synthesis of methyl 4-amino-4-(3-chloro-2-(1-methyl-1H-pyrazol-4-yl)phenyl)-2-methylenebutanoate hydrochloride

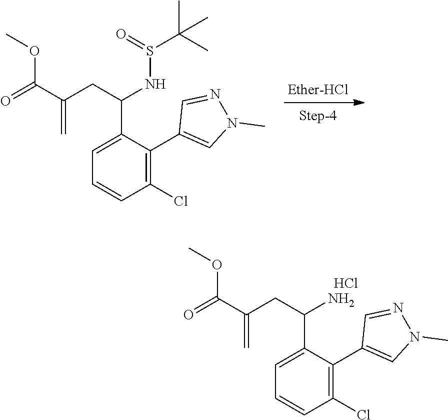

To a stirred solution of 4-((tert-butylsulfinyl)amino)-4-(5-chloro-2-(1-methyl-1H-pyrazol-4-yl)phenyl)-2-methylenebutanoate (0.15 g, 0.354 mmoL) in diethyl ether (2 mL) and, HCl (2M) in diethyl ether (1.5 mL, 10 V) was added at room temperature and the resulting mixture was stirred at RT for 15 min. Reaction progress was monitored by TLC. Upon disappearance of starting material, the reaction mixture was concentrated under reduced pressure and low temperature to afford product as methyl 4-amino-4-(3-chloro-2-(1-methyl-1H-pyrazol-4-yl)phenyl)-2-methylenebutanoate hydrochloride (0.12 g, 95%).

### Step-5: Synthesis of methyl 4-(3-chloro-2-(1-methyl-1H-pyrazol-4-yl)phenyl)-2-methylene-4-(methylsulfonamido)butanoate

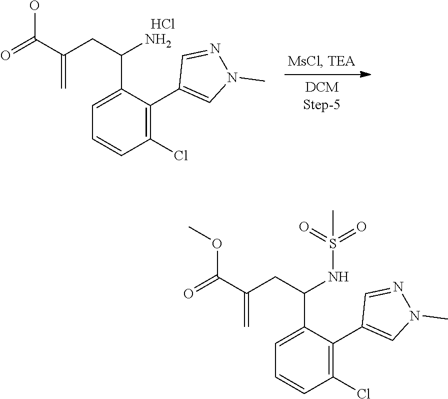

To a stirred solution of methyl MsCl (24 mg, 0.21 mmoL) in DCM (10 mL) was added TEA (42 mg, 0.42 mmoL). He reaction mixture was cooled to 0° C. 4-amino-4-(3-chloro-2-(1-methyl-1H-pyrazol-4-yl)phenyl)-2-methylenebutanoate hydrochloride (50 mg, 0.14 mmoL) in DCM (5 mL) and TEA (0.1 mL) was added to above reaction mixture at 0° C. and then warmed to room temperature. The resulting mixture was stirred at RT for 16 h. Reaction progress was monitored by TLC. Upon disappearance of starting material, the reaction mixture was concentrated under reduced pressure and residue was diluted with DCM (20 mL) and washed with water (2×10 mL). The organic layer was dried over Na₂SO₄, filtered and then the solvent was evaporated under reduced pressure to crude. The crude thus obtained was purified by column chromatography using silica mesh (mesh 60-120) and the required compound was eluted at 50-60% ethyl acetate in pet ether to get methyl 4-(3-chloro-2-(1-methyl-1H-pyrazol-4-yl)phenyl)-2-methylene-4-(methylsulfonamido)butanoate off white solid (12 mg, 21%).

¹H-NMR (400 MHz, CDCl₃): 7.49-7.46 (m, 2H), 7.36 (m, 1H), 7.33-7.31 (m, 1H), 6.18 (brs, 2H), 5.41-5.40 (d, 1H), 4.84-4.82 (m, 1H), 4.02 (s, 3H), 3.72 (s, 3H), 3.12-3.02 (m, 1H), 2.60-2.54 (m, 1H), 2.49-2.45 (m, 1H)

m/z: 397.9 (M+H)

### Methyl (vinylsulfonyl)-D-phenylalaninate

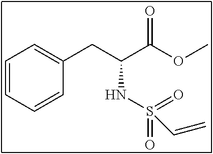 [SMILES: C=CS(=O)(=O)N[C@H](Cc1ccccc1)C(=O)OC]

¹H-NMR-CDCl₃: 7.32-7.27 (m, 3H), 7.16-7.14 (m, 2H), 6.31-6.24 (m, 1H), 6.17-6.13 (m, 1H), 5.81-5.78 (m, 1H), 4.84-4.82 (d, 1H), 4.27-4.21 (m, 1H), 3.73 (s, 3H), 3.15-3.04 (m, 1H).

LCMS (M+H=270.0, 82.13%).

### Methyl acryloyl-L-tryptophanate

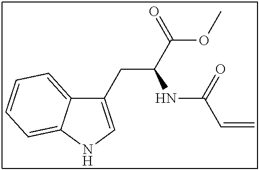 [SMILES: C=CC(=O)N[C@@H](Cc1c[nH]c2ccccc12)C(=O)OC]

¹H-NMR-CDCl₃: 8.15 (brs, 1H), 7.52-7.50 (m, 1H), 7.36-7.34 (d, 1H), 7.20-7.16 (m, 1H), 7.12-7.08 (m, 1H), 6.977-6.972 (d, 1H), 6.30-6.25 (m, 1H), 6.13-6.01 (m, 2H), 5.66-5.63 (m, 1H), 5.06-5.01 (m, 1H), 3.702 (s, 3H), 3.38-3.36 (m, 2H).

LCMS (M+H=273.0, 92.13%).

### Methyl (S)-2-phenyl-2-(vinylsulfonamido)acetate

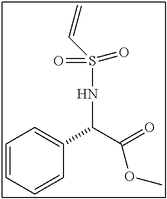

¹H-NMR-CDCl₃: 7.37-7.32 (m, 5H), 6.34-6.28 (m, 1H), 6.15-6.11 (m, 1H), 5.77-5.75 (m, 1H), 5.51-5.49 (d, 1H), 5.09-5.07 (m, 1H), 3.74 (s, 3H).

LCMS (M+H=254.0, 93.14%).

### Synthesis of 3-benzylidene-5-(2-(1-methyl-1H-pyrazol-4-yl)phenyl)dihydrofuran-2 (3H)-one

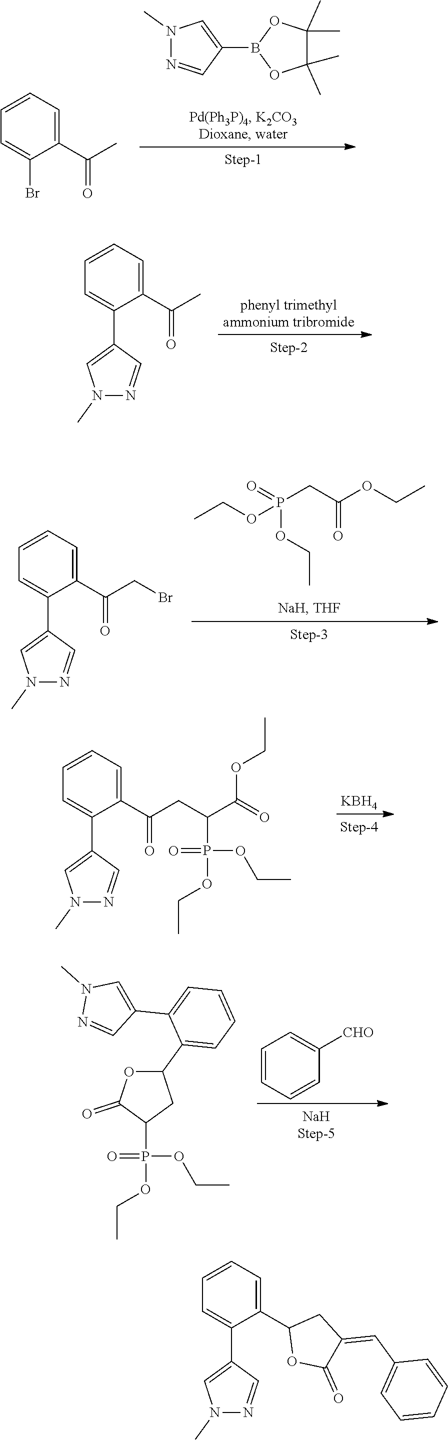

### Step-1: Synthesis of 1-(2-(1-methyl-1H-pyrazol-4-yl)phenyl)ethan-1-one

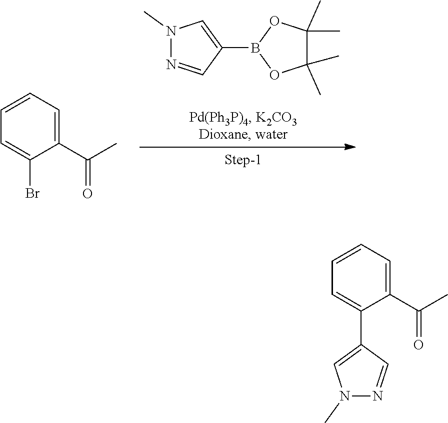

To a stirred solution of 2-bromo acetophenone (1 g, 5.024 mmol) and 1-methyl-4-(4,4,5,5-tetramethyl-1,3,2-dioxaborolan-2-yl)-1H-pyrazole (1.4 g, 6.53 mmoL) in 1,4-dioxane (15 mL), potassium carbonate (1.805 g, 13.06 mmol) in water (15 mL) was added, the resulting mixture was degassed for 5 min, after that Tetrakis(triphenylphosphine)-palladium (100 mg, 10% mol) was added, and then degassed for further 5 min. The resulting reaction mixture was heated at 70° C. for 2 h. The reaction progress was monitored by TLC. Upon disappearance of starting material, the reaction mixture was diluted with ethyl acetate (25 mL) and washed with water (2×20 mL). The organic layer was dried over Na₂SO₄, filtered and then solvent was evaporated under reduced pressure, the obtained crude was purified by column chromatography using silica gel (mesh: 60-120) required compound was eluted at 25-30% ethyl acetate in petroleum ether to afford 1-(2-(1-methyl-1H-pyrazol-4-yl)phenyl)ethan-1-one pale yellow gummy (1.1 g, 98%).

### Step-2: Synthesis 2-bromo-1-(2-(1-methyl-1H-pyrazol-4-yl)phenyl)ethan-1-one

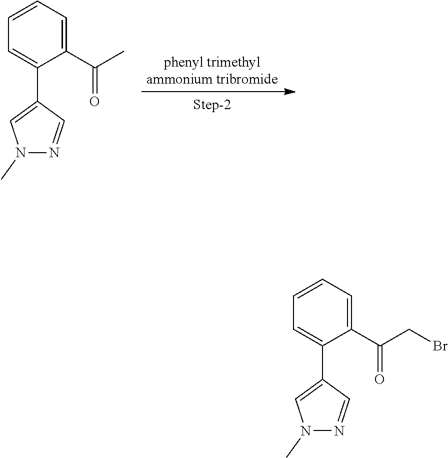 [SMILES: CC(=O)c1ccccc1-c1cnn(C)c1 ; Cn1cc(-c2ccccc2C(=O)CBr)cn1]

To a stirred solution of 1-(2-(1-methyl-1H-pyrazol-4-yl)phenyl)ethan-1-one (1.1 g, 5.49 mmol) in THF (50 mL), phenyl trimethylammonium tribromide (2.26 g, 6.04 mmol) was added at 0° C. and the resulting mixture was stirred at RT for 16 h. The reaction progress was monitored by TLC. Upon disappearance of starting material, the reaction mixture was filtered and filtrate was evaporated under reduced pressure to afford crude methyl 4-(3-chloro-2-(1-methyl-1H-pyrazol-4-yl)phenyl)-4-hydroxy-2-methylenebutanoateas a yellow gummy (170 mg, 90%). The obtained crude was purified by column chromatography using silica gel (mesh: 230-400) required compound was eluted at 2-4% ethyl acetate and petroleum ether to afford 2-bromo-1-(2-(1-methyl-1H-pyrazol-4-yl)phenyl)ethan-1-one as off white solid (750 mg, 49%).

### Step-3: Synthesis of ethyl 2-(diethoxyphosphoryl)-4-(2-(1-methyl-1H-pyrazol-4-yl)phenyl)-4-oxobutanoate

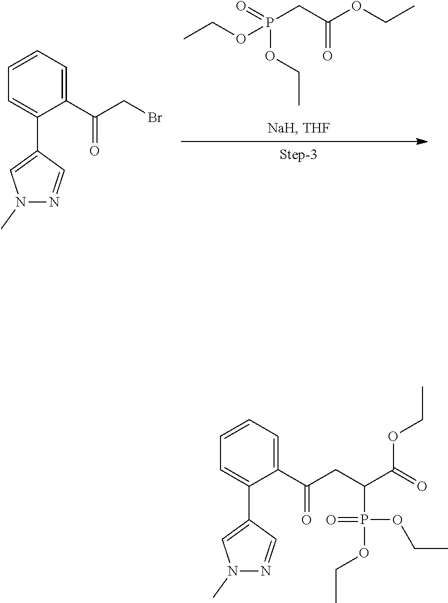 [SMILES: Cn1cc(-c2ccccc2C(=O)CBr)cn1 ; CCOC(=O)CP(=O)(OCC)OCC ; CCOC(=O)C(CC(=O)c1ccccc1-c1cnn(C)c1)P(=O)(OCC)OCC]

To a stirred solution of 2-bromo-1-(2-(1-methyl-1H-pyrazol-4-yl)phenyl)ethan-1-one (750 mg, 0.54 mmol) in THF (10 mL)ethyl 2-(diethoxy phosphoryl)acetate (783 mg, 3.49 mmoL) was added followed by K₂CO₃ (558 mg, 4.03 mmoL) at RT and the reaction mixture was stirred at 70° C. for 16 h. Reaction progress was monitored by TLC. Upon disappearance of starting material, the reaction mixture was filtered and filtrate was concentrated at reduced pressure to obtain crude as pale yellow gummy. The obtained crude was purified by column chromatography using silica gel (mesh: 100-200) required compound was eluted at 70-80% ethyl acetate and petroleum ether to afford ethyl 2-(diethoxyphosphoryl)-4-(2-(1-methyl-1H-pyrazol-4-yl)phenyl)-4-oxobutanoate as a pale yellow gummy (400 mg, 35%).

### Step-4: Synthesis of diethyl (5-(2-(1-methyl-1H-pyrazol-4-yl)phenyl)-2-oxotetrahydrofuran-3-yl)phosphonate

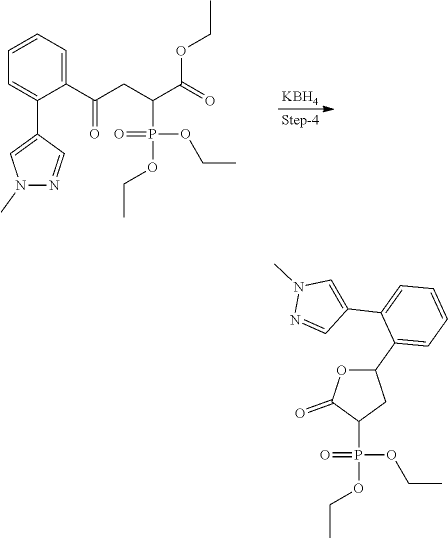 [SMILES: CCOC(=O)C(CC(=O)c1ccccc1-c1cnn(C)c1)P(=O)(OCC)OCC ; "KBH4" ; CCOP(=O)(OCC)C1CC(c2ccccc2-c2cnn(C)c2)OC1=O]

To a stirred solution of ethyl 2-(diethoxyphosphoryl)-4-(2-(1-methyl-1H-pyrazol-4-yl)phenyl)-4-oxobutanoate (400 mg, 0.95 mmol) in MeOH (10 mL) potassium borohydride (77 mg, 1.42 mmoL) was added at 0° C. and the reaction mixture was stirred at 0° C. for 2 h. Reaction progress was monitored by TLC. Upon disappearance of starting material, the reaction mixture was concentrated at reduced pressure to obtain crude as pale yellow gummy. The obtained crude was purified by column chromatography using silica gel (mesh: 100-200) required compound was eluted at 2-3% MeOH in DCM to afford diethyl (5-(2-(1-methyl-1H-pyrazol-4-yl)phenyl)-2-oxotetrahydrofuran-3-yl)phosphonateas a pale yellow gummy (130 mg, 36%).

### Step-5: Synthesis of (Z)-3-benzylidene-5-(2-(1-methyl-1H-pyrazol-4-yl)phenyl)dihydrofuran-2 (3H)-one

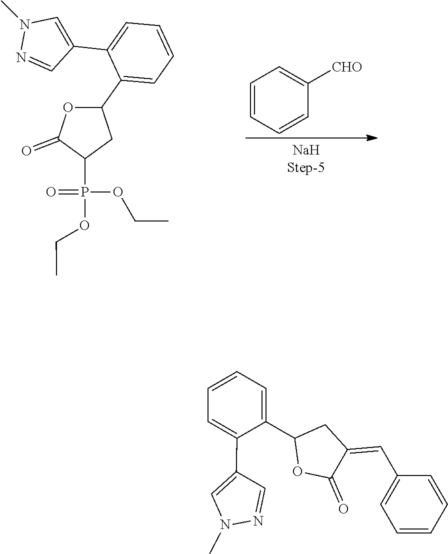 [SMILES: CCOP(=O)(OCC)C1CC(c2ccccc2-c2cnn(C)c2)OC1=O ; O=Cc1ccccc1 ; Cn1cc(-c2ccccc2C2C/C(=C/c3ccccc3)C(=O)O2)cn1]

To a stirred solution of (5-(2-(1-methyl-1H-pyrazol-4-yl)phenyl)-2-oxotetrahydrofuran-3-yl)phosphonate (130 mg, 0.34 mmol) in THF (10 mL) benzaldehyde (48 mg, 0.44 mmoL) was added followed by K₂CO₃ (72 mg, 0.51) at RT and the reaction mixture was stirred at 70° C. for 16 h. The reaction progress was monitored by TLC. Upon disappearance of starting material, the reaction mixture was filtered, and filtrate was concentrated at reduced pressure to obtain crude as pale-yellow gummy. The obtained crude was purified by column chromatography using silica gel (mesh: 100-200) required compound was eluted at 10-15% ethyl acetate and petroleum ether to afford (Z)-3-benzylidene-5-(2-(1-methyl-1H-pyrazol-4-yl)phenyl)dihydrofuran-2 (3H)-one as a pale-yellow gummy (24 mg, 17%).

¹H-NMR-CDCl₃: 7.87-7.85 (d, 1H), 7.64-7.62 (m, 1H), 7.57-7.55 (m, 2H), 7.54-7.52 (m, 3H), 7.50-7.45 (m, 4H), 7.44-7.39 (m, 6H), 5.85-5.81 (m, 1H), 5.78-5.74 (m, 1H), 3.95 (s, 3H), 3.50-3.35 (m, 1H), 3.34-3.33 (m, 1H), 3.25-3.14 (m, 2H).

LCMS (M+H=331.2, 95.5%).

### (R)-3-acryloyl-4-benzyloxazolidin-2-one

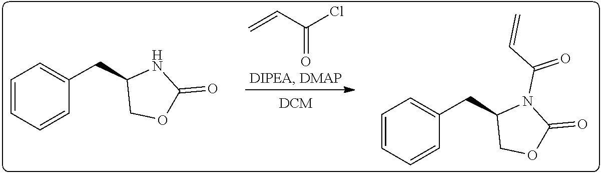

To a stirred solution of (R)-4-benzyloxazolidin-2-one (250 mg, 1.41 mmol) in DCM (20 mL) DIPEA (273.4 mg, 2.11 mmoL) was added followed by DMAP (18 mg, 0.14 mmoL). The reaction mixture was cooled to 0° C. and acryloyl chloride (154.19 mg, 1.71 mmoL) was added drop wise. Then the resulting reaction mixture was stirred at RT for 16 h. The reaction progress was monitored by TLC. Upon disappearance of starting material, the reaction mixture was quenched with water (20 mL) and extracted with ethyl acetate (2×20 mL). The organic layer was dried over Na₂SO₄, filtered and then solvent was evaporated under reduced pressure and temperature, the obtained crude was purified by column chromatography using silica gel (mesh: 100-200) required compound was eluted at 30-40% ethyl acetate and petroleum ether to afford (R)-3-acryloyl-4-benzyloxazolidin-2-oneaspale-yellow gummy (45 mg, 14%).

¹H-NMR-CDCl3: 7.55-7.48 (m, 1H), 7.36-7.32 (m, 2H), 7.44-7.39 (m, 1H), 7.29-7.28 (m, 1H), 7.23-7.21 (m, 2H), 6.63-6.58 (m, 1H), 5.95-5.92 (m, 1H), 4.76-4.72 (m, 1H), 4.26-4.18 (m, 2H), 3.37-3.33 (m, 1H), 2.84-2.78 (m, 1H).

LCMS (M+H=232.0, 99.14%).

### 5-(4,5-difluoro-2-(1-methyl-1H-pyrazol-4-yl)phenyl)-3-methylenedihydrofuran-2 (3H)-one

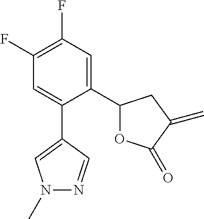 [SMILES: C=C1CC(c2cc(F)c(F)cc2-c2cnn(C)c2)OC1=O]

¹H-NMR (400 MHz, CDCl₃): 7.49 (s, 1H), 7.45 (s, 1H), 7.26-7.25 (d, 1H), 7.13 (m, 1H), 6.32-6.31 (t, 1H) 5.70-5.69 (t, 1H), 5.63-5.60 (t, 1H), 3.97 (s, 3H), 3.25-3.10 (m, 1H), 2.89-2.82 (m, 1H).

LCMS (M+H=291.0, 98.09%).

### 3-methylene-5-(2-(6-methylpyridin-3-yl)phenyl)dihydrofuran-2 (3H)-one

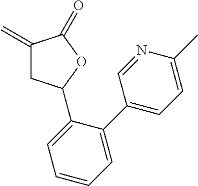

¹H-NMR (400 MHz, CDCl₃): 8.44 (s, 1H), 7.58-7.56 (d, 1H), 7.51-7.40 (m, 4H), 7.24 (s, 1H), 6.28-6.27 (d, 1H) 5.64-5.63 (d, 1H), 5.52-5.48 (t, 1H), 3.16-3.09 (m, 1H), 2.90-2.85 (m, 1H), 2.63 (s, 3H).

LCMS (M+H=266.0, 96.75%).

### 5-(2-(1-(difluoromethyl)-1H-pyrazol-4-yl)phenyl)-3-methylenedihydrofuran-2 (3H)-one

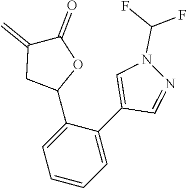 [SMILES: C=C1CC(c2ccccc2-c2cnn(C(F)F)c2)OC1=O]

¹H-NMR (400 MHz, CDCl₃): 7.90 (s, 1H), 7.73 (s, 1H), 7.48-7.45 (t, 1H), 7.43-7.38 (m, 3H), 7.33-7.31 (d, 1H), 6.32 (s, 1H), 5.07 (s, 1H), 5.66-5.63 (m, 1H), 3.26-3.20 (m, 1H), 2.97-2.93 (d, 1H).

LCMS (M+H=291.0, 97.15%).

### 5-(4-fluoro-2-(1-methyl-1H-pyrazol-4-yl)phenyl)-3-methylenedihydrofuran-2 (3H)-one

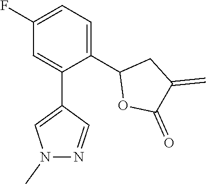 [SMILES: C=C1CC(c2ccc(F)cc2-c2cnn(C)c2)OC1=O]

¹H-NMR (400 MHz, CDCl₃): 7.53 (s, 1H), 7.49 (s, 1H), 7.42-7.39 (s, 1H), 7.07-7.00 (m, 2H), 6.31 (s, 1H), 5.69-5.61 (m, 2H), 3.98 (s, 3H), 3.26-3.20 (m, 1H), 2.95-2.89 (m, 1H).

LCMS (M+H=272.9, 99.12%).

### 3-methylene-5-(2-morpholinopyridin-3-yl)dihydrofuran-2 (3H)-one

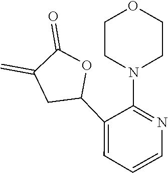 [SMILES: C=C1CC(c2cccnc2N2CCOCC2)OC1=O]

¹H-NMR (400 MHz, CDCl₃): 8.37-8.35 (m, 1H), 7.68-7.66 (m, 1H), 7.10-7.06 (s, 1H), 7.36-7.35 (m, 1H), 5.84-5.80 (m, 1H), 5.74-5.73 (m, 1H), 3.89-3.81 (m, 4H), 3.51-3.45 (m, 1H), 3.26-3.20 (m, 2H), 3.07-3.01 (m, 2H), 2.95-2.90 (m, 1H).

LCMS (M+H=261.0, 96.93%).

### 5-(2-(6-methoxypyridin-3-yl)phenyl)-3-methylenedihydrofuran-2 (3H)-one

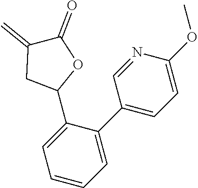 [SMILES: C=C1CC(c2ccccc2-c2ccc(OC)nc2)OC1=O]

¹H-NMR (400 MHz, CDCl₃): 8.10-8.09 (d, 1H), 7.57-7.54 (m, 1H), 7.50-7.38 (m, 3H), 7.24-7.23 (m, 1H), 6.84-6.82 (d, 1H), 6.28-6.27 (t, 1H), 5.64-5.63 (t, 1H), 5.55-5.52 (t, 1H), 3.99 (s, 3H), 3.17-3.11 (m, 1H), 2.90-2.84 (m, 1H).

LCMS (M+H=282.1, 99.23%).

### 5-(2-(2-methoxypyrimidin-5-yl)phenyl)-3-methylenedihydrofuran-2 (3H)-one

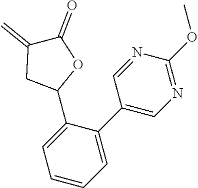 [SMILES: C=C1CC(c2ccccc2-c2cnc(OC)nc2)OC1=O]

¹H-NMR (400 MHz, CDCl₃): 8.51 (s, 2H), 7.53-7.42 (m, 3H), 7.24 (s, 1H), 6.31-6.30 (t, 1H), 5.68-5.67 (t, 1H), 5.48-5.45 (t, 1H), 4.08 (s, 3H), 3.49-3.48 (d, 1H), 3.21-3.14 (m, 1H), 2.94-2.88 (m, 1H).

LCMS (M+H=283.2, 97.22%).

### 3-(1-methyl-1H-pyrazol-4-yl)-4-(4-methylene-5-oxotetrahydrofuran-2-yl)benzoic acid

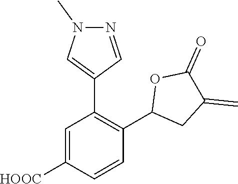 [SMILES: C=C1CC(c2ccc(C(=O)O)cc2-c2cnn(C)c2)OC1=O]

¹H-NMR (400 MHz, DMSO): 13.4 (bs, 1H), 7.95 (s, 1H), 7.89-7.87 (d, 2H), 7.62 (d, 1H), 7.44-7.42 (d, 1H), 6.13 (s, 1H), 5.86-583 (m, 1H), 5.77 (s, 1H), 3.90 (s, 3H), 3.50-3.40 (m, 1H), 2.86-2.81 (m, 1H).

LCMS (M+H=299.1, 94.55%).

### 3-methylene-5-(2-(1-(tetrahydrofuran-3-yl)-1H-pyrazol-4-yl)phenyl)dihydrofuran-2 (3H)-one

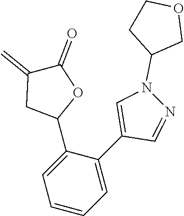 [SMILES: C=C1CC(c2ccccc2-c2cnn(C3CCOC3)c2)OC1=O]

¹H-NMR (400 MHz, CDCl₃): 7.56-7.55 (m, 2H), 7.44-7.41 (m, 1H), 7.38-7.33 (m, 2H), 7.31-7.29 (m, 1H), 6.30-6.29 (m, 1H), 5.72-5.67 (m, 1H), 5.68-5.66 (m, 1H), 5.04-5.0 (m, 1H), 4.19-4.11 (m, 3H), 4.09-3.94 (m, 1H), 3.25-3.18 (m, 1H), 2.97-2.91 (m, 1H), 2.54-2.38 (m, 2H).

LCMS (M+H=311.1, 96.59%).

### 5-(isoquinolin-1-yl)-3-methylenedihydrofuran-2 (3H)-one

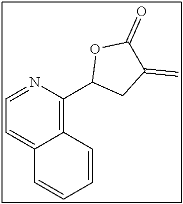 [SMILES: C=C1CC(c2nccc3ccccc23)OC1=O]

¹H-NMR (300 MHz, CDCl₃) δ: 8.50 (d, J=6 Hz, 1H), 8.28 (d. J=9 Hz, 1H), 7.88 (d. J=9 Hz, 1H), 7.75-7.64 (m, 1H), 6.37-6.33 (m, 2H), 5.79-5.74 (m, 1H), 4.06-3.97 (m, 1H), 3.36-3.27 (m, 1H),

m/z 226.11[M+H]⁺.

### 5-(2-(1-methyl-1H-pyrazol-3-yl)phenyl)-3-methylenedihydrofuran-2 (3H)-one

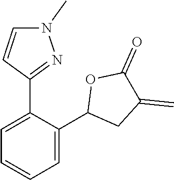 [SMILES: C=C1CC(c2ccccc2-c2ccn(C)n2)OC1=O]

1H-NMR (300 MHz, CDCl₃) δ: 7.54-7.51 (m, 1H), 7.48-7.45 (m, 1H), 7.41-7.40 (m, 1H), 7.37-7.34 (m, 2H), 6.44 (d, J=3 Hz, 1H), 6.27 (t, J=3 Hz, 1H), 6.20-6.10 (1H, m), 5.61 (t, J=3 Hz), 3.95 (s, 3H), 3.54-3.46 (m, 1H), 2.86-2.78 (m, 1H).

m/z 255.17 [M+H]⁺.

### 5-(2-(1-methyl-1H-pyrazol-4-yl)phenyl)-3-methylenepyrrolidin-2-one

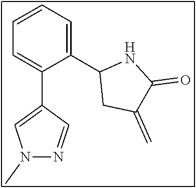 [SMILES: C=C1CC(c2ccccc2-c2cnn(C)c2)NC1=O]

¹H-NMR (300 MHz, CDCl₃) δ: 7.48-7.25 (m, 6H), 6.09-6.06 (m, 1H), 5.38 (bs, 1H), 5.01-4.97 (m, 1H), 3.98 (s, 3H), 3.26-3.20 (m, 1H), 2.74-2.61 (m, 1H),

m/z 254.24 [M+H]⁺.

### 1-methyl-5-(2-(1-methyl-1H-pyrazol-4-yl)phenyl)-3-methylenepyrrolidin-2-one

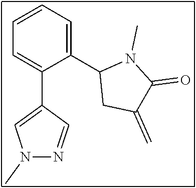 [SMILES: C=C1CC(c2ccccc2-c2cnn(C)c2)N(C)C1=O]

¹H-NMR (300 MHz, CDCl₃) δ: 7.48 (s, 1H), 7.37-7.26 (m, 4H), 7.13-7.10 (m, 1H), 6.06 (s, 1H), 5.33-5.29 (m, 1H), 4.85-4.81 (m, 1H), 3.98 (s, 3H), 3.35-3.14 (m, 1H), 2.70 (s, 3H), 2.66-2.59 (m, 1H),

m/z 268.21 [M+H]⁺.

### 5-(5-methyl-2-(1-(2-morpholinoethyl)-1H-pyrazol-4-yl)phenyl)-3-methylenedihydrofuran-2 (3H)-one

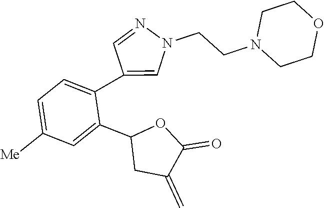 [SMILES: C=C1CC(c2cc(C)ccc2-c2cnn(CCN3CCOCC3)c2)OC1=O]

¹H-NMR (400 MHz, CDCl₃): 8.62 (s, 1H), 7.90 (s, 1H), 7.56 (s, 1H), 7.26-7.24 (d, 1H), 7.19 (brs, 1H), 6.54 (s, 2H), 6.12 (brs, 1H), 5.79-5.76 (m, 2H), 4.28-4.24 (m, 2H), 3.54-3.53 (m, 4H), 2.73-2.671 (m, 2H), 2.41 (brs, 4H), 2.32 (s, 3H).

LCMS (M+H=368.1).

### N-methyl-N-(1-(2-(1-methyl-1H-pyrazol-4-yl)phenyl)ethyl)ethenesulfonamide

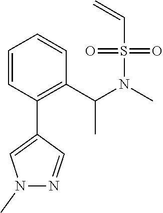 [SMILES: C=CS(=O)(=O)N(C)C(C)c1ccccc1-c1cnn(C)c1]

¹H NMR (400 MHz, CDCl₃): δ=7.66 (s, 1H), 7.55 (s, 1H), 7.46-7.44 (m, 1H), 7.37 (s, 3H), 6.15 (d, 1H, J=16.4 Hz), 6.02-5.95 (m, 1H), 5.87 (d, 1H, J=10 Hz), 5.50-5.45 (q, 1H, J=9.6 Hz), 4.026 (d, 3H, J=4.0 Hz), 2.58 (s, 3H), 1.54 (d, 3H, J=6.8 Hz).

m/z 306.1 [M+1]⁺

### 2-(4-(2-(4-methylene-5-oxotetrahydrofuran-2-yl)phenyl)-1H-pyrazol-1-yl)-N-(oxetan-3-yl)acetamide

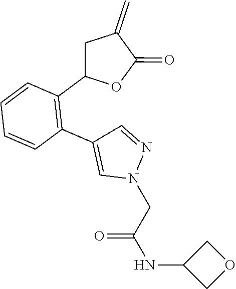 [SMILES: C=C1CC(c2ccccc2-c2cnn(CC(=O)NC3COC3)c2)OC1=O]

1H NMR (400 MHz, CDCl 3): δ=7.72 (s, 1H), 7.60 (s, 1H), 7.47-7.44 (m, 1H), 7.43-7.38 (m, 2H), 7.38-7.27 (m, 1H), 7.21-7.20 (m, 1H), 6.33 (t, 1H, J=2.8 Hz), 5.72-7.67 (m, 2H), 5.10-5.01 (m, 1H), 4.93 (t, 2H, J=14.4 Hz), 4.87 (s, 2H), 4.54-4.50 (m, 2H), 3.30-3.23 (m, 1H), 3.04-2.97 (m, 1H).

LCMS: m/z 354.1 [M⁺+H]; (93.11% purity).

Additional compounds that were synthesized:

### 3-methylene-5-(2-(1-(2, 2, 2-trifluoroethyl)-4-yl) phenyldihydrofuran-2 (3H)-one

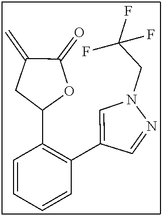 [SMILES: C=C1CC(c2ccccc2-c2cnn(CC(F)(F)F)c2)OC1=O]

¹H-NMR (400 MHz, CDCl₃): 7.64-7.61 (d, 2H), 7.46-7.41 (m, 1H), 7.40-7.38 (s, 2H), 7.35-7.25 (m, 1H), 6.31-6.29 (t, 1H), 5.68-5.64 (m, 2H), 4.81-4.79 (m, 2H), 3.24-3.16 (m, 1H), 2.97-2.96 (m, 1H).

### 5-(5-methyl-2-(1-methyl-1H-pyrazol-4-yl)phenyl)-3-methylenedihydrofuran-2 (3H)-one

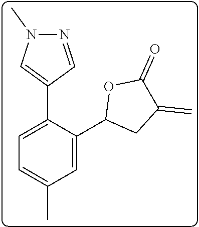

¹H-NMR (400 MHz, CDCl₃): 7.50 (s, 1H), 7.43 (s, 1H), 7.22 (s, 1H), 7.19-7.14 (m, 2H), 6.30-6.29 (t, 1H), 5.71-5.66 (m, 2H), 3.97 (s, 3H), 3.25-3.17 (m, 1H), 2.95-2.88 (m, 1H), 2.39 (s, 3H).

### 5-(2-methyl-6-(1-methyl-1H-pyrazol-4-yl)phenyl)-3-methylenedihydrofuran-2 (3H)-one

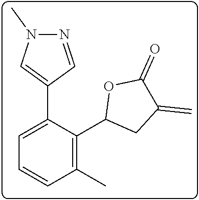

LCMS: m/z: 269.31 (M+1)

### 5-(5-(4-methoxyphenyl)-1-methyl-1H-pyrazol-3-yl)-3-methylenedihydrofuran-2 (3H)-one

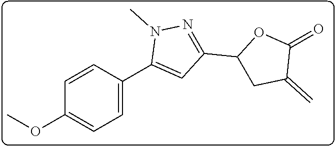 [SMILES: C=C1CC(c2cc(-c3ccc(OC)cc3)n(C)n2)OC1=O]

¹H-NMR (400 MHz, CDCl₃): 7.69-7.65 (m, 2H), 6.94-6.90 (m, 2H), 6.46 (s, 1H), 6.36-6.35 (t, 1H), 5.81-5.79 (t, 1H), 5.62-5.58 (m, 1H), 3.96 (s, 3H), 3.83 (s, 3H), 3.45-3.39 (m, 1H), 3.25-3.20 (m, 1H).

### 5-(1′,2-dimethyl-1′H, 2H-[3,4′-bipyrazol]-5-yl)-3-methylenedihydrofuran-2 (3H)-one

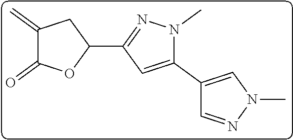

¹H-NMR (400 MHz, CDCl₃): 7.72-7.65 (d, 2H), 6.35-6.27 (m, 2H), 5.79 (brs, 1H), 5.59-5.56 (t, 1H), 3.92 (s, 6H), 3.20-3.17 (m, 1H), 2.95-2.44 (d, 1H).

### 5-(4′-fluoro-[1,1′-biphenyl]-2-yl)-3-methylenedihydrofuran-2 (3H)-one

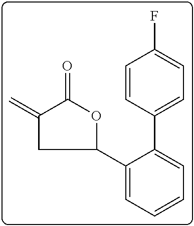 [SMILES: C=C1CC(c2ccccc2-c2ccc(F)cc2)OC1=O]

¹H-NMR (400 MHz, CDCl₃): 7.48-7.36 (m, 3H), 7.32-7.22 (m, 3H), 7.16-7.11 (m, 2H), 6.3 (t, 1H), 5.62 (t, 1H), 5.58-5.50 (m, 1H), 3.12-3.02 (m, 1H), 2.90-2.80 (m, 1H).

### 5-(4-(1-methyl-1H-pyrazol-4-yl)pyridin-3-yl)-3-methylenedihydrofuran-2 (3H)-one

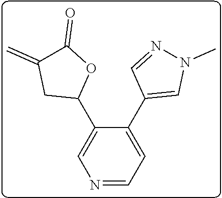

¹H-NMR (400 MHz, CDCl₃): 8.7 (s, 1H), 8.6 (d, 1H), 7.64-7.62 (s, 2H), 7.3 (d, 1H), 6.36-6.32 (s, 1H), 5.78-5.72 (m, 2H), 4.0-3.98 (s, 3H), (3.35-3.25 (m, 1H), 3.22-3.12 (m, 1H).

LCMS-(M+H=256.28, 98.5%).

### 5-(2-(1-methyl-1H-pyrazol-4-yl)pyridin-3-yl)-3-methylenedihydrofuran-2 (3H)-one

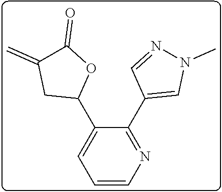

¹H-NMR (400 MHz, CDCl₃): 8.62-8.58 (d, 1H), 8.16-8.14 (s, 1H), 7.922-7.82 (m, 2H), 7.44-7.38 (m, 1H), 6.04-6.02 (t, 1H), 6.0-5.58 (m, 1H), 5.56 (s, 1H), 3.98 (s, 3H), 3.53-3.50 (m, 1H), 2.90-2.70 (m, 1H).

LCMS (M+H=256.78, 99%)

### 5-(3-(1-methyl-1H-pyrazol-4-yl)pyridin-4-yl)-3-methylenedihydrofuran-2 (3H)-one

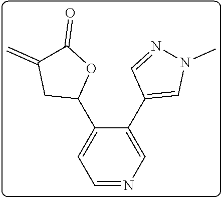

¹H-NMR (400 MHz, CDCl₃): 8.61-8.52 (m, 2H), 7.59-7.55 (m, 2H), 7.38 (d, 1H), 6.3 (t, 1H), 5.74-5.72 (m, 2H), 4.0 (s, 3H), 3.22-3.12 (m, 1H), 2.80-2.72 (m, 1H).

### 5-(2-(1-methyl-1H-indol-4-yl)phenyl)-3-methylenedihydrofuran-2 (3H)-one

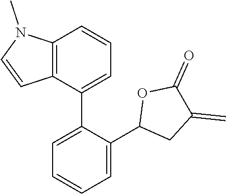 [SMILES: C=C1CC(c2ccccc2-c2cccc3c2ccn3C)OC1=O]

1H-NMR (300 MHz, CDCl₃) δ: 7.50-7.24 (m, 6H), 7.05 (d, J-=6 Hz, 1H), 6.22-6.12 (m, 2H), 5.62-5.48 (m, 2H), 3.82 (s, 3H), 2.89-2.65 (m, 2H), LCMS: Rt 2.65 min (97.74% purity), m/z 304.16 [M+H]⁺.

### 5-(2-(1-ethyl-1H-pyrazol-4-yl)phenyl)-3-methylenedihydrofuran-2 (3H)-one

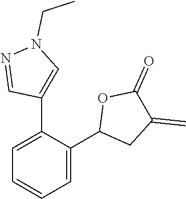 [SMILES: C=C1CC(c2ccccc2-c2cnn(CC)c2)OC1=O]

¹H-NMR (300 MHz, CDCl₃) δ: 7.51 (d, J=12 Hz, 2H), 7.44-7.41 (1H, m), 7.38-7.29 (m, 3H), 6.29 (t, J=3 Hz, 1H), 5.73 (q, J=6 Hz), 5.66 (t, 1H, 3 Hz), 4.23 (q, J=9 Hz, 2H), 3.27-3.17 (m, 1H), 2.98-2.89 (m, 1H), 1.56 (t, J=6 Hz, 3H) LCMS: Rt 2.65 min (96.9% purity), m/z 269.16 [M+H]⁺.

### 5-(2-(1-isobutyl-1H-pyrazol-4-yl)phenyl)-3-methylenedihydrofuran-2 (3H)-one

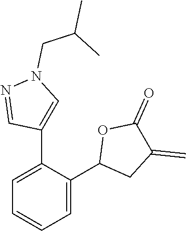 [SMILES: C=C1CC(c2ccccc2-c2cnn(CC(C)C)c2)OC1=O]

¹H-NMR (300 MHz, CDCl₃) δ: 7.69 (s, 1H), 7.55 (s, 1H), 7.47-7.38 (3H, m), 7.32-7.29 (m, 1H), 6.32 (t, J=3 Hz, 1H), 5.71 (t, J=6 Hz), 5.68-5.63 (m, 1H), 4.06 (d, J=6 Hz, 2H), 3.29-3.19 (m, 1H), 3.03-2.93 (m, 1H), 2.25-2.21 (1H, m), 0.95 (d, J=6 Hz, 6H) LCMS: Rt 2.65 min (96.9% purity), m/z 297.2 [M+H]⁺.

### 3-methylene-5-(2-(1,3,5-trimethyl-1H-pyrazol-4-yl)phenyl)dihydrofuran-2 (3H)-one

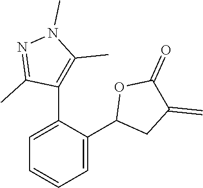

¹H-NMR (300 MHz, CDCl₃), Mixture of rotamers δ: 7.50-7.38 (m, 3H), 7.10 (t, J=6 Hz, 1H), 6.29 & 6.21 (2 t, J=3 Hz, 1H), 5.68 & 5.60 (2 t, J=3 Hz, 1H), 5.38 & 5.35 (2 t, J=3 Hz, 1H), 3.96 & 3.94 (2s, 3H), 3.33-3.30 (m, 1H), 6.32 (t, J=3 Hz, 1H), 5.71 (t, J=6 Hz), 5.68-5.63 (m, 1H), 4.06 (d, J=6 Hz, 2H), 3.29-3.19 (m, 1H), 2.80-2.55 (m, 1H), 2.18-2.11 (m, 6H), m/z 283.2 [M+H]⁺.

### 5-(2-(1-benzyl-1H-pyrazol-4-yl)phenyl)-3-methylenedihydrofuran-2 (3H)-one

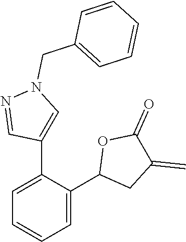 [SMILES: C=C1CC(c2ccccc2-c2cnn(Cc3ccccc3)c2)OC1=O]

¹H-NMR (300 MHz, CDCl₃) δ: 7.58-7.26 (m, 11H), 6.29-6.28 (m, 1H), 5.72-5.63 (m, 4H), 3.21-3.13 (m, 1H), 2.93-2.85 (m, 1H), m/z 331.18 [M+H]⁺.

### 5-(2-(1-methyl-1H-pyrazol-5-yl)phenyl)-3-methylenedihydrofuran-2 (3H)-one

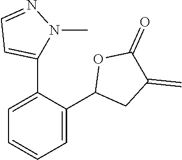 [SMILES: C=C1CC(c2ccccc2-c2ccnn2C)OC1=O]

¹H-NMR (300 MHz, CDCl₃) δ: 7.56-7.51 (m, 3H), 7.46-7.40 (m, 1H), 7.29-7.28 (m, 1H), 6.29-6.24 (m, 2H), 5.64 (d, J=3 Hz, 1H), 5.38 (t, J=6 Hz, 1H), 3.13-3.04 (m, 1H), 2.80-2.71 (m, 1H), m/z 255.57 [M+H]⁺.

### 5-(2-(1-methyl-1H-indazol-4-yl)phenyl)-3-methylenedihydrofuran-2 (3H)-one

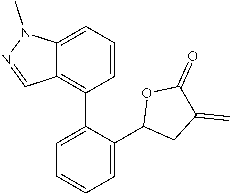 [SMILES: C=C1CC(c2ccccc2-c2cccc3c2cnn3C)OC1=O]

¹H-NMR (300 MHz, CDCl₃) δ: 7.71 (bs, 1H), 7.56-7.34 (m, 7H), 6.21 (t, J=3 Hz, 1H), 5.53 (t, 3 Hz, 1H), 5.53 (bm, 1H), 4.13 (s, 3H), 2.98-2.78 (m, 2H), m/z 305.18 [M+H]⁺.

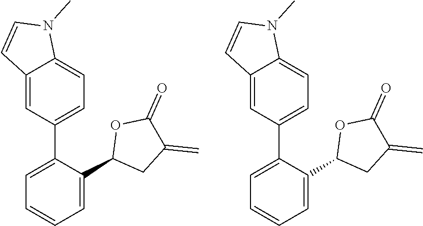 [SMILES: C=C1C[C@@H](c2ccccc2-c2ccc3c(ccn3C)c2)OC1=O ; C=C1C[C@H](c2ccccc2-c2ccc3c(ccn3C)c2)OC1=O]

Tentative assigned stereochemistry

### (S)-5-(2-(1-methyl-1H-indol-5-yl)phenyl)-3-methylenedihydrofuran-2 (3H)-one

¹H-NMR (300 MHz, CDCl₃) δ: 7.53-7.32 (m, 6H), 7.17-7.12 (m, 2H), 6.52-6.51 (m, 1H), 6.23-6.21 (m, 1H), 5.70-5.65 (m, 1H), 5.56 (t, J=3 Hz, 1H), 3.85 (s, 1H), 3.01-2.98 (m, 1H), 2.88-2.78 (m, 1H) m/z 304.15 [M+H]⁺.

### (R)-5-(2-(1-methyl-1H-indol-5-yl)phenyl)-3-methylenedihydrofuran-2 (3H)-one

¹H-NMR (300 MHz, CDCl₃) δ: 7.53-7.32 (m, 6H), 7.17-7.12 (m, 2H), 6.52-6.51 (m, 1H), 6.23-6.21 (m, 1H), 5.70-5.65 (m, 1H), 5.56 (t, J=3 Hz, 1H), 3.85 (s, 1H), 3.01-2.98 (m, 1H), 2.88-2.78 (m, 1H), m/z 304.15 [M+H]⁺.

### tert-butyl 2-(4-(2-(4-methylene-5-oxotetrahydrofuran-2-yl)phenyl)-1H-pyrazol-1-yl)acetate

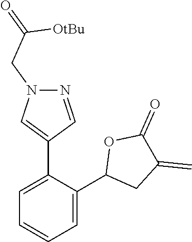 [SMILES: C=C1CC(c2ccccc2-c2cnn(CC(=O)OC(C)(C)C)c2)OC1=O]

¹H-NMR (300 MHz, CDCl₃) δ: 7.57 (d, J=6 Hz, 2H), 7.46-7.30 (m, 4H), 6.26 (t, J=3 Hz, 1H), 5.72 (t, J=6 Hz, 1H), 5.65 (t, J=3 Hz, 1H), 4.86 (s, 2H), 3.27-3.21 (m, 1H), 2.94-2.81 (m, 1H), 1.47 (s, 9H),) m/z 355.25 [M+H]⁺.

### 3-methylene-5-(2-phenoxyphenyl)dihydrofuran-2 (3H)-one

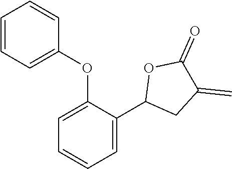 [SMILES: C=C1CC(c2ccccc2Oc2ccccc2)OC1=O]

¹H-NMR (300 MHz, CDCl₃) δ: 7.44 (dd, J=3 Hz, 1H), 7.37-7.24 (m, 3H), 7.16-7.10 (m, 2H), 7.00-6.97 (m, 2H), 6.88-6.85 (m, 1H), 6.27 (t, J=3 Hz, 1H), 5.80 (t, J=6 Hz, 1H), 5.62 (t, J=3 Hz, 1H), 3.41-3.33 (m, 1H), 2.94-2.81 (m, 1H).

M/z 267.19 [M+H]⁺.

### 3-methylene-5-(2-(thiophen-2-yl)phenyl)dihydrofuran-2 (3H)-one

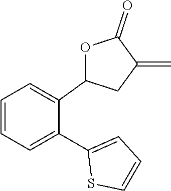 [SMILES: C=C1CC(c2ccccc2-c2cccs2)OC1=O]

¹H-NMR (300 MHz, CDCl₃) δ: 7.46-7.36 (m, 5H), 7.12-7.09 (m, 1H), 7.04-7.02 (m, 1H), 6.28 (t, J=3 Hz, 1H), 5.79 (t, J=6 Hz, 1H), 5.64 (t, J=3 Hz, 1H), 3.73-3.16 (m, 1H), 2.890-2.87 (m, 1H), m/z 257.10 [M+H]⁺.

### 3-methylene-5-(2-(2-morpholinoethoxy)phenyl)dihydrofuran-2 (3H)-one

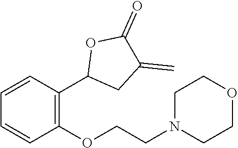 [SMILES: C=C1CC(c2ccccc2OCCN2CCOCC2)OC1=O]

¹H-NMR (300 MHz, CDCl₃) δ: 7.29 (t, 2H), 6.99-6.87 (m, 2H), 6.27 (t, J=3 Hz, 1H), 5.71 (t, J=6 Hz, 1H), 5.62 (t, J=3 Hz, 1H), 4.16-4.09 (m, 2H), 3.75-3.69 (m, 4H), 3.46-3.43 (m, 1H), 2.94-2.84 (m, 1H), 2.79-2.75 (m, 2H), 2.56-2.53 (m, 4H), m/z 304.25 [M+H]⁺.

### Synthesis of (S)-2-phenyl-2-(vinylsulfonamido)acetic acid

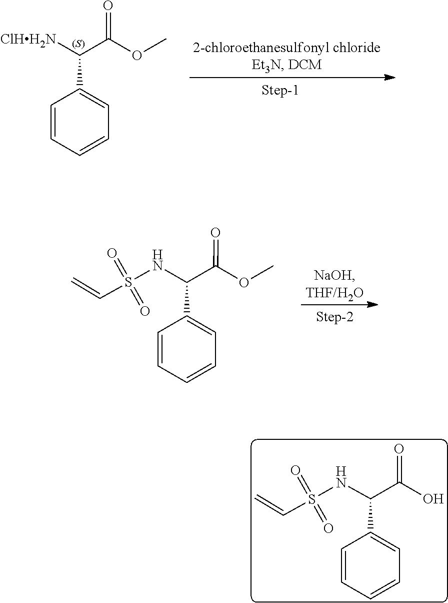

### Step-1: Methyl (S)-2-phenyl-2-(vinylsulfonamido) acetate

To a solution of methyl (S)-2-amino-2-phenylacetate hydrochloride (0.3 g, 1.487 mmol) in DCM (10 mL) were added TEA (0.9 ml, 6.691 mmol) and 2-Chloro ethane sulfonyl chloride (0.15 ml, 1.487 mmol) at 0° C. Reaction mixture was stirred at 0° C. for 2 h. Reaction progress was monitored by TLC. Upon disappearance of starting material, the reaction mixture was diluted with DCM (2×30 mL), washed with water (2×30 mL). The organic layer was dried over Na₂SO₄, filtered, evaporated under reduced pressure, and purified by column chromatography. Required compound was eluted in 80% Ethyl acetate and hexane to afford Methyl (S)-2-phenyl-2-(vinylsulfonamido)acetate. Yield 0.329 g (86.80%).

### Step-2: (S)-2-phenyl-2-(vinylsulfonamido)acetic acid

To a stirred solution of Methyl (S)-2-phenyl-2-(vinylsulfonamido)acetate (0.329 g, 1.288 mmol) in THF (3 mL) and Water (3 ml) was added NaOH (0.077 g, 1.932 mmol) at 0° C. Reaction mixture was stirred at RT for 2 h. Reaction progress was monitored by TLC. Upon disappearance of starting material, the reaction mixture was diluted with DCM (2×30 mL), washed with water (2×30 mL). Aqueous layer was acidified with 1N HCl and extracted with 20% MeOH/DCM. Organic layer was dried over Na₂SO₄, filtered, evaporated under reduced pressure to afford (S)-2-phenyl-2-(vinylsulfonamido)acetic acid. Yield 0.1 g (32.25%).

¹H NMR (400 MHz, dmso) δ 8.02 (d, J=48.6 Hz, 1H), 7.41-7.26 (m, 5H), 6.58 (dd, J=16.1, 9.9 Hz, 1H), 5.97 (d, J=16.3 Hz, 1H), 5.86 (d, J=9.7 Hz, 1H), 4.72 (s, 1H).

MS (m/z): 240 [M−H]⁺.

### Synthesis of (S)—N, 2-diphenyl-2-(vinylsulfonamido)acetamide

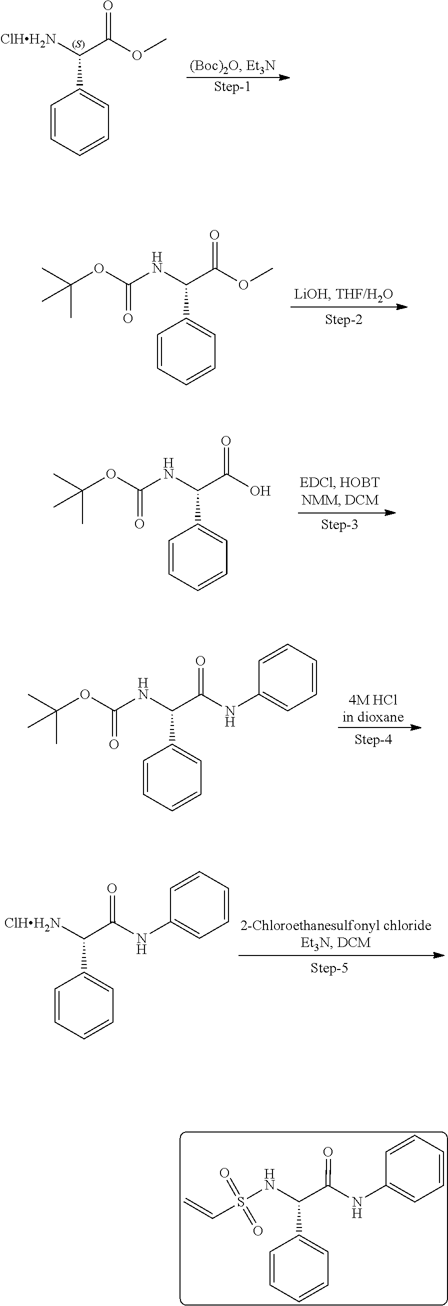

### Step-1: methyl (S)-2-((tert-butoxycarbonyl)amino)-2-phenylacetate

To a solution of methyl (S)-2-amino-2-phenylacetate hydrochloride (1 g, 4.959 mmol) in DCM (10 mL) were added TEA (2.073 ml, 14.877 mmol) and (Boc)₂O (1.365 ml, 5.950 mmol) at 0° C. Reaction mixture was stirred at RT for 12 h. Reaction progress was monitored by TLC. Upon disappearance of starting material, the reaction mixture was diluted with DCM (2×50 mL), washed with water (2×50 mL). The organic layer was dried over Na₂SO₄, filtered, evaporated under reduced pressure, and purified by column chromatography. Required compound was eluted in 10% Ethyl acetate and hexane to afford methyl (S)-2-((tert-butoxycarbonyl)amino)-2-phenylacetate. Yield 1.1 g (83.65%).

### Step-2: (S)-2-((tert-butoxycarbonyl)amino)-2-phenylacetic acid

To a stirred solution of methyl (S)-2-((tert-butoxycarbonyl)amino)-2-phenylacetate (1.1 g, 4.150 mmol) in THF (10 mL) and Water (10 ml) was added LiOH·H₂O (0.209 g, 4.98 ml) at 0° C. Reaction mixture was stirred at RT for 4 h. Reaction progress was monitored by TLC. Upon disappearance of starting material, the reaction mixture was diluted with EtOAc (100 mL), washed with water (2×50 mL). The organic layer was dried over Na₂SO₄, filtered, evaporated under reduced pressure to afford (S)-2-((tert-butoxycarbonyl)amino)-2-phenylacetic acid. Yield 0.85 g (81.49%).

### Step-3: tert-butyl (S)-(2-oxo-1-phenyl-2-(phenylamino)ethyl)carbamate

To a stirred solution of (S)-2-((tert-butoxycarbonyl)amino)-2-phenylacetic acid (0.4 g, 1.593 mmol) in DCM (15 mL) was added Aniline (0.148 g, 1.593 mmol), HoBt (0.258 g, 1.911) and NMM (0.161 g, 1.593 mmol) at 0° C. Reaction mixture was stirred at 0° C. for 30. Then added EDC·HCl (0.305 g, 1.593 mmol), stirred the reaction mixture at 0° C. for 3 h and RT for 12 h. Reaction progress was monitored by TLC. Upon disappearance of starting material, the reaction mixture was diluted with DCM (2×50 mL), washed with water (2×50 mL). The organic layer was dried over Na₂SO₄, filtered, evaporated under reduced pressure, and purified by column chromatography. Required compound was eluted in 50% Ethyl acetate and hexane to afford tert-butyl (S)-(2-oxo-1-phenyl-2-(phenylamino)ethyl)carbamate. Yield 0.3 g (57.69%).

### Step-4: (S)-2-amino-N, 2-diphenylacetamide hydrochloride

To a stirred solution of tert-butyl (S)-(2-oxo-1-phenyl-2-(phenylamino)ethyl)carbamate (0.3 g, 0.920 mmol) in DCM (5 mL) was added 4M HCl in 1,4-Dioxane (3 ml) at 0° C. Reaction mixture was stirred at RT for 1 h. Reaction progress was monitored by TLC. Upon disappearance of starting material, the reaction mixture was evaporated under reduced pressure, washed with Diisopropyl ether and evaporated to afford (S)-2-amino-N, 2-diphenylacetamide hydrochloride. Yield 0.22 g (91.28%).

### Step-5: (S)—N, 2-diphenyl-2-(vinylsulfonamido)acetamide

To a stirred solution of (S)-2-amino-N, 2-diphenylacetamide hydrochloride (0.220 g, 0.837 mmol) in DCM (10 mL) was added TEA (0.525 ml, 3.766 mmol) and 2-Chloro ethane sulfonyl chloride (0.096 ml, 0.920 mmol) at 0° C. Reaction mixture was stirred at 0° C. for 2 h. Reaction progress was monitored by TLC. Upon disappearance of starting material, the reaction mixture was diluted with DCM (2×30 mL), washed with water (2×30 mL). The organic layer was dried over Na₂SO₄, filtered, evaporated under reduced pressure, and purified by column chromatography. Required compound was eluted in 20% Ethyl acetate and hexane to afford (S)—N, 2-diphenyl-2-(vinylsulfonamido)acetamide. Yield 0.08 g (30.30%).

¹H NMR (400 MHz, cdcl₃) δ 7.49 (s, 1H), 7.46-7.34 (m, 7H), 7.30 (t, J=7.9 Hz, 2H), 7.13 (t, J=7.4 Hz, 1H), 6.33 (dd, J=16.5, 9.8 Hz, 1H), 6.15 (d, J=16.5 Hz, 1H), 5.87 (d, J=5.7 Hz, 1H), 5.79 (d, J=9.8 Hz, 1H), 5.04 (d, J=5.9 Hz, 1H).

MS (m/z): 316.9 [M+H]⁺.

### Synthesis of (S)—N-(2-morpholino-2-oxo-1-phenylethyl)ethenesulfonamide

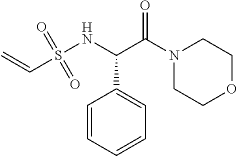 [SMILES: C=CS(=O)(=O)N[C@H](C(=O)N1CCOCC1)c1ccccc1]

¹H NMR (400 MHz, cdcl₃) δ 7.43-7.28 (m, 5H), 6.18 (dd, J=16.5, 9.7 Hz, 1H), 6.12-6.04 (m, 2H), 5.66 (d, J=9.7 Hz, 1H), 5.23 (d, J=7.3 Hz, 1H), 3.78-3.45 (m, 5H), 3.43-3.31 (m, 1H), 3.24-3.15 (m, 1H), 3.14-3.04 (m, 1H). MS (m/z): 310.9 [M+H]⁺.

### (S)—N-(2-oxo-1-phenyl-2-(pyrrolidin-1-yl)ethyl)ethenesulfonamide

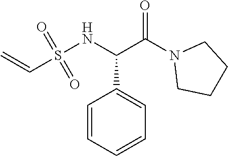 [SMILES: C=CS(=O)(=O)N[C@H](C(=O)N1CCCC1)c1ccccc1]

¹H NMR (400 MHz, cdcl₃) δ 7.41-7.29 (m, 5H), 6.20-6.06 (m, 2H), 6.02 (d, J=16.5 Hz, 1H), 5.61 (d, J=9.7 Hz, 1H), 5.08 (d, J=7.2 Hz, 1H), 3.62-3.52 (m, 1H), 3.51-3.38 (m, 2H), 2.99 (dt, J=9.4, 6.7 Hz, 1H), 1.98-1.72 (m, 4H). MS (m/z): 295 [M+H]⁺.

### N-(1-(4-chlorophenyl)-2-oxo-2-(pyrrolidin-1-yl)ethyl)ethenesulfonamide

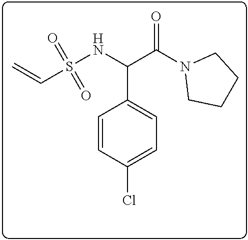 [SMILES: C=CS(=O)(=O)NC(C(=O)N1CCCC1)c1ccc(Cl)cc1]

¹H NMR (400 MHz, cdcl₃) δ 7.32 (q, J=8.5 Hz, 4H), 6.21 (dd, J=16.5, 9.8 Hz, 1H), 6.08 (d, J=7.6 Hz, 2H), 5.68 (d, J=9.8 Hz, 1H), 5.05 (d, J=7.1 Hz, 1H), 3.61-3.52 (m, 1H), 3.44 (td, J=13.3, 7.3 Hz, 2H), 2.97 (dt, J=9.6, 6.6 Hz, 1H), 2.00-1.73 (m, 4H).

MS (m/z): 329.1 [M+H]⁺.

### N-(1-(3-(1-methyl-1H-pyrazol-4-yl)phenyl)-2-oxo-2-(pyrrolidin-1-yl)ethyl)ethenesulfonamide

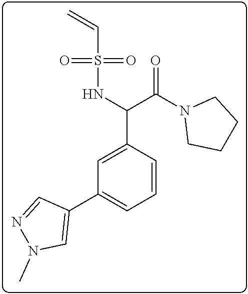 [SMILES: C=CS(=O)(=O)NC(C(=O)N1CCCC1)c1cccc(-c2cnn(C)c2)c1]

¹H NMR (400 MHz, cdcl₃) δ 7.75 (s, 1H), 7.65 (s, 1H), 7.44 (dd, J=4.0, 1.5 Hz, 2H), 7.33 (dd, J=9.7, 6.0 Hz, 1H), 7.18 (d, J=7.6 Hz, 1H), 6.19 (dd, J=16.5, 9.8 Hz, 1H), 6.11 (d, J=7.2 Hz, 1H), 6.04 (d, J=16.5 Hz, 1H), 5.62 (d, J=9.8 Hz, 1H), 5.08 (d, J=7.2 Hz, 1H), 3.95 (s, 3H), 3.63-3.54 (m, 1H), 3.51-3.40 (m, 2H), 3.04 (dt, J=9.4, 6.6 Hz, 1H), 1.96-1.74 (m, 4H).

MS (m/z): 375.5 [M+H]⁺.

### N-(1-(2-chlorophenyl)-2-oxo-2-(pyrrolidin-1-yl)ethyl)ethenesulfonamide

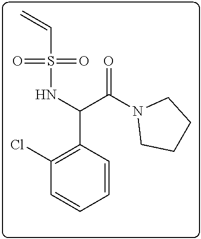 [SMILES: C=CS(=O)(=O)NC(C(=O)N1CCCC1)c1ccccc1Cl]

¹H NMR (400 MHz, cdcl₃) δ 7.44-7.36 (m, 2H), 7.31-7.27 (m, 2H), 6.33 (dd, J=16.5, 9.8 Hz, 1H), 6.15 (d, J=16.5 Hz, 1H), 6.10 (d, J=7.3 Hz, 1H), 5.71 (d, J=9.8 Hz, 1H), 5.57 (d, J=7.4 Hz, 1H), 3.56 (dt, J=12.2, 5.4 Hz, 2H), 3.47-3.36 (m, 1H), 2.89 (dt, J=10.0, 7.0 Hz, 1H), 1.99-1.68 (m, 4H).

MS (m/z): 329.1 [M+H]⁺.

### (R)—N-(2-oxo-1-phenyl-2-(pyrrolidin-1-yl)ethyl)ethenesulfonamide

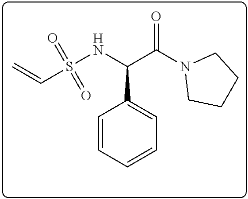 [SMILES: C=CS(=O)(=O)N[C@@H](C(=O)N1CCCC1)c1ccccc1]

1H NMR (400 MHz, cdcl₃) δ 7.39-7.30 (m, 5H), 6.15 (dd, J=16.5, 9.7 Hz, 1H), 6.09 (d, J=7.1 Hz, 1H), 6.02 (d, J=16.5 Hz, 1H), 5.61 (d, J=9.7 Hz, 1H), 5.08 (d, J=7.2 Hz, 1H), 3.62-3.53 (m, 1H), 3.50-3.39 (m, 2H), 2.99 (dt, J=9.3, 6.7 Hz, 1H), 1.95-1.73 (m, 4H).

MS (m/z): 295.3 [M+H]⁺.

### (S)—N-(1-oxo-3-phenyl-1-(pyrrolidin-1-yl)propan-2-yl)ethenesulfonamide

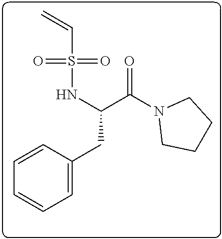 [SMILES: C=CS(=O)(=O)N[C@@H](Cc1ccccc1)C(=O)N1CCCC1]

¹H NMR (400 MHz, cdcl₃) δ 7.33-7.22 (m, 3H), 7.22-7.15 (m, 2H), 6.32 (dd, J=16.5, 9.8 Hz, 1H), 6.16-6.08 (m, 1H), 5.75 (d, J=9.8 Hz, 1H), 5.47 (d, J=9.5 Hz, 1H), 4.22 (td, J=8.6, 6.7 Hz, 1H), 3.47-3.37 (m, 1H), 3.29 (ddd, J=11.9, 11.3, 6.5 Hz, 2H), 3.07-2.93 (m, 2H), 2.59 (dt, J=9.7, 6.5 Hz, 1H), 1.75 (ddd, J=14.7, 12.0, 3.1 Hz, 2H), 1.58 (ddd, J=16.9, 8.7, 4.8 Hz, 1H).

MS (m/z): 309.2 [M+H]⁺.

### N-(1-(2-(1-methyl-1H-pyrazol-4-yl)phenyl)-2-oxo-2-(pyrrolidin-1-yl)ethyl) ethene sulfonamide

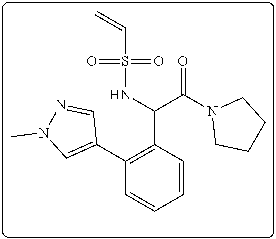 [SMILES: C=CS(=O)(=O)NC(C(=O)N1CCCC1)c1ccccc1-c1cnn(C)c1]

¹H NMR (400 MHz, cdcl₃) δ 7.98 (s, 1H), 7.58 (s, 1H), 7.40 (d, J=7.6 Hz, 1H), 7.35 (d, J=4.0 Hz, 2H), 7.32-7.27 (m, 1H), 6.47 (dd, J=16.5, 9.9 Hz, 1H), 6.09 (d, J=16.5 Hz, 1H), 5.74 (d, J=9.9 Hz, 2H), 5.29 (d, J=9.0 Hz, 1H), 4.00 (s, 3H), 3.42 (dd, J=12.1, 6.5 Hz, 1H), 3.34 (dd, J=12.0, 7.0 Hz, 1H), 2.94 (dd, J=9.9, 6.1 Hz, 1H), 2.59-2.51 (m, 1H), 1.71 (ddd, J=15.2, 13.0, 7.6 Hz, 4H).

### Synthesis of 2-benzyl-4-hydroxy-5-methyleneisothiazolidine 1,1-dioxide

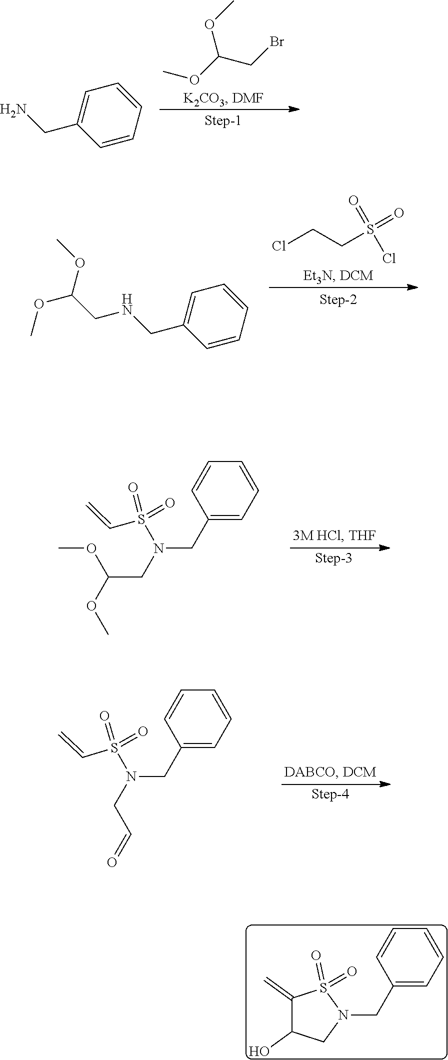

### Step-1: N-benzyl-2,2-dimethoxyethan-1-amine

To a solution of Phenylmethanamine (1 g, 9.331 mmol) in DMF (10 mL) were added K₂CO₃ (3.869 ml, 27.993 mmol) and 2-Bromo-1-1-dimethoxy ethane (1.1 ml, 9.331 mmol) at 0° C. Reaction mixture was stirred at 80° C. for 16 h. Reaction progress was monitored by TLC. Upon disappearance of starting material, the reaction mixture was diluted with EtOAc (2×50 mL), washed with water (2×50 mL) and brine solution (2×50 mL). The organic layer was dried over Na₂SO₄, filtered, evaporated under reduced pressure, and purified by column chromatography. Required compound was eluted in 10% Ethyl acetate and hexane to afford N-benzyl-2,2-dimethoxyethan-1-amine. Yield 1.281 g (70.30%).

### Step-2: N-benzyl-N-(2,2-dimethoxyethyl)ethenesulfonamide

To a stirred solution of N-benzyl-2,2-dimethoxyethan-1-amine (1.281 g, 6.560 mmol) in DCM (20 mL) was added TEA (4.114 ml, 29.52 mmol) and 2-Chloro ethane sulfonyl chloride (0.754 ml, 7.216 mmol) at 0° C. Reaction mixture was stirred at 0° C. for 2 h. Reaction progress was monitored by TLC. Upon disappearance of starting material, the reaction mixture was diluted with DCM (2×50 mL), washed with water (2×50 mL). The organic layer was dried over Na₂SO₄, filtered, evaporated under reduced pressure, and purified by column chromatography. Required compound was eluted in 6% Ethyl acetate and hexane to afford N-benzyl-N-(2,2-dimethoxyethyl)ethenesulfonamide. Yield 0.872 g (46.58%).

### Step-3: N-benzyl-N-(2-oxoethyl)ethenesulfonamide

To a stirred solution of N-benzyl-N-(2,2-dimethoxyethyl)ethenesulfonamide (0.3 g, 1.051 mmol) in THF (1.8 mL) was added was added 3M HCl (1.8 ml) at RT. Reaction mixture was stirred at RT for 70° C. for 3 h. Reaction progress was monitored by TLC. Upon disappearance of starting material, the reaction mixture was diluted with EtOAc (2×30 mL), washed with water (2×50 mL). The organic layer was dried over Na₂SO₄, filtered, evaporated under reduced pressure, and purified by column chromatography. Required compound was eluted in 40% Ethyl acetate and hexane to afford N-benzyl-N-(2-oxoethyl)ethenesulfonamide. Yield 0.226 g (90.03%).

### Step-4: 2-benzyl-4-hydroxy-5-methyleneisothiazolidine 1,1-dioxide

To a stirred solution of N-benzyl-N-(2-oxoethyl)ethenesulfonamide (0.132 g, 0.551 mmol) in DCM (10 mL) was added DABCO (0.006 g, 0.0551 mmol) at 0° C. Reaction mixture was stirred at 0° C. for 4 h. Reaction progress was monitored by TLC. Upon disappearance of starting material, the reaction mixture was evaporated under reduced pressure, and purified by column chromatography. Required compound was eluted in 40% Ethyl acetate and hexane to afford 2-benzyl-4-hydroxy-5-methyleneisothiazolidine 1,1-dioxide. Yield 0.05 g (37.87%). ¹H NMR (400 MHz, cdcl₃) δ 7.41-7.29 (m, 5H), 6.26 (t, J=2.0 Hz, 1H), 6.00 (t, J=1.9 Hz, 1H), 4.80 (s, 1H), 4.23 (s, 2H), 3.37 (dd, J=10.3, 6.6 Hz, 1H), 2.98 (dd, J=10.3, 4.6 Hz, 1H), 2.42 (s, 1H). MS (m/z): 240.1 [M+H]

### 4-hydroxy-5-methylene-2-phenylisothiazolidine 1,1-dioxide

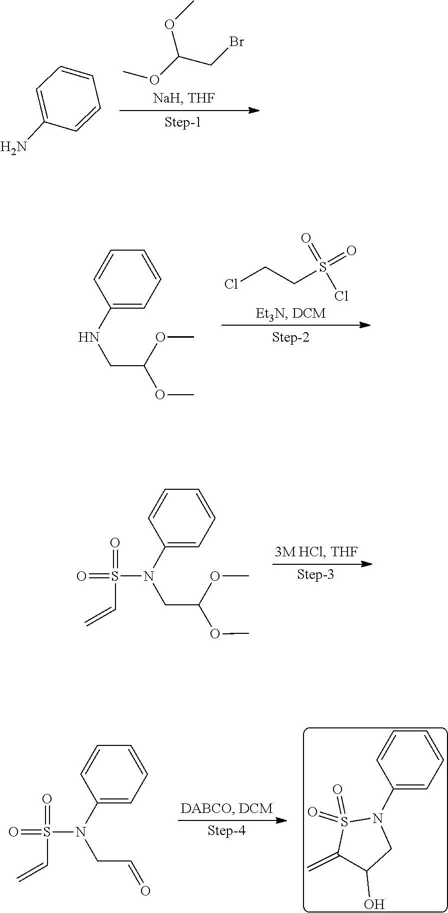

### Step-1: N-(2,2-dimethoxyethyl)aniline

To a solution of Aniline (1 g, 10.737 mmol) in THF (10 mL) were added NaH (0.644 g, 16.105 mmol) and 2-Bromo-1-1-dimethoxy ethane (1.522 ml, 12.884 mmol) at 0° C. Reaction mixture was stirred at 70° C. for 16 h. Reaction progress was monitored by TLC. Upon disappearance of starting material, the reaction mixture was quenched with ice cold water (2×50 mL) and extracted with EtOAc (2×50 mL). The organic layer was dried over Na₂SO₄, filtered, evaporated under reduced pressure, and purified by column chromatography. Required compound was eluted in 2% Ethyl acetate and hexane to afford N-(2,2-dimethoxyethyl)aniline. Yield 0.684 g (35.14%).

### Step-2: N-(2,2-dimethoxyethyl)-N-phenylethenesulfonamide

To a stirred solution of N-(2,2-dimethoxyethyl)aniline (0.684 g, 3.774 mmol) in DCM (10 mL) was added TEA (2.367 ml, 16.983 mmol) and 2-Chloro ethane sulfonyl chloride (0.4 ml, 4.151 mmol) at 0° C. Reaction mixture was stirred at 0° C. for 2 h. Reaction progress was monitored by TLC. Upon disappearance of starting material, the reaction mixture was diluted with DCM (2×50 mL), washed with water (2×50 mL). The organic layer was dried over Na₂SO₄, filtered, evaporated under reduced pressure, and purified by column chromatography. Required compound was eluted in 25% Ethyl acetate and hexane to afford N-(2,2-dimethoxyethyl)-N-phenylethenesulfonamide. Yield 0.861 g (84.16%).

### Step-3: N-(2-oxoethyl)-N-phenylethenesulfonamide

To a stirred solution of N-(2,2-dimethoxyethyl)-N-phenylethenesulfonamide (0.312 g, 1.149 mmol) in THF (1.87 mL) was added was added 3M HCl (1.87 ml) at RT. Reaction mixture was stirred at RT for 70° C. for 3 h. Reaction progress was monitored by TLC. Upon disappearance of starting material, the reaction mixture was diluted with EtOAc (2×30 mL) and washed with water (2×30 mL). The organic layer was dried over Na₂SO₄, filtered, evaporated under reduced pressure, and purified by column chromatography. Required compound was eluted in 30% Ethyl acetate and hexane to afford N-(2-oxoethyl)-N-phenylethenesulfonamide. Yield 0.236 g (91.11%).

### Step-4: 4-hydroxy-5-methylene-2-phenylisothiazolidine 1,1-dioxide

To a stirred solution of N-(2-oxoethyl)-N-phenylethenesulfonamide (0.130 g, 0.577 mmol) in DCM (10 mL) was added DABCO (0.006 g, 0.0577 mmol) at 0° C. Reaction mixture was stirred at 0° C. for 4 h. Reaction progress was monitored by TLC. Upon disappearance of starting material, the reaction mixture was evaporated under reduced pressure and purified by column chromatography. Required compound was eluted in 40% Ethyl acetate and hexane to afford 4-hydroxy-5-methylene-2-phenylisothiazolidine 1,1-dioxide. Yield 0.05 g (38.46%).

¹H NMR (400 MHz, cdcl₃) δ 7.44-7.37 (m, 2H), 7.35 (t, J=4.4 Hz, 2H), 7.25-7.21 (m, 1H), 6.32 (t, J=2.1 Hz, 1H), 6.07 (t, J=1.9 Hz, 1H), 5.04 (dt, J=8.1, 6.4 Hz, 1H), 4.00 (dd, J=9.7, 6.5 Hz, 1H), 3.63 (dd, J=9.7, 5.0 Hz, 1H), 2.58 (d, J=8.3 Hz, 1H).

MS (m/z): 226.1 [M+H]⁺.

### 4-hydroxy-2-(2-(1-methyl-1H-pyrazol-4-yl)phenyl)-5-methyleneisothiazolidine 1,1-dioxide

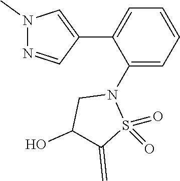 [SMILES: C=C1C(O)CN(c2ccccc2-c2cnn(C)c2)S1(=O)=O]

¹H NMR (400 MHz, dmso) δ 8.09 (s, 1H), 7.85 (s, 1H), 7.64 (d, J=7.7 Hz, 1H), 7.55 (d, J=7.9 Hz, 1H), 7.42 (t, J=7.2 Hz, 1H), 7.32 (t, J=7.6 Hz, 1H), 6.34 (d, J=5.1 Hz, 1H), 6.21 (s, 1H), 6.06 (s, 1H), 4.93 (d, J=6.2 Hz, 1H), 3.85 (s, 3H), 3.69 (dd, J=9.6, 6.9 Hz, 1H), 3.27 (dd, J=9.7, 5.4 Hz, 1H).

MS (m/z): 306.2 [M+H]⁺.

### 4-hydroxy-2-methyl-5-methylene-3-phenylisothiazolidine 1,1-dioxide

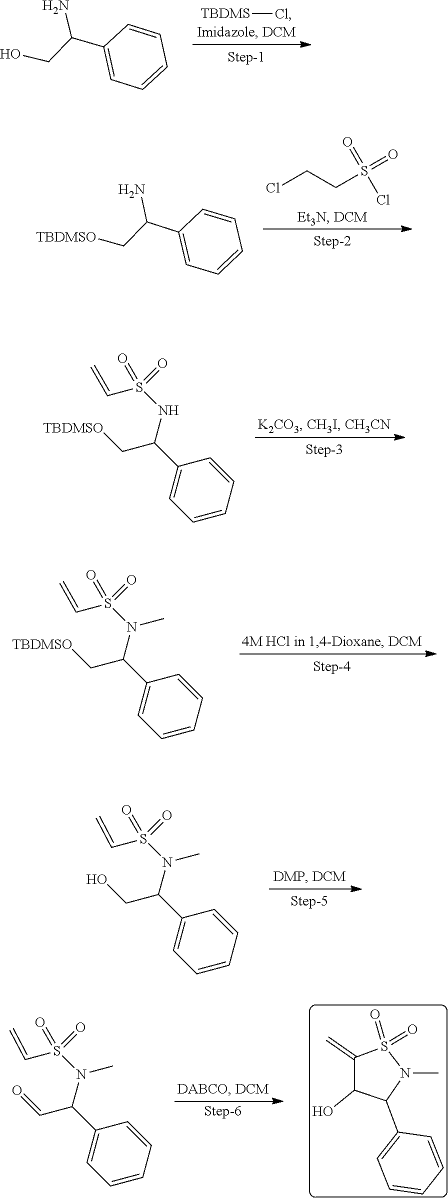

### Step-1: 2-((tert-butyldimethylsilyl)oxy)-1-phenylethan-1-amine

To a stirred solution of 2-amino-2-phenylethan-1-ol (1 g, 7.289 mmol) in DCM (20 mL) were added Imidazole (1.240 g, 18.222 mmol) and TBDMS-Cl (1.318 g, 8.746 mmol) at 0° C. Reaction mixture was stirred at RT for 2 h. Reaction progress was monitored by TLC. Upon disappearance of starting material, the reaction mixture was diluted with DCM (2×50 mL) and washed with water (2×50 mL). The organic layer was dried over Na₂SO₄, filtered and evaporated under reduced pressure to afford 2-((tert-butyldimethylsilyl)oxy)-1-phenylethan-1-amine. Yield 1.6 g (87.33%).

### Step-2: N-(2-((tert-butyldimethylsilyl)oxy)-1-phenylethyl)ethenesulfonamide

To a stirred solution of 2-((tert-butyldimethylsilyl)oxy)-1-phenylethan-1-amine (1 g, 3.976 mmol) in DCM (15 mL) was added TEA (2.49 ml, 17.892 mmol) and 2-Chloro ethane sulfonyl chloride (0.457 ml, 4.373 mmol) at 0° C. Reaction mixture was stirred at 0° C. for 2 h. Reaction progress was monitored by TLC. Upon disappearance of starting material, the reaction mixture was diluted with DCM (2×50 mL) and washed with water (2×50 mL). The organic layer was dried over Na₂SO₄, filtered, evaporated under reduced pressure, and purified by column chromatography. Required compound was eluted in 20% Ethyl acetate and hexane to afford N-(2-((tert-butyldimethylsilyl)oxy)-1-phenylethyl)ethenesulfonamide. Yield 0.6 g (44.18%).

### Step-3: N-(2-((tert-butyldimethylsilyl)oxy)-1-phenylethyl)-N-methylethenesulfonamide

To a stirred solution of N-(2-((tert-butyldimethylsilyl)oxy)-1-phenylethyl)ethenesulfonamide (0.5 g, 1.463 mmol) in ACN (10 mL) was added K₂CO₃ (0.606 g, 4.389 mmol) and CH₃I (0.091 ml, 2.926 mmol) at RT. Reaction mixture was stirred at 80° C. for 4 h. Reaction progress was monitored by TLC. Upon disappearance of starting material, the reaction mixture was diluted with DCM (2×30 mL) and washed with water (2×30 mL). The organic layer was dried over Na₂SO₄, filtered, evaporated under reduced pressure, and purified by column chromatography. Required compound was eluted in 15% Ethyl acetate and hexane to afford N-(2-((tert-butyldimethylsilyl)oxy)-1-phenylethyl)-N-methylethenesulfonamide. Yield 0.5 g (96.15%).

### Step-4: N-(2-hydroxy-1-phenylethyl)-N-methylethenesulfonamide

To a stirred solution of N-(2-((tert-butyldimethylsilyl)oxy)-1-phenylethyl)-N-methylethenesulfonamide (0.5 g, 1.406 mmol) in DCM (10 mL) was added 4M HCl in 1,4-Dioxane (2 ml) at 0° C. Reaction mixture was stirred at RT for 2 h. Reaction progress was monitored by TLC. Upon disappearance of starting material, the reaction mixture was evaporated under reduced pressure to afford N-(2-hydroxy-1-phenylethyl)-N-methylethenesulfonamide. Yield 0.3 g (88.49%).

### Step-5: N-methyl-N-(2-oxo-1-phenylethyl)ethenesulfonamide

To a stirred solution of N-(2-hydroxy-1-phenylethyl)-N-methylethenesulfonamide (0.2 g, 0.828 mmol) in DCM (10 mL) was added DMP (0.703 g, 1.656 mmol) at 0° C. Reaction mixture was stirred at RT for 2 h. Reaction progress was monitored by TLC. Upon disappearance of starting material, the reaction mixture was diluted with DCM (2×50 mL) and washed with water (2×50 mL). The organic layer was dried over Na₂SO₄, filtered, evaporated under reduced pressure, and purified by column chromatography. Required compound was eluted in 20% Ethyl acetate and hexane to afford N-methyl-N-(2-oxo-1-phenylethyl)ethenesulfonamide. Yield 0.19 g (95.95%).

### Step-6: 4-hydroxy-2-methyl-5-methylene-3-phenylisothiazolidine 1,1-dioxide

To a stirred solution of N-methyl-N-(2-oxo-1-phenylethyl)ethenesulfonamide (0.19 g, 0.794 mmol) in DCM (20 mL) was added DABCO (0.0088 g, 0.079 mmol) at 0° C. Reaction mixture was stirred at RT for 12 h. Reaction progress was monitored by TLC. Upon disappearance of starting material, the reaction mixture was evaporated under reduced pressure, and purified by column chromatography. Required compound was eluted in 40% Ethyl acetate and hexane to afford 4-hydroxy-2-methyl-5-methylene-3-phenylisothiazolidine 1,1-dioxide. Yield 0.03 g (15.78%).

1H NMR (400 MHz, cdcl3) δ 7.48-7.35 (m, 5H), 6.30 (dd, J=4.7, 2.4 Hz, 1H), 5.98 (t, J=2.1 Hz, 1H), 4.68 (s, 1H), 3.89 (d, J=7.2 Hz, 1H), 2.54 (s, 3H), 2.38 (d, J=13.7 Hz, 1H).

MS (m/z): 240.1 [M+H]⁺.

### Synthesis of 4-hydroxy-2-(2-(1-methyl-1H-pyrazol-4-yl)benzyl)-5-methyleneisothiazolidine 1,1-dioxide

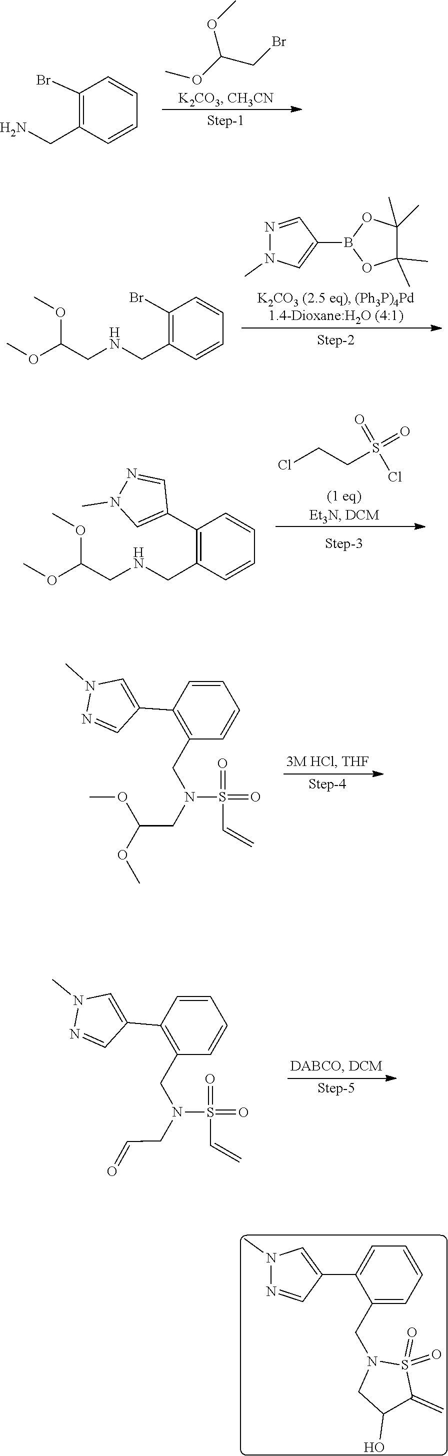

### Step-1: N-(2-bromobenzyl)-2,2-dimethoxyethan-1-amine

To a solution of (2-bromophenyl)methanamine (0.5 g, 2.687 mmol) in ACN (20 mL) were added K₂CO₃ (1.114 g, 8.061 mmol) and 2-Bromo-1-1-dimethoxy ethane (0.317 ml, 2.687 mmol) at RT. Reaction mixture was stirred at 80° C. for 16 h. Reaction progress was monitored by TLC. Upon disappearance of starting material, the reaction mixture was diluted with EtOAc (2×50 mL), washed with water (2×50 mL) and brine solution (2×50 mL). The organic layer was dried over Na₂SO₄, filtered, evaporated under reduced pressure, and purified by column chromatography. Required compound was eluted in 8% Ethyl acetate and hexane to afford N-(2-bromobenzyl)-2,2-dimethoxyethan-1-amine. Yield 0.3 g (40.76%).

### Step-2: 2,2-dimethoxy-N-(2-(1-methyl-1H-pyrazol-4-yl)benzyl)ethan-1-amine

To a solution of N-(2-bromobenzyl)-2,2-dimethoxyethan-1-amine (0.3 g, 1.094 mmol) in 1,4-Dioxane (4 mL) and H₂O (1 mL) were added K₂CO₃ (0.378 g, 2.735 mmol) and 1-methyl-4-(4,4,5,5-tetramethyl-1,3,2-dioxaborolan-2-yl)-1H-pyrazole (0.273 g, 2.687 mmol) at RT. Reaction mixture was degassed with Nitrogen for 10 min. Then added Tetrakis (0.126 g, 0.1094) and again degassed with Nitrogen for 10 min. Reaction mixture was stirred at 90° C. for 16 h. Reaction progress was monitored by TLC. Upon disappearance of starting material, the reaction mixture was diluted with EtOAc (2×50 mL), washed with water (2×50 mL) and brine solution (2×50 mL). The organic layer was dried over Na₂SO₄, filtered, evaporated under reduced pressure, and purified by column chromatography. Required compound was eluted in 25% Ethyl acetate and hexane to afford 2,2-dimethoxy-N-(2-(1-methyl-1H-pyrazol-4-yl)benzyl)ethan-1-amine. Yield 0.2 g (66.44%).

### Step-3: N-(2,2-dimethoxyethyl)-N-(2-(1-methyl-1H-pyrazol-4-yl)benzyl)ethenesulfonamide

To a stirred solution of 2,2-dimethoxy-N-(2-(1-methyl-1H-pyrazol-4-yl)benzyl)ethan-1-amine (0.2 g, 0.726 mmol) in DCM (10 mL) was added TEA (0.455 ml, 3.267 mmol) and 2-Chloro ethane sulfonyl chloride (0.083 ml, 0.798 mmol) at 0° C. Reaction mixture was stirred at 0° C. for 2 h. Reaction progress was monitored by TLC. Upon disappearance of starting material, the reaction mixture was diluted with DCM (2×30 mL), and washed with water (2×30 mL). The organic layer was dried over Na₂SO₄, filtered, evaporated under reduced pressure, and purified by column chromatography. Required compound was eluted in 15% Ethyl acetate and hexane to afford N-(2,2-dimethoxyethyl)-N-(2-(1-methyl-1H-pyrazol-4-yl)benzyl)ethenesulfonamide. Yield 0.08 g (30.18%).

### Step-4: N-(2-(1-methyl-1H-pyrazol-4-yl)benzyl)-N-(2-oxoethyl)ethenesulfonamide

To a stirred solution of N-(2,2-dimethoxyethyl)-N-(2-(1-methyl-1H-pyrazol-4-yl)benzyl)ethenesulfonamide (0.08 g, 0.218 mmol) in THF (1.8 mL) was added 3M HCl (1.8 ml) at RT. Reaction mixture was stirred at RT for 70° C. for 3 h. Reaction progress was monitored by TLC. Upon disappearance of starting material, the reaction mixture was diluted with EtOAc (2×30 mL) and washed with water (2×50 mL). The organic layer was dried over Na₂SO₄, filtered, evaporated under reduced pressure, and purified by column chromatography. Required compound was eluted in 45% Ethyl acetate and hexane to afford N-(2-(1-methyl-1H-pyrazol-4-yl)benzyl)-N-(2-oxoethyl)ethenesulfonamide. Yield 0.06 g (85.83%).

### Step-5: 4-hydroxy-2-(2-(1-methyl-1H-pyrazol-4-yl)benzyl)-5-methyleneisothiazolidine 1,1-dioxide

To a stirred solution of N-(2-(1-methyl-1H-pyrazol-4-yl)benzyl)-N-(2-oxoethyl)ethenesulfonamide (0.06 g, 0.187 mmol) in DCM (6 mL) was added DABCO (0.002 g, 0.0187 mmol) at 0° C. Reaction mixture was stirred at RT for 3 h. Reaction progress was monitored by TLC. Upon disappearance of starting material, the reaction mixture was evaporated under reduced pressure, and purified by column chromatography. Required compound was eluted in 30% Ethyl acetate and hexane to afford 4-hydroxy-2-(2-(1-methyl-1H-pyrazol-4-yl)benzyl)-5-methyleneisothiazolidine 1,1-dioxide. Yield 0.015 g (25%).

¹H NMR (400 MHz, cdcl₃) δ 7.65 (s, 1H), 7.57 (s, 1H), 7.44-7.28 (m, 4H), 6.27 (t, J=2.0 Hz, 1H), 6.01 (t, J=1.9 Hz, 1H), 4.79 (ddd, J=8.5, 4.4, 2.0 Hz, 1H), 4.27 (d, J=3.1 Hz, 2H), 3.94 (s, 3H), 3.33 (dd, J=10.2, 6.5 Hz, 1H), 2.95 (dd, J=10.3, 4.6 Hz, 1H), 1.82 (s, 1H).

MS (m/z): 320.1 [M+H]⁺.

### 4-methoxy-5-methylene-2-phenylisothiazolidine 1,1-dioxide

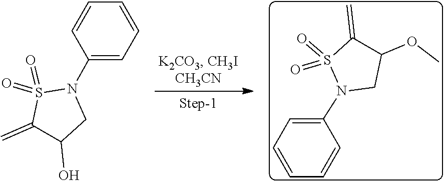 [SMILES: C=C1C(O)CN(c2ccccc2)S1(=O)=O ; C=C1C(OC)CN(c2ccccc2)S1(=O)=O]

### Step-1: 4-methoxy-5-methylene-2-phenylisothiazolidine 1,1-dioxide

To a stirred solution of 4-hydroxy-5-methylene-2-phenylisothiazolidine 1,1-dioxide (0.15 g, 0.665 mmol) in ACN (10 mL) was added K2CO3 (0.460 g, 3.325 mmol) and CH3I (0.124 ml, 1.995 mmol) at RT. Reaction mixture was stirred at 80° C. for 16 h. Reaction progress was monitored by TLC. Upon disappearance of starting material, the reaction mixture was diluted with DCM (2×30 mL) and washed with water (2×30 mL). The organic layer was dried over Na₂SO₄, filtered, evaporated under reduced pressure, and purified by column chromatography. Required compound was eluted in 20% Ethyl acetate and hexane to afford 4-methoxy-5-methylene-2-phenylisothiazolidine 1,1-dioxide. Yield 0.01 g (6.28%).

¹H NMR (400 MHz, cdcl₃) δ 7.44-7.31 (m, 4H), 7.22 (t, J=7.1 Hz, 1H), 6.36 (s, 1H), 6.02 (s, 1H), 4.67-4.61 (m, 1H), 3.98 (dd, J=9.7, 6.1 Hz, 1H), 3.72 (dd, J=9.7, 3.9 Hz, 1H), 3.51-3.48 (s, 3H).

MS (m/z): 240.1 [M+H]⁺.

### 4-methoxy-2-methyl-5-methylene-3-phenylisothiazolidine 1,1-dioxide

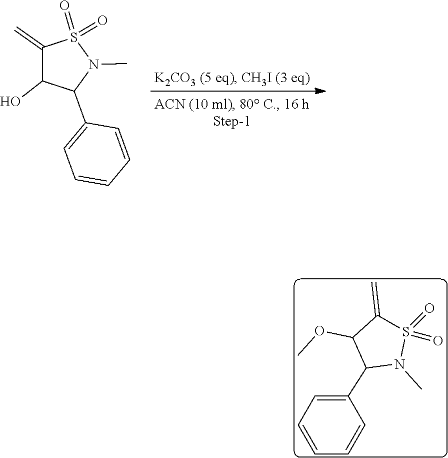 [SMILES: C=C1C(O)C(c2ccccc2)N(C)S1(=O)=O ; C=C1C(OC)C(c2ccccc2)N(C)S1(=O)=O]

### Step-1: 4-methoxy-2-methyl-5-methylene-3-phenylisothiazolidine 1,1-dioxide

To a stirred solution of 4-hydroxy-2-methyl-5-methylene-3-phenylisothiazolidine 1,1-dioxide (0.06 g, 0.250 mmol) in ACN (10 mL) was added K2CO3 (0.173 g, 1.25 mmol) and CH3I (0.046 ml, 0.75 mmol) at RT. Reaction mixture was stirred at 80° C. for 16 h. Reaction progress was monitored by TLC. Upon disappearance of starting material, the reaction mixture was diluted with DCM (2×30 mL) and washed with water (2×30 mL). The organic layer was dried over Na₂SO₄, filtered, evaporated under reduced pressure, and purified by column chromatography. Required compound was eluted in 20% Ethyl acetate and hexane to afford 4-methoxy-2-methyl-5-methylene-3-phenylisothiazolidine 1,1-dioxide. Yield 0.015 g (23.80%).

¹H NMR (400 MHz, cdcl₃) δ 7.45-7.36 (m, 5H), 6.34 (t, J=1.9 Hz, 1H), 5.92 (t, J=1.8 Hz, 1H), 4.33 (dt, J=5.7, 2.0 Hz, 1H), 4.05 (d, J=5.7 Hz, 1H), 3.31 (s, 3H), 2.55 (s, 3H).

MS (m/z): 254.5 [M+H]⁺.

### 4-hydroxy-2-(3-(1-methyl-1H-pyrazol-4-yl)phenyl)-5-methyleneisothiazolidine 1,1-dioxide

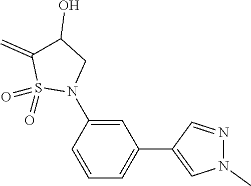

¹H NMR (400 MHz, cdcl₃) δ 7.76 (s, 1H), 7.64 (s, 1H), 7.44 (s, 1H), 7.41-7.31 (m, 2H), 7.18 (d, J=7.5 Hz, 1H), 6.34 (s, 1H), 6.09 (s, 1H), 5.07 (s, 1H), 4.04 (dd, J=9.5, 6.5 Hz, 1H), 3.95 (s, 3H), 3.66 (dd, J=9.5, 4.9 Hz, 1H), 2.70 (d, J=7.3 Hz, 1H).

LCMS (m/z): 306.2 [M+H]⁺.

### Synthesis of 4-(4-chloro-2-(1-methyl-1H-pyrazol-4-yl)phenyl)-4-hydroxy-2-methylenebutanenitrile

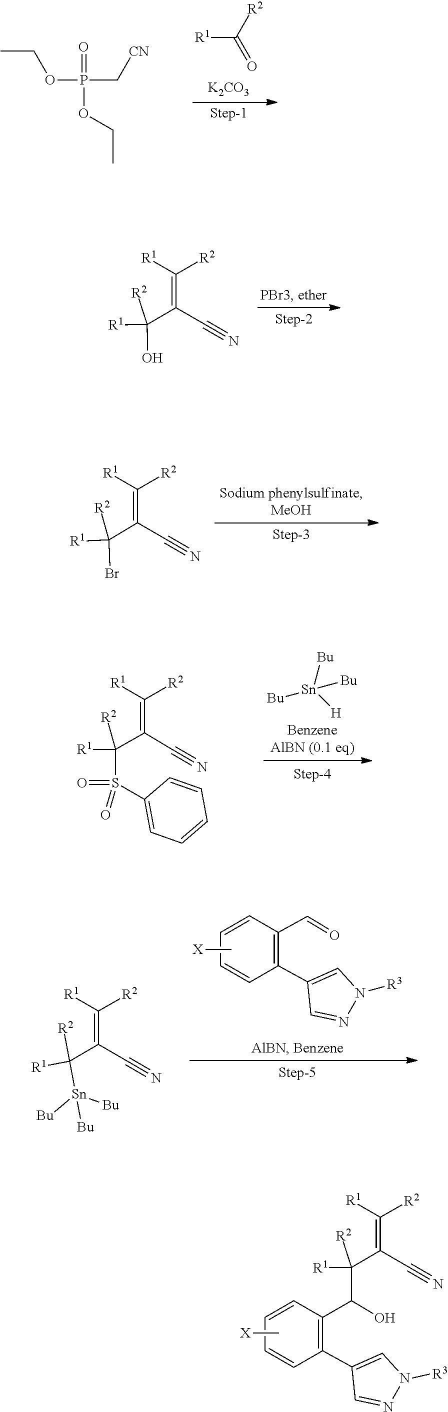

Wherein R¹ and R²: H, D, Alkyl, cycloakyl. Wherein R¹ and R² together can make a cyclic ring.

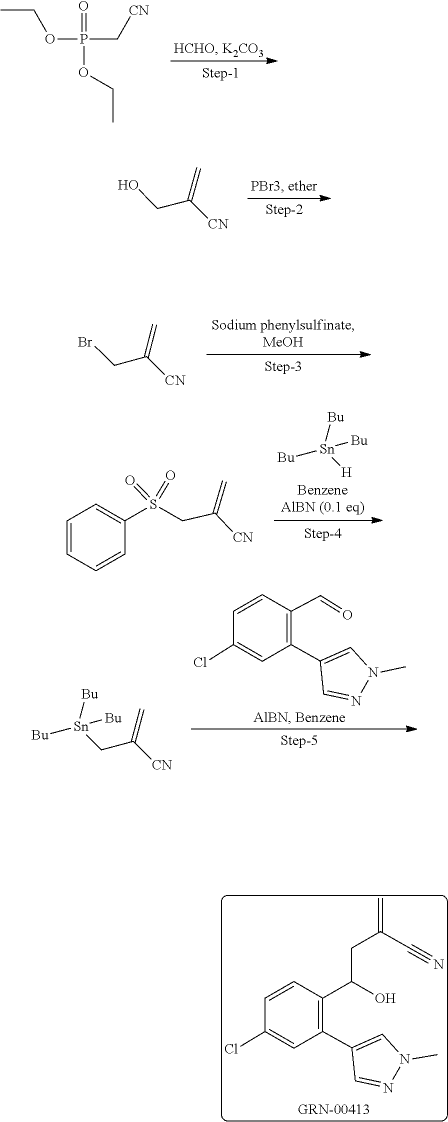

### Step-1: 2-(hydroxymethyl)acrylonitrile

To a solution of diethyl (cyanomethyl)phosphonate (5 g, 28.248 mmol) in Water (20 mL) were added Aq.HCHO (3.389 g, 112.992 mmol) and K2CO3 (7.016 g, 50.846 mmol) at 0° C. Reaction mixture was stirred at RT for 3 h. Reaction progress was monitored by TLC. Upon disappearance of starting material, the reaction mixture was diluted with ether (2×200 mL) and washed with water (2×100 mL). The organic layer was dried over Na₂SO₄, filtered, evaporated under reduced pressure, and purified by column chromatography. The desired compound was eluted in 20% ethyl acetate and hexane to afford 2-(hydroxymethyl)acrylonitrile. Yield 2.1 g (89%).

### Step-2: 2-(bromomethyl)acrylonitrile

To a stirred solution of 2-(hydroxymethyl)acrylonitrile (2.1 g, 25.301 mmol) in Diethyl ether (20 mL) was added PBr₃ (1.19 ml, 12.65 mmol) at 0° C. Reaction mixture was stirred at 0° C. for 1 h. Reaction progress was monitored by TLC. Upon disappearance of starting material, the reaction mixture was diluted with ether (2×100 mL) and washed with water (2×100 mL). The organic layer was dried over Na₂SO₄, filtered, evaporated under reduced pressure, and purified by column chromatography. The desired compound was eluted in 20% ethyl acetate and hexane to afford 2-(bromomethyl)acrylonitrile. Yield 2 g (54%).

### Step-3: 2-((phenylsulfonyl)methyl)acrylonitrile

To a stirred solution of 2-(bromomethyl)acrylonitrile (2 g, 13.698 mmol) in methanol (20 mL) was added sodium phenyl sulfinate (3.36 g, 20.547 mmol) at 0° C. The reaction mixture was stirred at 0° C. for 2 h. The progress of the reaction was monitored by TLC. Upon disappearance of starting material, the reaction mixture was diluted with ethylacetate (2×100 mL) and washed with water (2×50 mL). The organic layer was dried over Na₂SO₄, filtered, evaporated under reduced pressure and purified by column chromatography. The desired compound was eluted in 40% ethyl acetate in hexane to afford 2-((phenylsulfonyl)methyl)acrylonitrile. Yield 0.62 g (21%).

### Step-4: 2-((tributylstannyl)methyl)acrylonitrile

To a stirred solution of 2-((phenylsulfonyl)methyl)acrylonitrile (0.62 g, 2.991 mmol) in benzene (6 mL) was added AIBN (0.049 g, 0.299 mmol) and tributylstannane (1.306 g, 4.487 mmol) at RT. The reaction mixture was stirred at 80° C. for 3 h. The reaction progress was monitored by TLC. Upon disappearance of starting material, the reaction mixture was evaporated under reduced pressure and purified by column chromatography. The desired compound was eluted in 10% ethyl acetate and hexane to afford 2-((tributylstannyl)methyl)acrylonitrile. Yield 0.40 g (37%).

### Step-5: 4-(4-chloro-2-(1-methyl-1H-pyrazol-4-yl)phenyl)-4-hydroxy-2-methylenebutanenitrile

To a stirred solution of 4-chloro-2-(1-methyl-1H-pyrazol-4-yl)benzaldehyde (0.1 g, 0.453 mmol) in benzene (2 mL) was added AIBN (0.0074 g, 0.045 mmol) and 2-((tributylstannyl)methyl)acrylonitrile (0.484 g, 1.359 mmol) at RT. Reaction mixture was stirred at 80° C. for 16 h. The progress of the reaction was monitored by TLC. Upon disappearance of starting material, the reaction mixture was evaporated under reduced pressure, and purified by column chromatography. Acetonitrile (6 ml) and concentrated HCl (0.5 ml) were added to the reaction mixture at 0° C. It was stirred for 10 min, then extracted with Et₂₀ (2×50 ml) and washed with saturated NaHCO₃ solution. The organic layer was treated with DBU (0.5 ml) for 10 min. Reaction mixture was evaporated under reduced pressure and purified by column chromatography. The desired compound was eluted in 35% Ethyl acetate and hexane to afford 4-(4-chloro-2-(1-methyl-1H-pyrazol-4-yl)phenyl)-4-hydroxy-2-methylenebutanenitrile.

Yield 0.105 g (80.76%).

¹H NMR (400 MHz, cdcl3) δ 7.56 (d, J=8.4 Hz, 1H), 7.51 (d, J=2.4 Hz, 2H), 7.36-7.27 (m, 2H), 5.95 (s, 1H), 5.78 (s, 1H), 5.21 (dt, J=8.2, 4.2 Hz, 1H), 3.96 (s, 3H), 2.60-2.54 (m, 2H), 2.19 (d, J=3.7 Hz, 1H).

MS (m/z): 288.1 [M+H]⁺.

### 5-(4-chloro-2-(1H-pyrazol-4-yl)phenyl)-3-methylenedihydrofuran-2 (3H)-one

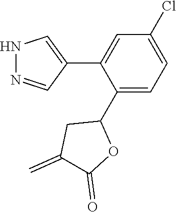 [SMILES: C=C1CC(c2ccc(Cl)cc2-c2cn[nH]c2)OC1=O]

### Synthetic Scheme

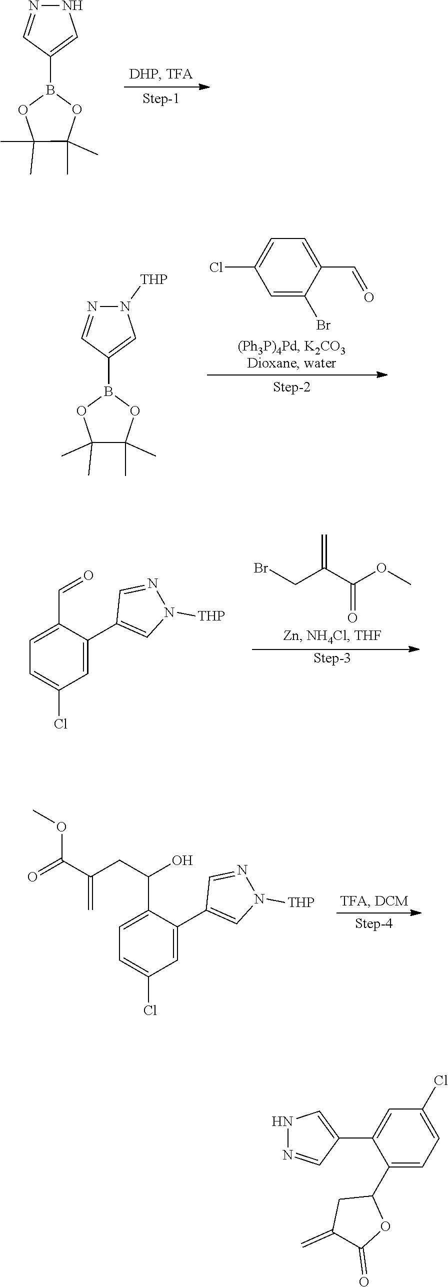

### Step-1: Synthesis of 1-(tetrahydro-2H-pyran-2-yl)-4-(4,4,5,5-tetramethyl-1,3,2-dioxaborolan-2-yl)-1H-pyrazole

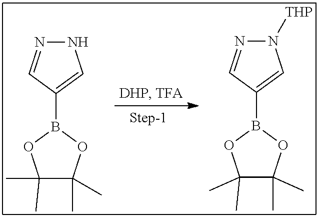

### Procedure:

To a stirred solution of 4-(4,4,5,5-tetramethyl-1,3,2-dioxaborolan-2-yl)-1H-pyrazole (400 mg, 2.68 mmoL) and DHP (248.37 mg, 2.95 mmoL) in toluene (5 mL), TFA (0.01 mL, 0.13 mmoL) was added. The resulting reaction mixture was heated at 80° C. for 2 h. The reaction progress was monitored by TLC. Upon disappearance of starting material, the reaction mixture was diluted with ethyl acetate (15 mL) and washed with water (2×10 mL). The organic layer was dried over Na₂SO₄, filtered and then the solvent was evaporated under reduced pressure to obtained 1-(tetrahydro-2H-pyran-2-yl)-4-(4,4,5,5-tetramethyl-1,3,2-dioxaborolan-2-yl)-1H-pyrazole as a pale yellow oil (600 mg, 75%).

### Step-2: Synthesis of 4-chloro-2-(1-(tetrahydro-2H-pyran-2-yl)-1H-pyrazol-4-yl)benzaldehyde

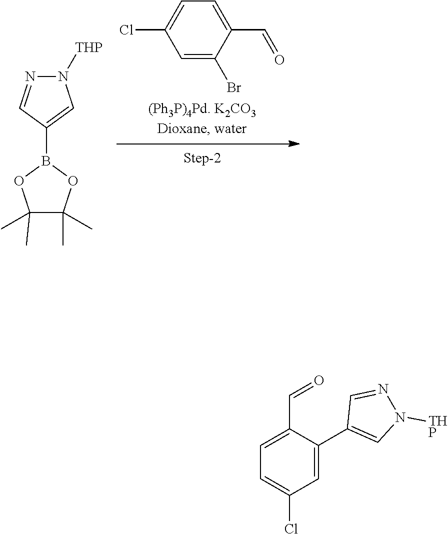

### Procedure:

To a stirred solution of 2-bromo-4-chlorobenzaldehyde (0.3 g, 1.37 mmol) and 1-(tetrahydro-2H-pyran-2-yl)-4-(4,4,5,5-tetramethyl-1,3,2-dioxaborolan-2-yl)-1H-pyrazolein (226 mg, 1.78 mmoL) in 1,4-dioxane (15 mL), potassium carbonate (500 mg, 3.57 mmol) in water (5 mL) was added. The resulting mixture was degassed for 5 min, and Tetrakis(triphenylphosphine)-palladium (30 mg, 10% mol) was added. The reaction mixture was again degassed for further 5 min. The resulting reaction mixture was heated at 70° C. for 2 h. The reaction progress was monitored by TLC. Upon disappearance of starting material, the reaction mixture was diluted with ethyl acetate (15 mL) and washed with water (2×10 mL). The organic layer was dried over Na₂SO₄, filtered and then the solvent was evaporated under reduced pressure. The crude product thus obtained was purified by column chromatography using silica gel (mesh: 60-120) using 25-30% ethyl acetate and petroleum ether as eluent to afford 4-chloro-2-(1-(tetrahydro-2H-pyran-2-yl)-1H-pyrazol-4-yl)benzaldehyde as a pale yellow solid (380 mg, 95%).

### Step-3: Synthesis of methyl 4-(4-chloro-2-(1-(tetrahydro-2H-pyran-2-yl)-1H-pyrazol-4-yl)phenyl)-4-hydroxy-2-methylenebutanoate

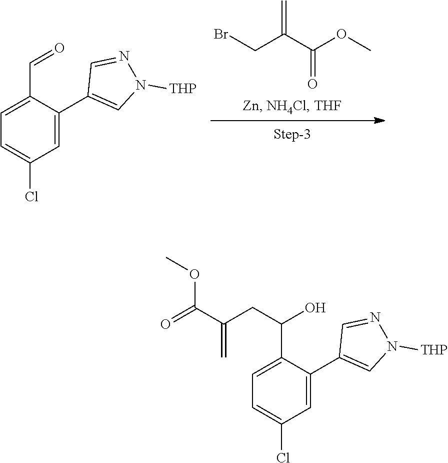

### Procedure:

To a stirred solution of 4-chloro-2-(1-(tetrahydro-2H-pyran-2-yl)-1H-pyrazol-4-yl)benzaldehyde (300 mg, 1.03 mmol) and methyl 2-(bromomethyl)acrylate (241 mg, 1.34 mmoL) in THF (20 mL), Zinc (243 mg, 3.71 mmol) and saturated NH4Cl in water (1 mL) was added. The resulting mixture was stirred at RT for 2 h. The reaction progress was monitored by TLC. Upon disappearance of starting material, the reaction mixture was filtered and filtrate was extracted with ethyl acetate (20 mL) and washed with water (2×10 mL). The organic layer was dried over Na₂SO₄, filtered and the solvent was evaporated under reduced pressure to afford methyl-4-(4-chloro-2-(1-(tetrahydro-2H-pyran-2-yl)-1H-pyrazol-4-yl)phenyl)-4-hydroxy-2-methylenebutanoate as a colorless gum (350 mg, 86%).

### Step-4: Synthesis of 5-(4-chloro-2-(1H-pyrazol-4-yl)phenyl)-3-methylenedihydrofuran-2 (3H)-one

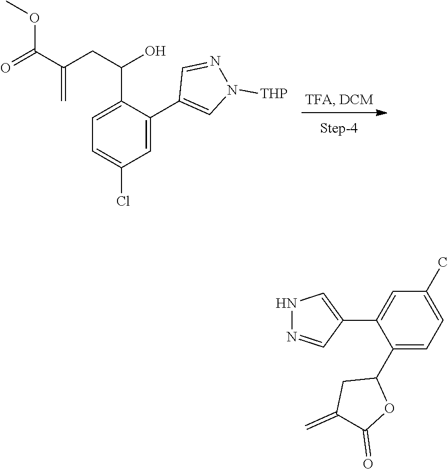

### Procedure:

To a stirred solution of methyl 4-(4-chloro-2-(1-(tetrahydro-2H-pyran-2-yl)-1H-pyrazol-4-yl)phenyl)-4-hydroxy-2-methylenebutanoate (300 g, 0.77 mmol) in DCM (15 mL), trifluro acetic acid (1 mL) was added at RT and the reaction mixture was stirred at RT for 16 h. Reaction progress was monitored by TLC. Upon disappearance of starting material, the reaction mixture was concentrated under reduced pressure to obtain brown gum. The crude product was purified by prep-HPLC to afford 5-(4-chloro-2-(1H-pyrazol-4-yl)phenyl)-3-methylenedihydrofuran-2 (3H)-one as an off white gum (15 mg, 6%).

1H-NMR (400 MHz, CDCl3): 7.69 (s, 2H), 7.40-7.33 (m, 2H), 6.31-6.30 (t, 1H), 5.68-5.63 (m, 2H), 3.23-3.16 (m, 1H), 2.92-2.84 (m, 1H).

LCMS (M+H=275.0, 96.42%).

### 5-(5-chloro-2-(1H-pyrazol-4-yl)phenyl)-3-methylenedihydrofuran-2 (3H)-one

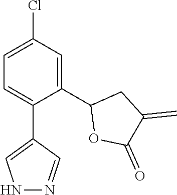 [SMILES: C=C1CC(c2cc(Cl)ccc2-c2cn[nH]c2)OC1=O]

¹H-NMR (400 MHz, CDCl₃): 7.67 (s, 2H), 7.445-7.440 (m, 1H), 7.36-7.33 (m, 1H), 7.27-7.24 (m, 1H), 6.33-6.31 (t, 1H), 5.69-5.65 (m, 1H), 5.64-5.62 (m, 1H), 3.19-3.17 (m, 1H), 2.91-2.89 (m, 1H).

LCMS (M+H=275.1, 90.58%)

### 5-(5-fluoro-2-(1H-pyrazol-4-yl)phenyl)-3-methylenedihydrofuran-2 (3H)-one

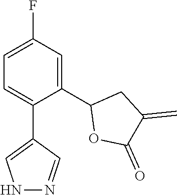 [SMILES: C=C1CC(c2cc(F)ccc2-c2cn[nH]c2)OC1=O]

¹H-NMR (400 MHz, CDCl₃): 7.67 (s, 2H), 7.30-7.27 (m, 1H), 7.18-7.15 (m, 1H), 7.09-7.04 (m, 1H), 6.32-6.30 (t, 1H), 5.68-5.62 (m, 2H), 3.22-3.15 (m, 1H), 2.89-2.61 (m, 1H).

LCMS (M+H=259.1, 90.21%)

### 5-(3-chloro-2-(1H-pyrazol-4-yl)phenyl)-3-methylenedihydrofuran-2 (3H)-one

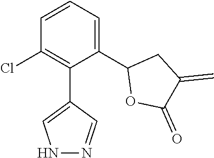 [SMILES: C=C1CC(c2cccc(Cl)c2-c2cn[nH]c2)OC1=O]

¹H-NMR (400 MHz, CDCl₃): 7.62 (s, 2H), 7.58-7.46 (m, 1H), 7.44-7.32 (m, 2H), 6.28-6.26 (m, 1H), 5.63-5.62 (t, 1H), 5.48-5.45 (m, 1H), 3.11-3.04 (m, 1H), 2.81-2.66 (m, 1H).

LCMS (M+H=275.0, 98.22%)

### 5-(4,5-difluoro-2-(1H-pyrazol-4-yl)phenyl)-3-methylenedihydrofuran-2 (3H)-one

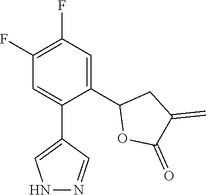 [SMILES: C=C1CC(c2cc(F)c(F)cc2-c2cn[nH]c2)OC1=O]

¹H-NMR (400 MHz, CDCl₃): 7.72 (s, 2H), 7.29-7.24 (m, 1H), 7.16-7.11 (m, 1H), 6.33-6.23 (t, 1H), 5.70-5.69 (t, 1H), 5.61-5.55 (m, 1H), 3.49-3.16 (m, 1H), 2.89-2.81 (m, 1H).

LCMS (M+H=277.0, 95.34%).

### 5-(4,5-dichloro-2-(1H-pyrazol-4-yl)phenyl)-3-methylenedihydrofuran-2 (3H)-one

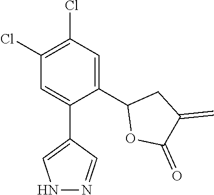 [SMILES: C=C1CC(c2cc(Cl)c(Cl)cc2-c2cn[nH]c2)OC1=O]

¹H-NMR (400 MHz, CDCl₃): 7.68 (s, 2H), 7.53 (s, 1H), 7.42 (s, 1H), 6.33-6.32 (t, 1H), 5.70-5.69 (t, 1H), 5.62-5.58 (m, 1H), 3.23-3.17 (m, 1H), 2.88-2.82 (m, 1H).

LCMS (M⁺+H): 309.14, (96.44%).

### 4-hydroxy-5-(2-(1-methyl-1H-pyrazol-4-yl)phenyl)-3-methylenedihydrofuran-2 (3H)-one

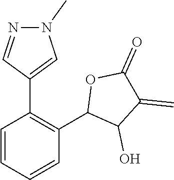 [SMILES: C=C1C(=O)OC(c2ccccc2-c2cnn(C)c2)C1O]

### Step-1

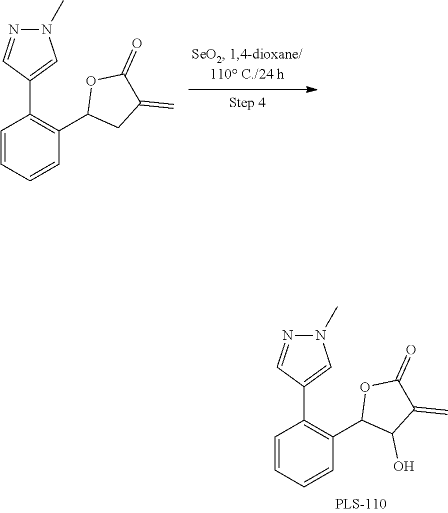 [SMILES: C=C1CC(c2ccccc2-c2cnn(C)c2)OC1=O ; C=C1C(=O)OC(c2ccccc2-c2cnn(C)c2)C1O]

### Procedure:

To a stirred solution of 5-(2-(1-methyl-1H-pyrazol-4-yl)phenyl)-3-methylenedihydrofuran-2 (3H)-one (0.087 g 0.31 mmol) in dioxane (5 mL) and water (1 mL) was added selenium dioxide (0.053 g 0.47 mmol) at room temperature. Then reaction mixture stirred at 100° C. for 24 h. The progress of the reaction was monitored by TLC. After completion of the starting material, it was diluted with H₂O (50 mL) and extracted with EtOAc (2×20 mL). The combined organic layer was dried over Na₂SO₄, filtered and evaporated under reduced pressure to get the crude compound. The crude obtained was purified by SiO₂ column chromatography (eluent: 5% methanol and DCM) to afford 4-hydroxy-5-(2-(1-methyl-1H-pyrazol-4-yl) phenyl)-3-methylenedihydrofuran-2 (3H)-one as brown solid (0.015 g, 17.4%).

¹H-NMR (300 MHz, CDCl₃) δ 7.60 (d, J=12.0 Hz, 2H), 7.38-7.31 (m, 4H), 6.50 (d, J=2.1 Hz, 1H), 6.00 (d, J=1.8 Hz, 1H), 5.55 (d, J=4.2 Hz, 1H), 4.84 (t, J=2.1 Hz, 1H), 3.96 (s, 3H),

LCMS: Rt 2.03 min (90.38% purity), m/z 271.14 [M+H]⁺.

### 5-(4-chloro-2-(1-methyl-1H-pyrazol-4-yl)phenyl)-4-hydroxy-3-methylenedihydrofuran-2 (3H)-one

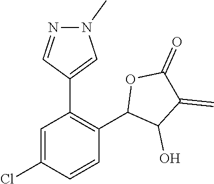 [SMILES: C=C1C(=O)OC(c2ccc(Cl)cc2-c2cnn(C)c2)C1O]

¹H-NMR (300 MHz, CDCl₃) δ 7.66-7.58 (m, 2H), 7.38-7.20 (m, 3H), 6.50 (bs, 1H), 6.01 (d, J=1.2 Hz, 1H), 5.50 (d, J=4.4 Hz, 1H), 4.84 (bs, 1H), 3.96 (s, 3H), 3.0 (bs, 1H).

LCMS (M++H): 326.8 (sodium adduct), (98.24%).

### 5-methyl-5-(2-(1-methyl-1H-pyrazol-4-yl)phenyl)-3-methylenedihydrofuran-2 (3H)-one

### Synthetic Scheme

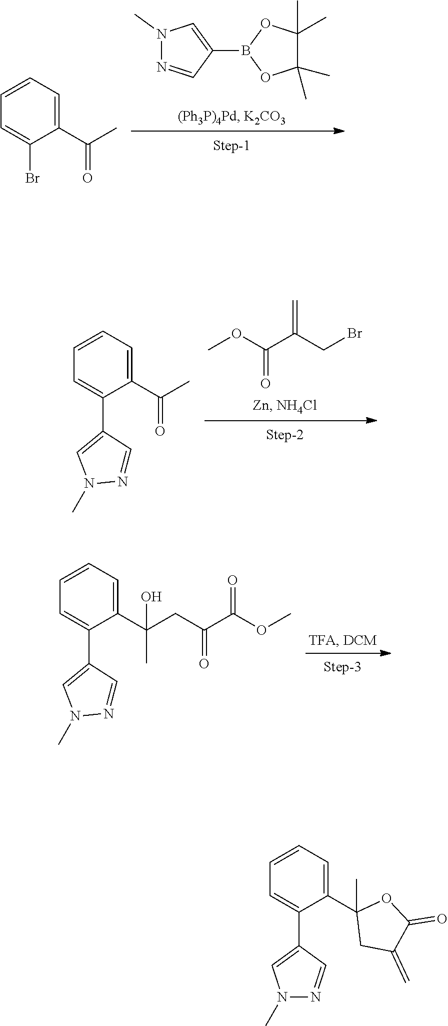

### Step-1: synthesis of 1-(-2-(1-methyl-1H-pyrazol-4-yl)phenyl)etan-1-one

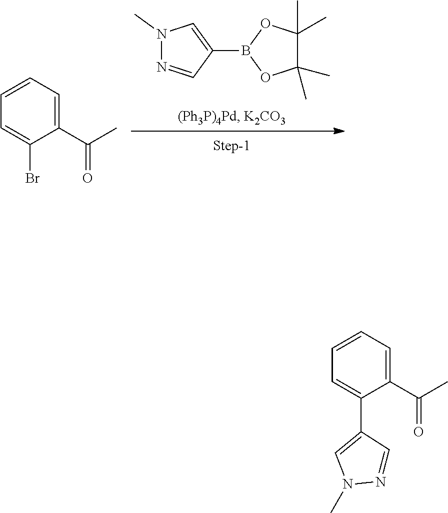

### Procedure:

To a stirred solution of 1-(2-bromophenyl)ethan-1-one (1 g, 5.02 mmol) and 1-(1-methyl-1H-pyrazol-4-yl)boronic acid (760 mg, 6.02 mmoL) in 1,4-dioxane (20 mL), potassium carbonate (1.7 g, 12.55 mmol) in water (20 mL) was added. The resulting mixture was degassed for 5 min, and tetrakis(triphenylphosphine)-palladium (500 mg, 10% mol) was added. The reaction mixture was degassed again for further 5 min. The resulting reaction mixture was heated at 70° C. for 2 h. The reaction progress was monitored by TLC. Upon disappearance of starting material, the reaction mixture was diluted with ethyl acetate (50 mL) and washed with water (2×20 mL). The organic layer was dried over Na₂SO₄, filtered and the solvent was evaporated under reduced pressure to obtain the crude product. It was purified by column chromatography using silica gel (mesh: 60-120) and 40-50% ethyl acetate and petroleum ether as eluent to afford 1-(-2-(1-methyl-1H-pyrazol-4-yl)phenyl)ethan-1-one as an off-white solid (820 mg, 82%).

### Step-2: Synthesis of methyl 4-hydroxy-4-(2-(1-methyl-1H-pyrazol-4-yl)phenyl)-2-methylenepentanoate

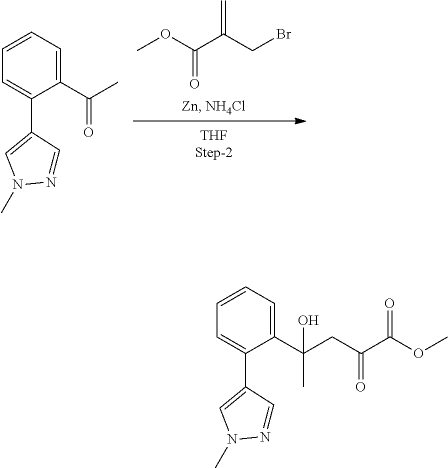

### Procedure:

To a stirred solution of 1-(-2-(1-methyl-1H-pyrazol-4-yl)phenyl)etan-1-one (0.5 g, 2.50 mmol) and methyl 2-(bromomethyl)acrylate (580 mg, 3.24 mmoL) in THF (10 mL), Zinc (1.07 g, 16.25 mmol) and saturated NH₄Cl in water (5 mL) was added. The resulting mixture was stirred at RT for 2 h. The reaction progress was monitored by TLC. Upon disappearance of starting material, the reaction mixture was filtered and filtrate was extracted with ethyl acetate (20 mL) and washed with water (2×10 mL). The organic layer was dried over Na₂SO₄, filtered and the solvent was evaporated under reduced pressure to obtain crude product. It was purified by column chromatography using silica gel (mesh: 60-120) and 50-60% ethyl acetate and petroleum ether as eluent to afford methyl 4-hydroxy-4-(2-(1-methyl-1H-pyrazol-4-yl)phenyl)-2-methylenepentanoate as a light brown gum (150 mg, 20%).

### Step-3: Synthesis of 5-methyl-5-(2-(1-methyl-1H-pyrazol-4-yl)phenyl)-3-methylenedihydrofuran-2 (3H)-one

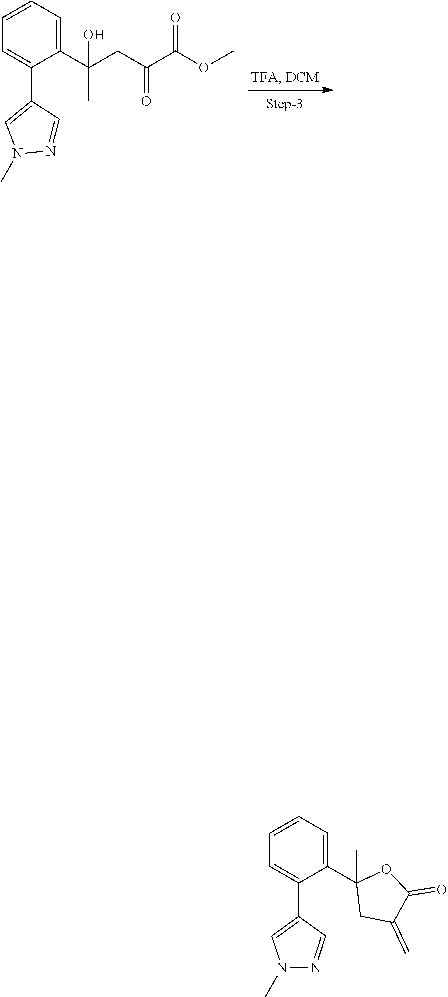

### Procedure:

To a stirred solution of methyl 4-hydroxy-4-(2-(1-methyl-1H-pyrazol-4-yl)phenyl)-2-methylenepentanoate (150 mg, 0.49 mmol) in DCM (10 mL), trifluoroacetic acid (1 mL) was added and the reaction mixture was stirred at RT for 16 h. The reaction progress was monitored by TLC. Upon disappearance of starting material, the reaction mixture was concentrated under reduced pressure to obtain crude product. It was purified by column chromatography using silica gel (mesh: 60-120) and 40-50% ethyl acetate and petroleum ether as eluent to afford 5-methyl-5-(2-(1-methyl-1H-pyrazol-4-yl)phenyl)-3-methylenedihydrofuran-2 (3H)-one as an off-white gum (64 mg, 47%).

¹H-NMR (400 MHz, CDCl₃): 7.73-7.72 (d, 1H), 7.42-7.38 (m, 1H), 7.369-7.367 (m, 2H), 7.32-7.28 (m, 1H) 7.168-7.165 (d, 1H), 6.18-6.16 (t, 1H), 5.53-5.52 (t, 1H), 3.97 (s, 3H), 3.10-3.04 (m, 1H), 2.76-2.71 (m, 1H), 1.6 (s, 3H).

LCMS: 269.25 (M++H), 99.72% purity.

### 5-(4-chloro-2-(1-methyl-1H-pyrazol-4-yl)phenyl)-5-methyl-3-methylenedihydrofuran-2 (3H)-one

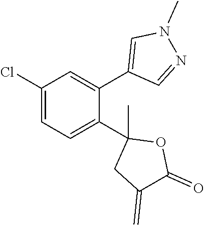 [SMILES: C=C1CC(C)(c2ccc(Cl)cc2-c2cnn(C)c2)OC1=O]

¹H-NMR (400 MHz, CDCl₃): 7.64-7.66 (m, 1H); 7.43 (s, 1H); 7.32-7.36 (m, 2H), 7.17 (d, 1H, 3 Hz); 6.17-6.19 (m, 1H); 5.53-5.54 (m, 1H); 3.96 (s, 3H); 3.00-3.05 (m, 1H), 2.70-2.75 (m, 1H); 1.68 (s, 3H)

LCMS (M+H): 303.4, 99.93%.

### 5-(4-chloro-2-(1-methyl-1H-pyrazol-4-yl)phenyl)-4-methyl-2-methylenedihydrofuran-3 (2H)-one

### Synthetic Scheme

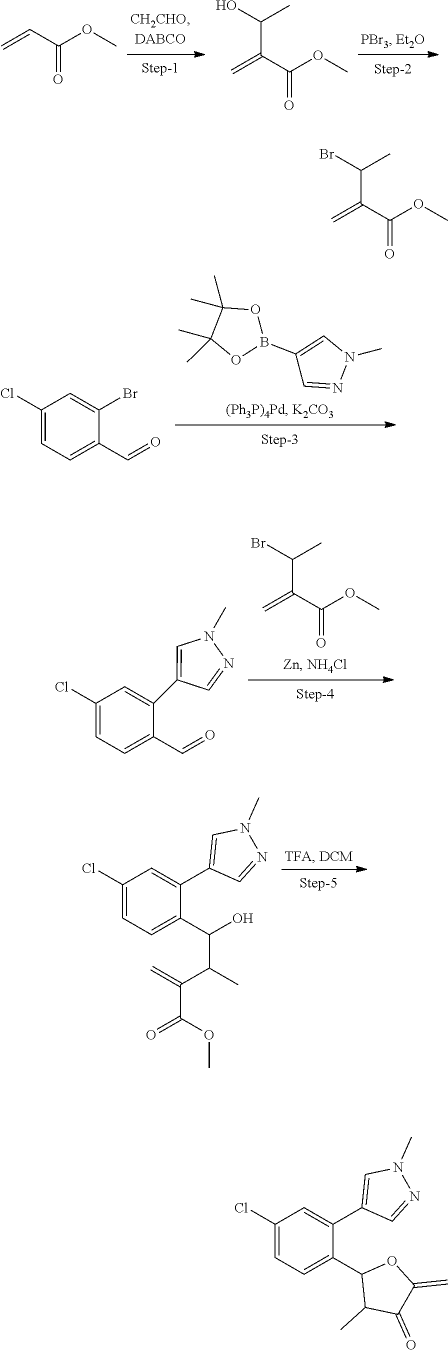

### Step-1: Synthesis of Methyl 3-hydroxy-2-methylenebutanoate

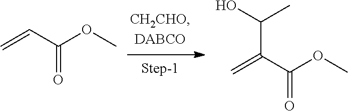

### Procedure:

To a stirred solution of acetaldehyde (5 g, 0.11 mmoL) and methyl acrylate (19.2 g, 0.15 mmoL) in 1,4-dioxane (20 mL) and water (20 mL), DABCO (19.2 g, 0.18 mmoL) was added at room temperature. The resulting reaction mixture was stirred at RT for 16 h. The reaction progress was monitored by TLC. The reaction mixture was diluted with ethyl acetate (40 mL) and washed with water (2×30 mL). The organic layer was dried over Na₂SO₄, filtered and then solvent was evaporated under reduced pressure to obtain crude product as yellow oil. The crude was purified by column chromatography (silica gel: 60+120, 5-10% ethyl acetate in petroleum ether) to obtain methyl 3-hydroxy-2-methylenebutanoate as pale-yellow oil (8 g, 95%)

### Step-2: Synthesis of methyl 3-bromo-2-methylenebutanoate

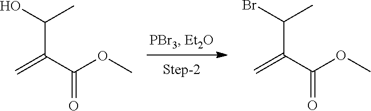

### Procedure:

To a stirred solution of methyl 3-hydroxy-2-methylenebutanoate (8 g, 0.06147 mmol) in diethyl ether (30 mL), PBr₃ (3 mL, 0.0307 mmol) was added drop wise at 0° C. The resulting reaction mixture was stirred at 0° C. for 1 h. The reaction progress was monitored by TLC. Upon disappearance of starting material, the reaction mixture was quenched with ice cold water and extracted with diethyl ether (2×25 mL). The organic layer was dried over Na₂SO₄, filtered and then solvent was evaporated under reduced pressure to obtain methyl 3-bromo-2-methylenebutanoate as a thick brown liquid (4 g, 35%).

### Step-3: Synthesis of 4-chloro-2-(1-methyl-1H-pyrazol-4-yl)benzaldehyde

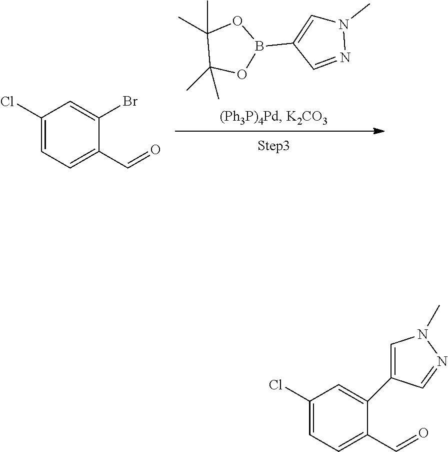

### Procedure:

To a stirred solution of 2-bromo-4-chlorobenzaldehyde (400 mg, 1.82 mmoL) and 1-methyl-4-(4,4,5,5-tetramethyl-1,3,2-dioxaborolan-2-yl)-1H-pyrazole (496 mg, 2.36 mmoL) in 1,4-dioxane (8 mL), potassium carbonate (656 mg, 4.74 mmol) in water (5 mL) was added. The resulting mixture was degassed for 5 min, and tetrakis(triphenylphosphine)-palladium (40 mg, 10 mol %) was added. The reaction mixture was degassed for further 5 min. The resulting reaction mixture was heated at 70° C. for 2 h. The reaction progress was monitored by TLC. Upon disappearance of starting material, the reaction mixture was diluted with ethyl acetate (15 mL) and washed with water (2×10 mL). The organic layer was dried over Na₂SO₄, filtered and then solvent was evaporated under reduced pressure to obtain crude product. It was purified by column chromatography using silica gel (mesh: 60-120) and the required compound was eluted at 25-30% ethyl acetate and petroleum ether to afford 4-chloro-2-(1-(tetrahydro-2H-pyran-2-yl)-1H-pyrazol-4-yl)benzaldehyde as a pale yellow solid (300 mg, 74%).

### Step-4: Synthesis of methyl 4-(4-chloro-2-(1-methyl-1H-pyrazol-4-yl)phenyl)-4-hydroxy-3-methyl-2-methylenebutanoate

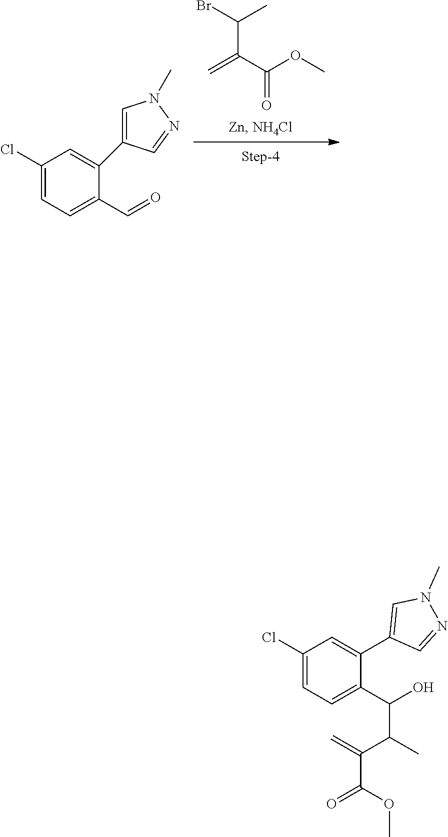

### Procedure:

To a stirred solution of 4-chloro-2-(1-(tetrahydro-2H-pyran-2-yl)-1H-pyrazol-4-yl)benzaldehyde (300 mg, 1.36 mmol) and methyl 3-bromo-2-methylenebutanoate (360 mg, 1.76 mmoL) in THF (10 mL), Zinc (320 mg, 4.88 mmol) and saturated NH₄Cl in water (1 mL) was added. The resulting mixture was stirred at RT for 2 h. The reaction progress was monitored by TLC. Upon disappearance of starting material, the reaction mixture was filtered and filtrate was extracted with ethyl acetate (20 mL) and washed with water (2×10 mL). The organic layer was dried over Na₂SO₄, filtered and then solvent was evaporated under reduced pressure to afford product methyl 4-(4-chloro-2-(1-methyl-1H-pyrazol-4-yl)phenyl)-4-hydroxy-3-methyl-2-methylenebutanoate as a white gum (300 mg, 65%).

### Step-5: 4-(4-chloro-2-(1-methyl-1H-pyrazol-4-yl)phenyl)-4-hydroxy-3-methyl-2-methylenebutanoic acid

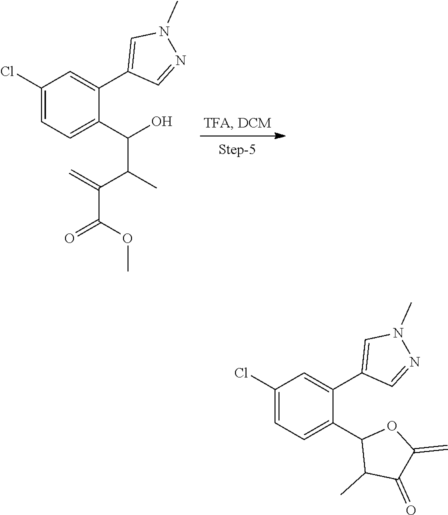 [SMILES: C=C(C(=O)OC)C(C)C(O)c1ccc(Cl)cc1-c1cnn(C)c1 ; C=C1OC(c2ccc(Cl)cc2-c2cnn(C)c2)C(C)C1=O]

### Procedure:

To a stirred solution of 4-(4-chloro-2-(1-methyl-1H-pyrazol-4-yl)phenyl)-4-hydroxy-3-methyl-2-methylenebutanoic acid (150 mg, 0.47 mmol) in DCM (15 mL), trifluro acetic acid (1 mL) was added at RT and the reaction mixture was stirred at RT for 16 h. The reaction progress was monitored by TLC. Upon disappearance of starting material, the reaction mixture was concentrated under reduced pressure to afford the crude product as brown gum. It was purified by column chromatography (silica gel: 100-200, 25-30% ethyl acetate in petroleum ether) to obtain the desired product 5-(4-chloro-2-(1-methyl-1H-pyrazol-4-yl)phenyl)-4-methyl-2-methylenedihydrofuran-3 (2H)-one as an pale yellow gummy (28 mg, 21%).

¹H-NMR (400 MHz, CDCl₃): 7.53 (s, 1H), 7.48 (s, 1H), 7.35 (m, 1H), 7.31-7.26 (m, 2H), 6.31-6.28 (m, 1H), 5.593-5.57 (m, 1H), 3.99 (s, 3H), 3.24-3.19 (m, 1H), 1.29-1.28 (d, 3H).

LCMS (M+H=303.1, 99.91%)

### 5-(4-chloro-2-(1-methyl-1H-pyrazol-4-yl)phenyl)-4-cyclopropyl-3-methylenedihydrofuran-2 (3H)-one

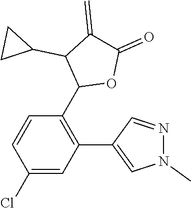 [SMILES: C=C1C(=O)OC(c2ccc(Cl)cc2-c2cnn(C)c2)C1C1CC1]

¹H-NMR (400 MHz, CDCl₃): 7.52-7.77 (m, 1H); 7.36-7.39 (m, 1H); 7.30-7.32 (m, 2H); 6.35-6.37 (m, 1H); 5.89 (d, 0.5H, 3 Hz); 5.80 (d, 0.5H, 3 Hz); δ 60 (d, 0.5H, 6 Hz) & 5.78 (d, 0.5H, 6 Hz); 4.07 (s, 3H); 2.37-2.62 (m, 1H); 0.01-0.58 (m, 5H).

LCMS (M+H): 329.4, 97.53%.

### 5-(4-chloro-2-(1H-pyrazol-4-yl)phenyl)-4-methyl-3-methylenedihydrofuran-2 (3H)-one

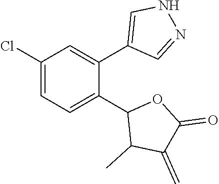 [SMILES: C=C1C(=O)OC(c2ccc(Cl)cc2-c2cn[nH]c2)C1C]

¹H-NMR (400 MHz, CDCl₃): 7.65 (bs, 2H); 7.28-7.36 (m, 3H); 6.31 (d, 1H, J=1.5 Hz), 5.80 (d, 1H, J=6 Hz); 5.57 (d, 1H, J=1.5 Hz); 3.18-3.22 (m, 1H); 0.75 (d, 3H, J=6 Hz).

LCMS (M+H): 289.4, 93%.

### 5-(5-chloro-2-(1-methyl-1H-pyrazol-4-yl)phenyl)-4-methyl-3-methylenedihydrofuran-2 (3H)-one

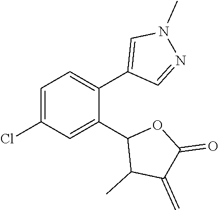 [SMILES: C=C1C(=O)OC(c2cc(Cl)ccc2-c2cnn(C)c2)C1C]

¹H-NMR (400 MHz, CDCl₃): 7.47 (bs, 1H); 7.38 (s, 1H); 7.35-7.36 (m, 1H); 7.29-7.31 (m, 1H); 7.18-7.20 (m, 1H); 6.32 (d, 1H, J=1.5 Hz), 5.80 (d, 1H, J=6 Hz); 5.60 (d, 1H, J=1.5 Hz); 3.97 (s, 3H); 3.18-3.22 (m, 1H); 0.80 (d, 3H, J=6 Hz).

LCMS (M+H)=303.4, 96.81%.

### 5-(5-chloro-2-(1H-pyrazol-4-yl)phenyl)-4-methyl-3-methylenedihydrofuran-2 (3H)-one

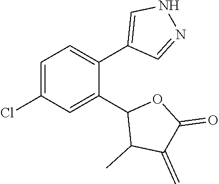 [SMILES: C=C1C(=O)OC(c2cc(Cl)ccc2-c2cn[nH]c2)C1C]

¹H-NMR (400 MHz, CDCl₃): 7.67 (bs, 2H); 7.31-7.38 (m, 2H); 7.21-7.26 (m, 1H); 6.32 (d, 1H, J=1.5 Hz), 5.78 (d, 1H, J=6 Hz); 5.59 (d, 1H, J=1.5 Hz); 3.18-3.22 (m, 1H); 0.80 (d, 3H, J=6 Hz).

LCMS (M+H)=289.3, 96.81%.

### 5-(5-fluoro-2-(1-methyl-1H-pyrazol-4-yl)phenyl)-4-methyl-3-methylenedihydrofuran-2 (3H)-one

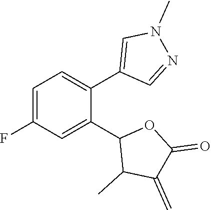 [SMILES: C=C1C(=O)OC(c2cc(F)ccc2-c2cnn(C)c2)C1C]

¹H-NMR (400 MHz, CDCl₃): 7.46 (bs, 1H); 7.37 (s, 1H); 7.35-7.36 (m, 1H); 7.29-7.31 (S, 1H); 7.20-7.26 (m, 1H); 7.00-7.5 (m, 1H); 6.30 (d, 1H, J=1.5 Hz); 5.80 (d, 1H, J=6 Hz); 5.60 (d, 1H, J=1.5 Hz); 3.96 (s, 3H); 3.17-3.220 (m, 1H); 0.80 (d, 3H, J=6 Hz).

LCMS (M+H)=287.3, 95.02%.

### 5-(4,5-dichloro-2-(1-methyl-1H-pyrazol-4-yl)phenyl)-4-methyl-3-methylenedihydrofuran-2 (3H)-one

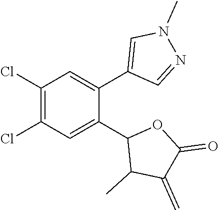 [SMILES: C=C1C(=O)OC(c2cc(Cl)c(Cl)cc2-c2cnn(C)c2)C1C]

¹H-NMR (400 MHz, DMSO): 8.02 (s, 1H); 7.71 (s, 1H); 7.66 (s, 1H); 7.38 (S, 1H); 6.18 (d, J=3 Hz, 1H); 5.99 (d, 1H, J=6 Hz); 5.80 (d, 1H, J=3 Hz); 3.89 (s, 3H); 3.47-3.42 (m, 1H), 0.69 (d, 3H, J=6 Hz).

LCMS (M⁺+H): 337.5, 97.08%.

### 5-(5-fluoro-2-(1H-pyrazol-4-yl)phenyl)-4-methyl-3-methylenedihydrofuran-2 (3H)-one

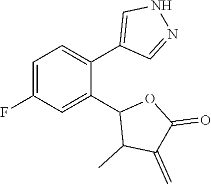 [SMILES: C=C1C(=O)OC(c2cc(F)ccc2-c2cn[nH]c2)C1C]

¹H-NMR (400 MHz, CDCl₃): 7.67 (bs, 2H); 7.08-7.25 (m, 3H); 6.30 (d, 1H, J=1.5 Hz); 5.77 (d, 1H, J=6 Hz); 5.58 (d, 1H, J=1.5 Hz); 3.13-3.17 (m, 1H); 0.80 (d, 3H, J=6 Hz).

LCMS (M+H)=273.2, 97.7%.

### 4-(4-chloro-2-(1-methyl-1H-pyrazol-4-yl)phenyl)-4-hydroxy-3-methyl-2-methylenebutanoic acid

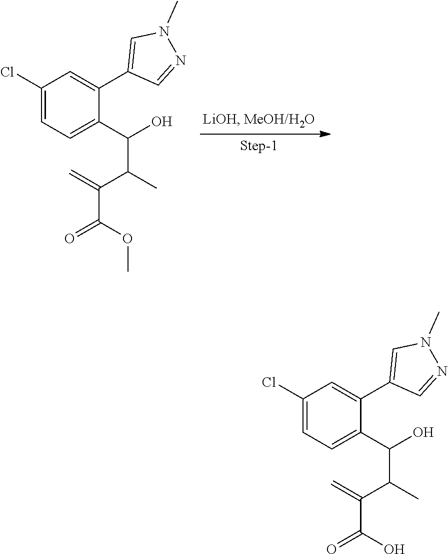 [SMILES: C=C(C(=O)OC)C(C)C(O)c1ccc(Cl)cc1-c1cnn(C)c1 ; C=C(C(=O)O)C(C)C(O)c1ccc(Cl)cc1-c1cnn(C)c1]

### Procedure:

To a stirred solution of methyl 4-(4-chloro-2-(1-methyl-1H-pyrazol-4-yl)phenyl)-4-hydroxy-3-methyl-2-methylenebutanoate (300 mg, 0.89 mmol) in THF (10 mL), MeOH (2 mL), was added LiOH·H₂O (76 mg, 1.79 mmoL) in water (4 mL) at RT. The reaction mixture was stirred at RT for 2 h. The reaction progress was monitored by TLC. Upon disappearance of starting material, the reaction mixture was concentrated under reduced pressure to obtain crude product. It was diluted with water and washed with ethyl acetate (2×10 mL). The aqueous layer was acidified with 6N HCl and again extracted with ethyl acetate (2×15 mL). The organic layer was dried over Na₂SO₄, filtered and then solvent was evaporated under reduced pressure to afford 4-(4-chloro-2-(1-methyl-1H-pyrazol-4-yl)phenyl)-4-hydroxy-3-methyl-2-methylenebutanoic acid as an off-white solid (95 mg, 34%).

¹H-NMR (400 MHz, CDCl₃): 12.25 (brs, 1H), 7.88 (brs, 1H), 7.59 (s, 1H), 7.57-7.50 (d, 1H), 7.30-7.22 (m, 1H), 7.24-7.23 (d, 1H), 5.91 (brs, 2H), 4.80-4.79 (d, 1H), 3.89 (s, 3H), 2.99-2.96 (t, 1H), 1.04-1.03 (d, 3H).

LCMS (M⁺+H: 321.1, 90.13% purity).

### 4-(4-chloro-2-(1-methyl-1H-pyrazol-4-yl)phenyl)-4-hydroxy-2-methylenebutanenitrile

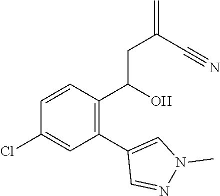 [SMILES: C=C(C#N)CC(O)c1ccc(Cl)cc1-c1cnn(C)c1]

¹H NMR (400 MHz, CDCl₃) δ 7.56 (d, J=8.4 Hz, 1H), 7.51 (d, J=2.4 Hz, 2H), 7.36-7.27 (m, 2H), 5.95 (s, 1H), 5.78 (s, 1H), 5.21 (dt, J=8.2, 4.2 Hz, 1H), 3.96 (s, 3H), 2.60-2.54 (m, 2H), 2.19 (d, J=3.7 Hz, 1H).

MS (m/z): 288.1 [M+H]⁺.

### 5-(4-chloro-2-(1-methyl-1H-pyrazol-4-yl)phenyl)-3-methylenedihydrofuran-2 (3H)-one

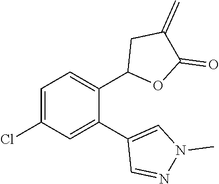 [SMILES: C=C1CC(c2ccc(Cl)cc2-c2cnn(C)c2)OC1=O]

¹H-NMR (400 MHz, CDCl₃): 7.97 (s, 1H), 7.66 (s, 1H), 7.43-7.41 (m, 3H), 6.12 (t, 1H), 5.85-5.75 (m, 2H), 3.89 (s, 3H), 3.50-3.40 (m, 1H), 2.90-2.80 (m, 1H).

HPLC: 98.18%.

### 5-(5-chloro-2-(1-methyl-1H-pyrazol-4-yl)phenyl)-3-methylenedihydrofuran-2 (3H)-one

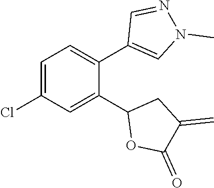

¹H-NMR (400 MHz, CDCl₃): 7.49 (s, 1H), 7.45 (s, 1H), 7.419-7.413 (d, 1H), 7.33-7.30 (d, 1H), 7.24-7.21 (d, 1H), 6.32-6.31 (t, 1H), 5.69-5.64 (m, 2H), 3.97 (s, 3H), 3.25-3.18 (m, 1H), 2.92-2.85 (m, 1H).

LCMS: m/z: 289.25 (M+H): 97.96%)

### 5-(2-chloro-6-(1-methyl-1H-pyrazol-4-yl)phenyl)-3-methylenedihydrofuran-2 (3H)-one

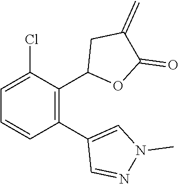 [SMILES: C=C1CC(c2c(Cl)cccc2-c2cnn(C)c2)OC1=O]

¹H-NMR (400 MHz, CDCl₃): 7.83 (s, 1H), 7.51 (s, 1H), 7.47-7.46 (d, 1H), 7.44-7.38 (m, 1H), 7.30-7.28 (m, 1H), 6.10-6.09 (t, 1H), 5.93-5.89 (m, 1H), 5.73 (s, 1H), 3.86 (s, 3H), 3.49-3.43 (m, 1H), 3.16-3.08 (m, 1H).

LCMS: m/z: 288.9 (M+H): 99.44%).

### 5-(3-chloro-2-(1-methyl-1H-pyrazol-4-yl)phenyl)-3-methylenedihydrofuran-2 (3H)-one

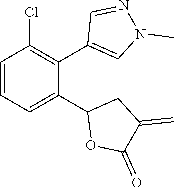

¹H-NMR (400 MHz, CDCl₃): 7.46-7.44 (m, 2H), 7.40 (s, 1H), 7.35-7.32 (m, 2H), 7.27-7.26 (t, 1H), (t, 1H), 5.63-5.62 (t, 1H), 5.52-5.48 (t, 1H), 3.99 (s, 3H), 3.14-3.08 (m, 1H), 2.80-2.74 (m, 1H)/

LCMS: m/z: 289.25 (M+H): 98.52%).

### 4-(2-(1-(tert-butyl)-1H-pyrazol-4-yl)-4-chlorophenyl)-4-hydroxy-2-methylenebutanoic acid

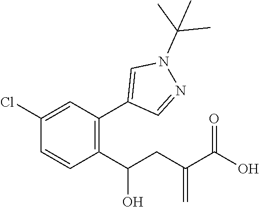 [SMILES: C=C(CC(O)c1ccc(Cl)cc1-c1cnn(C(C)(C)C)c1)C(=O)O]

¹H-NMR (400 MHz, CDCl₃): 12.4-12.3 (brs, 1H), 8.027 (s, 1H), 7.65-7.59 (m, 2H), 7.35-7.30 (m, 2H), 6.076-6.072 (d, 1H), 5.58 (s, 1H), 5.27-5.21 (brs, 1H), 5.05-4.95 (brs, 1H), 2.6-2.51 (m, 2H), 1.56 (s, 9H).

LCMS: m/z 349.1 [M⁺+H]; (99.42% purity)

### 4-(4-chloro-2-(1-(difluoromethyl)-1H-pyrazol-4-yl)phenyl)-4-hydroxy-2-methylenebutanoic acid

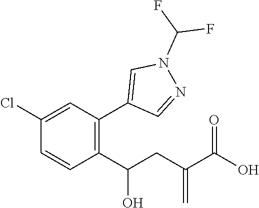

¹H-NMR (400 MHz, CDCl₃): 12.5-12.3 (brs, 1H), 8.01 (s, 1H), 7.84 (s, 1H), 7.70-7.59 (m, 2H), 7.45-7.35 (m, 2H), 6.04 (d, 1H), 4.91-4.85 (m, 1H), 2.6-2.51 (m, 2H).

LCMS: m/z 340.9 [M⁺−H]; (99.95% purity).

### (4-(4-chloro-2-(1-methyl-1H-pyrazol-4-yl)phenyl)-4-hydroxy-2-methylenebutanoyl)glycine

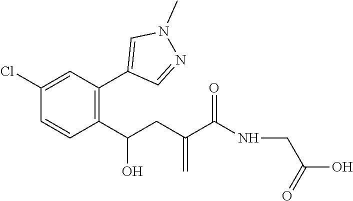

¹H-NMR (400 MHz, CDCl₃): 8.03 (s, 1H), 7.64 (s, 1H), 7.59 (d, 1H), 7.35-7.30 (m, 2H), 5.62 (s, 1H), 5.24 (d, 1H), 4.90-4.85 (m, 1H), 3.88 (s, 3H), 3.45 (s, 2H), 2.70-2.40 (m, 2H).

LCMS: m/z 361.9 [M⁺−H]; (98.15% purity).

### 4-(4-chloro-2-(1-methyl-1H-pyrazol-4-yl)phenyl)-4-hydroxy-N-methyl-2-methylenebutanamide

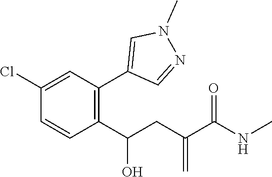

¹H-NMR (400 MHz, CDCl₃): 8.03 (s, 1H), 7.96 (s, 1H), 7.70-7.55 (m, 2H), 7.45-7.30 (m, 2H), 5.61 (s, 1H), 5.37 (d, 1H), 5.25 (s, 1H), 4.90-4.85 (m, 1H), 3.88 (s, 3H), 2.70-2.40 (m, 5H).

LCMS: m/z 317.8 [M⁺−H]; (98.83% purity).

### 4-(4-chloro-2-(1-ethyl-1H-pyrazol-4-yl)phenyl)-4-hydroxy-2-methylenebutanoic acid

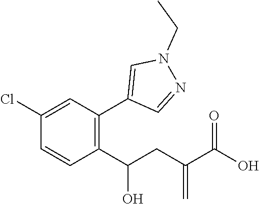 [SMILES: C=C(CC(O)c1ccc(Cl)cc1-c1cnn(CC)c1)C(=O)O]

¹H-NMR (400 MHz, CDCl₃): 12.5-12.3 (brs, 1H), 8.02 (s, 1H), 7.65 (s, 1H), 7.59 (d, 1H), 7.35-7.30 (m, 2H), 6.06 (d, 1H), 5.56 (s, 1H), 4.91-4.85 (m, 1H), 4.2 (q, 2H), 2.6-2.51 (m, 2H), 1.4 (t, 3H).

LCMS: m/z 340.9 [M⁺−H]; (99.95% purity).

### 4-(4-chloro-2-(1-cyclopropyl-1H-pyrazol-4-yl)phenyl)-4-hydroxy-2-methylenebutanoic acid

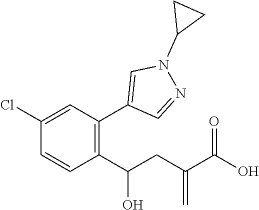

¹H-NMR (400 MHz, CDCl₃): 12.45-12.35 (brs, 1H), 8.02 (s, 1H), 7.63 (s, 1H), 7.56 (d, 1H), 7.35-7.27 (m, 2H), 6.05 (bs, 1H), 5.54 (s, 1H), 4.91-4.85 (m, 1H), 3.81-3.70 (m, 1H), 2.6-2.41 (m, 2H), 1.1-0.90 (m, 4H).

MS: m/z 333.0 [M⁺−H].

### 4-(4-chloro-2-(1-isopropyl-1H-pyrazol-4-yl)phenyl)-4-hydroxy-2-methylenebutanoic acid

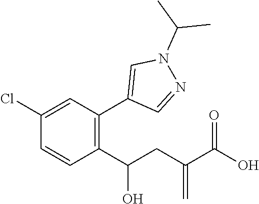

¹H-NMR (400 MHz, CDCl₃): 12.45-12.3 (brs, 1H), 8.03 (s, 1H), 7.66 (s, 1H), 7.59 (d, 1H), 7.35-7.30 (m, 2H), 6.07 (d, 1H), 5.56 (s, 1H), 5.31-5.20 (bs, 1H), 4.98-4.91 (m, 1H), 4.55-4.48 (m, 1H), 2.6-2.51 (m, 2H), 1.46 (s, 3H), 1.45 (s, 3H).

LCMS: m/z 335.0 [M⁺+H].

### 4-(4-chloro-2-(1-cyclopentyl-1H-pyrazol-4-yl)phenyl)-4-hydroxy-2-methylenebutanoic acid

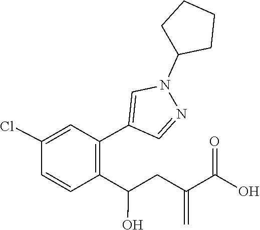 [SMILES: C=C(CC(O)c1ccc(Cl)cc1-c1cnn(C2CCCC2)c1)C(=O)O]

¹H-NMR (400 MHz, CDCl₃): 8.03 (s, 1H), 7.66 (s, 1H), 7.59 (d, 1H), 7.35-7.30 (m, 2H), 6.04 (d, 1H), 5.53 (s, 1H), 4.98-4.91 (m, 1H), 4.72-4.68 (m, 1H), 2.6-2.51 (m, 2H), 2.2-1.6 (m, 8H).

LCMS: m/z 361.1 [M⁺+H].

### 4-(4-chloro-2-(1-(cyclobutylmethyl)-1H-pyrazol-4-yl)phenyl)-4-hydroxy-2-methylenebutanoic acid

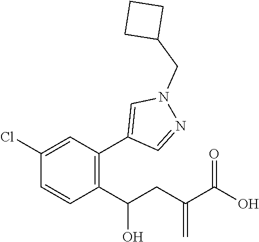

¹H-NMR (400 MHz, CDCl₃): 12.45-12.3 (brs, 1H), 7.99 (s, 1H), 7.66 (s, 1H), 7.59 (d, 1H), 7.35-7.30 (m, 2H), 6.07 (d, 1H), 5.56 (s, 1H), 4.98-4.91 (m, 1H), 4.55-4.48 (m, 1H), 4.14 (d, 2H), 2.8-2.65 (m, 1H), 2.6-2.51 (m, 2H), 2.2-1.6 (m, 6H).

LCMS: m/z 361.1 [M⁺+H]; (97.82% purity).

### 4-(4-chloro-2-(1-phenyl-1H-pyrazol-4-yl)phenyl)-4-hydroxy-2-methylenebutanoic acid

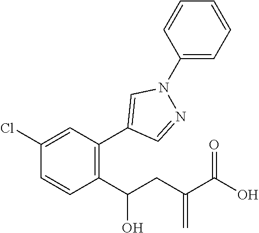 [SMILES: C=C(CC(O)c1ccc(Cl)cc1-c1cnn(-c2ccccc2)c1)C(=O)O]

¹H-NMR (400 MHz, CDCl₃): 12.45-12.3 (brs, 1H), 8.72 (s, 1H), 7.99 (s, 1H), 7.89 (d, 1H), 7.66-7.25 (m, 6H), 6.07 (d, 1H), 5.59 (s, 1H), 5.35-5.30 (bs, 1H), 5.15-5.05 (m, 1H), 2.6-2.51 (m, 2H).

LCMS: m/z 369.0 [M⁺+H]; (90.54% purity).

### 4-(4-chloro-2-(1-(cyclohexylmethyl)-1H-pyrazol-4-yl)phenyl)-4-hydroxy-2-methylenebutanoic acid

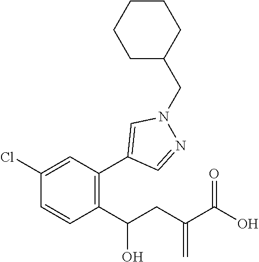 [SMILES: C=C(CC(O)c1ccc(Cl)cc1-c1cnn(CC2CCCCC2)c1)C(=O)O]

¹H-NMR (400 MHz, CDCl₃): 12.45-12.3 (brs, 1H), 7.97 (s, 1H), 7.66 (s, 1H), 7.59 (d, 1H), 7.35-7.27 (m, 2H), 6.07 (s, 1H), 5.58 (s, 1H), 5.25-5.20 (bs, 1H), 4.98-4.91 (m, 1H), 3.97 (d, 2H), 2.6-2.51 (m, 2H), 1.8-0.9 (m, 10H).

LCMS: m/z 389.1 [M⁺+H]; (92.5% purity).

### 4-(4-chloro-2-(1-(cyclopentylmethyl)-1H-pyrazol-4-yl)phenyl)-4-hydroxy-2-methylenebutanoic acid

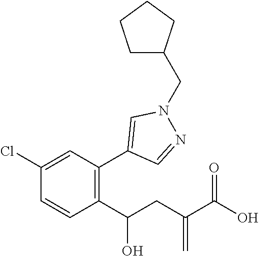 [SMILES: C=C(CC(O)c1ccc(Cl)cc1-c1cnn(CC2CCCC2)c1)C(=O)O]

¹H-NMR (400 MHz, CDCl₃): 12.45-12.3 (brs, 1H), 8.01 (s, 1H), 7.65 (s, 1H), 7.59 (d, 1H), 7.35-7.27 (m, 2H), 6.07 (s, 1H), 5.56 (s, 1H), 4.98-4.91 (m, 1H), 4.04 (d, 2H), 2.6-2.51 (m, 2H), 1.7-1.2 (m, 8H).

LCMS: m/z 375.1 [M⁺+H]; (94.68% purity).

### 4-(4,5-difluoro-2-(1-methyl-1H-pyrazol-4-yl)phenyl)-4-hydroxy-2-methylenebutanoic acid

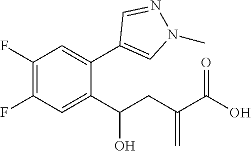 [SMILES: C=C(CC(O)c1cc(F)c(F)cc1-c1cnn(C)c1)C(=O)O]

¹H-NMR (400 MHz, CDCl₃): δ=7.46-7.44 (m, 2H), 7.40 (s, 1H), 7.35-7.26 (m, 2H), 6.27-6.26 (t, 1H), 5.63-5.62 (t, 1H), 3.99 (s, 3H), 3.14-3.08 (m, 1H), 2.80-2.74 (m, 1H).

LCMS: m/z 309 [M⁺+H]; (93.51% purity)

### 4-(4-chloro-2-(1-methyl-1H-pyrazol-4-yl)phenyl)-4-hydroxy-2-methylenebutanoic acid

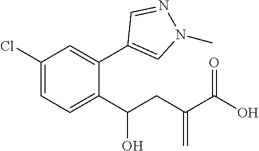 [SMILES: C=C(CC(O)c1ccc(Cl)cc1-c1cnn(C)c1)C(=O)O]

¹H-NMR (400 MHz, CDCl₃): 12.4-12.2 (brs, 1H), 7.93 (s, 1H), 7.58 (s, 2H), 7.30-7.29 (m, 2H), 6.076-6.072 (d, 1H), 5.59 (s, 1H), 5.3-5.2 (brs, 1H), 3.99 (s, 3H), 2.56-2.51 (m, 1H), 2.28 (m, 1H).

LCMS: m/z 307.1 [M⁺+H]; (92.99% purity)

### 4-(4-chloro-2-(1-(2-morpholinoethyl)-1H-pyrazol-4-yl)phenyl)-4-hydroxy-2-methylenebutanoic acid

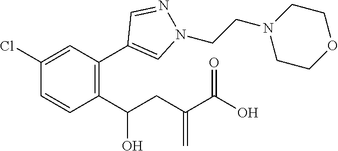 [SMILES: C=C(CC(O)c1ccc(Cl)cc1-c1cnn(CCN2CCOCC2)c1)C(=O)O]

¹H-NMR (400 MHz, CDCl₃): 7.60-7.58 (d, 1H), 7.43-7.41 (d, 2H), 7.34-7.31 (m, 1H), 7.17 (d, 1H), 6.23 (brs, 2H), 5.939-5.937 (s, 1H), 5.34-5.31 (m, 1H), 5.18 (s, 1H), 4.37-4.34 (m, 2H), 3.95 (brS, 5H), 3.83-3.74 (m, 4H), 3.16-3.10 (m, 1H), 3.05-2.99 (m, 1H), 2.89 (brs, 4H), 2.70-2.65 (m, 1H), 2.44-2.39 (m, 1H).

LCMS (M+H=406.2, 99.48%).

### 4-hydroxy-2-(3-(1-methyl-1H-pyrazol-4-yl)phenyl)-5-methyleneisothiazolidine 1,1-dioxide

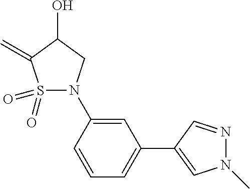

¹H NMR (400 MHz, CDCl₃) δ 7.76 (s, 1H), 7.64 (s, 1H), 7.44 (s, 1H), 7.41-7.31 (m, 2H), 7.18 (d, J=7.5 Hz, 1H), 6.34 (s, 1H), 6.09 (s, 1H), 5.07 (s, 1H), 4.04 (dd, J=9.5, 6.5 Hz, 1H), 3.95 (s, 3H), 3.66 (dd, J=9.5, 4.9 Hz, 1H), 2.70 (d, J=7.3 Hz, 1H).

LCMS (m/z): 306.2 [M⁺+H].

### 5-(4-chloro-2-(1-methyl-1H-pyrazol-4-yl)phenyl)-3-(2-fluorobenzylidene)dihydrofuran-2 (3H)-one

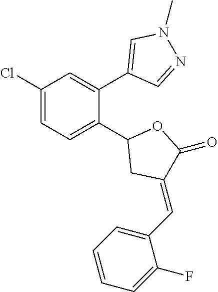 [SMILES: Cn1cc(-c2cc(Cl)ccc2C2C/C(=C\c3ccccc3F)C(=O)O2)cn1]

¹H-NMR (400 MHz, CDCl₃): 7.53 (s, 1H); 7.50 (s, 1H); 7.31-7.39 (m, 5H); 711-7.20 (m, 2H), 5.73-5.76 (m, 1H); 3.97 (s, 3H) 3.39-3.46 (1H, m); 3.06-3.13 (m, 1H).

MS (M++H): 383.1

### 4-(4-chloro-2-(1-(2-(dimethylamino)ethyl)-1H-pyrazol-4-yl)phenyl)-4-hydroxy-2-methylenebutanenitrile

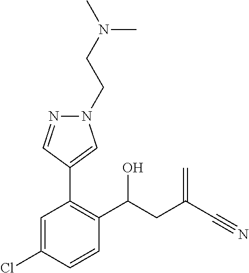 [SMILES: C=C(C#N)CC(O)c1ccc(Cl)cc1-c1cnn(CCN(C)C)c1]

¹H NMR (400 MHz, CDCl₃) δ 8.068 (s, 1H), 7.68 (s, 1H), 7.61 (d, J=8.4 Hz, 1H), 7.40 (d, J=8.4 Hz, 1H), 7.31 (m, 1H), 6.02 (s, 1H), 5.91 (s, 1H), 5.64 (s, 1H), 4.98-4.90 (m, 1H), 4.44 (bs, 2H), 3.15 (bs, 2H), 2.60-2.54 (m, 2H), 2.45-2.40 (m, 6H).

LCMS (M++H): 345.6, 98.28%.

### 4-(4,5-difluoro-2-(1-methyl-1H-pyrazol-4-yl)phenyl)-4-hydroxy-2-methylenebutanenitrile

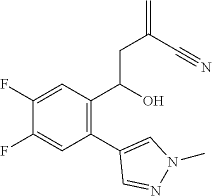 [SMILES: C=C(C#N)CC(O)c1cc(F)c(F)cc1-c1cnn(C)c1]

¹H NMR (400 MHz, CDCl₃) δ 7.48-7.50 (m, 3H), 7.12-7.07 (m, 1H), 5.96 (s, 1H), 5.80 (s, 1H), 5.21-5.15 (m, 1H), 3.96 (s, 3H), 2.60-2.54 (m, 2H), 2.15 (d, J=3.6 Hz, 1H).

LCMS (M⁺+H): 289.9, 97.62%.

### 3-(4-chloro-2-(1-methyl-1H-pyrazol-4-yl)phenyl)-4-hydroxy-2-methyl-5-methyleneisothiazolidine 1,1-dioxide

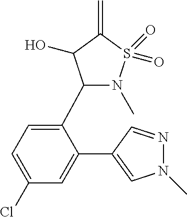 [SMILES: C=C1C(O)C(c2ccc(Cl)cc2-c2cnn(C)c2)N(C)S1(=O)=O]

¹H-NMR (400 MHz, CDCl₃): 7.2 (s, 2H); 7.44-7.47 (m, 1H); 7.35-7.39 (m, 2H); 6.29 (t, 1H), 6.97 (t, 1H); 4.72-4.74 (m, 1H); 4.40 (d, 1H, J=6 Hz); 3.95 (s, 3H); 2.41 (s, 3H).

MS (M⁺+H): 354.1

### 4-hydroxy-2-(3-(1-methyl-1H-pyrazol-4-yl)phenyl)-5-methyleneisothiazolidine 1,1-dioxide

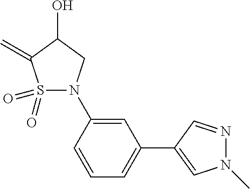

¹H-NMR (400 MHz, CDCl₃): 7.75 (s, 1H); 7.63 (s, 1H); 7.44 (s, 1H); 7.32-7.38 (m, 2H); 7.16-7.18 (m, 1H); 6.33 (bs, 1H); 6.09 (bs, 1H); 5.07 (bs, 1H); 4.03-4.05 (m, 1H); 3.94 (s, 3H); 3.64-3.67 (m, 1H); 2.69-2.67 (m, 1H)

LCMS (M⁺+H): 306.2, 98.4%.

### 4-hydroxy-2-methyl-3-(2-(1-methyl-1H-pyrazol-4-yl)phenyl)-5-methyleneisothiazolidine 1,1-dioxide

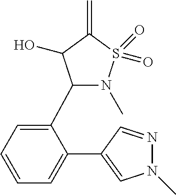 [SMILES: C=C1C(O)C(c2ccccc2-c2cnn(C)c2)N(C)S1(=O)=O]

¹H-NMR (400 MHz, CDCl₃): 7.50-7.56 (m, 3H); 7.33-7.43 (m, 3H); 6.28 (t, 1H); 5.95 (t, 1H); 4.75-4.79 (m, 1H); 4.44-4.45 (m, 1H); 3.94 (s, 3H); 3.48 (s, 1H); 2.83-2.83 (m, 1H); 2.41 (s, 3H)

LCMS (M⁺+H): 320.1, 98.54%.

### 3-(4-chloro-2-(1-methyl-1H-pyrazol-4-yl)phenyl)-2-ethyl-4-hydroxy-5-methyleneisothiazolidine 1,1-dioxide

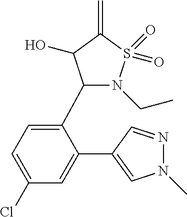 [SMILES: C=C1C(O)C(c2ccc(Cl)cc2-c2cnn(C)c2)N(CC)S1(=O)=O]

¹H-NMR (400 MHz, CDCl₃): 7.61 (s, 2H); 7.47-7.49 (m, 1H); 7.37-7.38 (m, 1H); 7.35 (s, 1H); 6.26 (t, 1H); 5.92 (t, 1H); 4.74 (bs, 1H); 4.57 (d, 1H, J=6 Hz); 3.97 (s, 3H); 3.03 (m, 1H); 2.81-2.86 (m, 1H); 0.95 (t, 3H)

LCMS (M⁺+H): 368.1, 98.0%.

### 4-(3-chloro-2-(1-methyl-1H-pyrazol-4-yl)phenyl)-4-hydroxy-2-methylenebutanenitrile

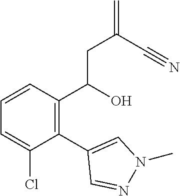 [SMILES: C=C(C#N)CC(O)c1cccc(Cl)c1-c1cnn(C)c1]

¹H NMR (400 MHz, CDCl₃) δ 7.55 (d, J=7.6 Hz, 1H), 7.46-7.32 (m, 4H), 5.91 (s, 1H), 5.72 (s, 1H), 5.01-4.96 (m, 1H), 3.99 (s, 3H), 2.49 (d, J=6.4 Hz, 2H), 2.22 (d, J=3.2 Hz, 1H).

LCMS (M⁺+H): 288.0, 97.86%.

### 4-(3-chloro-2-(1H-pyrazol-4-yl)phenyl)-4-hydroxy-2-methylenebutanenitrile

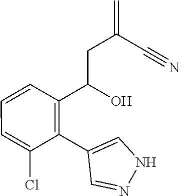 [SMILES: C=C(C#N)CC(O)c1cccc(Cl)c1-c1cn[nH]c1]

¹H NMR (400 MHz, CDCl₃) δ 7.60 (m, 2H), 7.56 (d, J=8 Hz, 1H), 7.44 (d, J=8 Hz, 1H), 7.35 (t, J=8 Hz, 1H), 5.90 (s, 1H), 5.71 (s, 1H), 4.97 (t, J=6.4 Hz, 1H), 2.49 (d, J=6 Hz, 2H).

LCMS (M⁺+H): 274.0, 97.72%.

### 4-(4-fluoro-2-(1-methyl-1H-pyrazol-4-yl)phenyl)-4-hydroxy-2-methylenebutanenitrile

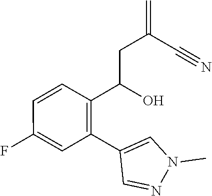 [SMILES: C=C(C#N)CC(O)c1ccc(F)cc1-c1cnn(C)c1]

¹H NMR (400 MHz, CDCl₃) δ 7.62-7.48 (m, 3H), 7.10-6.98 (m, 2H), 5.95 (s, 1H), 5.78 (s, 1H), 5.23-5.19 (m, 1H), 3.97 (s, 3H), 2.54-2.50 (m, 2H), 2.02 (d, J=3.6 Hz, 1H).

MS (m/z): 272.3 [M+H]⁺.

### 5-(2-(1-methyl-1H-pyrazol-4-yl)phenyl)-3-(2-methylpropylidene)dihydrofuran-2 (3H)-one

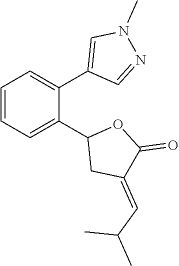

¹H-NMR (400 MHz, CDCl₃): 7.53 (s, 1H); 7.47 (s, 1H); 7.41-7.43 (m, 1H); 7.28-7.40 (m, 3H); 6.62-6.66 (m, 1H); 5.70-5.73 (m, 1H); 3.97 (s, 3H); 3.10-3.17 (m, 1H); 2.78-2.85 (m, 1H); 2.41-2.47 (m, 1H); 1.02-1.12 (m, 6H)

LCMS (M++H): 297.1, 91.57%.

### 5-(4-chloro-2-(1-methyl-1H-pyrazol-4-yl)phenyl)-3-(4-fluorobenzylidene)dihydrofuran-2 (3H)-one

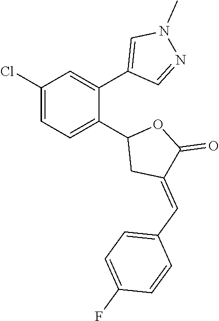

¹H-NMR (400 MHz, CDCl₃): 7.59 (t, 1H); 7.56 (s, 1H); 7.51 (s, 1H); 7.44-7.46 (m, 2H); 7.31-7.37 (m, 3H); 7.10-7.15 (m, 1H); 5.76-5.79 (m, 1H); 3.97 (s, 3H); 3.43-3.40 (m, 1H); 3.09-3.15 (m, 1H)

LCMS (M⁺+H): 382.1, 95.64%.

### 4-(4-chloro-2-(1-methyl-1H-pyrazol-4-yl)phenyl)-2-methylene-4-oxobutanenitrile

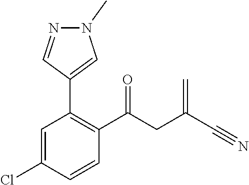 [SMILES: C=C(C#N)CC(=O)c1ccc(Cl)cc1-c1cnn(C)c1]

¹H NMR (400 MHz, CDCl₃) δ 7.60 (s, 1H), 7.47 (s, 1H), 7.44-7.42 (bs, 1H), 7.37-7.32 (m, 2H), 6.05 (s, 1H), 5.69 (s, 1H), 3.96 (s, 3H), 3.40 (s, 2H).

LCMS (M⁺+H): 286.1, 95.15%.

### 4-(4-chloro-2-(1H-pyrazol-4-yl)phenyl)-4-hydroxy-2-methylenebutanenitrile

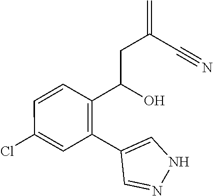 [SMILES: C=C(C#N)CC(O)c1ccc(Cl)cc1-c1cn[nH]c1]

¹H NMR (400 MHz, CDCl₃) δ 7.70-7.62 (m, 2H), 7.57 (d, J-6.4 Hz, 1H), 7.40-7.30 (m, 2H), 5.94 (s, 1H), 5.77 (s, 1H), 5.21-5.17 (m, 1H), 2.64-2.50 (m, 2H).

MS (m/z): 274.2 [M+H]⁺.

### 4-(4-chloro-2-(1-(2-hydroxyethyl)-1H-pyrazol-4-yl)phenyl)-4-hydroxy-2-methylenebutanenitrile

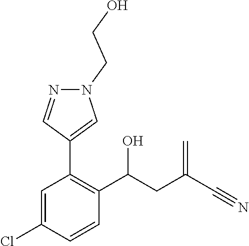 [SMILES: C=C(C#N)CC(O)c1ccc(Cl)cc1-c1cnn(CCO)c1]

¹H NMR (400 MHz, CDCl₃) δ 7.62-7.54 (m, 3H), 7.42-7.35 (m, 1H), 7.30-7.25 (m, 1H), 5.92 (s, 1H), 5.77 (s, 1H), 5.26-5.19 (m, 1H), 4.31 (t, J=4.4 Hz, 1H), 4.08-4.0 (m, 2H), 3.52-3.46 (bs, 2H), 3.07 (t, J=6.4 Hz, 1H), 2.60-2.48 (m, 2H).

LCMS (M⁺+H): 318.4, 93.67%.

### Example 2

### Proliferation Assay:

10000 cells/well were seeded in a 96-well assay plate in a volume of 50 μl (four different types of cells were seeded separately containing both telomerase positive cell lines viz., MOLM-13 and telomerase negative cells lines viz., Saos-2 and MRC-9 respectively). The required concentrations of the compounds, the reference compounds, and the positive control (doxorubicin) were added at 6-point concentrations starting from 100 μM followed by one log dilutions in duplicates. 10 μl of this 10× diluted compound stocks were added into each well and adjusted to 100 μl with the cell culture media (for MOLM-13 used RPMI 1640; for Saos-2 and MRC-9 used DMEM containing 10% FBS kept in 5% CO₂ at 37° C. to maintain physiological pH). Untreated cells were used as controls to calculate IC₅₀ values based on no inhibition/cell death observed in absence of treatment. After 72 hrs incubation in 5% CO2 at 37° C., 100 μl of the cell-titer glo reagent was added directly to each well and the entire content of the well was transferred into white plates for reading. The luminescence was read and measured 10 minutes after incubation. Doxorubicin (10 μM) was used as a positive control for cell death. The percent inhibition was calculated based on the percent reduction in the luminescence compared to control wells without treatment. IC₅₀ values were determined by fitting percent inhibition data in GraphPad Prism (version-9.3.1) software.

### Telomerase Enzyme Assay

0.4×10\hat{ }6 cells/well were seeded in a 6-well plate. The required concentration of 10× compounds were added in duplicates containing 2 ml total volume per well. Untreated cells were used as positive control. Heat-inactivated cells were used as a negative control. The cells were incubated for 72 hrs at 37° C. in 5% CO₂ and were collected to determine telomerase activity using the TeloTAGGG Telomerase PCR ELISAPLUS kit (Roche, UK). After 72 hrs the samples were centrifuged, washed followed by lysis (in cold) in Lysis buffer provided by the kit. After 30 min in cold the lysate was centrifuged at 16000 rpm for 20 mins to obtain the sample for PCR. Some cells were treated with RNAse and heat inactivated and these were used as negative control having no telomerase activity. Untreated cells were used as a positive control to determine the % inhibition. The high control (supplied in the kit) was used as a reference standard in place of the sample wells. The PCR was carried out with 30 μl of Reaction Mixture and 3 μl of the cell extract in a total volume of 50 μl made up with distilled water. The PCR reaction of 30 cycles of denaturation based on kit instructions, annealing and polymerization was carried out after elongation and inactivation and finally terminated with 10 mins extension using the thermos cycler. The PCR samples were then taken up for ELISA using the kit protocol using the detection by HRP-TMB substrate conjugate. The reaction was finally stopped and read with an absorbance of 450 nm and ref of 690 nm using the Synergy H1 microplate reader (BioTek instrument, UK). The percent inhibition was calculated based on complete telomerase inhibition with heat inactivated cells and no telomerase inhibition seen in untreated cells. IC₅₀ values were determined by fitting percent inhibition data in GraphPad Prism (version-9.3.1) software.

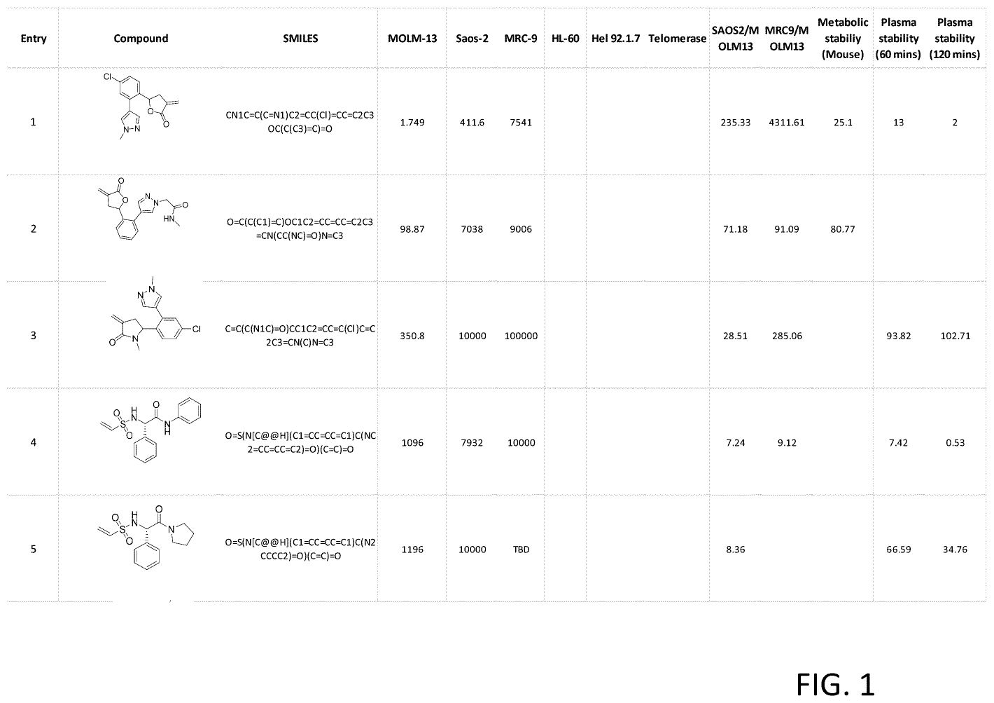

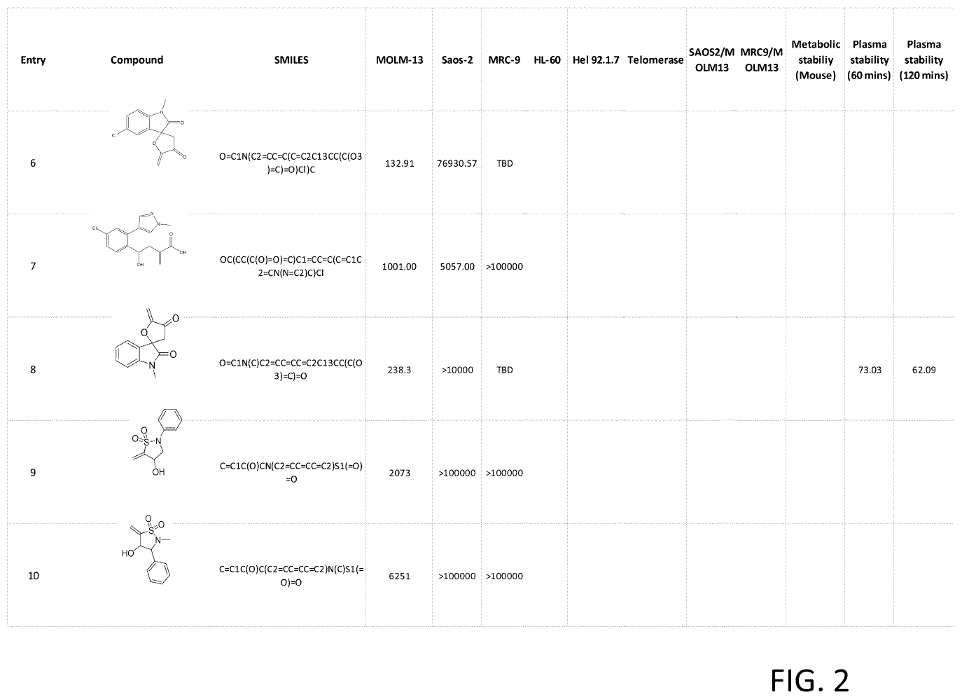

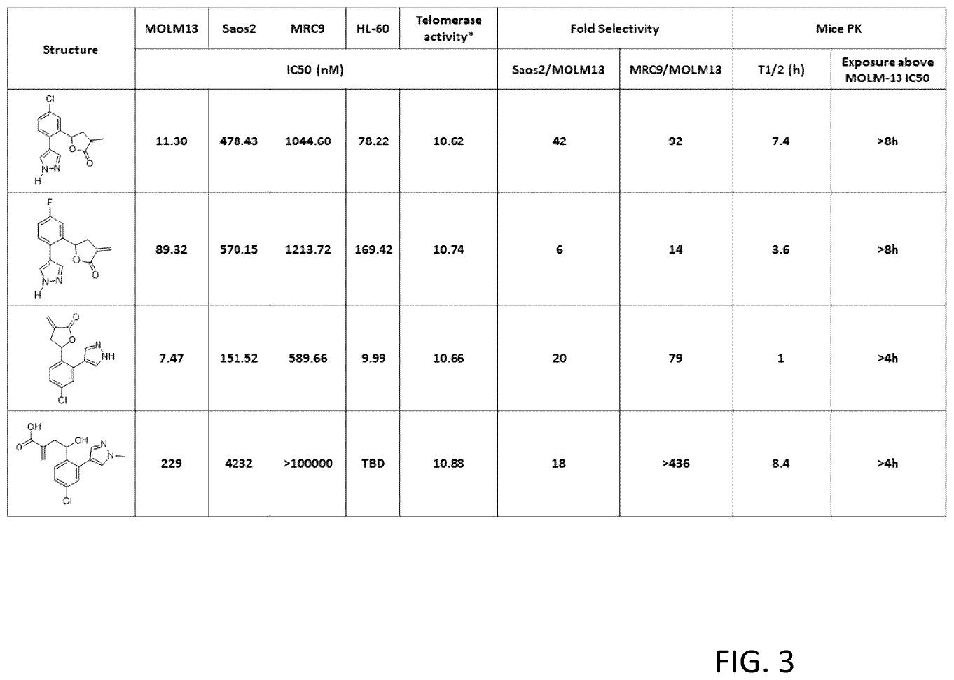

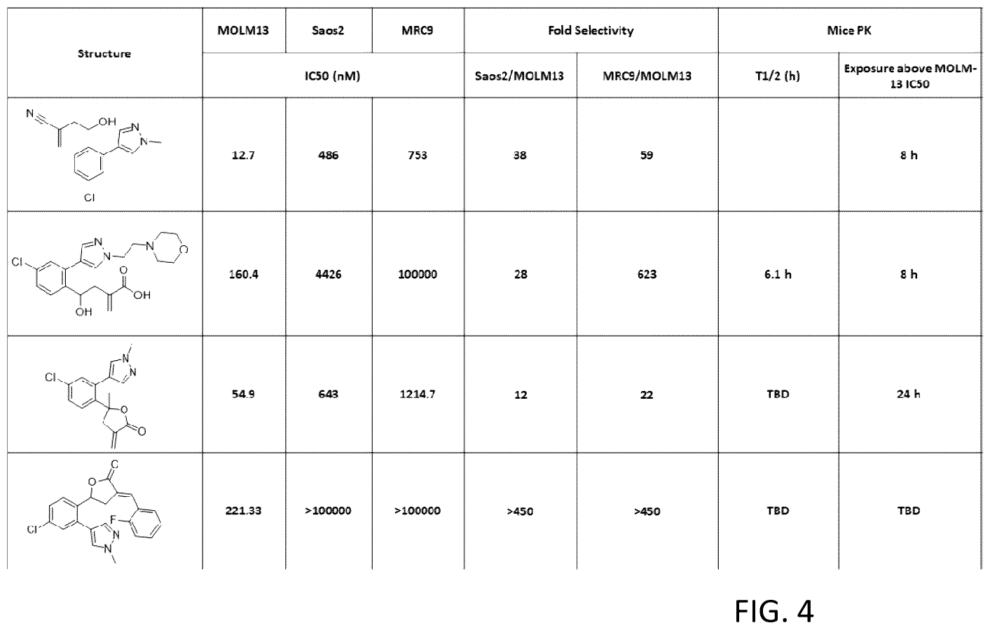

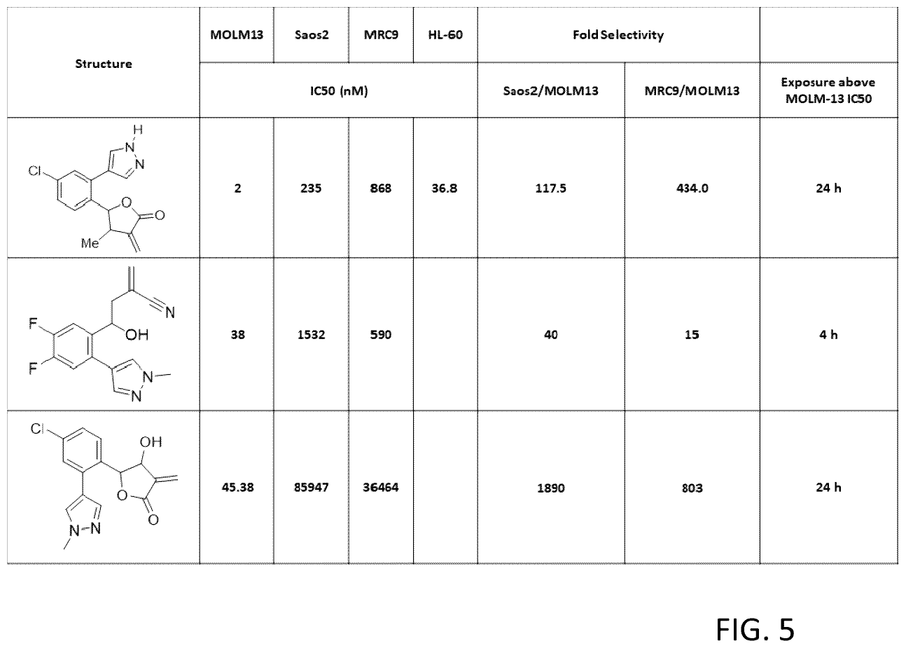

The data from such analyses is shown in FIGS. 1 and 2, along with FIGS. 3, 4, and 5.

### Example 3

Screening of anticancer activity of compounds in an efficacy model using Luc-MOLM13 cell line

3.0 Test System:

Species: mouse

Strain: NOD-SCID (NOD.CB17-Prkdcscid/NCrCrl)

Gender: male

Age: 6-7 week at start of acclimatization

Body weight range: 27-28 g at start of acclimatization

Number of groups: 5 (additional 2 animals as controls)

Number of animals per group: 7

Total number of animals: 37

NOD-SCID mice are the standard laboratory rodent species used for pre-clinical efficacy assessment based on the indication therapy of the test items

Animals were housed in a controlled environment in the animal room with a temperature of 22±2° C., relative humidity of 40-70%, photoperiod of 12-12 hours and 10-12 air changes per hour.

Throughout the study period, animals were housed (six per cage) in sterile polysulfone greenline mouse IVC from Techniplast (GM500) with an overall dimension of 7.75″L×14.75″W×5.25″H. Cages were covered with stainless steel grid top mesh and with provision for water bottles and feed. Autoclaved corncob and paper shred were used as bedding and nestling material.

The floor and work surface of the experimental room were mopped twice daily on working days and at least once daily on holidays with 0.03% sodium hypochlorite.

3.5 Feed and Water. The animals were fed ad libitum with gamma-irradiated pellet feed from Altromin, Germany. Autoclaved RO water was provided ad libitum to all animals via autoclaved water bottles during the experiment.

3.6 Acclimatization. Animals were acclimatized for 7 days before the initiation of treatment. A thorough physical examination was performed before selecting the animals. Animals without any visual signs of illness were used for the study. All animals were observed for cage side clinical signs at least once daily during acclimatization.

Individual animals were identified by the universal ear-notching method during acclimatization and post-randomization. Additionally, cages were also identified with cage cards.

Acclimatization Cage Card had the details like; Study protocol No., Species and Strain, Sex, Cage No., Animal No., Acclimatization Start Date, Acclimatization End Date, Sign and Date. Experimental Cage Card had the details like; Study protocol No., Test Item Code, Species and Strain, Sex, Cage No., Group No. and Dose, Animal No., Treatment Start Date, Remarks, Date of Necropsy, Sign and Date.

Randomization was done one day prior to the dosing based on the body weight.

### 4.0 Materials and Methods

Animals: A total of 37 animals were used across groups [7 animals/group for test compounds and 1 animal each as control blank and control compound blank (both without any cells)].

LUC-MOLM13 Cells: The cells were procured from Creative Biolabs, NY 11967. USA (Catalog no IOC-02P004).

Reagents: LUC-MOLM13 media (RPMI) was procured from Gibco (catalog no-11875093); Luciferin potassium salt (CAS-NO:115144-35-9) was procured from SRL, India.

Complete media was prepared by adding 20% Australian FBS (35-086-CV-FBS-corning, catalog No-30029020) and 1 μg/mL Puromycin (Puromycin dihydrochloride: CAS 58-58-2, Sigma).

### Preparation of Compound Formulation:

Dose: 50 mg/Kg, 25 mg/Kg and 10 mg/Kg.

Dose Volume: 0.1 mL

Composition of Formulation for 50, 25 and 10 mg/Kg of test items:

| | Com- | Com- | Com- | BIBR- |
|---|---|---|---|---|
| Excipient | pound 1 | pound 3 | pound 2 | 1532 |
| DMSO | 2% | 2% | 2% | 2% |
| Carboxy methyl | 97% | 97% | 97% | 97% |
| cellulose (CMC) 0.5% | | | | |
| Tween 80 | 1% | 1% | 1% | 1% |
| Dose (mg/kg) | 50/25/10 | 50/10 | 50/25/10 | 50 |
| Formulation | 5/5/2 | 5/2 | 5/5/2 | 5 |
| concentration (mg/ml) | | | | |
| Volume of formulation | 250/125/125 | 250/125 | 250/125/125 | 250 |
| dosed (μL) | | | | |

Dose Volume: 0.1 mL

5.1 cell culture. LUC-MOLM13 cell lines supplied by Creative Biolabs were used. The cells were cultured in a complete media of RPMI with 20% Australian FBS in the absence of antibiotics at 37° C. in 5% CO2 until the cells reached full confluency. After 7-10 days based on their growth phase, the cells were cultured into 1 μg/ml Puromycin containing media at a seeding density of 2×106 cells per 75 mm flask. The cells were grown in massive quantity and were checked for the luciferase signal using imaging on an IVIS spectrum imager. Once confirmed in the in vitro imaging, the cells were considered ready for inoculation into the animals.

5.2 study protocol. The efficacy study was conducted to assess the tumor regression of Compound 1, Compound 2, and Compound 3 shown below in male NOD-SCID mice using LUC-MOLM13 cell line. Individual animals were assigned identification markings on the ear. The study population and dosing regimen has been described in the section 5.3 and 5.6. Initially compounds were dosed at 50 mg/Kg but due to overdose and loss of few animals, the doses were reduced to 10 mg/Kg for all test compounds except BIBR-1532. Following the administration of the test compounds, the study endpoints were assessed in accordance with the study protocol mentioned in the section 5.3.

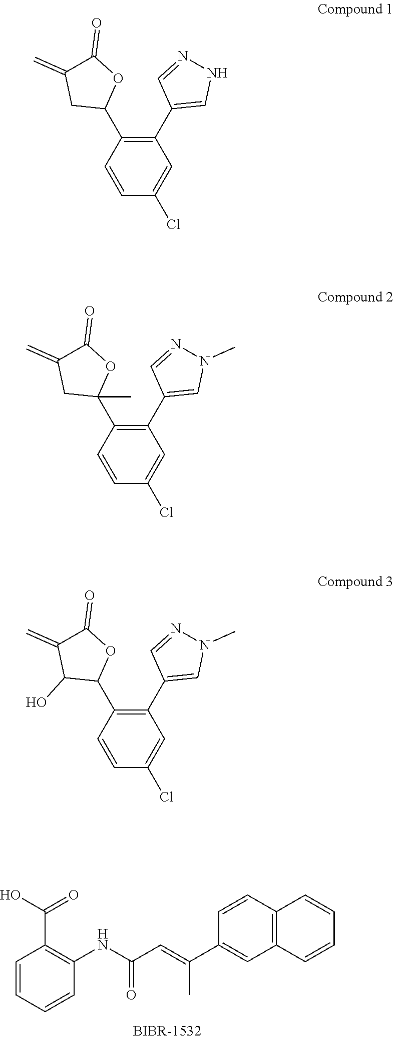

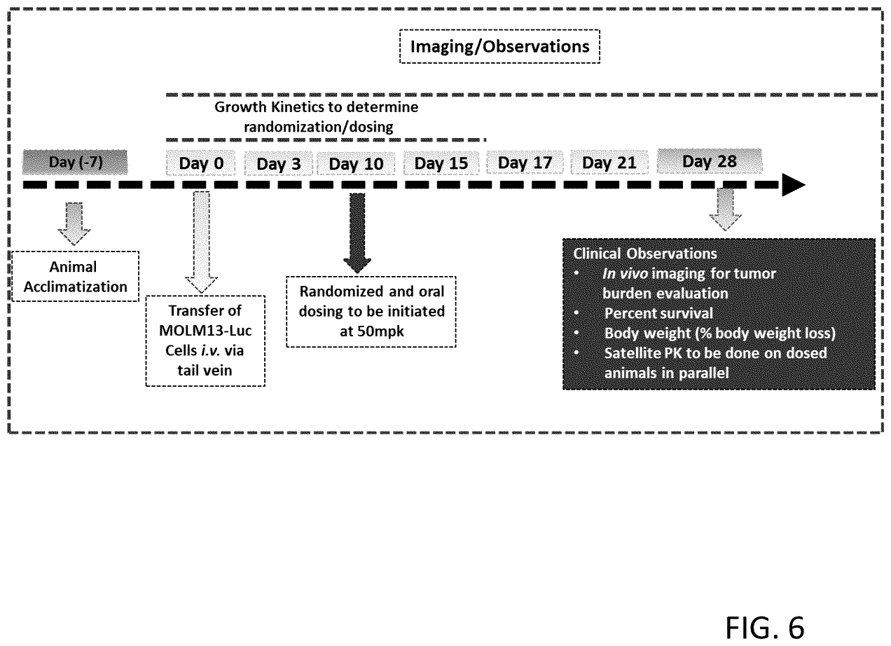

5.2 Study Design, as shown in FIG. 6.

The study included 7 groups: 5 groups consisting Vehicle, Compound 1, Compound 3, Compound 2, reference compound BIBR-1532 and 2 groups (blank control and compound control, with no cells). Each mouse was intravenously injected with 1 million LUC-MOLM13 tumor cells in a total volume of 0.1 mL in DPBS and this would be considered as Day 0. Luciferin (150 mg/Kg body weight) was planned to be injected (JP) 5 mins before imaging and sequential 1-2 images of mice were planned every 1 minute. The animals were planned to be dosed with test compounds from Day 10 onwards by oral route at doses of 50 mg/Kg every alternate day.

Following the administration of the test compounds, the study endpoints will be assessed in accordance with the study protocol mentioned in the section 5.3.

### (A) Efficacy Study Procedure

The efficacy study was conducted to assess the inhibitory effect of Compound 1, Compound 3 and Compound 2 on leukemia cell expansion in male NOD-SCID mice using LUC-MOLM13 cell line.

1 million of LUC-MOLM13 cells/100 μL were suspended in DPBS and were inoculated per mouse via intravenous route in the lateral tail vein in NOD SCID male mice (Fed animals) of 6 to 8 weeks old after 7 days of acclimatization. This was considered as Day-0 of the study.

Luciferin (150 mg/Kg body weight) was injected (IP) 5 mins before imaging and sequential 1-2 images of mice were taken every 1 minute.

Individual animals were assigned identification markings on the ear. The study population and dosing regimen has been described in the section 5.4. Every animal was weighed before dosing and formulation was given based on individual body weights.

Bioluminescence imaging for tumor evaluation was done on Days 0, 5, 10, 15, 18, 21 and 28 respectively along with regular body weight monitoring. The following observations were monitored at regular intervals: body weight (% body weight loss), frequencies of leukemic cells (LUC-MOLM13) in hematopoietic organs and non-lymphoid tissues using imaging on an IVIS spectrum imager for tumor burden evaluation.

Other observations included any clinical signs/observations upon dosing. Animals were euthanized post survival curve evaluation and tissues were collected from two animals from each group, except for the group Compound 1, where tissues were collected from one remaining animal. A range of tissues were collected including brain, axillary lymph node, eyes, branchial lymph node, lungs, heart, stomach, liver, kidney, spleen, cervical lymph node, mesenteric lymph node, inguinal lymph node, GI tract and male reproductive organ. Growth kinetics of Luc-MOLM13 cells was monitored based on BLI along with survival curve.

The deviation in the procedure is included based on overall animal health into the initial study plan of dosing in Section 5.3.

### (B) Protocol for Pharmacokinetic Study in NOD-SCID Mice:

NOD-SCID mice were acclimatized for 7 days. Animal were given 10 mg/Kg of Compound 2 on Day-8 by Per oral route. The plasma was collected at different points: 0.08, 0.25, 0.50, 1, 2, 4, 6 and 24 h and frozen at −80° C.

The plasma samples were retrieved from the deep freezer and allowed them to thaw. The calibration curve was prepared using the standard samples.

The thawed samples were vortexed to ensure complete mixing of contents. 50 μL of each sample was transferred into the ria vials and 200 μL of acetonitrile containing internal standards was added to all the samples and vortexed.

Samples were kept on the shaker for 5 min to ensure complete mixing of contents and subsequently centrifuged at 10000 rpm at 20° C. for 10 min.

The 20 μL of supernatant were transferred into the vials and loaded the onto the auto sampler for LC-MS/MS analysis.

The calibration curve range was 10 to 20000.0 ng/mL. Calibration Curve (CC) standards samples which were run along with samples, met the acceptance criteria, demonstrating satisfactory performance of the method during the analysis of samples.

Note: Results of satellite PK in NOD-SCID mice have been included in Annexure-2.

5.3 Protocol Deviation. Initial plan was to dose from Day 10 onwards by oral route at doses of 50 mg/Kg every alternate day; however, we initiated the dosing from Day-8 onwards based on few reports [7, 8]. After the first dose at 50 mg/Kg on Day 8, we observed deterioration of health in most of the animals and loss of one animal from Group-5 on Day-9. Hence, the doses were reduced to 25 mg/Kg for Groups Compound 1 and Compound 2 and 10 mg/Kg for Group Compound 3 on Day-10 and Day-12 respectively. BIBR-1532 dosing was maintained at 50 mg/Kg every alternate day. However, we still observed deaths from group-5 (1 animal on Day-11), group-6 (2 animals on Day-12) and Group-4 (one animal each on Day 12 and Day-13). Dosing was stopped for the next 4 days for all test compounds and BIBR-1532 considering the animal health condition. From Day-17 onwards, the animals were dosed on alternate days at 10 mg/Kg for all treatment groups (Group 4: Compound 1, Group 5: Compound 3. Group 6: Compound 2) and at 50 mg/Kg for reference standard (Group 7: BIBR-1532).

5.4 Dosage. Animals in G3 group received vehicle, served as vehicle control, G4, G5, G6 and G7 groups received respective compounds Compound 1, Compound 3, Compound 2 and BIBR-1532 (reference standard).

5.5 Justification for Selection of Dose and Route of Administration. The dose was selected based on the PK profile of these compounds in Balb/c animals and the tolerability of compounds in NOD-SCID mice.

5.6 Study Animal Group.

| | | Number of BALB/c |
|---|---|---|
| Group | Group Type | mice per group |
| 1 | Blank with no cells or compound | 1 |
| 2 | Blank with no cells and with Compound 2 | 1 |
| 3 | Vehicle Control | 7 |
| 4 | Compound 1 PO | 7 |
| 5 | Compound 3 PO | 7 |
| 6 | Compound 2 PO | 7 |
| 7 | BIBR-1532 (Reference standard) PO- | 7 |
| | 50 mg/Kg | |

5.7 Study Drug Administration. The test compounds Compound 1, Compound 3, Compound 2 and BIBR-1532 were dosed via peroral route, as mentioned in sections 5.3 and 5.4.

6.0 Calculations. The data for bioluminescence imaging levels (BLI (Avg Radiance) was captured from the instrument across the individual groups for the ROI (region of interest (ROI) and the average radiance was calculated to understand the levels as compared to vehicle control (Group 3).

7.0 Results and Discussion. Based on the 28-Day bioluminescence imaging levels (BLI) BLI evaluation across groups, a varying level of inhibition reduction in leukemia cells was seen across the test groups as compared to the vehicle control. The details have been described below:

7.1 Body Weight. The body weight data of NOD-SCID mice used for this study involving different treatment groups (Vehicle Control, 10 mg/Kg of the three treatment groups and 50 mg/Kg of the reference standard) was monitored every alternate day. Initial loss of animals due to overdose in each of the treatment groups (Group Compound 1, Compound 3 and Compound 2 early on in the study) showed some reduction in body weights (we lost two animals each in Group-4: Compound 1, Group5: Compound 3, and Group-6: Compound 2). We observed loss in body weight during the initial days of the study across the groups including vehicle control. The exact reason is unknown; however, this could be due to stress in the animals. (A consistent body weight was observed upon subsequent dosing of 10 mg/Kg. All groups showed a relatively stable mean body weight over the later course of the study. No significant abnormalities in body weight were observed, suggesting good tolerability across all treatment groups at alternate day dosing of 10 mg/kg. In short, the body weights remained consistent across the experiment in all the groups after the initial overdosing

7.2 Clinical Signs. All animals were observed for clinical signs at least once daily during the experiment. Based on initial observations with high doses of 50 mg/Kg, few deaths were seen along with shivering pattern across the groups. Few animals also stopped feeding. Considering these observations, the doses were reduced subsequently to 10 mg/Kg and animal heath was stabilized. Thus, the clinical signs observed initially were due to overdosing which was reversed on reducing the dose and their health stabilized subsequently.

7.3 Leukemia cell burden Bioluminescence imaging levels (BLI) evaluation across groups showed a varying level of leukemia cell as compared to the control vehicle with the following observations:

Group Compound 2 exhibited the most significant reduction in leukemia cell burden, coupled with a favorable overall health outcome for the animals.

Group Compound 3 demonstrated a comparable impact on leukemia cell reduction to that of BIBR-1532, albeit at a lower dosage.

Group Compound 1 was found to be inferior among all groups.

The average BLI vs days showed a gradual increase in the BLI with increase in days indicating a progression of disease across the groups which was repressed as the days progressed in the treatment groups including the reference standard (BIBR-1532), compared to the vehicle control group.

Interpretation of BLIs across different groups has been mentioned below:

### Average Radiance on Day 0:

The animals showed distribution of cells after LUC-MOLM13 inoculation and the cells appeared to localize in the abdomen close to the heart region.

### Average Radiance on Day 5:

The bioluminescence on the Day 5 disappears to negligible levels with non-significant BLI seen in few animals.

### Average Radiance on Day 10:

The bioluminescence reappeared on Day 10 in most of the animals in Group 3 (vehicle control) with prominent presence in the Group 3 and Group 7 while Group 4 showed its prominence only in the brain. Also, not all animals in each treatment group showed the appearance of the bioluminescence signal. Some appeared later in time, say by Day 18 or 21.

### Average Radiance on Day 15:

The bioluminescence showed prominent presence in the Group 3 and Group 7 on Day 15 while Group 4 showed it prominence only in the brain (similar to observations seen on Day-10).

### Average Radiance on Day 18:

The bioluminescence showed significant regression across the groups as compared to the control with prominent regression seen in Group 6 with no signal seen in any of the animals.

### Average Radiance on Day 21:

The bioluminescence signal was enhanced in the Group 3 (vehicle control) and in group 7 (BIBR-1532) with regression in the tumor seen with all the test compounds. But this was most prominently seen in Group 6. However, few animals in Group 6 (Compound 2) showed re-appearance of signal after Day 18.

### Average Radiance on Day 28:

The bioluminescence signal showed prominent appearance in almost all groups. But looking at an overall regression in the tumor, most prominent reduction was seen in Group 6 up to Day-28 as compared to the Group 3 (control vehicle group).

7.4 Tissue Imaging. The necropsy was done on Day 34 after implantation of LUC-MOLM13 cells. In the vehicle control group, prominent and highest bioluminescence of LUC-MOLM13 implant was found in Brain, Axillary lymph node, Branchial lymph node and Lungs. Animals showed prominent tissue response in Group Compound 2—in line with the BLI observed on the 28th day compared to the control. The lymph nodes showed complete disappearance of bio-luminescence compared to the control group.

7.5 Survival Curve. After initial fatalities observed in treatment groups due to compound overdosing, subsequent stabilization of overall health was achieved by reducing the dose. Beyond Day 15, the groups treated with compounds exhibited enhanced protection compared to the vehicle control. Notably, in the treatment groups, Compound 1 recorded one death on Day 31 and two on Day 34. Both Compound 3 and BIBR-1532 experienced one death on Day 31 and another on Day 34, while Compound 2, post 10 mg/kg dosing, displayed no deaths throughout the study. Assessing overall health across groups, animals from Group 6 (Compound 2) exhibited the highest well-being. Group 5 (Compound 3) demonstrated a death ratio similar to BIBR-1532 but at significantly lower doses compared to the reference standard. By Day 28, the health of both the vehicle control and BIBR-1532 groups deteriorated and the study was terminated on Day 34 due to overall animal health concerns, with one death in the control group and most animals in a moribund state in both groups. In summary, the survival curve indicated rapid deterioration in the overall health of both the control vehicle and BIBR-1532 groups. However, animals from Group-6: Compound 2 maintained robust health, exhibiting complete survival until Day 34 without any animals in a moribund state, in contrast to the control vehicle group.

8.0 Conclusion. The small molecule telomerase inhibitor, Compound 2, exhibited remarkable suppression of tumors, as evidenced by both bioluminescence imaging of animals and tissue samples. Notably, it led to the complete disappearance of bioluminescence, particularly in the lymph nodes. This highlights the efficacy of Compound 2 in suppressing the growth of orthotopically growing LUC-MOLM13 tumors.

Compound 3 also stands out in being similar to the BIBR-1532 in displaying significant tumor load suppression at lower doses. Conversely, Compound 1 performed poorly in achieving the study endpoint, exhibiting both suboptimal survival rates and inadequate tumor load suppression compared to the other two compounds.

9.0 Data. Experimental data from the study is shown in the figures.

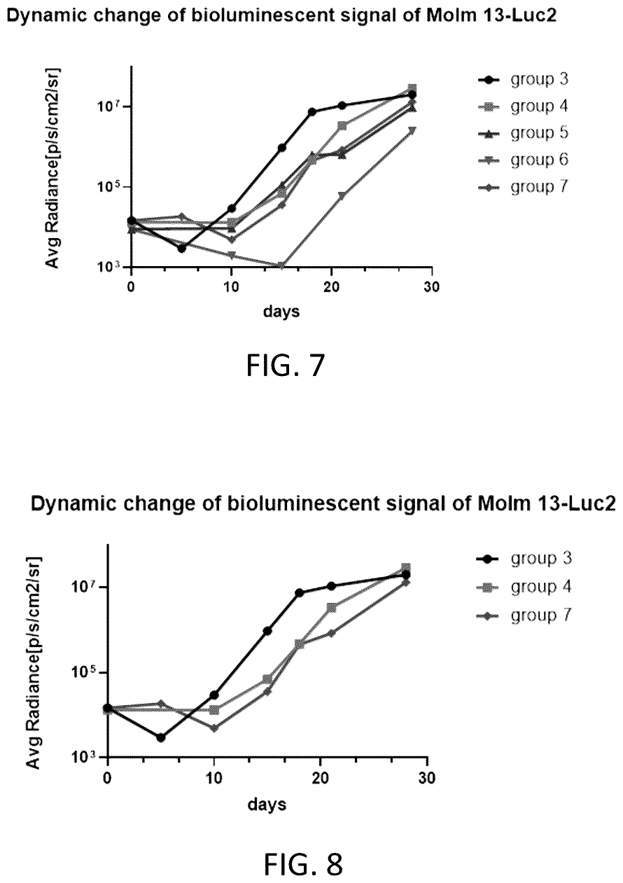

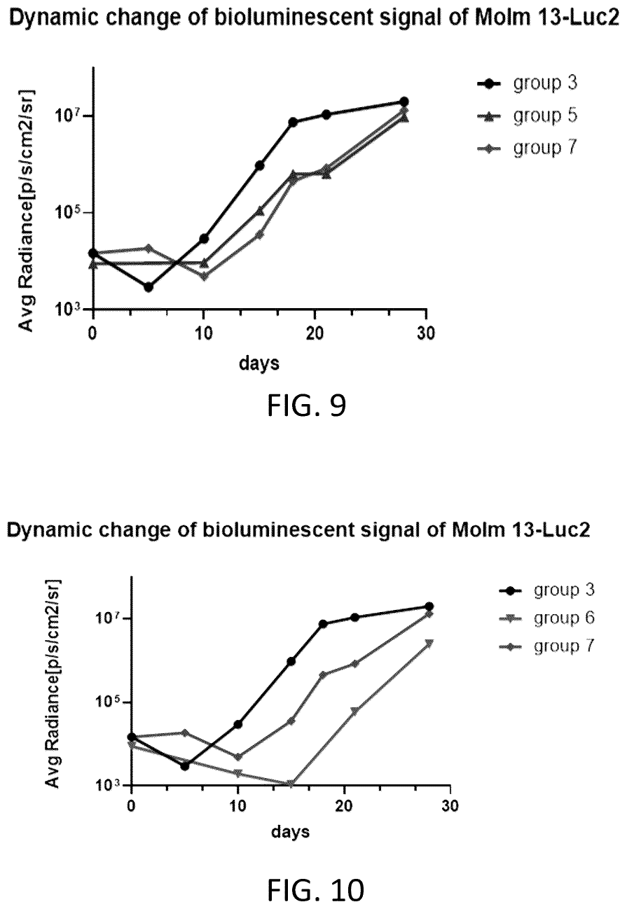

FIGS. 7-10 shows effects of telomerase inhibition over three weeks on the bioluminescence signal (BLI) of LUC-MOLM13 tumor-bearing mice (7) Overall group (8) on oral dose of Group 4 (Compound 1) (9) On oral dose of Group 5 (Compound 3) (10) On oral dose of Group 6 (Compound 2)

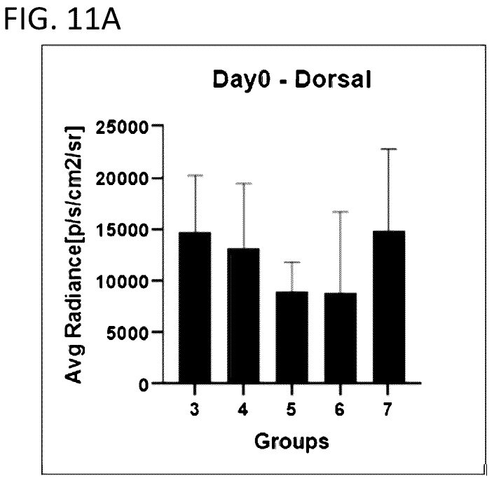

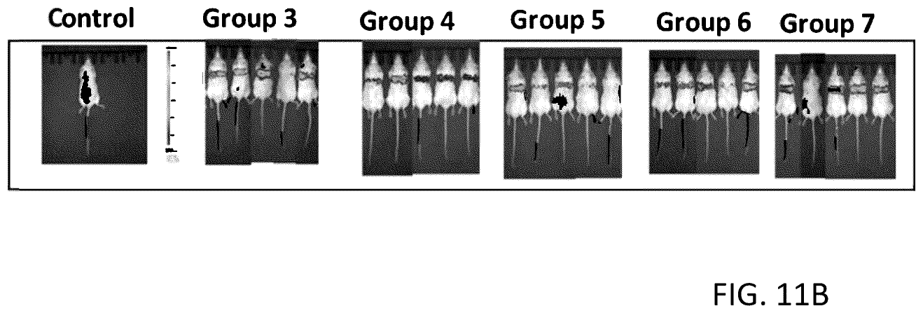

FIGS. 11A-11B shows effect of telomerase inhibitors administered on the bioluminescence signal of LUC-MOLM13 tumor-bearing mice on Day 0 (a) Average BLI across groups (b) Distribution of orthotropically growing LUC-MOLM13 tumors across groups. In FIG. 11B, fluorescence in the top chest area is visible.

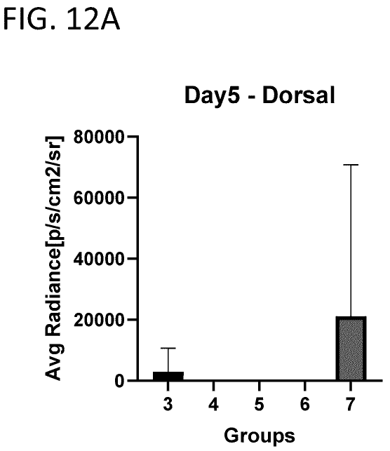

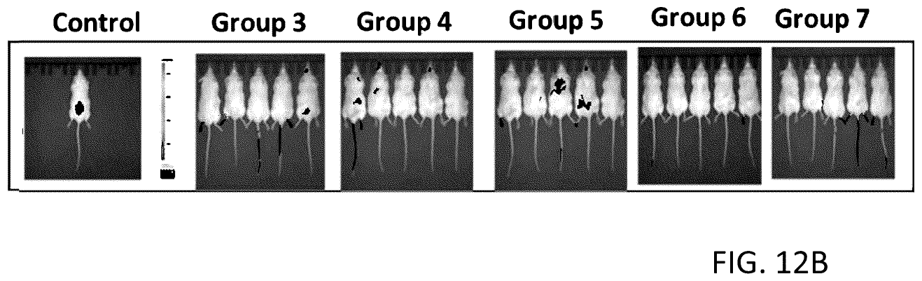

FIGS. 12A-12B shows effect of telomerase inhibitors administered on the bioluminescence signal of LUC-MOLM13 tumor-bearing mice on Day 5 (a) Average BLI across groups (b) Distribution of orthotopically growing LUC-MOLM13 tumors across groups. In FIG. 12B, little or zero fluorescence is shown.

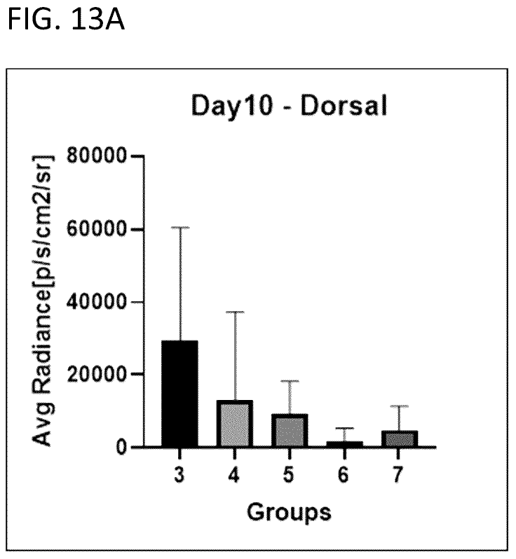

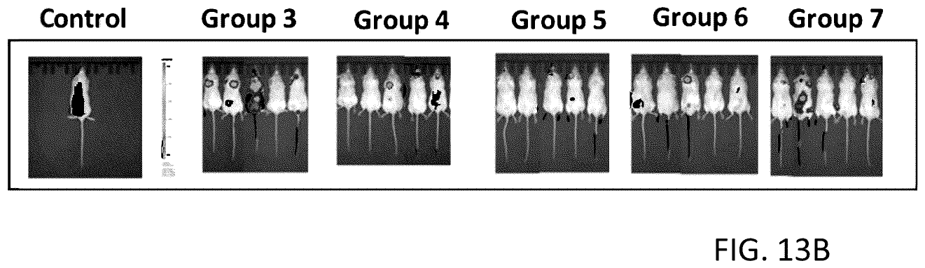

FIGS. 13A-13B show effect of telomerase inhibitors administered on the bioluminescence signal of LUC-MOLM13 tumor-bearing mice on Day 10 (a) Average BLI across groups (b) Distribution of orthotopically growing LUC-MOLM13 tumors across groups.

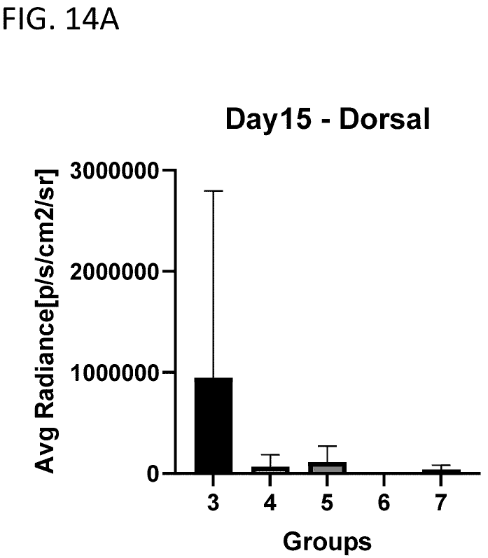

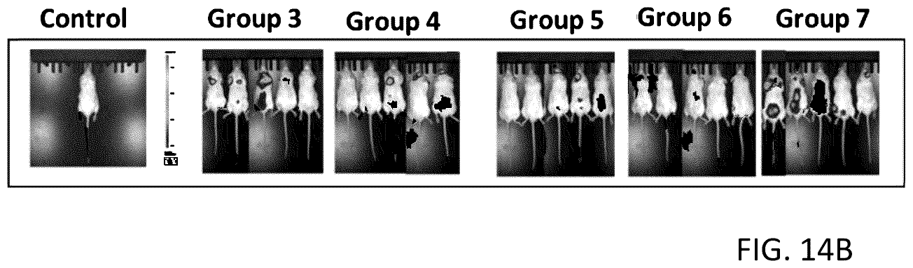

FIGS. 14A-14B show effect of telomerase inhibitors administered on the bioluminescence signal of LUC-MOLM13 tumor-bearing mice on Day 15 (a) Average BLI across groups (b) Distribution of orthotopically growing LUC-MOLM13 tumors across groups.

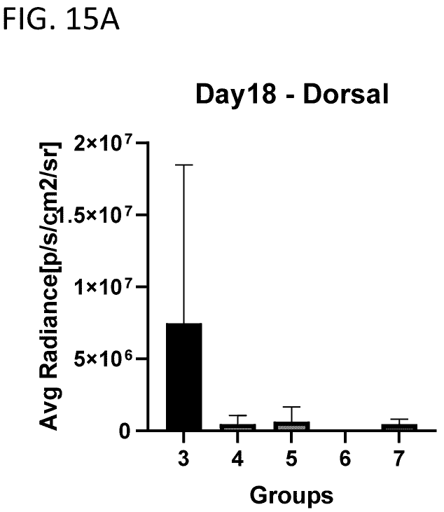

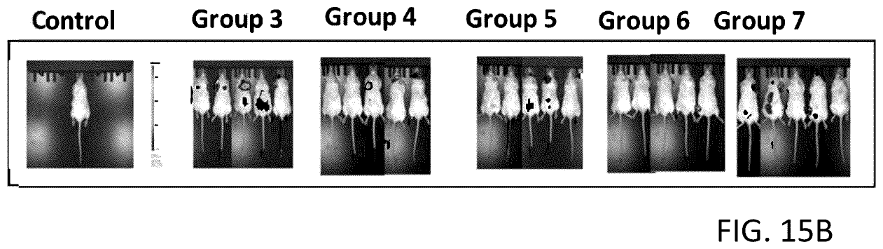

FIGS. 15A-15B show Effect of telomerase inhibitors administered on the bioluminescence signal of LUC-MOLM13 tumor-bearing mice on Day 18 (a) Average BLI across groups (b) Distribution of orthotopically growing LUC-MOLM13 tumors across groups.

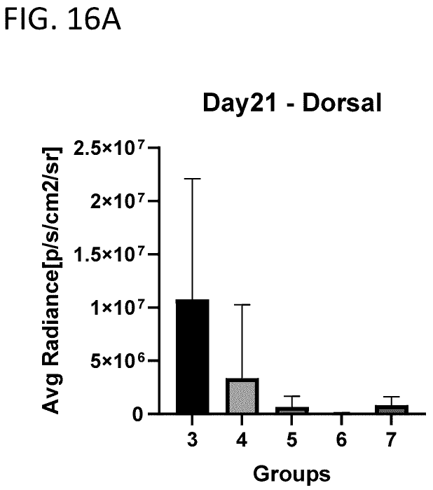

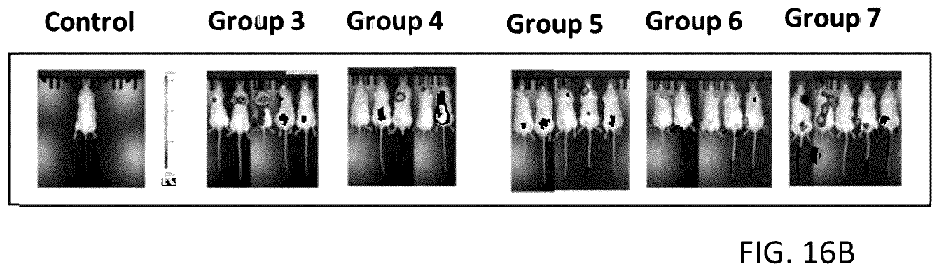

FIGS. 16A-16B show Effect of telomerase inhibitors administered on the bioluminescence signal of LUC-MOLM13 tumor-bearing mice on Day 21 (a) Average BLI across groups (b) Distribution of orthotopically growing LUC-MOLM13 tumors across groups.

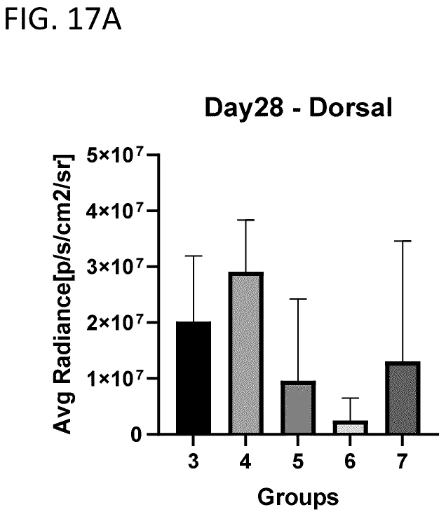

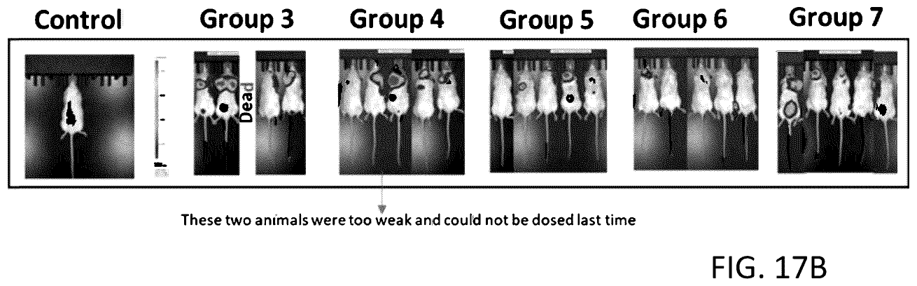

FIGS. 17A-17B show Effect of telomerase inhibitors administered on the bioluminescence signal of LUC-MOLM13 tumor-bearing mice on Day 28 (a) Average BLI across groups (b) Distribution of orthotopically growing LUC-MOLM13 tumors across groups.

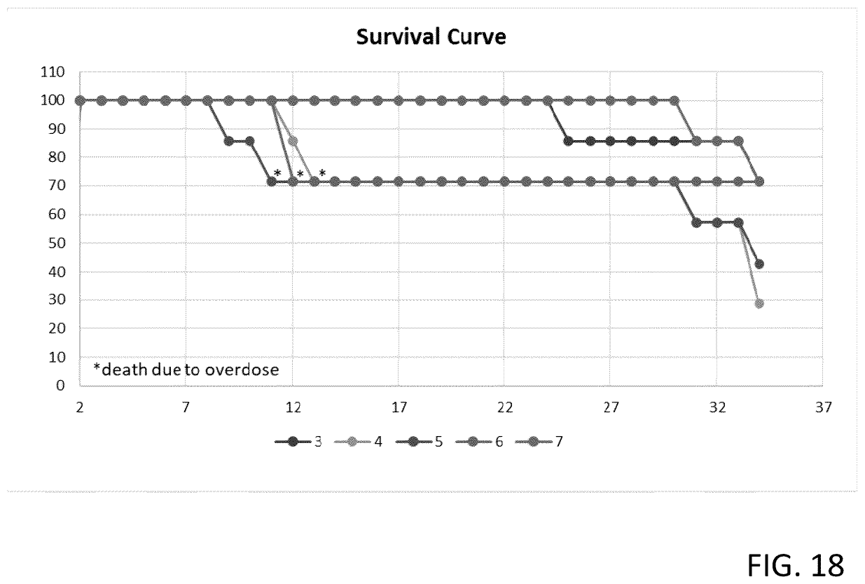

FIG. 18 shows Effect of telomerase inhibitors administered on the life span of LUC-MOLM13 tumor-bearing mice.

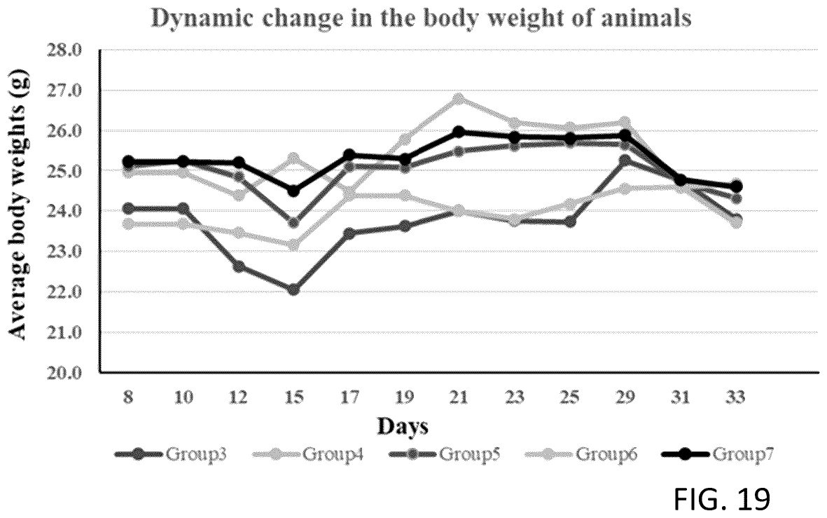

FIG. 19 shows Effect of telomerase inhibitors administered on average body weights of LUC-MOLM13 tumor-bearing mice.

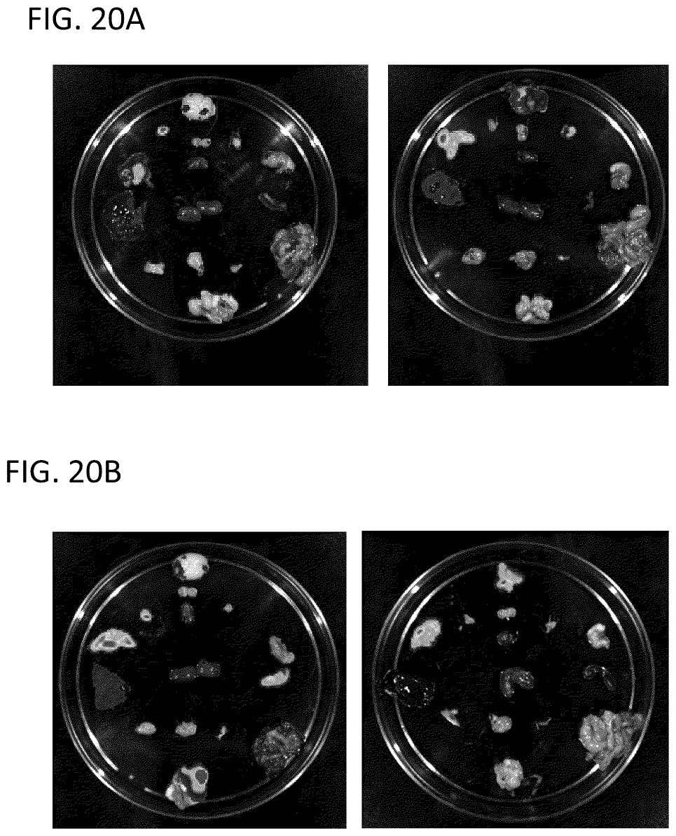

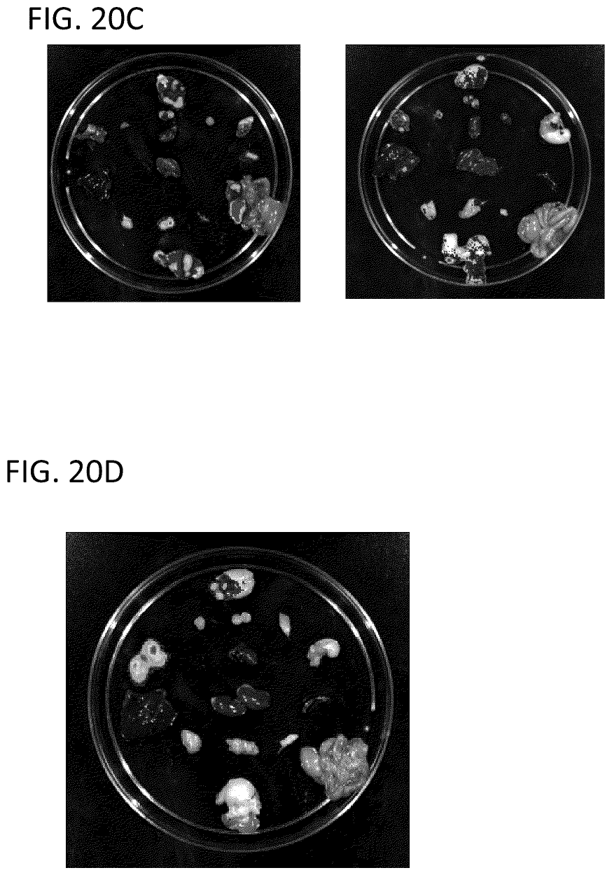

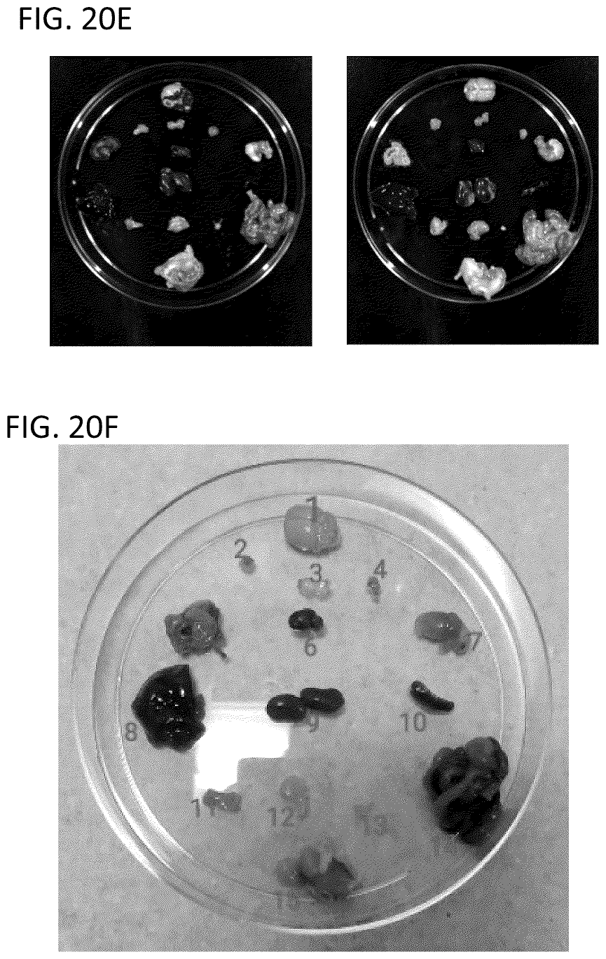

FIGS. 20A-20F show bioluminescent signal from tissues obtained from the effect of telomerase inhibitors on Day 34 of necropsy. FIG. 20A is group-control, FIG. 20B is Group 7 (BIRB-1532), FIG. 20C is Group 5 (Compound 3), FIG. 20D is Group 4 (Compound 1), FIG. 20E is Group 6 (Compound 2), and FIG. 20F is Representative tissue labeling: 1. Brain 2. Axillary lymph node 3. Eyes 4. Branchial lymph node 5. Lungs 6. Heart 7. Stomach 8. Liver 9. Kidney 10. Spleen 11. Cervical lymph node 12. Mesenteric lymph node 13. Inguinal lymph node 14. GI tract 15. Male reproductive organ.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) not invoked.

1. A compound of formula (I): 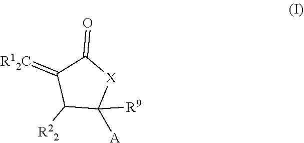
wherein:
X is O or NR^{X};
R^{X} is selected from the group consisting of H, D, alkyl, alkenyl, alkynyl, cycloalkyl, heterocyclyl, aryl, heteroaryl, acyl, carbonyl, carboxy, and substituted versions thereof;
each R¹ and R² is independently selected from the group consisting of H, D, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, thiol, thioether, and substituted versions thereof;
R⁹ is H, D, or alkyl; and
A is a 5- or 6-membered aromatic ring that is optionally substituted or a heterocyclic ring that is optionally substituted.

2. The compound of claim 1, wherein X is O.

3. The compound of claim 1, wherein X is NR^{X}.

4. The compound of claim 1, wherein the compound has formula (Ia-A): 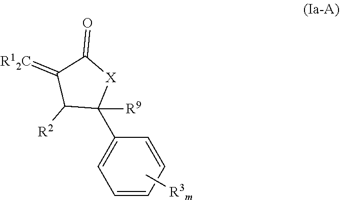
wherein:
m is 0 or an integer from 1 to 5; and
each R³ is independently alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof, provided that two adjacent R³ groups along with the atoms to which they are attached can form a cyclic ring.

5. The compound of claim 1, wherein the compound has formula (Ib-A) 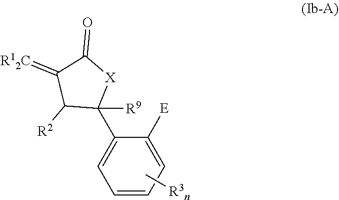
wherein:
n is 0 or an integer from 1 to 4; and
E is selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

6. The compound of claim 5, wherein E is selected from the group consisting of alkyl, aryl, heteroaryl, alkyloxy, aryloxy, heteroaryloxy, heterocyclyl, and substituted versions thereof.

7. The compound of claim 6, wherein E is a 5-membered heteroaryl or substituted heteroaryl group.

8. The compound of claim 7, wherein E is pyrazole or substituted pyrazole.

9. The compound of claim 8, wherein the compound has formula (Ib-1-A): 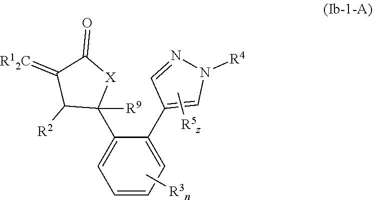
wherein:
R⁴ is selected from the group consisting of H, alkyl, alkenyl, alkynyl, cycloalkyl, heterocyclyl, aryl, heteroaryl, acyl, carbonyl, carboxy, and substituted versions thereof;
z is 0, 1, or 2; and
each R⁵ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

10. The compound of claim 7, wherein E is selected from the group consisting of triazole, substituted triazole, 6-membered aryl, 6-membered substituted aryl, 6-membered heteroaryl, and 6-membered substituted heteroaryl.

11. The compound of claim 1, wherein the compound has formula (Ic-A): 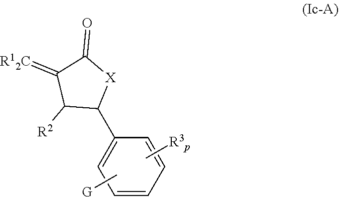
wherein:
p is 0 or an integer ranging from 1 to 4; and
G is selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

12. The compound of claim 11, wherein G is selected from the group consisting of alkyl, aryl, heteroaryl, alkyloxy, aryloxy, heteroaryloxy, heterocyclyl, and substituted versions thereof.

13. The compound of claim 1, wherein the compound has formula (Id-A): 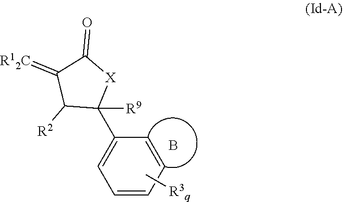
wherein:
q is 0 or an integer from 1 to 3; and
ring B is a 5- or 6-membered aryl or heteroaryl ring.

14. The compound of claim 13, wherein ring B is heteroaryl.

15. The compound of claim 14, wherein ring B is pyridine, pyrrole, imidazole, or pyrrolidine.

16. The compound of claim 1, wherein the compound has a structure selected from the group consisting of: 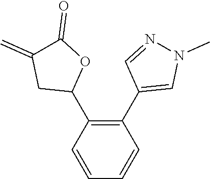
5-(2-(1-methyl-1H-pyrazol-4-yl)phenyl)-3-methylenedihydrofuran-2 (3H)-one 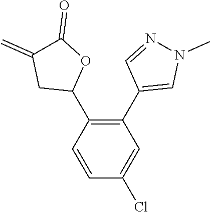
5-(4-chloro-2-(1-methyl-1H-pyrazol-4-yl)phenyl)-3-methylenedihydrofuran-2 (3H)-one 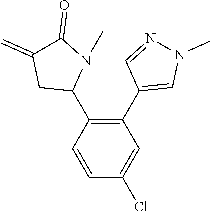
5-(4-chloro-2-(1-methyl-1H-pyrazol-4-yl)phenyl)-1-methyl-3-methylenepyrrolidin-2-one 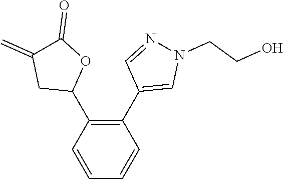 [SMILES: C=C1CC(c2ccccc2-c2cnn(CCO)c2)OC1=O]
5-(2-(1-(2-hydroxyethyl)-1H-pyrazol-4-yl)phenyl)-3-methylenedihydrofuran-2 (3H)-one 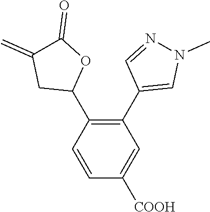
3-(1-methyl-1H-pyrazol-4-yl)-4-(4-methylene-5-oxotetrahydrofuran-2-yl)benzoic acid 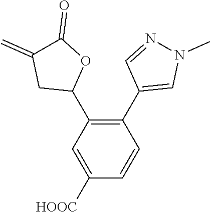
4-(1-methyl-1H-pyrazol-4-yl)-3-(4-methylene-5-oxotetrahydrofuran-2-yl)benzoic acid 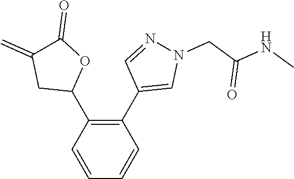
N-methyl-2-(4-(2-(4-methylene-5-oxotetrahydrofuran-2-yl)phenyl)-1H-pyrazol-1-yl)acetamide 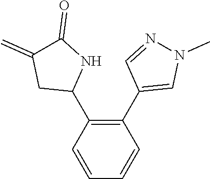
5-(2-(1-methyl-1H-pyrazol-4-yl)phenyl)-3-methylenepyrrolidin-2-one 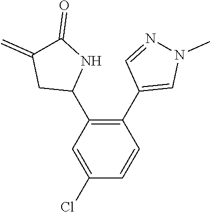
5-(5-chloro-2-(1-methyl-1H-pyrazol-4-yl)phenyl)-3-methylenepyrrolidin-2-one 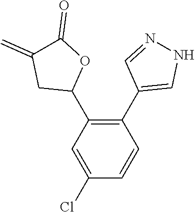 [SMILES: C=C1CC(c2cc(Cl)ccc2-c2cn[nH]c2)OC1=O]
5-(5-chloro-2-(1H-pyrazol-4-yl)phenyl)-3-methylenedihydrofuran-2 (3H)-one 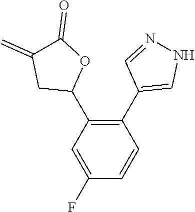 [SMILES: C=C1CC(c2cc(F)ccc2-c2cn[nH]c2)OC1=O]
5-(5-fluoro-2-(1H-pyrazol-4-yl)phenyl)-3-methylenedihydrofuran-2 (3H)-one 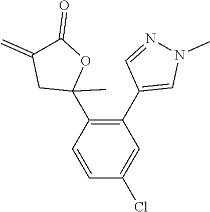
5-(4-chloro-2-(1-methyl-1H-pyrazol-4-yl)phenyl)-5-methyl-3-methylenedihydrofuran-2 (3H)-one 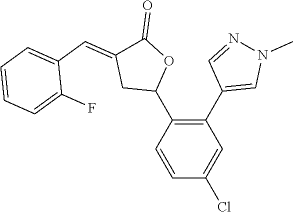 [SMILES: Cn1cc(-c2cc(Cl)ccc2C2C/C(=C\c3ccccc3F)C(=O)O2)cn1]
(E)-5-(4-chloro-2-(1-methyl-1H-pyrazol-4-yl)phenyl)-3-(2-fluorobenzylidene)dihydrofuran-2 (3H)-one 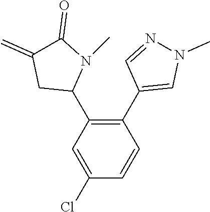
5-(5-chloro-2-(1-methyl-1H-pyrazol-4-yl)phenyl)-1-methyl-3-methylenepyrrolidin-2-one 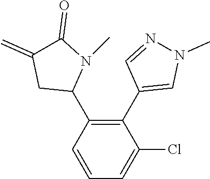
5-(3-chloro-2-(1-methyl-1H-pyrazol-4-yl)phenyl)-1-methyl-3-methylenepyrrolidin-2-one 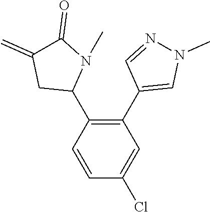
5-(4-chloro-2-(1-methyl-1H-pyrazol-4-yl)phenyl)-1-methyl-3-methylenepyrrolidin-2-one 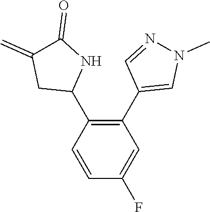
5-(4-fluoro-2-(1-methyl-1H-pyrazol-4-yl)phenyl)-3-methylenepyrrolidin-2-one 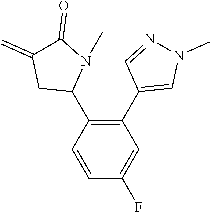
5-(4-fluoro-2-(1-methyl-1H-pyrazol-4-yl)phenyl)-1-methyl-3-methylenepyrrolidin-2-one 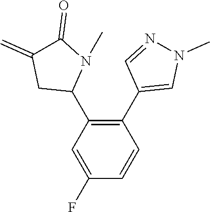
5-(5-fluoro-2-(1-methyl-1H-pyrazol-4-yl)phenyl)-1-methyl-3-methylenepyrrolidin-2-one 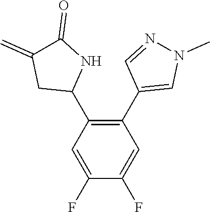
5-(4,5-difluoro-2-(1-methyl-1H-pyrazol-4-yl)phenyl)-3-methylenepyrrolidin-2-one 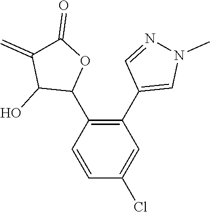
5-(4-chloro-2-(1-methyl-1H-pyrazol-4-yl)phenyl)-4-hydroxy-3-methylenedihydrofuran-2 (3H)-one 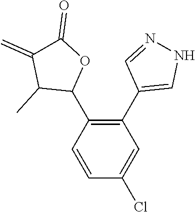 [SMILES: C=C1C(=O)OC(c2ccc(Cl)cc2-c2cn[nH]c2)C1C]
5-(4-chloro-2-(1H-pyrazol-4-yl)phenyl)-4-methyl-3-methylenedihydrofuran-2 (3H)-one 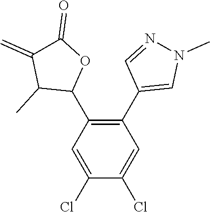
5-(4,5-dichloro-2-(1-methyl-1H-pyrrol-3-yl)phenyl)-4-methyl-3-methylenedihydrofuran-2 (3H)-one 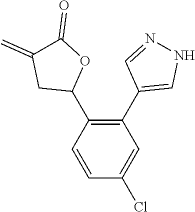 [SMILES: C=C1CC(c2ccc(Cl)cc2-c2cn[nH]c2)OC1=O]
5-(4-chloro-2-(1H-pyrazol-4-yl)phenyl)-3-methylenedihydrofuran-2 (3H)-one 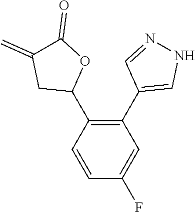 [SMILES: C=C1CC(c2ccc(F)cc2-c2cn[nH]c2)OC1=O]
5-(4-fluoro-2-(1H-pyrazol-4-yl)phenyl)-3-methylenedihydrofuran-2 (3H)-one, and 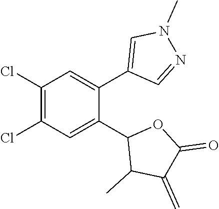 [SMILES: C=C1C(=O)OC(c2cc(Cl)c(Cl)cc2-c2cnn(C)c2)C1C]
5-(4,5-dichloro-2-(1-methyl-1H-pyrazol-4-yl)phenyl)-4-methyl-3-methylenedihydrofuran-2 (3H)-one.

17. A compound of formula (II): 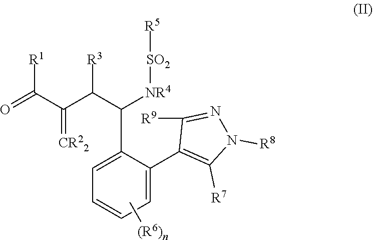
wherein:
n is 0, 1, 2, or 3;
each R¹, R², R⁶, R⁷, and R⁹ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and
each R³, R⁴, R⁵, and R⁸ is independently selected from the group consisting of H, alkyl, alkenyl, alkynyl, cycloalkyl, heterocyclyl, aryl, heteroaryl, acyl, carbonyl, carboxy, and substituted versions thereof.

18.-27. (canceled)

28. A compound of formula (III): 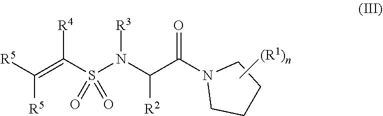
wherein:
n is 0 or an integer ranging from 1 to 4;
each R¹, R⁴, and R⁵ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof;
R² is selected from the group consisting of aryl, substituted aryl, heteroaryl, and substituted heteroaryl; and
R³ is selected from the group consisting of H, alkyl, alkenyl, alkynyl, cycloalkyl, heterocyclyl, aryl, heteroaryl, acyl, carbonyl, carboxy, and substituted versions thereof.

29.-36. (canceled)

37. A compound of formula (IV): 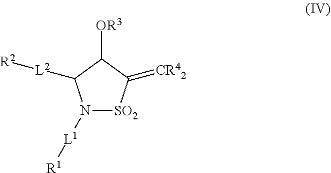
wherein:
R¹ and R² are each independently selected from H, alkyl, alkenyl, alkynyl, cycloalkyl, heterocyclyl, aryl, heteroaryl, acyl, and substituted versions thereof, provided that at least one of R¹ and R² is aryl, heteroaryl, or a substituted version thereof;
L¹ and L² are each independently absent or an alkylene group;
R³ is selected from the group consisting of H, alkyl, alkenyl, alkynyl, cycloalkyl, heterocyclyl, aryl, heteroaryl, acyl, and substituted versions thereof; and
R⁴ is independently selected from the group consisting of alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

38.-53. (canceled)

54. A compound of formula (V): 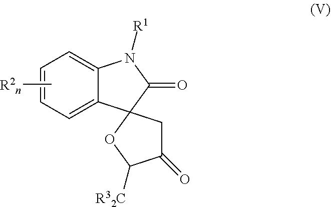
wherein:
R¹ is selected from the group consisting of H, alkyl, alkenyl, alkynyl, cycloalkyl, heterocyclyl, aryl, heteroaryl, acyl, carbonyl, carboxy, and substituted versions thereof;
each R² and R³ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and
n is 0 or an integer from 1 to 4.

55.-59. (canceled)

60. A compound of formula (VI): 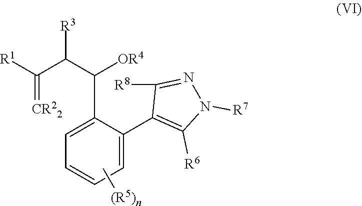
wherein:
n is 0, 1, 2, or 3;
each R¹, R², R³, R⁵, R⁶, and R⁸ is independently selected from the group consisting of H, alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof; and
R⁴ and R⁷ are independently selected from the group consisting of H, alkyl, alkenyl, alkynyl, cycloalkyl, heterocyclyl, aryl, heteroaryl, acyl, carbonyl, carboxy, and substituted versions thereof.

61.-67. (canceled)

68. A pharmaceutical composition comprising a compound of claim 1 and a pharmaceutically acceptable excipient.

69. The pharmaceutical composition of claim 68, further comprising a second compound.

70. (canceled)

71. A method of treating a patient for a telomerase-related condition, the method comprising:
administering a pharmaceutical composition of claim 68 to the patient.

72. (canceled)

73. The method of claim 71, wherein the telomerase-related condition is a cancer.

74. The method of claim 73, wherein the cancer is a hematological malignancy.

75. The method of claim 73, wherein the cancer is selected from the group consisting of acute and chronic leukemias, lymphomas, multiple myeloma and myelodysplastic syndromes, myeloproliferative neoplasms (MPNs), essential thrombocythemia (ET), polycythemia vera (PV), Chronic Myelogenous Leukemia (CML), myelofibrosis (MF), acute myelogenous leukemia (AML), and myelodysplastic syndromes (MDS).

76. A method of treating a patient for a telomerase-related condition, the method comprising:
administering a compound of claim 1 to the patient.

77. (canceled)

78. The method of claim 76, wherein the telomerase-related condition is a cancer.

79. The method of claim 78, wherein the cancer is a hematological malignancy.

80. The method of claim 79, wherein the cancer is selected from the group consisting of acute and chronic leukemias, lymphomas, multiple myeloma and myelodysplastic syndromes, myeloproliferative neoplasms (MPNs), essential thrombocythemia (ET), polycythemia vera (PV), Chronic Myelogenous Leukemia (CML), myelofibrosis (MF), acute myelogenous leukemia (AML), and myelodysplastic syndromes (MDS).
